(19) United States
(12) Patent Application Publication
(10) Pub. No.: US 2002/0187512 A1
(43) Pub. Date: Dec. 12, 2002

(54) Crystal structure of human interleukin-22

(76) Inventors: Ronaldo Alves Pinto Nagem, Campinas (BR); Igor Polikarpov, Sao Carlos (BR); Jean Christophe Renauld, New York, NY (US); Didier Colau, New York, NY (US); Laure Dumoutier, New York, NY (US)

Correspondence Address: Einar Stole, FULBRIGHT & JAWORSKI L.L.P., Market Square, 801 Pennsylvania Avenue, N.W., Washington, DC, 20004-2615, US

(21) Appl. No.: 10/050,552

(22) Filed: Jan. 18, 2002

Related U.S. Application Data
(60) Provisional application No. 60/317,937, filed on Sep. 10, 2001.
(60) Provisional application No. 60/333,150, filed on Nov. 27, 2001.

(51) Int. Cl. G01N 33/53; G06F 19/00; G01N 33/48; G01N 33/50; C12P 21/04

(52) U.S. Cl. 435/7.1; 435/69.52; 702/19

(57) ABSTRACT

This invention provides the three dimensional structure of human IL-22 and recombinant human IL-22 with mutations in the receptor binding regions and the dimerization interface and nucleic acid molecule encoding same. This invention also relates to methods of using pharmaceutical formulations and mimetics of the recombinant IL-22 and to methods for generating mutants based on the crystalline structure of IL-22.

This application claims priority to provisional application No. 60/317,937 filed Sep. 10, 2001 and provisional application No. 60/333,150 filed Nov. 27, 2001 both incorporated herein by reference in their entirety.

### FIELD OF THE INVENTION

The present invention relates to the fields of molecular biology, protein purification, protein crystallization, X-ray diffraction analysis, three-dimensional-structure determination, rational drug design and molecular modeling of related proteins and mutants. The present invention provides crystallization methods and crystallized human interleukin-22 (IL-22). The crystallized IL-22 is physically analyzed by X-ray diffraction techniques. The resulting X-ray diffraction patterns are of sufficiently high resolution to be useful for determining the three-dimensional structure of IL-22, molecular modeling of related proteins and mutants.

### BACKGROUND AND PRIOR ART

1. Interleukins.

The last decade has seen knowledge of the immune system and its regulation expand tremendously. One area of particular research interest has focused on the regulatory proteins and glycoproteins of the immune system. One of the best known families of these regulatory molecules is the cytokines. These are molecules which are involved in the “communication” of cells with each other. The individual members of the cytokine family have been found to be involved in a wide variety of pathological conditions, such as cancer and allergies. Whereas sometimes the cytokines are involved in the pathology of the condition, they are also known as being therapeutically useful.

Interleukins are one type of cytokines. The literature on interleukins is vast. An exemplary, but by no means exhaustive listing of the patents in this area includes U.S. Pat. No. 4,778,879 to Mertelsmann et al.; U.S. Pat. No. 4,490,289 to Stern; U.S. Pat. No. 4,518,584 to Mark et al.; and U.S. Pat. No. 4,851,512 to Miyaji et al., all of which involve interleukin-2 or “IL-2.” Additional patents have issued which relate to interleukin-1 (“IL-1”), such as U.S. Pat. No. 4,808,611 to Cosman. The disclosure of all of these patents are incorporated by reference herein. More recent patents on different interleukins include U.S. Pat. Nos. 5,694,234 (IL-13); 5,650,492 (IL-12); 5,700,664, 5,371,193 and 5,215,895 (IL-11); 5,728,377, 5,710,251, 5,328,989 (IL-10); 5,580,753, 5,587,302, 5,157,112, 5,208,218 (IL-9); 5,194,375, 4,965,195 (IL-7); 5,723,120, 5,178,856 (IL-6), and 5,017,691 (IL-4). Even a cursory review of this patent literature shows the diversity of the properties of the members of the interleukin family. One can assume that the larger cytokines family shows even more diversity. See, e.g., Aggarwal et al., ed., Human Cytokines: Handbook For Basic And Clinical Research (Blackwell Scientific Publications, 1992); Paul, ed., Fundamental Immunology (Raven Press, 1993), pp. 763-836. All cited references are incorporated by reference herein.

2. Interleukin-9.

The lymphokine IL-9, previously referred to as “P40,” is a T-cell derived molecule which was originally identified as a factor that sustained permanent antigen independent growth of T4 cell lines. See, e.g., Uyttenhove et al. (1988) Proc. Natl. Acad. Sci._USA 85: 6934; Van Snick et al. (1989) J. Exp. Med. 169: 363; Simpson et al. (1989) Eur. J. Biochem. 183: 715; all of which are incorporated herein by reference.

IL-9 activity was at first observed on T4-restricted cell lines. IL-9 does not, however, show activity on CTLs or freshly isolated T cells. See, e.g., Uyttenhove et al., supra, Schmitt et al. (1989) Eur. J Immunol. 19: 2167. Subsequent experiments demonstrated that T-cell-growth factor III (TCGF III) is identical to mast cell growth enhancing activity (MEA), a factor that potentiates the proliferative response of bone-marrow-derived mast cells to IL-3. Studies on IL-9 have shown that it also supports erythroid colony formation (Donahue et al. (1990) Blood 75(12): 2271-2275); promotes the proliferation of myeloid erythroid burst formation (Williams et al. (1990) Blood 76: 306-311); supports clonal maturation of burst-forming-unit-erythrocytes (BFU-E) of adult and fetal origin (Holbrook et al. (1991) Blood 77(10): 2129-2134); and stimulates proliferation of megakaryoblastic leukemia cells (Yang et al. (1989) Blood 74: 1880). IL-9 expression has also been implicated in Hodgkin's disease and large cell anaplastic lymphoma (Merz et al. (1990) Blood 78(8): 1311-1317). Genetic analyses of mice susceptible or resistant to the development of bronchial hyperresponsiveness have linked the IL-9 gene and its expression to bronchial hyperresponsiveness susceptibility. See, e.g., Nicolaides et al. (1997) Proc. Natl. Acad. Sci. USA 94: 13175-13180. Studies with IL-9-transgenic mice demonstrate that increased IL-9 expression produces lung mastocytosis, hypereosinophilia, bronchial hyperresponsiveness and high levels of IgE. See, e.g., Temann et al., J. Exp. Med. 188: 1307-1320, 1998; Godfraind et al (1998) J. Immunol. 160: 3989-3996; McLane et al. (1999) Am. J Resp. Cell. Mol. 19: 713-720. Genetic studies in humans have also linked IL-9 and IL-9R genes to asthma. See, e.g., Doull et al. (1996) Am. J Respir. Crit. Care Med. 153: 1280-1284; Holroyd et al. (1998) Genomics 52: 233-235, 1998. In combination, these observations strongly suggest that IL-9 plays a major role in bronchial hyperresponsiveness, asthma and allergies. See, e.g., PCT Application U.S. Pat. No. 96/12757 (Levitt, et al.), and PCT Application U.S. Pat. No. 97/21992 (Levitt, et al.), both of which are incorporated herein by reference.

IL-9 is known to affect the levels of other molecules in subjects. See e.g., Louahed et al. 1995) J. Immunol. 154: 5061-5070; Demoulin et al. (1996) Mol. Cell. Biol. 16: 4710-4716; both of which are incorporated herein by reference. It will be recognized that the molecules affected have their own functions in biological systems. For example, many of the known activities of IL-9 are mediated by activation of STAT transcription factors. See e.g., Louahed et al. (1995) J. Immunol. 154: 5061-5070; Demoulin et al. (1996) Mol. Cell. Biol. 16: 4710-4716; both of which are incorporated herein by reference. As such, there is continued interest in trying to identify molecules whose presence and/or level is affected by other molecules, such as cytokines.

3. Interleukin-22

Interleukin-22 (IL-22) is a cytokine that is induced by IL-9 in T cells and mast cells. See, e.g., Dumoutier et al. (2000) J. Immunol. 164: 1814-1819; WO 00/24758 and U.S. application Ser. No. 09/419,568, which are all incorporated herein by reference. The induction of IL-22 expression by IL-9 is rapid-within 1 hour. IL-22 is a 20 ka protein that has an N-terminal hydrophobic signal peptide and shares amino-acid-sequence homology to interleukin-10 (IL-10). In addition, IL-22 binds two receptors that are members of the class-11-cytokine-receptor family. See, e.g., Xie et al. (2000) J. Biol. Chem. 275: 31335-31339; Kotenko et al. (2001) J. Biol. Chem. 276: 2725-2732. Recent results demonstrate that the functional IL-22 receptor complex consists of two receptor chains, the CRF2-9 (IL22R) chain and the CRF2-4 (IL-10R2 or IL-1ORβ) chain. See, e.g., Xie et al. (2000) J. Biol. Chem. 275: 31335-31339; Kotenko et al. (2001) J. Biol. Chem. 276: 2725-2732. Interestingly, CRF2-4, which binds an IL-10 homodimer, is a functional component of the IL-10 signaling complex. See, e.g., Temann, et al. (1998) J. Exp. Med. 188: 1307-1320. Although this is the first example of the involvement of a class-II-cytokine-receptor in multiple distinct cytokine signaling complexes, sharing of the gamma-common chain is observed in IL-2, IL-4, IL-7, IL-9 and IL-15 receptor complexes. Other members of the class-II-receptor family include the two interferon-γ (IFN-γ) receptor chains (Rα and Rβ, the two chains of the IFN-α/β receptor, and tissue factor. In contrast, the growth hormone (GH) and prolactin receptors, for example, are members of the class-I-cytokine-receptor family.

Human and mouse IL-22 (IL-22 and mIL-22, respectively) comprise 179 amino-acid residues, including four cysteine residues, and share about 79% sequence identity. In contrast, IL-22 shares only 25% sequence identity with human IL-10 (hIL-10), and mIL-22 shares only 22% sequence identity with hIL-10. The regions of highest sequence identity are located in the C-terminal half of IL-22 and hIL-10. The fact that this region is critical for IL-10 activity, suggests that IL-22 and IL-10 share common or related biological activities.

Although IL-22 appears to play a critical role in immune function, in vivo studies in mice have demonstrated that lipopolysaccharide (LPS) induces the expression of IL-22 in numerous organs. See, e.g., Dumoutier et al. (2000) Proc. Natl. Acad. Sci. U.S.A. 97: 10144-10149. IL-22 also activates signal transducer and activator of transcription factors (STAT), specifically STAT-1 and STAT-3, in several hepatoma cell lines. The stimulation of HepG2-human-hepatoma cells up-regulates the production of acute-phase reactants such as serum amyloid A, α-1-antichymotrypsin and haptoglobin. See, e.g., Dumoutier et al. (2000) Proc. Natl. Acad. Sci. U.S.A. 97: 10144-10149. A similar induction of acute-phase reactants was observed upon injection of IL-22 into mouse liver. These findings suggest that IL-22 plays a role in the inflammatory response. Importantly, the IL-22 gene is located on human chromosome 12q near a cluster of genetic loci linked to asthma. See, e.g., Xie et al. (2000) J. Biol. Chem. 275: 31335-31339; Kotenko, et al. (2001) J. Biol. Chem. 276: 2725-2732. Thus, these findings, the induction of IL-22 by IL-9, and the association of IL-9 with inflammation and airway hyperreactivity disorders, in combination, implicate IL-22 in the etiology of asthma and allergy.

The present invention discloses a refined three-dimensional structure of IL-22 of sufficient resolution to identify the IL-22 dimerization interface and the specific amino acid residues that are involved in stabilizing the IL-22 dimer. Although both IL-10 and IL-22 form dimers, and IL-10 binds its receptor as a dimer, the present invention demonstrates that IL-22 binds the IL-22 receptor as a monomer. The present invention provides mutant IL-22 wherein the mutation or mutations destabilize the dimer. These IL-22 mutants provide IL-22 in its biologically active form and are useful as therapeutic agents. The three-dimensional structure of IL-22 of the present invention is also of sufficient resolution to allow the identification of the specific amino acids involved in binding the IL-22 receptor. In addition the present invention provides mutant IL-22 wherein the mutation(s) modify the ability of the mutant IL-22 to bind its receptor. Human IL-22 mutants with increased affinity for the IL-22 receptor are therapeutically useful agonists and antagonists. Furthermore, the present invention provides a crystal structure of sufficient quality for use in methods of rational drug design to produce therapeutically relevant molecules.

### SUMMARY OF THE INVENTION

The present invention provides methods for identifying a mammalian IL-22 mutant with modified ability to dimerize, said method comprising the steps of: (a) constructing a three-dimensional structure of IL-22 defined by the atomic coordinates shown in Table 4; (b) employing the three-dimensional structure and modeling methods to identify an amino acid involved in stabilizing a dimer of IL-22; (c) producing a mammalian IL-22 having a mutation at an amino acid identified in (b); and (d) assaying said mutant IL-22 to determine the ability of said mutant to dimerize as compared to an IL-22 control, wherein a difference in dimerization between said mutant and said control is indicative of a modified ability to dimerize. As used herein, “IL-22”, “T-cell-inducible factor (TIF)” and “IL-TIF/IL-22” each refer to a cytokine of about 20 kDa that has an N-terminal hydrophobic signal peptide amino acid sequence homology to interleukin 10 (IL-10), and is upregulated by interleukin-9 (IL-9) in T cells and mast cells. See, e.g., Dumoutier et al. (2000) J. Immunol. 164: 1814-1819. As used herein, “mammalian IL-22” or “IL-22” refers to a mammalian cytokine of about 20 kDa, which has an N-terminal hydrophobic signal peptide, amino acid sequence homology to interleukin 10 (IL-10), and is upregulated by interleukin-9 (IL-9) in T cells and mast cells. Preferably, mammalian IL-22 is from, for example, human, horses, cows, sheep, goats, cats, dogs, pigs, rats and mice. More preferably, mammalian IL-22 is human IL-22 (IL-22). In a preferred embodiment, “human IL-22” consists of the amino acid sequence of SEQ ID NO: 2.

As used herein, “ability to dimerize” refers to the ability of two IL-22 monomers to form an IL-22 dimer. Mutations that either strengthen inter-monomer contacts or weaken the inter-monomer interactions modify the ability of IL-22 to dimerize. As used herein, “stabilizing the dimer” refers to the effect of an energetically favorable mutation that strengthens inter-monomer contacts. As used herein, an amino acid is “involved” in stabilizing the dimer when the amino acid directly or indirectly contributes to the stability of the dimer—either sterically or through non-covalent bonding (i.e. van der Waals interactions, hydrogen bonding, hydrophobic interactions, etc.), and the like. As used herein, “mutation site” refers to a single amino acid of an IL-22. The IL-22 mutant, however, includes IL-22 molecules that contain mutations at one or more mutation sites. As used herein, “mutation” or “mutations” refers to a substitution of one or more amino acids; a deletion of one or more amino acids; or the addition of one or more amino acids. Preferably, a mutation of the present invention is the substitution, deletion or addition of a single amino acid at one or more mutation sites.

In a preferred embodiment, the “mutation site”, that is identified by the three-dimensional structure of IL-22 and modeling methods of the present invention, is an amino acid at a position that is at or near the dimerization interface. More preferably, the “mutation site” is one or more amino acids that are located at the dimerization interface. As used herein, “dimerization interface” refers to the contact area between the two monomers of a dimer. In a preferred embodiment, the contact area between the two monomers of a dimer include amino acid positions 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 166, 167, 168, 169, 170, 171, 172, 173, 174, 175, 176, 177, 178, 179, at least two of these amino acid positions or all of these amino acid positions of SEQ ID NO: 2. More preferably, the dimerization interface comprises amino acids at positions corresponding to positions 44, 48, 49, 57, 61, 64, 73, 75, 83, 166, 168, 175, 176, or 179 of SEQ ID NO: 2, at least two of these amino acid positions, or all of these amino acid positions.

The present invention also provides an isolated peptide selected from the group consisting of:

(a) an amino acid sequence consisting essentially of amino acids 61-71 of SEQ ID NO: 2;

(b) an amino acid sequence consisting essentially of amino acids 61-162 of SEQ ID NO: 2;

(c) an amino acid sequence consisting essentially of amino acids 61-169 of SEQ ID NO: 2;

(d) an amino acid sequence consisting essentially of amino acids 162-169 of SEQ ID NO: 2;

(e) an amino acid sequence consisting essentially of amino acids 98-104 of SEQ ID NO: 2; and

(f) an amino acid sequence consisting essentially of amino acids 98-157 of SEQ ID NO: 2.

In a preferred embodiment the amino acid sequence of the isolated peptide contains a mutation at one or more positions corresponding to position 61, 70, 71, 98-104, 154-157, 162, 166, and 169 of SEQ ID NO: 2.

Another embodiment of the present invention provides mimetics of peptides corresponding to Region 1 or Region 2, mimetics of fragments of peptides corresponding to Region 1 or Region 2 that bind an IL-22 receptor or an IL-22 receptor chain CRF2-4 and/or CRF2-9 or mutants of peptides corresponding to Region 1 or Region 2 and/or mutants thereof.

The mimetics of the present invention includes peptide-containing molecules that mimic elements of protein secondary structure. See e.g., Johnson et al., In: Biotechnology And Pharmacy (Pezzuto et al., eds.; Chapman and Hall, New York, (1993); Coligan et al. (1991) Current Protocols in Immunology 1(2): Chapter 5; both incorporated by reference herein. The underlying rationale behind the use of peptide mimetics is that the peptide backbone of proteins exists chiefly to orient amino acid side chains in such a way as to facilitate molecular interactions, such as those of antibody and antigen or receptor and ligand. A peptide mimetic permits molecular interactions similar to the natural molecule. These principles may be used, in conjunction with the principles outline above, to engineer second generation molecules having IL-22-receptor-binding properties that are improved as compared to unmodified IL-22.

As used herein, the terms “peptidomimetic” and “mimetic” is intended to include peptide analogues which serve as appropriate substitutes for peptides in interactions with, for example, receptors. The peptidomimetic must possess not only affinity, but also efficacy and substrate function. That is, a peptidomimetic exhibits functions of a peptide, without restriction of structure to amino acid constituents. Peptidomimetics, methods for their preparation and use are described in Morgan et al. (1989). See e. g., Morgan et al. In: Ann. Rep. Med. Chem. (Virick F. J., et al., eds.; Academic Press, San Diego, Calif., 1989) pp. 243-253; incorporated by reference herein. Peptidomimetics and the mutant polypeptides of the present invention may also include targeting moieties or molecules that direct the mimetics and polypeptides to specific tissues and cells. Many targeting moieties are known, and include, for example, asialoglycoproteins (See e.g., U.S. Pat. No. 5,166,320 to Wu) and other ligands which are transported into cells via receptor-mediated endocytosis.

Peptide combinatorial libraries are particularly useful for identifying the mimetics of the present invention (Simon et al. (1992) Proc. Natl. Acad. Sci. USA 89: 9367; incorporated herein by reference) and can be used to generate chemically diverse libraries of novel molecules. Once the peptide libraries are generated, they can be screened, for example, by using antibodies—polyclonal or monoclonal antibodies—that are specific to the mutant peptides corresponding to Region 1 and Region 2 of an IL-22, or mutant peptides of the present invention. These antibodies may be added to mimetics derived from the peptide libraries. After a period of incubation and a wash to remove unbound antibody, the presence of bound antibody is determined by standard ELISA assays. See, e.g., Harlow & Lane Antibodies: A Laboratory Manual (Cold Spring Harbor Laboratory; Cold Spring Harbor, N.Y., 1988) pg. 553. Wells that do not contain bound antibody indicate the presence of peptide mimetics that bind to the antibody. Methods for identifying active compounds in pools of small molecules include fractionating the pool by reverse phase HPLC or affinity selection/mass spectroscopy. See, e.g., Nedved et al. (1996) Anal. Chem. 68: 4228; Zuckermann et al. (1994) J. Med. Chem. 37: 2678; both incorporated herein by reference.

As used herein, the term “mimetic”, is not limited to peptide-based mimetics or peptidomimetics. As used herein, the term “mimetics”, refers to any molecule capable of mimicking IL-22 and the biological properties of IL-22 (i.e., binding activity and/or and inducing a receptor-mediated downstream biological effect characteristic of IL-22). The mimetics of the present invention may be a protein, peptide, or non-peptidyl based organic molecule. Accordingly, the term “mimetic” embraces any substance having IL-22-like activity, regardless of the chemical or biochemical nature thereof. The mimetics of the present invention may be a simple or complex substance produced by a living system or through chemical or biochemical synthetic techniques. A mimetic of the present invention can be a large molecule, e.g., a mutant IL-22 dimer or monomer, as described herein, or a small molecule, e.g., an organic molecule prepared de novo according to the principles of rational drug design. The mimetics of the present invention that are based on mutants of IL-22 also include any substance that structurally resembles a solvent-exposed surface epitope of IL-22 and binds an IL-22 receptor or IL-22 receptor chains. Methods of modeling, identifying and producing the mimetics of the present invention are disclosed in U.S. Pat. Nos. 5,835,382; 6,090,609; 6,242,201; 6,251,620; 6,273,598; and 6,303,287; all incorporated herein by reference.

The present invention also provides methods for identifying and producing mimetics of an IL-22 receptor or IL-22 receptor chain comprising the steps of: a) constructing a three-dimensional structure of hIL-22 defined by the atomic coordinates shown in Table 4; b) employing the three-dimensional structure and modeling methods to identify one or more surface accessible amino acids or one or more amino acids involved in receptor binding; c) producing a mimetic that binds or interacts with the IL-22 at one or more amino acids identified in (b); and c) assaying said mimetic to determine the ability of said mimetic to prevent or reduce the binding of IL-22 to an IL-22 receptor or receptor chain as compared an IL-22 control, wherein a difference in IL-22 binding between said mimetic and said control is indicative of an IL-22 receptor or IL-22 receptor chain mimetic. In a preferred embodiment, the surface accessible amino acids comprise one or more amino acids selected from the group consisting the amino acids listed in Table 5. In another embodiment, the one or more amino acids involved in IL-22 receptor or IL-22 receptor chain binding are preferably the amino acids comprising Region 1 and/or Region 2. More preferably, the one or more amino acids involved in IL-22 receptor or IL-22 receptor chain binding are selected from the group consisting of the amino acid at a position corresponding to position 61, 70, 71, 162, 166, 169, 98, 99, 100, 101, 102, 103, 104, 154, 155, 156 and 157 of SEQ ID NO: 2.

The present invention also provides a mimetic of an IL-22 receptor or IL-22 receptor chain that is produced by a method comprising the steps of: a) constructing a three-dimensional structure of hIL-22 defined by the atomic coordinates shown in Table 4; b) employing the three-dimensional structure and modeling methods to identify one or more surface accessible amino acids or one or more amino acids involved in receptor binding; c) producing a mimetic that binds or interacts with the IL-22 at one or more amino acids identified in (b); and c) assaying said mimetic to determine the ability of said mimetic to prevent or reduce the binding of IL-22 to an IL-22 receptor or receptor chain as compared o an IL-22 control, wherein a difference in IL-22 binding between said mimetic and said control is indicative of an IL-22 receptor or IL-22 receptor chain mimetic. In a preferred embodiment, the surface accessible amino acids comprise one or more amino acids selected from the group consisting the amino acids listed in Table 5. In another embodiment, the one or more amino acids involved in IL-22 receptor or IL-22 receptor chain binding are preferably the amino acids comprising Region 1 and/or Region 2. More preferably, the one or more amino acids involved in IL-22 receptor or IL-22 receptor chain binding are selected from the group consisting of the amino acid at a position corresponding to position 61, 70, 71, 162, 166, 169, 98, 99, 100, 101, 102, 103, 104, 154, 155, 156 and 157 of SEQ ID NO: 2.

The present invention also provides antibodies or fragments thereof that specifically bind to one or more epitopes in a region comprising an IL-22 dimerization interface and/or a region involved in IL-22 receptor or IL-22 receptor chain binding. In a preferred embodiment, the antibodies of the present invention are polyclonal antibodies. In a more preferred embodiment, the antibodies of the present invention are monoclonal antibodies. The antibodies of the present invention bind one or more epitopes in a region comprising an IL-22 dimerization interface and/or a region involved in IL-22 receptor or IL-22 receptor chain binding and preferably prevent or interfere with the formation of IL-22 dimers and/or prevent or interfere with the binding of IL-22 to an IL-22 receptor or IL-22 receptor chain, respectively. In a preferred embodiment, the one or more epitopes are located in a region comprising the IL-22 dimerization interface. In a more preferred embodiment, the one or more epitopes comprise one or more of the amino acids selected from the group consisting of amino acids corresponding to positions 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 166, 167, 168, 169, 170, 171, 172, 173, 174, 175, 176, 177, 178, or 179 of SEQ ID NO: 2. In a most preferred embodiment, the one or more epitopes comprise one or more of the amino acids selected from the group consisting of amino acids corresponding to positions 44, 48, 49, 57, 61, 64, 73, 75, 83, 166, 168, 175, 176, or 179 of SEQ ID NO: 2. In a preferred embodiment, the one or more epitopes are located in a region comprising the IL-22 receptor- or IL-22-receptor-chain-binding domains. In a more preferred embodiment, the one or more epitopes are located in Region 1 and/or Region 2. In a most preferred embodiment, the epitopes in Region 1 comprise one or more of the amino acids at positions corresponding to positions 61, 70, 71, 162, 166, and 169 of SEQ ID NO: 2. In a most preferred embodiment, the epitopes in Region 2 comprise one or more of the amino acids at positions corresponding to positions 98, 99, 100, 101, 102, 103, 104 154, 155, 156, or 157 of SEQ ID NO: 2.

The present invention also provides methods for identifying a mutant of a mammalian IL-22 with modified ability to bind an IL-22 receptor, said method comprising the steps of: (a) constructing a three-dimensional structure of IL-22 defined by the atomic coordinates shown in Table 4; (b) employing the three-dimensional structure and modeling methods to identify an amino acid involved in receptor binding; (c) producing any IL-22 having a mutation at an amino acid identified in (b); and (d) assaying said mutant IL-22 to determine the ability of said mutant to bind to the IL-22 receptor as compared to an IL-22 control, wherein a difference in binding between said mutant and said IL-22 control is indicative of a modified ability to bind the IL-22 receptor. As used herein, “IL-22 control” refers to an unmodified mammalian IL-22 that is identical to the mutant IL-22 prior to incorporation of the mutation.

In a preferred embodiment, the mutation site is located in an IL-22-receptor-binding site. More preferably, the IL-22-receptor-binding site is Region 1 or Region 2. As used herein, “Region 1” refers to the region of IL-22 that is formed by helix A, loop AB and helix F and binds to the IL-22-receptor chain, CRF2-4 and/or CRF2-9. As used herein, “Region 2” refers to the region of IL-22 that is formed by helix C and helix E and binds to the IL-22-receptor chain, CRF2-4. In a more preferred embodiment, the mutation site in Region 1 is selected from one or more of the amino acids at positions corresponding to positions 61, 70, 71, 162, 166, and 169 of SEQ ID NO: 2. In another embodiment, the mutation in site in Region 2 is selected from at least one of the amino acids at positions corresponding to positions 98, 99, 100, 101, 102, 103, 104 154, 155, 156, or 157 of SEQ ID NO: 2.

The present invention also provides a mutant IL-22 comprising at least one amino acid substitution in Region 1 or Region 2 or a combination thereof. More preferably, the mutant IL-22 comprises a mutation in Region 1 at one or more positions corresponding to position 44, 48, 49, 57, 61, 64, 73, 75, 83, 166, 168, 175, 176, and 179 of SEQ ID NO: 2, and/or a mutation in Region 2 at one or more positions corresponding to positions 98, 99, 100, 101, 102, 103, 104, 154, 155, 156, or 157 of SEQ ID NO: 2. The present invention also contemplates mutant IL-22 molecules that comprise Region 1, wherein the mutant IL-22 comprises a mutation at one or more positions corresponding to position 44, 48, 49, 57, 61, 64, 73, 75, 83, 166, 168, 175, 176, or 179 of SEQ ID NO: 2, and/or a mutant IL-22 molecule that comprises Region 2, wherein the mutant IL-22 comprises a mutation at one or more positions corresponding to position 98, 99, 100, 101, 102, 103, 104, 154, 155, 156, or 157 of SEQ ID NO: 2.

The present invention also provides a mutant IL-22 comprising at least one amino acid substitution at an IL-22 dimerization interface. Preferably, the dimerization interface comprises amino acids at positions corresponding to positions 41, 42, 43, 44, 45, 46, 47, 48, 49, 50, 51, 5, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 166, 167, 168, 169, 170, 171, 172, 173, 174, 175, 176, 177, 178, or 179 of SEQ ID NO: 2. More preferably, the dimerization interface comprises amino acids at positions corresponding to positions 44, 48, 49, 57, 61, 64, 73, 75, 83, 166, 168, 175, 176, or 179 of SEQ ID NO: 2.

In another embodiment, the present invention provides a mutant IL-22 comprising at least one amino acid substitution at a IL-22 dimerization interface, wherein the mutation(s) are at a position or positions that stabilize an IL-22 dimer. Preferably, the mutation or mutations are selected from one or more of the group consisting of:

(a) an amino acid at a position corresponding to position 166 or 175 of SEQ ID NO: 2;

(b) an amino acid at a position corresponding to position 57 or 176 of SEQ ID NO: 2;

(c) an amino acid at a position corresponding to position 73 or 83 of SEQ ID NO: 2;

(d) an amino acid at a position corresponding to position 44 or 64 of SEQ ID NO: 2;

(e) an amino acid at a position corresponding to position 168 or 175 of SEQ ID NO: 2;

(f) an amino acid at a position corresponding to position 75 or 176 of SEQ ID NO: 2;

(g) an amino acid at a position corresponding to position 48 or 61 of SEQ ID NO: 2;

(h) an amino acid at a position corresponding to position 44 or 166 of SEQ ID NO: 2;

(i) an amino acid at a position corresponding to position 61 or 179 of SEQ ID NO: 2; and

(j) an amino acid at a position corresponding to position 49 or 61of SEQ ID NO: 2.

More preferably, the mutation is at one or more amino acid positions corresponding to position 175 of SEQ ID NO: 2, wherein the substitution is any amino acid except arginine and lysine; position 166 of SEQ ID NO: 2, wherein the substitution is any amino acid except glutamate, aspartate, glutamine, asparagine, serine, threonine and cysteine; position 176 of SEQ ID NO: 2, wherein the substitution is any amino acid except arginine, lysine, asparagine and glutamine; position 73 of SEQ ID NO: 2, wherein the substitution is any amino acid except arginine and lysine; position 44 of SEQ ID NO: 2, wherein the substitution is any amino acid except arginine and lysine; position 64 of SEQ ID NO: 2, wherein the substitution is any amino acid except glutamate, aspartate, glutamine, asparagine, serine, threonine and cysteine; position 168 of SEQ ID NO: 2; wherein the substitution is any amino acid except glutamate, aspartate, glutamine, asparagine, serine, threonine and cysteine; position 61 of SEQ ID NO: 2, wherein the substitution is any amino acid except arginine and lysine; position 166 of SEQ ID NO: 2, wherein the substitution is any amino acid except glutamate, aspartate, glutamate, glutamine, asparagine, serine, threonine and cysteine; and position 49 of SEQ ID NO: 2, wherein the substitution is any amino acid except glutamine, asparagine, glutamate and aspartate.

The present invention also provides isolated polynucleotides that encode a mutant IL-22 comprising at least one amino acid substitution in Region 1 or Region 2. More preferably, the polynucleotides encode the mutant IL-22 that comprises a mutation in Region 1 at one or more positions corresponding to position 44, 48, 49, 57, 61, 64, 73, 75, 83, 166, 168, 175, 176, and 179 of SEQ ID NO: 2, and/or a mutation in Region 2 at one or more positions corresponding to positions 98, 99, 100, 101, 102, 103, 104, 154, 155, 156, or 157 of SEQ ID NO: 2. The present invention also contemplates polynucleotides that encode mutant IL-22 molecules that comprise Region 1, wherein the mutant IL-22 comprises at least one mutation at a position corresponding to position 44, 48, 49, 57, 61, 64, 73, 75, 83, 166, 168, 175, 176, or 179 of SEQ ID NO: 2, and/or a mutant IL-22 molecule that comprises Region 2, wherein the mutant IL-22 comprises at least one mutation at a position corresponding to position 98, 99, 100, 101, 102, 103, 104, 154, 155, 156, or 157 of SEQ ID NO: 2.

In another embodiment, the isolated polynucleotides encode mutant IL-22 comprising at least one amino acid substitution at a IL-22 dimerization interface. Preferably, the dimerization interface comprises amino acids at positions corresponding to positions 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 166, 167, 168, 169, 170, 171, 172, 173, 174, 175, 176, 177, 178, or 179 of SEQ ID NO: 2. More preferably, the dimerization interface comprises amino acids at positions corresponding to positions 44, 48, 49, 57, 61, 64, 73, 75, 83, 166, 168, 175, 176, or 179 of SEQ ID NO: 2.

In another embodiment, the present invention provides isolated polynucleotides that encode a mutant IL-22 comprising at least one amino acid substitution at an IL-22 dimerization interface, wherein the mutation or mutations are at a position or positions that stabilize an IL-22 dimer. Preferably, the mutation or mutations are selected from one of more of the group consisting of:

(a) an amino acid at a position corresponding to position 166 or 175 of SEQ ID NO: 2;

(b) an amino acid at a position corresponding to position 57 or 176 of SEQ ID NO: 2;

(c) an amino acid at a position corresponding to position 73 or 83 of SEQ ID NO: 2;

(d) an amino acid at a position corresponding to position 44 or 64 of SEQ ID NO: 2;

(e) an amino acid at a position corresponding to position 168 or 175 of SEQ ID NO: 2;

(f) an amino acid at a position corresponding to position 75 or 176 of SEQ ID NO: 2;

(g) an amino acid at a position corresponding to position 48 or 61 of SEQ ID NO: 2;

(h) an amino acid at a position corresponding to position 44 or 166 of SEQ ID NO: 2;

(i) an amino acid at a position corresponding to position 61 or 179 of SEQ ID NO: 2; and

(j) an amino acid at a position corresponding to position 49 or 61of SEQ ID NO: 2.

More preferably, the isolated polynucleotides encode an IL-22 mutant, wherein the mutation is at one or more amino acid positions corresponding to position 175 of SEQ ID NO: 2, wherein the substitution is any amino acid except arginine and lysine; position 166 of SEQ ID NO: 2, wherein the substitution is any amino acid except glutamate, aspartate, glutamine, asparagine, serine, threonine and cysteine; position 176 of SEQ ID NO: 2, wherein the substitution is any amino acid except arginine, lysine, asparagine and glutamine; position 73 of SEQ ID NO: 2, wherein the substitution is any amino acid except arginine and lysine; position 44 of SEQ ID NO: 2, wherein the substitution is any amino acid except arginine and lysine; position 64 of SEQ ID NO: 2, wherein the substitution is any amino acid except glutamate, aspartate, glutamine, asparagine, serine, threonine and cysteine; position 168 of SEQ ID NO: 2; wherein the substitution is any amino acid except glutamate, aspartate, glutamine, asparagine, serine, threonine and cysteine; position 61 of SEQ ID NO: 2, wherein the substitution is any amino acid except arginine and lysine; position 166 of SEQ ID NO: 2, wherein the substitution is any amino acid except glutamate, aspartate, glutamate, glutamine, asparagine, serine, threonine and cysteine; and position 49 of SEQ ID NO: 2, wherein the substitution is any amino acid except glutamine, asparagine, glutamate and aspartate.

The present invention also provides a mutant IL-22 comprising at least one amino acid substitution at one or more glycosylation sites, wherein the substitution prevents or reduces the glycosylation of IL-22. In a preferred embodiment, the at least one amino acid substitution is at a position selected from the group consisting of amino acid positions corresponding to position 54, 55, 56, 97, 98 or 99 of SEQ ID NO: 2. In a more preferred embodiment, the at least one amino acid substitution corresponds to position 54, 56, 97, or 99 of SEQ ID NO: 2, or a combination thereof.

In another embodiment, the mutant IL-22 comprises one or more amino acid substitutions, wherein the substitution or substitutions produce a glycosylation site at the dimerization interface. In a preferred embodiment, the glycosylation site consists of the amino acid sequence Asn-Xaa-Thr/Ser. In one embodiment, insertion of a glycosylation site increases the glycosylation of IL-22. In another embodiment, insertion of a glycosylation site increases the glycosylation of IL-22 and prevents or reduces the dimerization of IL-22 as compared to an unsubstituted IL-22.

In another embodiment, a mutant IL-22 of the present invention comprising a mutation in Region 1, Region 2, or at the dimerization interface, further comprises one or more amino acid substitutions, wherein the substitution or substitutions produce a glycosylation site at the dimerization interface. In a preferred embodiment, the glycosylation site consists of the amino acid sequence Asn-Xaa-Thr/Ser. In one embodiment, insertion of a glycosylation site increases the glycosylation of IL-22. In another embodiment, insertion of a glycosylation site increases the glycosylation of IL-22 and prevents or reduces the dimerization of IL-22 as compared to an unsubstituted IL-22.

The present invention also provides a computer system comprising: a) a memory comprising atomic coordinates shown in Table 4; and b) a processor in electrical communication with the memory; wherein the processor generates a molecular model having a three dimensional shape representative of at least a portion of a mammalian IL-22. In a preferred embodiment, the atomic coordinates shown in Table 4 are stored on a computer readable diskette.

The present invention also provides cloning and expression vectors that comprise the polynucleotides of the present invention. In another embodiment, host cells are transformed with the vectors of the present invention and are used in methods of producing the encoded mutant IL-22 that comprise culturing the host cells and isolating the mutant IL-22.

The present invention also provides pharmaceutical compositions comprising the mutant IL-22, peptides or mimetics of the present invention and a pharmaceutically acceptable carrier. As used herein, “pharmaceutically acceptable carrier” refers to any carrier, solvent, diluent, vehicle, excipient, adjuvant, additive, preservative, and the like, including any combination thereof, that is routinely used in the art.

Physiological saline solution, for example, is a preferred carrier, but other pharmaceutically acceptable carriers are also contemplated by the present invention. The primary solvent in such a carrier may be either aqueous or non-aqueous. The carrier may contain other pharmaceutically acceptable excipients for modifying or maintaining pH, osmolarity, viscosity, clarity, color, sterility, stability, rate of dissolution, and/or odor. Similarly, the carrier may contain still other pharmaceutically acceptable excipients for modifying or maintaining the stability, rate of dissolution, release, or absorption or penetration across the blood-brain barrier.

The pharmaceutical compositions of the present invention may be administered orally, topically, parenterally, rectally or by inhalation spray in dosage unit formulations that contain conventional non-toxic pharmaceutically acceptable carriers, adjuvants and vehicles. As used herein, “parenterally” refers to subcutaneous, intravenous, intramuscular, intrasternal, intrathecal, and intracerebral injection, including infusion techniques.

The pharmaceutical compositions may be administered parenterally in a sterile medium. The compositions, depending on the vehicle and concentration used, may be suspended or dissolved in the vehicle. Advantageously, adjuvants such as local anesthetics, preservatives and buffering agents can be dissolved in the vehicle. The most preferred route of parenteral administration of the pharmaceutical compositions of the present invention is subcutaneous, intramuscular, intrathecal or intracerebral. Other embodiments of the present invention encompass administration of the composition in combination with one or more agents that promote penetration of active ingredients across the blood-brain barrier, and/or slow-release of the active ingredient(s). Such excipients include those substances usually and customarily used to formulate dosages for parenteral administration in either unit dose or multi-dose form or for direct infusion into the CSF by continuous or periodic infusion from an implanted pump.

The desired or optimal dose of the compositions of the present invention may be obtained by parenteral administration that is repeated daily, more frequently, or less frequently. The compositions may also be infused continuously or periodically from an implanted pump. The frequency of dosing will depend on the pharmacokinetic parameters of the specific mutant IL-22, peptide or mimetic in the formulation and the route of administration.

In more preferred embodiments, the pharmaceutical compositions are administered as orally active formulations, inhalant spray or suppositories. The pharmaceutical compositions of the present invention may be in a form suitable for oral use, for example, as tablets, troches, lozenges, aqueous or oily suspensions, dispersible powders or granules, emulsions, hard or soft capsules, syrups or elixirs.

Active ingredient may be combined with the carrier materials in an amount to produce a single dosage form. The amount of the active ingredient will vary, depending upon the identity of the mutant, peptide, or mimetic, the host treated, and the particular mode of administration.

Regardless of the manner of administration, however, the specific dose is calculated according to approximate body weight or body surface area of the patient. Further refinement of the dosing calculations necessary to optimize dosing for each of the contemplated formulations is routinely conducted by those of ordinary skill in the art without undue experimentation, especially in view of the dosage information and assays disclosed herein.

The present invention also provides a method of treating a subject in need of IL-22, comprising the step of administering one of the pharmaceutical composition of the present invention, wherein the pharmaceutical composition is an IL-22-receptor agonist.

The present invention also provides a method of inhibiting IL-22 in a subject in need thereof, comprising the step of administering one of the pharmaceutical composition of the present invention, wherein the pharmaceutical composition inhibits the activation of an IL-22 receptor by IL-22.

### BRIEF DESCRIPTION OF THE DRAWINGS

FIG. 1. (A) Stereoview of the C_{α} trace of the dimeric structure of IL-22. (B) Schematic representation of the secondary structure of IL-22 monomer A, according to PROCHECK (Laskowski et al. (1993) J. Appl. Crystallogr. 26: 283-291; Polikarpov et al. (1997) Nucl. Instrum. Methods 405: 159-164), showing the location of the two disulfide bonds (Cys40-Cys132 and Cys89-Cys178). The figures were prepared using Molscript (Dauter, et al. (2000) Acta Cryst. D56: 232-237), Bobscript (Nagem et al. (2001) Acta Cryst. D57: 996-1002) and Raster3D (Perrakis et al. (1999) Nature Struct. Biol. 6: 458-463).

FIG. 2. Least-square fit of monomer A to monomer B. The root-mean-square deviation (rmsd) is shown as a function of residue numbers. Only main chain atoms were used in calculation.

FIG. 3. Contact surface of the IL-22 dimer, shaded according to residue hydrophobicity (A, B) and electrostatic potential (C, D). (A, C) show the interface of monomer A, whereas (B, D) show the interface of monomer B. In parts (A) and (B) the darker the stippled shading the greater the hydrophobicity. In parts (C) and (D) areas of negative, positive and neutral electrostatic potential are in medium stippling, dark stippling and light or no stippling, respectively. The figures were prepared with GRASP. See, e.g., Brünger, et al. (1998) Acta Cryst. D54: 905-921.

FIG. 4. Secondary structure diagram showing the superposition of an IL-22 monomer (in medium stippling) onto (A) a hIL-10 dimer (from helices A to D in dark stippling and from helices E′ to F′ in light stippling; helices A′ to D′, E and F were omitted) and (B) a hIFN-γ dimer (from helices A to D in light stippling and from helix E′ to F′ in black; helices A′ to D′, E and F were omitted). Superposition of an IL-22 dimer (in dark stippling and no stippling) onto (C) a hIL-10 dimer (in black and light stippling) and (D) a hIFN-γ dimer (in medium stippling and light stippling).

FIG. 5. Primary structure alignment of murine, and human IL-22 (SEQ ID NO: 3 and 2 respectively) and human IL-10 (SEQ ID NO: 1). Whenever possible, the three dimensional information was used to improve alignment. Disulfide bonds in IL-22 are marked with filled-in circles. The amino acid similarity between IL-22 and hIL-10, as calculated by the program ALSCRIPT (Nicholls et al. (1991) Struct. Funct. Genet. 11: 281-296), are boxed. Residues conserved in mIL-22 and IL-22 are boxed in the sequence of mIL-22. The loops and helices of human IL-22's secondary structure are depicted. The figure was drawn using the program ALSCRIPT (Nicholls et al. (1991) Struct. Funct. Genet. 11: 281-296).

FIG. 6. (A) Superposition of the hIFN-γ/hIFN-γRα complex (hIFN-γ light stippling and medium stippling; hIFN-γRα normal) onto IL-22 monomer (dark stippling). Superposition of (B) hIFN-γ (light stippling and darkest stippling) and (C) hIL-10 (darkest stippling and light stippling) onto IL-22 in a coil representation of the potential receptor binding site of IL-22 (medium stippling). Residues involved in direct interaction with a receptor chain are also shown.

### DETAILED DESCRIPTION OF THE INVENTION

The present invention provides methods for crystallizing human interleukin-22. The resultant crystals diffract X-rays with sufficiently high resolution to allow determination of the atomic coordinates and solve the three-dimensional structure of IL-22. The three-dimensional structure, as provided on computer-readable media described herein, is useful for rational drug design of IL-22-related mimetics, IL-22 mutants and ligands of the IL-22 receptor. Such mimetics, mutants and ligands are useful for treating and inhibiting IL-22-mediated processes or IL-22-related disorders and diseases such as asthma, inflammation and cancer.

1. IL-22 Crystallization.

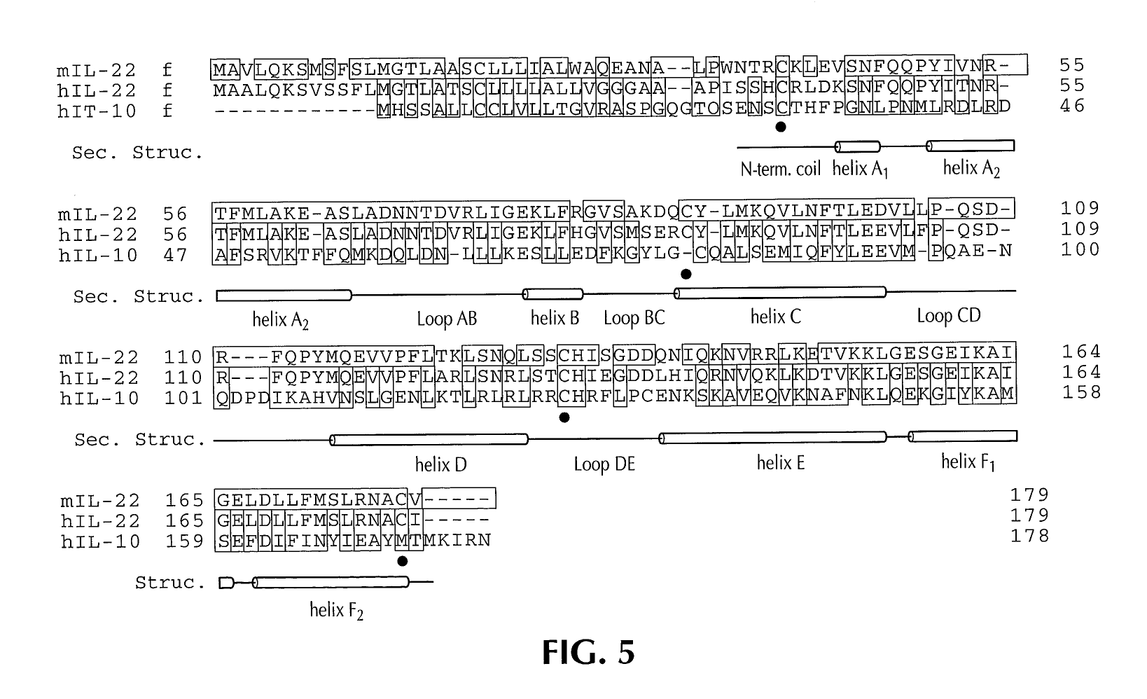

The isolation and purification of human IL-22, including polynucleotides, vectors and transformed or transfected host cells encoding IL-22, and recombinant methods of IL-22 production, are described in WO 00/24758 and U.S. application Ser. No. 09/419,568, which are both incorporated herein in their entirety. The amino acid sequences of mouse IL-22, human IL-22 and human IL-10 are presented in FIG. 5 as SEQ ID NO: 3, 2, and 1, respectively.

Recombinant IL-22 of the present invention may be produced by the following process or other recombinant protein expression methods:

a. constructing, by conventional molecular-biology methods, an expression vector comprising an operon that encodes IL-22, thereby producing a vector for the expression of IL-22;

b. transferring the expression vectors to a host cell by conventional molecular biology methods to produce a transfected or transformed host cell for the expression of IL-22; and

c. culturing the transfected or transformed cell by conventional molecular-biology methods so as to produce IL-22.

The IL-22 of the present invention may be produced using conventional molecular-biology methods. The term “conventional molecular biology methods” refers to techniques for manipulating polynucleotides that are well known to the person of ordinary skill in the art of molecular biology. Examples of such well known techniques can be found in Sambrook et al. Molecular Cloning: A Laboratory Manual, 3rd Edition (Cold Spring Harbor, N.Y.; 2001). Examples of conventional molecular biology techniques include, but are not limited to, in vitro ligation, restriction-endonuclease digestion, PCR, cellular transformation and transfection, hybridization, electrophoresis, DNA sequencing, and the like.

Specifically, the general methods for construction of the vector of the invention, transfection of cells to produce the host cell of the invention, and culturing of cells to produce the IL-22 of the present invention are all conventional molecular biology methods. Likewise, once produced, the IL-22 of the present invention may be purified by standard procedures of the art, including ammonium-sulfate precipitation, affinity-column chromatography, gel electrophoresis and the like.

The present invention also provides polynucleotide vectors for the replication, manipulation and expression of the isolated polynucleotides of the present invention. Preferably, the vectors allow expression of the isolated polynucleotides of the present invention in either prokaryotic or eukaryotic cells. Prokaryotic cells are selected from bacterial cells, e.g. Escherichia coli, and eukaryotic cells are selected from insect, fungal, e.g. Saccharomyces, Pichia pastoris, and mammalian cells, e.g. Chinese hamster ovary (CHO) and human. The vectors of the present invention may contain regulatory elements that allow inducible or constitutive expression of the operably-linked polynucleotide, confer antibiotic resistance, improve secretion, purification and detection, e.g. His and antigen tags, and the like.

The host cells may be either a bacterial cell such as Escherichia coli, or a eukaryotic cell. Mammalian cells such as Chinese hamster ovary cells, may also be used. Notably, the choice of expression vector is dependent upon the choice of host cell, and may be selected so as to have the desired expression and regulatory characteristics in the selected host cell.

The first prerequisite for solving the three-dimensional structure of a protein by X-ray crystallography is a well-ordered crystal that will strongly diffract X-rays. X-rays are directed onto a regular, repeating array of identical molecules so that the X-rays are diffracted from it in a pattern from which the structure of an individual molecule can be retrieved. Different crystal forms can be more or less well-ordered and hence give diffraction patterns of different quality. As a general rule, the more closely the protein molecules pack, and consequently the less water the crystals contain, the better is the diffraction pattern because the molecules are better ordered in the crystal. Well-ordered crystals of globular protein molecules are large, spherical, or ellipsoidal objects with irregular surfaces, and crystals thereof contain large holes or channels that are formed between the individual molecules. These channels, which usually occupy more than half the volume of the crystal, are filled with disordered solvent molecules. The protein molecules are in contact with each other at only a few small regions. This is one reason why structures of proteins determined by X-ray crystallography are generally the same as those for the proteins in solution.

The formation of crystals is dependent on a number of different parameters, including pH, temperature, protein, concentration, the nature of the solvent and precipitant, as well as the presence of added ions or ligands. Crystallization experiments may be needed to screen all these parameters for the few combinations that might give crystals suitable for X-ray diffraction analysis. Crystallization robots can automate and speed up the work of reproducibly setting up large number of crystallization experiments.

A pure and homogeneous protein sample is important for successful crystallization. Proteins obtained from cloned genes in efficient expression vectors can quickly be purified to homogeneity in large quantities in a few purification steps. A protein to be crystallized is preferably at least 93-99% pure, according to standard criteria of homogeneity. Crystals form when molecules are precipitated very slowly from supersaturated solutions. The most frequently used procedure for making protein crystals is the hanging-drop method, in which a drop of protein solution is brought very gradually to supersaturation by loss of water from the droplet to the larger reservoir that contains salt or polyethylene glycol solution.

In general, IL-22 is purified as described in WO 00/24758 and U.S. application Ser. No. 09/419,568, which are both incorporated herein by reference. The resulting IL-22 is in sufficiently pure and concentrated for crystallization. The purified IL-22 preferably runs as a single band under reducing or nonreducing polyacrylamide gel electrophoresis (PAGE) (nonreducing conditions are used to evaluate the presence of disulfide bonds). Purified IL-22 is preferably crystallized using the hanging drop method under varying conditions of at least one of the following: pH, buffer type, buffer concentration, salt type, polymer type, polymer concentration, other precipitating agents and concentration of purified and cleaved IL-22. See, e.g., the methods provided in a commercial kit, such as CRYSTAL SCREEN (Hampton Research, Riverside, Calif.); Taylor et al. (1992) J. Mol. Biol. 226:1287-1290; Takimoto et al. (1992), infra.

Crystallization conditions suitable to produce diffraction-quality crystals may be selected from a buffer containing, for example: between 1 and 100 mg/ml IL-22 in 10-200 mM buffer (pH 4-9) (e.g., phosphate, cacodylate, acetates, imidazole, Tris HCl, sodium HEPES); and optionally a salt (e.g., calcium chloride, sodium citrate, magnesium chloride, ammonium acetate, ammonium sulfate, potassium phosphate, magnesium acetate, zinc acetate; calcium acetate); and optionally 0-50% of a polymer (e.g., polyethylene glycol (PEG); average molecular weight 200-10,000); and optionally other precipitating agents (salts: potassium or sodium tartrate, ammonium sulfate, sodium acetate, lithium sulfate, sodium formate, sodium citrate, magnesium formate, sodium phosphate, potassium sulfate, ammonium phosphate); and optionally organics e.g., 2-propanol; non-volatile: 2-methyl-2,4-pentanediol).

The above mixtures are used and screened by varying at least one of pH, buffer type; buffer concentration, precipitating salt type or concentration, PEG type, PEG concentration, and protein concentration. Crystals ranging in size from 0.2-0.7 mm are formed in 1-7 days. From one to ten crystals are observed in one drop and crystal forms, such as, but not limited to, bipyramidal, rhomboid, and cubic, are suitable. Initial X-ray analyses indicate that such crystals diffract at moderately high to high resolution. When fewer crystals are produced in a drop, they can be much larger size, e.g., 0.4-0.9 mm. These crystals diffract X-rays to at least 3.5 Å resolution, such as 1.5-3.5 Å, or any range of value therein, such as 1.5, 1.6, 1.7, 1.8, 1.9, 2.0, 2.1, 2.2, 2.3, 2.4, 2.5, 2.6, 2.7, 2.8, 2.9, or 3.0, with 3.0 Å or less being preferred.

2. X-ray Diffraction and Structure Determination.

The X-ray diffraction patterns of the invention are of sufficiently high resolution for three-dimensional modeling of IL-22 and IL-22-related molecules, such as IL-22-receptor ligands and IL-22-receptor-chain mimics. Preferably the resolution is in the range of 1.5 to 3.5 Å, more preferably 1.5-3.0 Å and most preferably about 1.9 Å.

X-rays may be produced by high-voltage tubes in which an anode emits X-rays of a specific wavelength upon bombardment by accelerating electrons. More powerful X-ray beams can be produced in synchrotron storage rings where electrons (or positrons) travel near the speed of light. These particles emit very strong radiation at all wavelengths—from short gamma rays to visible light. When used as an X-ray source, however, only X-ray radiation is channeled from the storage ring. Polychromatic X-ray beams are produced by having a broad window that allows through X-ray radiation with wavelengths of 0.2-3.5 Å.

In diffraction experiments, a narrow and parallel beam of X-rays is taken out from the X-ray source and directed onto the crystal to produce diffracted beams. The incident primary beam causes damage to both protein and solvent molecules. The crystal is, therefore, usually cooled to prolong its lifetime (e.g., −220 to −50° C.). The primary beam must strike the crystal from many different directions to produce all possible diffraction spots, and so the crystal is rotated in the beam during the experiment.

The diffracted spots are recorded either on a film, the classical method, or by an electronic detector. The exposed film is measured and digitized by a scanning device, whereas electronic detectors feed the signals they detect directly in a digitized form into a computer. Electronic area detectors significantly reduce the time required for data collection.

When the primary beam from an X-ray source strikes the crystal, some of the X-rays interact with the electrons on each atom and cause them to oscillate. The oscillating electrons serve as a new source of X-rays, which are emitted in almost all directions, referred to as scattering. When atoms and their electrons are arranged in a regular three-dimensional array, the X-rays emitted from the oscillating electrons interfere with one another. In most cases, these X-rays, colliding from different directions, cancel each other out; those from certain directions, however, will add together to produce diffracted beams of radiation that can be recorded as a pattern on a photographic plate or detector.

The diffraction pattern obtained in an X-ray experiment is related to the crystal that caused the diffraction. X-rays that are reflected from adjacent planes travel different distances, and diffraction only occurs when the difference in distance is equal to the wavelength of the X-ray beam. This distance is dependent on the reflection angle, which is equal to the angle between the primary beam and the planes.

The relationship between the reflection angle (θ), the distance between the planes (d), and the wavelength (λ) is given by Bragg's law: 2d sinθ.λ. This relationship can be used to determine the size of the unit cell in the crystal. Briefly, the position on the film of the diffraction data relates each spot to a specific set of planes through the crystal. By using Bragg's law, these positions can be used to determine the size of the unit cell.

Each atom in a crystal scatters X-rays in all directions, and only those that positively interfere with one another, according to Bragg's law, give rise to diffracted beams that can be recorded as a distinct diffraction spot above background. Each diffraction spot is the result of interference of all X-rays with the same diffraction angle emerging from all atoms. For the protein crystal of myoglobin, for example, each of the about 20,000 diffracted beams that have been measured contain scattered X-rays from each of the around 1500 atoms in the molecule. To extract information about individual atoms from such a system requires considerable computation. The mathematical tool that is used to handle such problems is called the Fourier transform.

Each diffracted beam, which is recorded as a spot on the film, is defined by three properties: the amplitude, which we can measure from the intensity of the spot; the wavelength, which is set by the X-ray source; and the phase, which is lost in X-ray experiments. All three properties are needed for all of the diffracted beams, in order to determine the position of the atoms giving rise to the diffracted beams.

For larger molecules, protein crystallographers have determined the phases in many cases using a method called multiple isomorphous replacement (MIR) (including heavy metal scattering), which requires the introduction of new X-ray scatterers into the unit cell of the crystal. These additions are usually heavy atoms that contribute significantly to the diffraction pattern. Since such heavy metals contain many more electrons than the carbon, hydrogen, oxygen, nitrogen and sulfur atoms of the protein, they scatter X-rays more strongly. All diffracted beams would therefore increase in intensity after heavy-metal substitution if all interference were positive. In fact, however, some interference is negative; consequently, following heavy-metal substitution, some spots measurably increase in intensity, others decrease, and many show no detectable difference. Isomorphous replacement is usually done by diffusing different heavy-metal complexes into the channels of the preformed protein crystals. The protein molecules expose side chains (such as SH groups) into these solvent channels that are able to bind heavy metals. It is also possible to replace endogenous light metals in metalloproteins with heavier ones, e.g., zinc by mercury, or calcium by samarium.

Phase differences between diffracted spots can be determined from intensity changes following heavy-metal substitution. First, the intensity differences are used to deduce the positions of the heavy atoms in the crystal unit cell. Fourier summations of these intensity differences give maps of the vectors between the heavy atoms—the so-called Patterson maps. From these vector maps the atomic arrangement of the heavy atoms is deduced. From the positions of the heavy metals in the unit cell, one can calculate the amplitudes and phases of their contribution to the diffracted beams of protein crystals containing heavy metals.

This knowledge is then used to find the phase of the contribution from the protein in the absence of the heavy-metal atoms. As both the phase and amplitude of the heavy metals, the amplitude of the protein alone, and the amplitude of the protein plus heavy metals is known, one phase and three amplitudes are known. From this, the interference of the X-rays scattered by the heavy metals and protein can be calculated to see if it is constructive or destructive. The extent of positive or negative interference, with knowledge of the phase of the heavy metal, gives an estimate of the phase of the protein. Because two different phase angles are determined and are equally good solutions, a second heavy-metal complex can be used which also gives two possible phase angles. Only one of these will have the same value as one of the two previous phase angles; it therefore represents the correct phase angle. In practice, more than two different heavy-metal complexes are usually made in order to give a reasonably good phase determination for all reflections. Notably, each individual phase estimate contains experimental errors arising from errors in the measured amplitudes, and for many reflections, the intensity differences are too small to measure after one particular isomorphous replacement.

The amplitudes and the phases of the diffraction data from the protein crystals are used to calculate an electron-density map of the repeating unit of the crystal. This map then has to be interpreted as a polypeptide chain with a particular amino acid sequence. The interpretation of the electron-density map is complicated by several limitations of the data. First of all, the map itself contains errors, mainly due to errors in the phase angles. In addition, the quality of the map depends on the resolution of the diffraction data, which depends on crystal quality and degree of order. This directly influences the image that can be produced. The resolution is measured in Å ngstrom units—as this number decreases, the resolution increases and consequently, the amount of molecular detail observed also increases.

Building the initial model begins by determining how the polypeptide chain weaves its way through the electron-density map. The resulting chain trace constitutes a hypothesis, by which one tries to match the density of the side chains to the known sequence of the polypeptide. When a reasonable chain trace has finally been obtained, an initial model is built to give the best fit of the atoms to the electron density. Computer graphics are used both for chain tracing and for model building to present the data and manipulate the models.

The initial model will contain some errors. Provided the protein crystals diffract to a sufficiently high resolution—better than 3.5 Å—most or substantially all of the errors can be removed by crystallographic refinement of the model using computer algorithms. In this process, the model is modified to minimize the difference between the experimentally observed diffraction amplitudes and those calculated for a hypothetical crystal containing the model, instead of the real molecule. This difference is expressed as an R factor (residual disagreement), which is 0.0 for exact agreement and about 0.59 for total disagreement.

In general, the R factor is preferably between 0.15 and 0.35, and more preferably between about 0.24-0.28 for a well-determined protein structure. The residual difference is a consequence of errors and imperfections in the data. These derive from various sources, including slight variations in the conformation of the protein molecules, as well as inaccurate corrections both for the presence of solvent and for differences in the orientation of the microcrystals from which the crystal is built. This means that the final model represents an average of molecules that are slightly different both in conformation and orientation. In refined structures at high resolution, there are usually no major errors in the orientation of individual residues, and the estimated errors in atomic positions are usually around 0.1-0.2 Å, provided the amino acid sequence is known. Hydrogen bonds, both within the protein and to bound ligands, can be identified with a high degree of confidence.

Most X-ray structures are determined to a resolution between 1.7 Å and 3.5 Å. Electron-density maps with this resolution range are preferably interpreted by fitting the known amino acid sequences into regions of electron density in which individual atoms are not resolved.

The IL-22 crystals are analyzed using a suitable X-ray source and diffraction patterns are obtained. Crystals are preferably stable for at least 10 hrs in the X-ray beam. Frozen crystals (e.g., −220 to −50° C.) could also be used for longer X-ray exposures (e.g., 24-72 hrs), the crystals being relatively more stable to the X-rays in the frozen state. To collect the maximum number of useful reflections, multiple frames are optionally collected as the crystal is rotated in the X-ray beam, e.g., for 24-72 hrs. Larger crystals (>0.2 mm) are preferred, to increase the resolution of the X-ray diffraction. Alternatively, crystals may be analyzed using a synchrotron high-energy X-ray source. Using frozen crystals, X-ray diffraction data is collected on crystals that diffract to a relatively high resolution of 3.5 Å or less, sufficient to solve the three-dimensional structure of IL-22 in considerable detail, as presented herein. Specifically, crystals were soaked in different cryosoaking solutions, mounted in a rayon loop and finally flash-cooled to 80 K in a cold nitrogen stream. Data collection was performed at the Protein Crystallography beamline (LNLS, Campinas, Brazil; Polikarpov et al. (1997) J. Synchrotron Rad. 5: 72-76; Polikarpov et al. (1997) Nucl. Instrum. Methods A 405: 159-164) and at the X4A beamline (NSLS, Upton, USA), using a MAR345 image plate and a Quantum-4 CCD detector.

The heavy metal derivatives are used to determine the phase, e.g., by the isomorphous replacement method. Heavy atom isomorphous derivatives of IL-22 are used for X-ray crystallography, where the structure is solved using one or several derivatives, which, (when combined) improves the overall figure of merit. Derivatives are identified through Patterson maps and/or cross-phase difference Fourier maps, e.g., using commercially-available software, including the CCP4 package (SERC Collaborative Computing Project No. 4, Daresbury Laboratory, UK, 1979); SIRAS; SHARP [35]; DREAR [31] and SnB 2.1 [32]; and SOLOMON [36]. The program MLPHARE (Wolf et al., eds., Isomorphous Replacement and Anomalous Scattering: Proceedings of CCP4 Study Weekend, pp. 80-86, SERC Daresbury Lab., UK (1991)) is optionally used for refinement of the heavy atom parameters and the phases derived from them by comparing at least one of completeness (%), resolution (in Å), R^{r} (%), heavy atom concentration (mM), soaking time, heavy atom sites, phasing power (acentric, centric). Addition of heavy atom derivatives produce an MIR map with recognizable features.

Once the initial phases are calculated to 3.2 Å, they may be improved and extended to a higher resolution of 2.8 Å, using solvent flattening, histogram matching and/or Sayre's equation in the program DM. See e.g., Cowtan et al. (1993) Acta Crystallogr. 49: 148-157. The skeletonization of the DM procedure is optionally used to improve connectivity in the bulk of the protein envelope. Both the MIR and density modified maps are optionally used in subsequent stages, to provide sufficient resolution and/or modeling of surface structures.

Skeletonized representations of electron density maps are then computed. These maps are automatically or manually edited using suitable software, e.g., the graphics package FRODO (Jones et al. (1991), infra) to give a continuous C_{α} trace. The IL-22 sequence is then aligned to the trace. Initially pieces of idealized polypeptide backbone were placed into regions of the electron density map with obvious secondary structures (e.g., α-helix, β-sheet). After a polyalanine model was constructed for the protein, amino acid side-chains were added where density was present in the maps. The amino acid sequence of IL-22 was then examined for regions with distinct side-chain patterns (e.g., three consecutive aromatic rings). When a pattern in the sequence was found to match an area of the map, the correct side-chains were built onto the existing model. Eventually fragments containing recognizable sequence motifs were connected into a single chain, completing the tracing of the amino acid sequence into the maps. Cycles of simulated annealing against these data may be refined using the program X-PLOR for molecular dynamics for R-factor refinement. See e.g., Brunger et al. (1987) J. Mol. Biol. 203: 803-816. This refinement was followed by manual rebuilding with FRODO using experimental and 2Fₒ-F_{c} maps. The model may be further refined using a least- squares refinement program, such as TNT. See e.g., Tronrud et al (1987) Acta Crystallogr. A 43: 489-501. One or more of the above modeling steps may performed to provide a molecular 3-D model of IL-22. It is preferred that the IL-22 model has no residues in disallowed regions of the Ramachandran plot, and gives a positive 3D-1D profile (Luthy et al. (1992) Nature 356: 83-85; Kraulis (1991), infra), suggesting that all the residues are in acceptable environments.

Alternatively, a program such as ARP (Lamzin et al. (1993) Acta Cryst. D49: 129-147) may be used to add crystallographic waters and as a tool to check for bad areas in the model. The programs PROCHECK (Lackowski et al. (1993) J. Appl. Cryst. 26: 283-291), WHATIF (Vriend (1990) J. Mol. Graph. 8:52-56), PROFILE 3D (Luthy et al. (1992) Nature 356: 83-85), and ERRAT (Colovos et al. (1993) Protein Science 2: 1511-1519), as well as the geometrical analysis generated by X-PLOR were used to check the structure for errors. Anisotropic scaling between F_{obs} and F_{calc} may be applied after careful assessment of the quality and completeness of the data. The program DSSP may be used to assign the secondary structure elements (Kabsch et al. (1983) Biopolymers 22: 2577-2637). A program such as SUPPOS (from the BIOMOL crystallographic computing package) can be used for some or all of the least-squares superpositions of various models and parts of models. The program ALIGN (Cohen (1986) J. Mol. Biol. 190: 593-604) may be used to superimpose N- and C-terminal domains of IL-22. Solvent accessible surfaces and electrostatic potentials can be calculated using such programs as GRASP (Nicholls et al. (1991), infra).

3. Rational Drug Design and Molecular Modeling of IL-22 and IL-22-Related Proteins.

Three-dimensional modeling is performed using the diffraction coordinates from the X-ray diffraction patterns and atomic coordinates of the present invention. The coordinates are entered into one or more computer programs for molecular modeling, as known in the art. Such molecular modeling can utilize known X-ray diffraction molecular modeling algorithms or molecular modeling software to generate atomic coordinates corresponding to the three-dimensional structure of at least one IL-22 or a fragment thereof.

The entry of the coordinates of the X-ray diffraction patterns and the amino acid sequence into such programs results in the calculation of the most probable secondary, tertiary and quaternary structures of the protein, including overall atomic coordinates of a IL-22 or a fragment thereof. These structures are combined and refined by additional calculations using such programs to determine the probable or actual three-dimensional structure of the IL-22, including potential or actual active or binding sites of the protein.

Such molecular modeling and related programs useful for rational drug design of ligands or mimetics, are contemplated by the present invention. The drug design uses computer modeling programs which calculate how different molecules interact with the various sites of the IL-22, how IL-22 monomers interact with other IL-22 monomers, how IL-22 interacts with IL-22-receptor mimetics and IL-22 receptors. This procedure determines potential ligands or mimetics of a IL-22. The actual IL-22-ligand complexes or mimetics are crystallized and analyzed using X-ray diffraction. The diffraction pattern coordinates are similarly used to calculate the three-dimensional interaction of a ligand and the IL-22.

An amino acid sequence of a IL-22 protein and/or X-ray diffraction data, useful for computer molecular modeling of IL-22, can be “provided” in a variety of mediums to facilitate use thereof. As used herein, provided refers to a manufacture, which contains, for example, a IL-22 amino acid sequence and/or atomic coordinate/X-ray diffraction data of the present invention, e.g., an amino acid sequence of SEQ ID NO: 2, a representative fragment thereof, or an amino acid sequence having at least 80-100% overall identity to an amino acid sequence of SEQ ID NO: 2. Such a method provides the amino acid sequence and/or X-ray diffraction data in a form which allows a skilled artisan to analyze and molecular model the three-dimensional structure of a IL-22-related protein, including one or more subdomains thereof.

In one application of this embodiment, IL-22, or at least one subdomain thereof, amino acid sequence and/or X-ray diffraction data of the present invention is recorded on computer readable medium. As used herein, “computer readable medium” refers to any medium which can be read and accessed directly by a computer. Such media include, but are not limited to: magnetic storage media, such as floppy discs, hard disc storage medium, and magnetic tape; optical storage media such as optical discs or CD-ROM; electrical storage media such as RAM and ROM; and hybrids of these categories such as magnetic/optical storage media. A skilled artisan can readily appreciate how any of the presently known computer readable mediums can be used to create a manufacture comprising computer readable medium having recorded thereon an amino acid sequence and/or X-ray diffraction data of the present invention.

As used herein, “recorded” refers to a process for storing information on computer readable medium. A skilled artisan can readily adopt any known method for recording information on computer readable medium to generate manufactures comprising an amino acid sequence and/or atomic coordinate/X-ray diffraction data information of the present invention. A variety of data storage structures are available to a skilled artisan for creating a computer readable medium having recorded thereon an amino acid sequence and/or atomic coordinate/X-ray diffraction data of the present invention. The choice of the data storage structure will generally be based on the means chosen to access the stored information. In addition, a variety of data processor programs and formats can be used to store the sequence and X-ray data information of the present invention on computer readable medium. The sequence information can be represented in a word processing text file, formatted in commercially-available software such as WordPerfect and MICROSOFT Word, or represented in the form of an ASCII file, stored in a database application, such as DB2, Sybase, Oracle, or the like. A skilled artisan can readily adapt any number of dataprocessor structuring formats (e.g. text file or database) in order to obtain computer readable medium having recorded thereon the information of the present invention.

By providing computer readable medium having stored thereon an IL-22 or related sequence protein and/or atomic coordinates based on X-ray diffraction data, a skilled artisan can routinely access the sequence and atomic coordinate or X-ray diffraction data to model a IL-22 or related protein, a subdomain thereof, mimetic, or a ligand thereof. Computer algorithms are publicly and commercially available which allow a skilled artisan to access this data provided in a computer readable medium and analyze it for molecular modeling and/or RDD. See, e.g., Biotechnology Software Directory, MaryAnn Liebert Publ., New York (1995). A variety of comparing means can be used to compare a target sequence or target motif with the data storage means to identify structural motifs or electron density maps derived in part from the atomic coordinate/X-ray diffraction data. A skilled artisan can readily recognize that any one of the publicly available computer modeling programs can be used as the search means for the computer-based systems of the present invention.

Several approaches can be taken for the use of the crystal structure of a IL-22 in the rational design of a relevant activity similar to that of the unmutated IL-22. A computer-assisted, manual examination of an IL-22-receptor-binding site structure is optionally done. Software such as GRID (Goodford (1985) J. Med. Chem. 28: 849-857), a program that determines probable interaction sites between probes with various functional group characteristics and the protein surface, is used to analyze the surface sites to determine structures of similar inhibiting proteins or compounds. The GRID calculations, with suitable inhibiting groups on molecules (e.g., protonated primary amines) as the probe, are used to identify potential hotspots around accessible positions at suitable energy contour levels.

A therapeutic IL-22 or related protein of the present invention can be, but is not limited to, IL-22-receptor ligands that bind to IL-22 receptors as either agonists or antagonists; IL-22-receptor-chain mimetics or antibodies that bind to endogenous IL-22 and impairs the binding of IL-22 to endogenous receptors. The program DOCK (Kuntz et al. (1982) J. Mol. Biol. 161: 269-288) may be used to analyze receptor binding sites, dimerization interfaces and/or ligand binding site and suggest ligands or amino acid residues with complementary steric properties. Several methodologies for searching three-dimensional databases to test pharmacophore hypotheses and select compounds for screening are available. These include the program CAVEAT (Bacon et al. (1992) J. Mol. Biol. 225: 849-858), which uses databases of cyclic compounds which can act as “spacers” to connect any number of chemical fragments already positioned in the active site. This allows one skilled in the art to quickly generate hundreds of possible ways to connect the fragments already known or suspected to be necessary for tight binding. The program LUDI (Bohm et al. (1992) J. Comput.-Aid. Mol. Des. 6: 61-78) can determine a list of interactions sites into which to place both hydrogen bonding and hydrophobic fragments. LUDI then uses a library of approximately 600 linkers to connect up to four different interaction sites into fragments. Then smaller “bridging” groups such as —CH2— and —COO— are used to connect these fragments. For example, for the enzyme DHFR, the placements of key functional groups in the well-known inhibitor methotrexate were reproduced by LUDI. See also, Rothstein et al. (1992) J. Med. Chem. 36: 1700-1710.

Once IL-22-receptor ligands or mimetics are identified, crystallographic studies of, the IL-22 ligand and its receptor complex and the IL-22-receptor mimetic and its IL-22 complex may be performed to confnm and refine the ligand or mimetic properties. Direct measurements of receptor binding or complex formation provide further confirmation that the modeled mimetic and ligands are high affinity IL-22 agonists, antagonists or inhibitors. Any suitable assay for receptor binding or complex formation may be used. The atomic coordinates of IL-22 are useful in the generation of molecular models of related proteins and of IL-22-receptor mimetics and ligands. Utilizing CLUSTAL (a multiple sequence alignment program in PC-Gene) and the Homology module (a structure-based homology modeling program in InsightIl on a Silicon Graphics Incorporated workstation), molecular models (and the corresponding three-dimensional coordinates files) of numerous mimetics and ligands are generated. With these files, mutants and mimetics of the present invention are mapped and new ones designed. The results described herein demonstrate that tight-binding mimetics and ligands of an IL-22 receptor, or related protein, based on the crystal structure of IL-22, are provided by the present invention.

The term “antibody” as used herein, unless indicated otherwise, is used broadly to refer to both antibody molecules and a variety of antibody-derived molecules. Such antibody-derived molecules comprise at least one variable region (either a heavy chain of light chain variable region) and include molecules such as Fab fragments, F(ab)₂ fragments, single chain (sc) antibodies, diabodies, triabodies, tetrabodies, individual antibody light chains, individual antibody heavy chains, chimeric fusions between antibody chains and other molecules, and the like. As used herein “antigen-binding fragment” or “antigen-binding domain” or “Fab fragment” refer to the about 45 kDa fragment obtained by papain digestion of an immunoglobulin molecule and consist of one intact light chain linked by disulfide bond to the n-terminal portion of the contiguous heavy chain. As used herein, “F(ab)₂ fragment refers to the about 90 kDa protein produced by pepsin hydrolysis of an immunoglobulin molecule. It consists of the N-terminal pepsin cleavage product and contains both antigen binding fragments of a divalent immunoglobulin, such as IgD, IgE, and IgG. Neither the “antigen-binding fragment” nor “F(ab)₂ fragment” contain the about 50 kDa F_{c} fragment produced by papain digestion of an immunoglobulin molecule that contains the c-terminal halves of the immunoglobulin heavy chains, which are linked by two disulfide bonds, and contain sites necessary for compliment fixation.

As used herein, the term “humanized” antibody refers to a molecule that has its CDRs—complementarily determining regions—derived from a non-human-species immunoglobulin and the remainder of the antibody molecule derived mainly from a human immunoglobulin. As used herein “immunoglobulin” refers to any member of a group of glycoproteins occurring in higher mammals that are major components of the immune system. As used herein, “immunoglobulins” comprise four polypeptide chains-2 identical light chains and two identical heavy chains that are linked together by disulfide bonds. An immunoglobulin consists of the antigen binding domains, which are each comprised of the light chains and the end-terminal portion of the heavy chain, and the F_{c} region, which is necessary for a variety of functions, such as compliment fixation. There are five classes of immunoglobulins wherein the primary structure of the heavy chain, in the F_{c} region, determines the immunoglobulin class. Specifically, the alpha, delta, epsilon, gamma, and mu chains correspond to IgA, IgD, IgE, IgG and IgM, respectively. As used herein “immunoglobulin” includes all subclasses of alpha, delta, epsilon, gamma, and mu and also refers to any natural (e.g., IgA and IgM) or synthetic multimers of the four-chain immunoglobulin structure.

As used herein, “Fv or Fv fragment” refers to the N-terminal part of the Fab fragment of an immunoglobulin molecule, consisting of the variable region of the heavy chain and the variable region of the light chain. As used herein, “scFv” refers to a polypeptide comprising the heavy chain variable region and light chain variable region of a parent immunoglobulin, wherein the heavy chain variable region and the light chain variable region are linked by a peptide linker. As used herein, “diabody” refers to an scFv dimer. As used herein, “triabody” refers to an scFv trimer, and “tetrabody” refers to an scFV tetramer. As used herein, “heavy chain” refers to the heavier of the two types of polypeptide chain in immunoglobulin molecules that contain the antigenic determinants that differentiate the various Ig classes, e.g., IgA, IgD, IgE, IgG, IgM, and the domains necessary for compliment fixation placental transfer, mucosal secretion, and interaction with F_{c} receptor. As used herein, “heavy chain variable region” refers to the amino-terminal domain of heavy chain that is involved in antigen binding and combines with the light chain variable region to form the antigen binding domain of the immunoglobulin. As used herein, “light chain” refers to the shorter of the two types of polypeptide chain in an Ig molecule of any class. Light chains comprise variable and constant regions. As used herein, “light chain variable region” refers to the amino-terminal domain of the light chain and is involved in antigen binding and combines with the heavy chain to form the antigen binding region.

The term “variable region” as used herein in reference to immunoglobulin molecules has the ordinary meaning given to the term by the person of ordinary skill in the art of immunology. Both antibody heavy chains and antibody light chains may be divided into a “variable region” and a “constant region.” The point of division between a variable region and a constant region may readily be determined by the person of ordinary skill in the art by reference to standard texts describing antibody structure, e.g. Kabat et al. (1991) Sequences of Proteins of Immunological Interest. 5th Edition. U.S. Department of Health and Human Services, U.S. Government Printing Office.

The recombinant production of immunoglobulin molecules, including humanized antibodies are described in U.S. Pat. No. 4,816,397 (Boss et al.), U.S. Pat. No. 4,816,567 (Cabilly et al.) U.K. patent GB 2,188,638 (Winter et al.), and U.K. patent GB 2,209,757; all of which are incorporated herein by reference. Techniques for the recombinant expression of immunoglobulins, including humanized immunoglobulins, can also be found, among other places in Goeddel et al (1991) Gene Expression Technology, Methods in Enzymology Vol. 185, and Borreback (1992) Antibody Engineering, W. H. Freeman, all of which are incorporated herein by reference. Additional information concerning the generation, design and expression of recombinant antibodies can be found in Mayforth (1993) Designing Antibodies, Academic Press, San Diego and Harlow (1988) Antibodies—A laboratory manual. First Edition. Cold Spring Harbor Laboratory, all of which are incorporated herein by reference.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### EXAMPLES

### Example 1

Protein Expression and Purification.

A cDNA encoding IL-22 sequence lacking the signal peptide was subdloned into the E. coli expression vector pET2a, generating pEThTIF. The recombinant protein expressed from this vector contains a methionine at the N-terminus, followed by the amino acid sequence starting at Gln29 to the C-terminus. Vector pEThTIF was transformed into E. coli strain BL21 (DE3)-codon plus-RII. The resulting strain was maintained in LB medium containing Ampicillin (100 μg/ml) and Chloramphenicol (34 μg/ml). Induction of IL-22 express was performed at 37° C. for 4 hours with 1 mM IPTG, which was added when the cultures reached an OD₆₆₀ of approximately 1.0-1.3. Under these conditions, up to 50 mg/l of IL-22 were obtained. Cells were lysed by using a high pressure cell (French Press) and the inclusion bodies were washed once in buffer containing 50 mM Tris-HCl, pH 8.0, 100 mM NaCl, 1 mM EDTA, 1 mM DTT and 0.5% sodium deoxycholate and once in the same buffer lacking sodium deoxycholate. The inclusion bodies were solubilized in 25 mM MES pH 5.5, 8 M urea, 10 mM EDTA, and 0.1 mM DTT. Protein concentration was adjusted to 100 μg/ml and refolded by dialysis in buffer containing Tris-HCl pH 8.0, 0.5 M arginine, 1 mM reduced glutathione, 0.1 mM oxidized glutathione, 2 mM EDTA and 0.1 mnM PMSF. Refolding was performed for 20 h at 4° C. Refolded samples were concentrated 100 fold with a YM3 AMICON membrane and loaded onto a Superdex 75 10/30 HP column (Amersham-Pharmacia), which was eluted with buffer containing 25 mM MES pH 5.4 and 150 mM NaCl. Human IL-22 peak fractions were concentrated to 5 mg/ml with a YM3 AMICON membrane and desalted using a Hiprep 26/10 column (Amersham-Pharmacia) with elution buffer containing 10 mM MES pH 5.4. Human IL-22 was concentrated again to 5 mg/ml and lyophilized in 1 mg fractions.

### Example 2

Protein Crystallization.

Preliminary screening of the crystallization conditions was performed using a sparse-matrix screen at 291 K (Crystal Screen I and II, Hampton Research Corp.). Small crystals were found in the condition number 18, 26 and 29 of the Crystal Screen I kit. Several attempts to enhance crystal quality were performed, including pH and precipitant concentration refinement, detergent addition, and macroseeding. Well diffracting crystals were obtained in hanging drops equilibrated against a reservoir solution consisting of 0.9 M sodium tartrate, TRITON X-100 detergent and 0.1 M HEPES at pH 7.5. The crystallization drops contained equal volumes (1 μl) of reservoir and purified IL-22 (10 mg/ml in 20 mM MES buffer at pH 5.4) solutions. The protein crystallized in the space group P2₁2₁2₁, with unit-cell dimensions a=55.43, b=61.61, c=73.43 Å.

### Example 3

Data Collection.

Crystals were soaked in different cryosoaking solutions, mounted in a rayon loop and fmally flash-cooled to 80° K. in a cold-nitrogen stream. Data collection was performed at the Protein Crystallography beamline (LNLS, Campinas, Brazil; Dumoutier et al. (2000) Genes and Immunity 1: 488-494; Cookson, (2000) Nature 402s: B5-B11) and at the X4A beajmline (NSLS, Upton, USA), using a MAR345 image plate and a Quantum-4 CCD detector, respectively. Three diffraction datasets were collected to a resolution beyond 1.95 Å. Diffraction images were processed and scaled with the programs DENZO and SCALEPACK. See e.g., Walter et al. (1995) Biochemistry 34: 12118-12125.

### Example 4

Heavy-atom Derivatives and Phasing.

The structure was solved by SIRAS. An iodine derivative was obtained by soaking the crystal for 180 seconds in 2 μl of cryoprotectant solution containing 0.125 M sodium iodide following the novel “quick cryo soaking” derivatization procedure. See e.g., Kotenko et al. (1997) EMBO J. 16: 5894-5903; Zdanov et al. (1995) Structure 3: 591-601. The data sets of an iodine derivative (I-IL-22) and a native crystal (Nat-IL-22) were collected at the Protein Crystallography beamline (Dumoutier et al. (2000) Genes and Immunity 1: 488-494; Cookson (2000) Nature 402s: B5-B11) at LNLS (Campinas, Sao Paulo, Brazil). The heavy-atom positions of the iodine derivative were determined by direct methods with the programs DREAR (Ealick et al. (1991) Science 252: 698-702) and SnB 2.1 (Josephson et al. (2000) J. Biol. Chem. 275: 13552-13557). The bimodal distribution of the Rₘᵢₙ histogram was used to identify the correct solution (Treze (1999) The cytokine network and immune functions. Oxford University Press, Oxford; Barton, (1993) Protein Eng. 6: 37-40). The heavy-atom substructure obtained directly from SnB was initially refined with the CNS package using anomalous and isomorphous difference Fourier maps. Refined coordinates were then input into SHARP (Otwinowski et al. (1997) Methods Enzymol. 276: 307-326) for phase calculation, resulting in an overall figure of merit of 0.45 for all reflections in the range of 21.7-2.40 Å. Density modification with solvent flattening was performed using the program SOLOMON. See e.g., Blessing, et al. (1999) J. Appl. Cryst. 32: 664-670. Due to the high resolution and completeness of the I-IL-22 data set, and also the quality of solvent-flattened electron-density map, an automatic construction of an IL-22-hybrid model could be performed by the ARP/wARP program. See e.g., Thiel, et al. (2000) Structure 8: 927-936. The nucleotide-based IL-22 primary structure was used in the final-model-side-chain assignment. See e.g., Dumoutier et al. (2000) Proc. Natl. Acad. Sci. U.S.A. 97: 10144-10149.

One mercury derivative was also obtained using traditional methods of derivatization. The Hg derivative (Hg-IL-22) data set was collected at the X4A beamline at NSLS (Upton, New York, USA) and was used at latter stages of refinement and construction of disordered loops. This latter derivative was prepared using traditional methods for derivatization of protein crystals. Details of native and derivative crystal preparation, as well as data statistics, are summarized in Table 1.

### Example 5

Model Building.

1. Refinement.

A. The initial model was obtained without manual intervention after 6 ARP/wARP jobs and more than 4000 REFMAC cycles. See e.g., Weeks, et al. (1999) J. Appl. Cryst. 32: 120-124. In the last cycle, after almost 72 hours of uninterrupted CPU time in a Pentium III 500 MHz, 81.6% of the total amino acid residues were corrected traced.

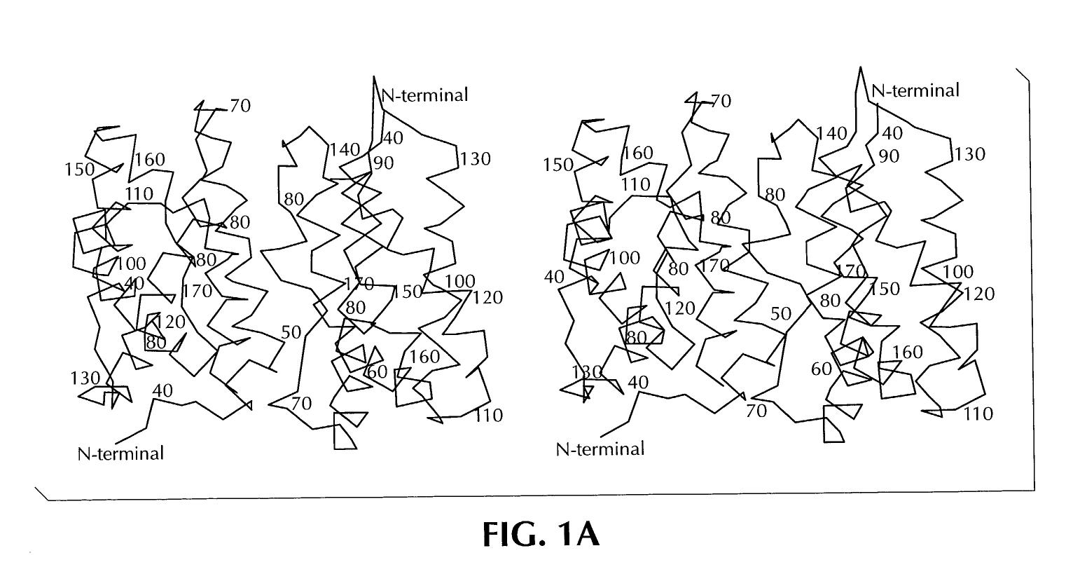

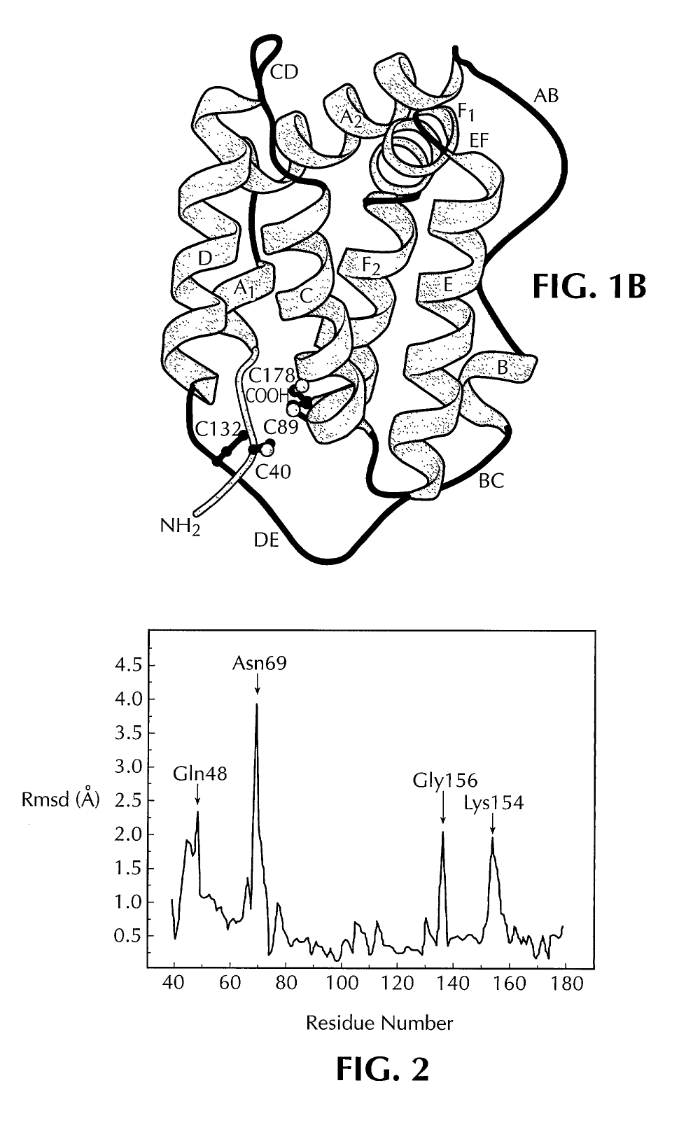

B. The initial structure of IL-22 was improved by a number of cycles of refinement and rebuilding using CNS package. See e.g., Polikarpov, et al. (1997) J. Synchrotron Rad. 5: 72-76. Interlaced refinement of model against Nat-IL-22, Hg-IL-22 and I-IL-22 data sets were used to allow a complete trace of main chain atoms through disordered regions. The initial model contained 231 amino acid residues (in nine distinct chains) and 809 water molecules. The isolated cDNA of IL-22 encodes a protein of 179 amino acids, of which the first 22 amino acids are predicted to function as a signal sequence. (Xie et al. (2000) J. Biol. Chem. 275: 31335-31339). The N-terminal amino acid analysis of IL-22 confirms that the mature protein begins at amino acid residue 34. Construction of disordered loops and filling of main chain gaps were performed manually using the program O. See e.g., Debaerdemaeker, et al. (1983) Acta Cryst. A39: 193-196. Finally, the model was refined against a Nat-IL-22 data set, starting with a simulated annealing protocol in the program CNS. After several iterations of energy minimization, B-factor refinement, and bulk-solvent and anisotropic corrections, the final R_{factor} and R_{free} were 0.191 and 0.225, respectively, for the Nat-IL-22 data in the resolution range of 21.7-1.92 Å. The final model includes 283 residues (two chains) and 189 water molecules. The refined model of IL-22,a dimer in the asymmetric unit (FIG. 1a), includes monomer A with 142 amino acid residues (Ser38-Ile179), monomer B with 141 amino acid residues (His39-Ile179) and 189 water molecules. About 93.8% and 6.2% of the amino acid residues adopt a conformation that corresponds to the most favored and additionally allowed regions of the Ramachandran plot, respectively. See e.g., Table 2 for further information about refinement and geometry statistics. No residues have been encountered in the disallowed regions of the Ramachandran plot.

As shown in FIG. 1b, each monomer of IL-22 model is characterized by six β-helices (A-F) that fold in a compact bundle. Helix A (amino acid residues Lys44-Ser64) is linked to a short helix B (Glu77-Pe80) by a large loop AB (Leu65-Gly76). Helix A has a kink at Gln48-Gln49, presumably due to a hydrogen bond between Nε-Gln49 and O-Ser45 (2.79 Å and 2.55 Å in monomers A and B, respectively). This divides helix A into unequal parts: A₁ and A₂. The loop BC (His81-Glu87) connects helix B to helix C (Arg88-Glu102). The helix C is joined to helix F by a disulfide bond between Cys89 and Cys178. Another loop (CD; Val103-Try114) links helix C to helix D (Met115-Leu129). According to PROCHECK (Laskowski et al. (1993) J. Appl. Crystallogr. 26: 283-291), a small difference in secondary structure between monomers is observed at the loop CD region. A small α-helix is observed between amino acid residues Phe105 and Gln107 of the monomer B. Helix D is connected to helix E by a disordered loop (DE; Ser130-Asp138). This loop is stabilized, at least in the vicinity of Cys132, by another disulfide bond between Cys132 and Cys40, the latter in the N-terminal coil. Finally, a simple junction EF (Gly156) joins the last two helices E (Leu139-Leu155) and F (Glu157-Cys178). Probably, as a consequence of a disulfide bond between Cys89 and Cys178, the latter belonging to the C-terminal of helix F, a kink at Glu166 divides helix F into two parts: F₁ and F₂.

2. Dimer formation.

An expressive part (61%) of the volume of the asymmetric unit (6.27×10⁴ Å³) is occupied by a dimer of IL-22. A small fraction of this volume (8%) is filled with ordered water molecules. The monomers are essentially equal, however, a number of significant differences in the main chain conformation are observed in the vicinity of amino acid residues Gln48, Asn69, Gln136 and Lys154 (FIG. 2). These differences are mostly explained by crystallographic and non-crystallographic contacts. The reason for a significant positional difference between monomers around Gln48 is the fact that this region in monomer A is involved in interface interactions, whereas the same region in monomer B is exposed to the solvent. In addition, the presence of two intramolecular interactions-Oδ1-Asp43/Oγ-Ser45 with 2.64 Å in monomer A and O-Asn46/Nε2-Gln49 with 2.55 Å in monomer B—contribute to a relative change in main-chain atoms positions between residues Leu42 and Pro50. A second conformational difference around Asn69 is a consequence of a crystallographic contact between side chain atoms of Asn69 and Thr70 of monomers A and B, respectively. Gly136 is localized in the disordered loop DE. This fact explains the root-mean-square-deviation (msd) around 2.0 Å in the vicinity of this amino acid residue. Finally, the last major difference between monomers is found close to Lys154. In this region, three distinct interactions of Lys153 and Lys154 from monomer B-Oε1-Glu102/Nζ-Lys153 with 2.68 Å, Oδ1-Asn46/Nζ-Lys153 with 2.78 Å and Oε1-Glu160/Nζ-Lys154 with 2.80 Å, which are absent in monomer A—are responsible for a high rmsd of main-chain atoms.

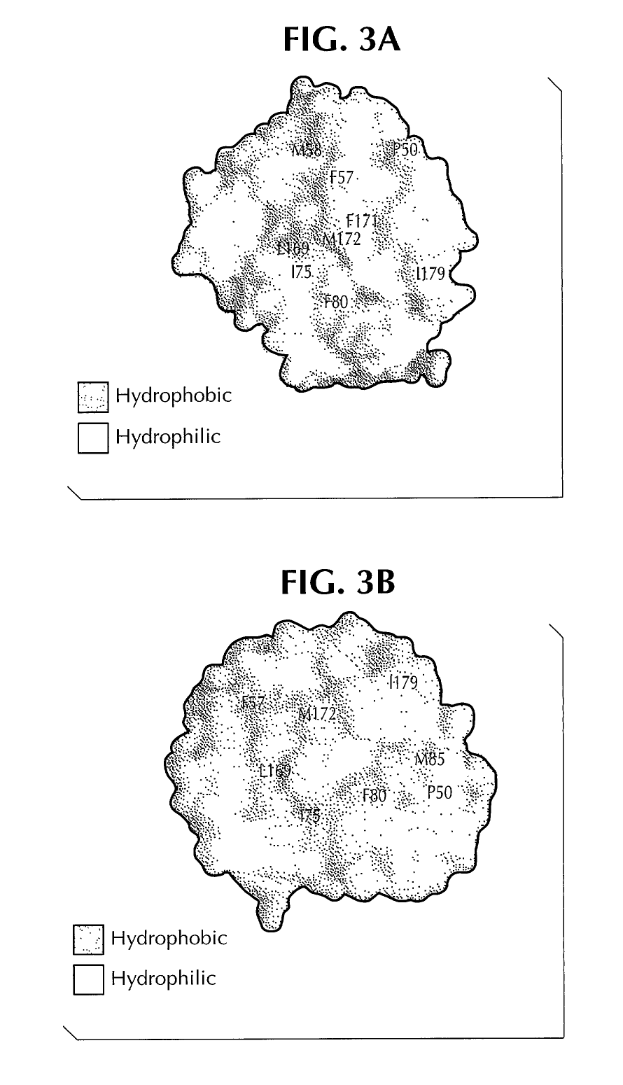

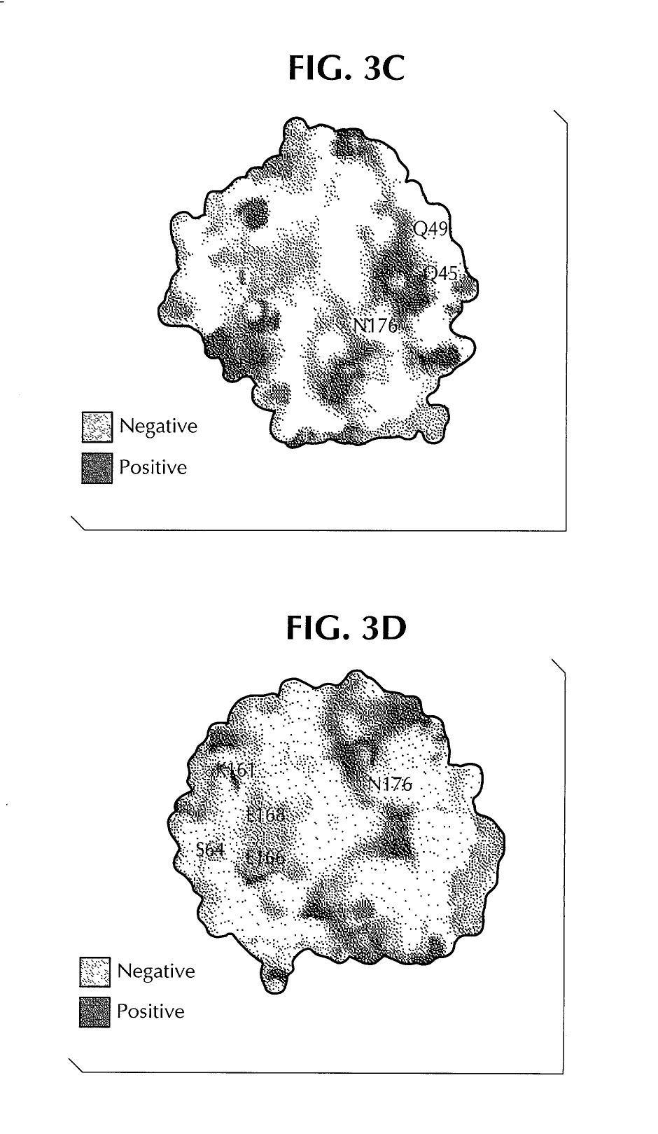

Unlike the hIL-10, the IL-22 dimeric structure formation does not require the intertwining of the main chain of each monomer (FIG. 1). An interface area of approximately 2250 Å², which corresponds to 30% of the total surface area of a monomer, is involved in the dimer formation. The buried surface for the chosen dimer conformation is at least two times larger than any other buried surface area (˜960 Å² or less). Also, the dimer interface, which is formed mostly by residues Arg41 to Phe80 and Asp168 to Ile179 in monomer A and Thr53 to Arg88 and Glu166 to Ile179 in monomer B, has a significant number of hydrophobic residues. Intermolecular interface contacts closer than 3.2 Å are listed in Table 3. The electrostatic and hydrophobic distribution of the IL-22 surface together with the position of the principal amino acid residues involved in the formation of the dimer are given in FIG. 3.

According to the predicted primary structure, human IL-22 has three potential glycosylation sties (Asn-Xaa-Thr/Ser) localized in helix A (Asn54-Arg55-Thr56) (site #1), loop AB (Asn68-Asn69-Thr70) (site #2) and helix C (Asn97-Phe98-Thr99) (site #3). Since the recombinant IL-22 used in crystallization is not glycosylated, we attempted the analysis of the possible interactions between oligosaccharides and IL-22 by calculating the accessible area of each residue in all three putative glycosylation sites. The results demonstrate that site #2, localized at the loop AB, is the one with the larger accessible area. A solvent-accessible area of approximately 37 Å² was found for Nδ2-Asn68 and for Oγ1-Thr70 atoms, indicating that there is no steric hindrance to their participation in N-glycosyl and O-glycosyl links, respectively. On the other hand, sites #1 and #3 seem to participate only in N-glycosyl linkages. The accessible area of Oγ1-Thr56 and Oγ1-Thr99 is 0 and 6 Å², whereas Nδ2-Asn54 and Nδ2-Asn97 atoms possess, respectively, the surface-accessible area of 24 and 18 Å². This structural analysis is in agreement with biochemical studies suggesting that these three sites are of N-glycosyl type. (Kotenko et al. (2001) J. Biol. Chem. 276: 2725-2732). Consistent with biophysical observations, the present structure shows that putative glycosylation sites #1 and #2 reside near the dimer interface, and that glycosylation at these positions would disrupt dimer formation.

3. Comparison of IL-22 to the structures of IL-10 and IFN-γ.

As shown in FIG. 2, the crystallographic structure of hIL-22 is a compact dimer, with a buried surface area of approximately 2250 Å². Several intermolecular interactions along the interface surface keep the monomers together. Each monomer is formed by six α-helices (A-F) from the same polypeptide chain. Quite in contrast, the crystallographic structures of hIL-10 (Levitt et al. (1999) J. Allergy Clin. Immunol. 103: S485-S491; Laskowski et al. (1993) J. Appl. Crystallogr. 26: 283-291; Kraulis et al. (1991) J. appl. Cryst. 24: 946-950) and hIFN-γ (Esnouf (1997) J. Mol. Graph. 15: 133-138; McLane et al. (1998) Am. J. respir. Cell Mol. Biol. 19: 713-720) revealed the presence of a homodimer composed of two α-helical domains formed by intertwining of α-helices donated by the first and the second monomer composing a dimer. The first four helices of one chain (A-D), together with the helices E′ and F′ from the second chain, compose the first domain. Helices A′ to D′, E and F form the second domain.

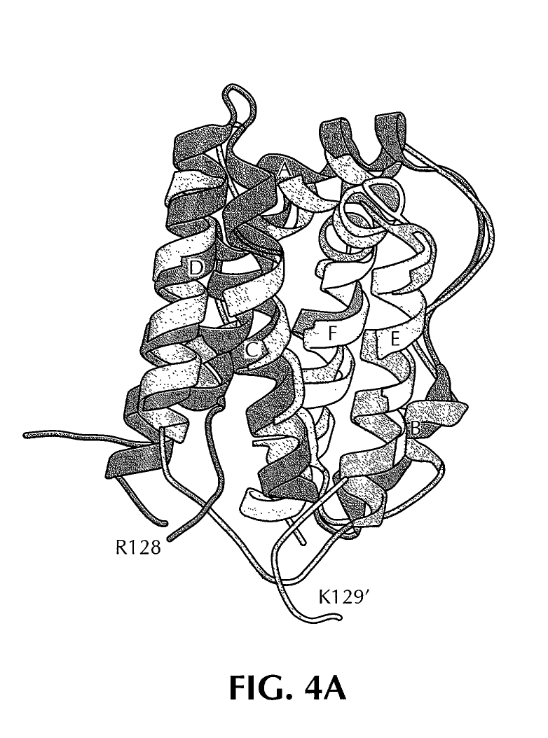

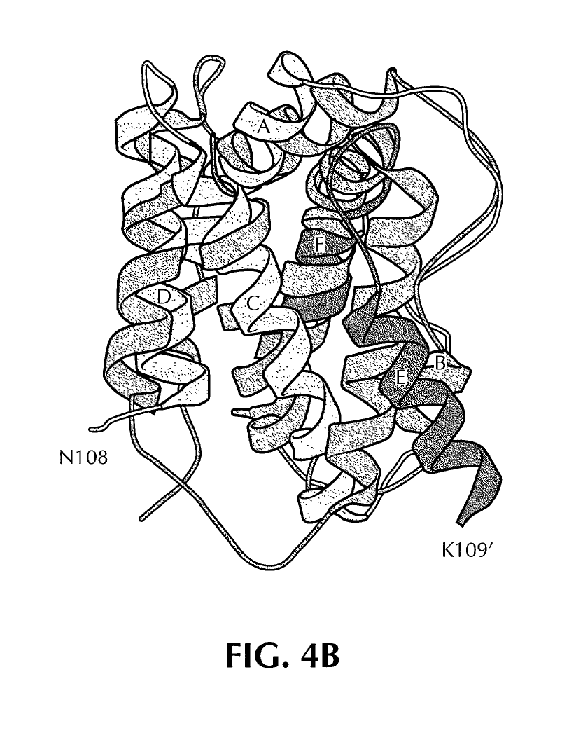

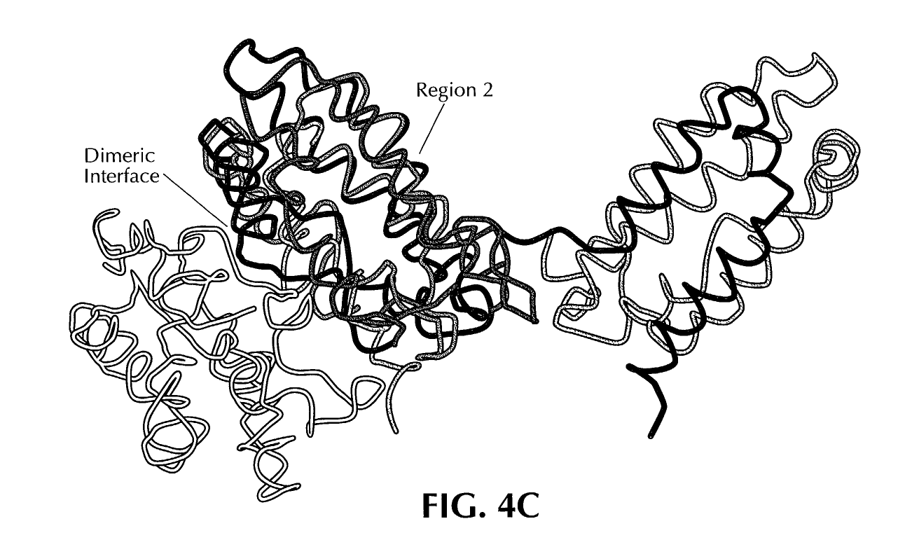

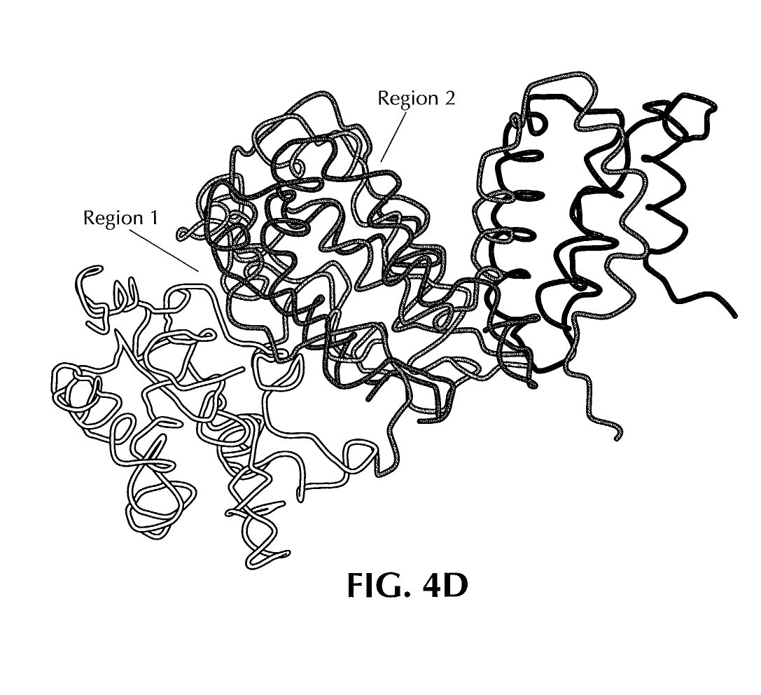

There are significant structural similarities between IL-22, IL-10 and IFN-γ (FIG. 4). In all these proteins, helices A to D of each monomer form a rigid frame with a highly hydrophobic depression in its middle. This depression is covered in IL-22 by helices E and F from the same monomer, whereas in hIL-10 and hIFN-γ this is accomplished by helices E′ and F′ (from the second monomer). The basic reason for these differences is in the loop DE. There are two cysteine residues Cys126 and Cys132 at the hIL-10 DE loop that make two distinct disulfide bonds with residues Cys30 and Cys80 , respectively. (Here we adopted the residue numbering according to the hIL-10 cDNA sequence). These two S-S bridges restrict the flexibility of the amino acid chain and the length of the loop DE in such a manner that helices E and F can not fold onto their respective monomer to occupy position of their counterparts E′ and F′. This leads to the intertwined dimer formation. See e.g., Levitt et al. (1999) J. Allergy Clin. Immunol. 103: S485-S491; Laskowski et al. (1993) J. Appl Crystallogr. 26: 283-291; Kraulis et al. (1991) J. appl Cryst. 24: 946-950. A monomeric form of hIL-10 could only possibly be created when the Cys80-Cys132 disulfide bond were to be reduced, or if a small amino acid chain were inserted after Cys132. See e.g., Levitt et al. (1999) J. Allergy Clin. Immunol. 103: S485-S491. The latter approach has been applied with success to hIL-10, where insertion of a small polypeptide linker in the loop that connects the swapped secondary structure elements led to the formation of a monomeric protein. See e.g., Merritt, et al. (1997) Methods Enzymol. 277: 505-524. Similarly, the hIFN-γ intertwined dimer is formed because the loop DE is not long enough to allow the fold of helix E and F into the same domain.

In IL-22, just one disulfide bond (Cys40-Cys132) exists at the loop DE, which allows sufficient flexibility and extension of the loop to bring helices E and F into close contact with helices A to D and to complete the folding of the monomer. A second disulfide bond, in the C-terminal of helix F (Cys89-Cys178), adds to a rigidity of a final IL-22 structure.

The best superposition of IL-22 onto hIL-10 and hIFN-γ was obtained using a single domain of the hIL-10 and hIFN-γ onto IL-22 yielding an rmsd of 1.9 Å and 2.3 Å for 432 and 300 pairs of main chain atoms, respectively. Helices A to D of the IL-22 monomer superimpose with helices A to D of one of the monomers of hIL-10 and hIFN-γ. Helices E and F fit nicely into the spatial position occupied by helices E′ and F′ of the second monomer. The three-dimensional superposition of the structures allowed us to perform a structure-based sequence alignment for IL-22 and IL-10 that is shown in FIG. 5. Inspection of the superposition of IL-22 and hIL-10 revealed strong similarities in the conformation of the main-chain trace of helices E (E′) and F (F′), and to a lesser extent, the conformation of several parts of loop AB, helix C and helix D. Each of these regions represent high sequence similarity. Some significant differences in the regions of the N-terminal coil, helix A, helix B, loop BC, loop CD and loop DE were also observed.

Reasonable superposition of hIL-10 or hIFN-γ dimers onto IL-22 dimer was proven to be impossible. In each case, dimer formation is so much different that only one domain of IL-22 could be superimposed with an hIL-10 (or hIFN-γ) domain. A second domain of each structure occupies completely different spatial positions (FIGS. 4c and 4d). Whereas the intertwining of α-helices is essential for the formation and integrity of molecules adapting a form of the V-shaped dimers (i.e., hIL-10 and hIFN-γ), in IL-22 dimer formation is not required for folding. It must be stressed that the buried surface on the hIL-2 interface coincides with the outer part of the hIL-10 and hIFN-γ V-shaped-dimer surfaces (FIGS. 4c and 4d).

4. Receptor binding sites.

Two receptor chains-CRF2-4 and CRF2-9-have been identified for IL-22. The CRD2-4 receptor chain is common between IL-22 and IL-10 and is necessary for signaling, whereas CRD2-9 is specific for IL-22. See e.g., Xie et al. (2000) J. Biol. Chem. 275: 31335-31339; Kotenko et al. (2001) J. Biol. Chem. 276: 2725-2732; both incorporated herein by reference. CRF2-9 bears primary sequence homology to the another receptor chain of IL-10-IL-10R1. The binding affinity of IL-22 and IL-10 to CRF2-4 is different. CRF2-4 alone is sufficient to bind IL-22, while the presence of a second receptor chain is required for efficient IL-10 binding. Moreover, both CRF2-9 and CRF2-4 share significant sequence homology to the IFN-γ receptor, IFN-γRα. The three-dimensional structure of hIFN-γRα was recently solved as a complex with its ligand (McLane et al. (1998) Am. J Respir. Cell Mol. Biol. 19: 713-720; incorporated herein by reference), and the structure of IL-22 was superimposed onto the structure of the hIFN-γ/hIFNRα complex to identify the residues involved in IL-22/receptor interactions. A similar structural comparison with the hGH/hGHBP complex has been used in receptor-binding-site analysis of IL-10. See e.g., Levitt, et al. (1999) J. Allergy Clin. Immunol. 103: S485-S491; incorporated herein by reference.

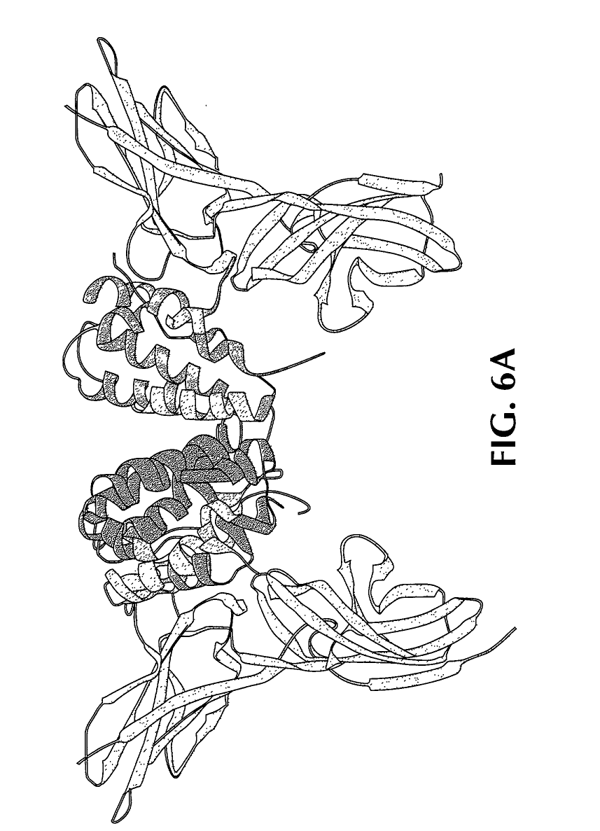

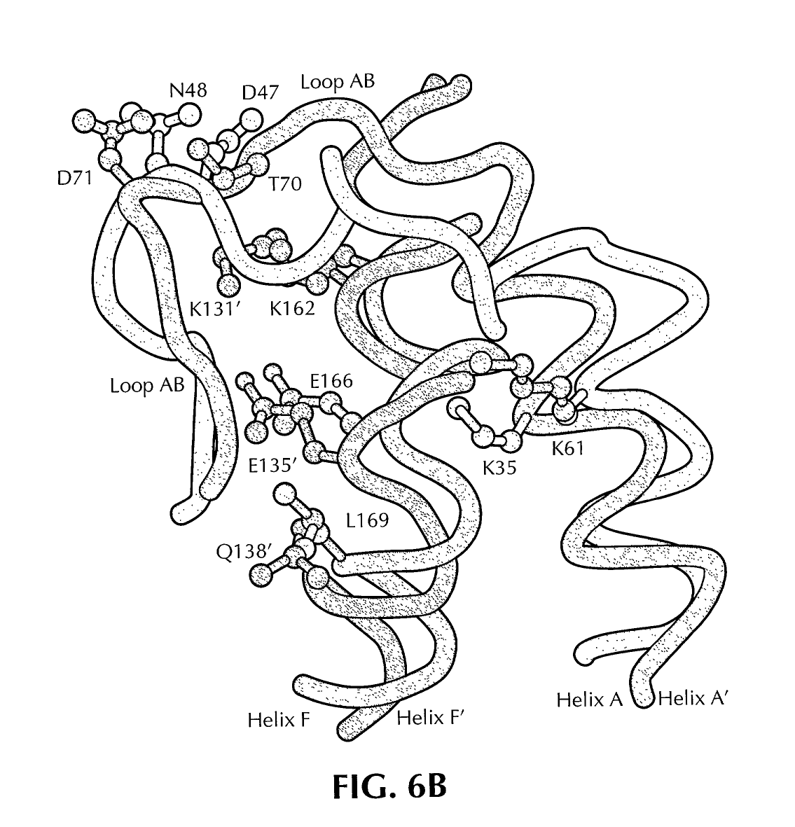

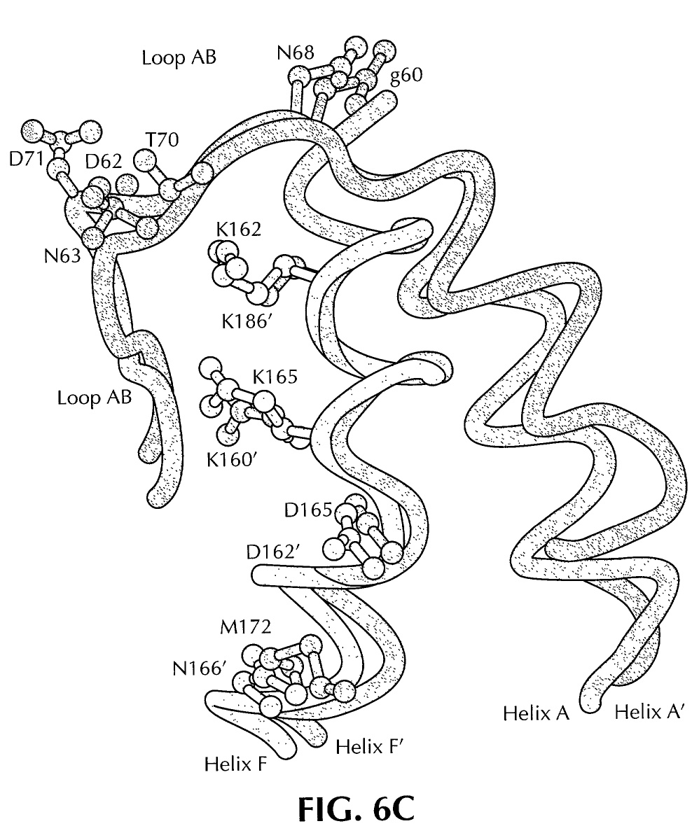

The superposition of IL-22 onto the hIFN-γ/hIFNRα complex indicates that one possible receptor binding site is localized in the region formed by helix A, loop AB and helix F of IL-22 (Region 1, R1; see FIGS. 4d and 6a). Among the 17 residues involved in hIFN-γ/hIFNRα interactions (closer than 3.4 Å), only two residues do not have their IL-22 structural counterparts localized in R1. Nine of the seventeen residues localized in R1 are not sufficiently close to their hIFN-γ counterparts, which may explain the inability of IL-22 to bind to hIFN-γRα The major differences between hIFN-γ and IL-22 within R1 are observed in the loop AB and distances of more than 7 Å are found between their main chains. As shown in FIG. 6b, six relatively conserved residues (Lys61, Thr70, Asp71, Lys162, Glu166 and Leu169), however, occupy almost the same spatial position as six hIFN-γ residues-Lys35, Asp47, Asn48, Lys131, Glu135, Gln138.

A comparison with the hIL-10 putative receptor binding site (Levitt, et al. (1999) J. Allergy Clin. Immunol. 103: S485-S491; incorporated herein by reference) shows that Region 1—helix A, loop AB and helix F′ in the case of hIL-10—is involved in receptor interactions. Amino acid residues Gln60, Asp62, Asn63, Lys156, Glu160, Asp162, Asp166 and Glu169 of the hIL-10 binding site having their IL-22 counterparts in residues Asn68, Thr70, Asp71, Lys162, Glu166, Asp168, Met172 and Arg175. Among these eight residues, Thr70, Asp71, Lys162 and Glu166 were also found in the IL-22:IFN-γ/INF-γRα comparison. The superposition of the hIL-10 putative binding Region 1 onto IL-22 is shown in FIG. 6c. The three-dimensional structure comparison of IL-22 with either IFN-γ/INF-γRα or hGH/hGHBP complexes demonstrates that Region 1 is the receptor binding site.

The three-dimensional similarities observed between IL-22 and hIL-10 in Region 1, especially between helices F and F′, also indicate that this region is the CRF2-4 binding site. In addition, the gylcosylation site in the IL-22 AB loop may interfere with receptor binding. The homology of IF-γRα CRF2-9 also suggests that R1 is the recognition/binding site for CRF2-9. Notably, in the present crystallographic model, the region 1 of each monomer is hidden at the dimer interface. Moreover, a few potential receptor-binding residues are directly involved in dimer formation (see Table 3). Therefore, a IL-22-receptor chain can only bind a monomer of IL-22, and thus, requires the dissociation of the dimer observed in the present crystallographic structure. In contrast, the hIL-10 dimer does not require disruption prior to interaction with the receptor, since the hIL-10-receptor-binding site is localized at the outer part of the B-shaped-dimer surface (FIGS. 4c and 4d).

Although the RZ binding site in IL-22 cannot be easily inferred from inspection of the interactions between hINF-γ and hINF-γRα region Z, which comprises the terminal portions of helices C and E of each IL-22 monomer, is a binding site for CRF2-4. A sequence comparison between IL-22 and several IL-10 identifies several amino acids that are conserved within the Region 2 (R2) region—FTLEEVL (SEQ ID NO: 4) and KLGE (SEQ ID NO: 5) in IL-22 helices C and E, respectively. Region 2 is localized at the surface of IL-22, which is opposite to R1. Localization of each binding region (R1 and R2) on the opposite sides of the IL-22 molecule allows IL-22 to interact with two receptor chains simultaneously. In hIL-10, the amino acids corresponding to the region R2 are localized at the inner part of the V-shaped dimer surface. The angle between each hIL-10 domain in the V-shaped diner is large enough to allow interaction of two CRF2-4 receptor chains with the two binding sites in RZ 2 (FIG. 4c).

TABLE 1
Details of the preparation and data-collection statistics of IL-22 crystals.
Statistical values for the highest resolution shells are shown in parentheses.

| | Nat-IL-22 | I-IL-22 | Hg-IL-22 |
|---|---|---|---|
| Space group | P2₁2₁2₁ | P2₁2₁2₁ | P2₁2₁2₁ |
| Unit cell parameters (Å) | a = 55.43; b = 61.61; | a = 56.05; b = 61.78; | a = 56.04; b = 61.71; |
| | c = 73.47 | c = 73.63 | c = 74.61 |
| Resolution (Å) | 21.7-1.92 | 21.8-1.92 | 22.4-1.90 |
| | (1.96-1.92) | (1.96-1.92) | (1.97-1.90) |
| No. of reflections | 67677 | 182876 | 55855 |
| No. of unique reflections¹ | 18139 | 37777 | 29854 |
| <I/σ(I)> | 14.4 (2.5) | 13.4 (3.1) | 8.2 (2.1) |
| Multiplicity | 3.7 (3.1) | 4.8 (4.3) | 1.9 (1.7) |
| Completeness | 91.2 (75.1) | 99.9 (99.7) | 75.9 (77.9) |
| R_{merge}² | 8.6 (50.8) | 11.7 (43.9) | 10.0 (49.9) |
| Data collected (degrees) | 103.2 | 248.6 | 70.0 |
| Cryoprotectant solution | Mother liquor | Mother liquor | Mother liquor |
| | 15% ethyl. glycol | 15% ethyl. glycol | 15% ethyl.glycol |
| | | 0.125 M NaI | 5 mM HgCl₂ |
| Soaking time | 30 seconds | 180 seconds | 10 hours |

¹Multiplicity of derivative (native) data sets calculated with Friedel-related reflections treated separately (as equivalent).
²R_{merge} = Σhkl |Iₕₖₗ − <Iₕₖₗ>| / Σₕₖₗ Iₕₖₗ

TABLE 2
Refinement statistics and quality of the IL-22 model.¹

| GENERAL INFORMATION | |
|---|---|
| Disulfide bonds | Cys40-Cys132 and Cys89-Cys178 |
| Cis-peptides | Pro 113 |
| Alternative conformations | Met 172 in monomer A |
| | Asp43, Ser45, Arg55, Ile75, His81, |
| | Arg124, Ile161 and Leu174 in monomer B |
| REFINEMENT STATISTICS (21.7-1.92 Å) | |
| Total number of reflections | 17238 |
| Working set number of reflections | 16372 |
| R-factor (%) | 19.1 |
| Test set number of reflections | 866 |
| R-free (%) | 22.5 |
| Total number of protein atoms | 2330 |
| Total number of water molecules | 189 |
| GEOMETRY STATISTICS | |
| Rmsd bond distances (Å) | 0.006 |
| Rmsd bond angles (°) | 1.1 |
| Average B factors | |
| residue atoms (Å²) (A, B) | 24.3 (22.3, 26.2) |
| mainchain atoms (Å²) (A, B) | 22.1 (20.2, 24.1) |
| sidechain atoms (Å²) (A, B) | 26.3 (24.4, 28.1) |
| water molecules (Å²) | 37.3 |
| Average rmsd B factor | |
| residue atoms (Å²) (A, B) | 2.5 (2.6, 2.5) |
| mainchain atoms (Å²) (A, B) | 1.0 (1.0, 1.0) |
| sidechain atoms (Å²) (A, B) | 1.9 (2.0, 1.8) |
| water molecules (Å²) | 11.4 |
| Ramachandran plot² | |
| residues in most favored region (%) | 93.8 |
| residues in additionally allowed regions (%) | 6.2 |
| residues in generously allowed regions (%) | 0.0 |
| residues in disallowed regions (%) | 0.0 |
| NON-CRYSTALLOGRAPHIC SYMMETRY³ | |
| Rmsd coordinates | |
| Cα atoms (Å) | 0.911 |
| mainchain atoms (Å) | 0.884 |
| all bonded atoms (Å) | 1.670 |
| Rmsd B factors | |
| Cα atoms (Å²) | 10.04 |
| mainchain atoms (Å²) | 10.05 |
| all bonded atoms (Å) | 10.77 |

¹Amino acid residues correspond to residues in human IL-22, SEQ ID NO: 2.
²Regions as defined by PROCHECK (Laskowski et al. (1993) J. Appl. Crystallogr. 26: 283-291).
³Non-crystallographic symmetry of subunits A and B.

TABLE 3
Intermolecular contacts (monomers A* and B**).
The distance cut-off of 3.2 Å was used.¹

| Residue* | Atom* | Residue** | Atom** | Distance (Å) |
|---|---|---|---|---|
| Arg175 | Nη2 | Glu166 | Oε1 | 2.57 |
| Phe57 | O | Asn176 | Nδ2 | 2.64 |
| Arg73 | Nη2 | Val83 | O | 2.71 |
| Lys44 | Nζ | Ser64 | Oγ | 2.85 |
| Arg175 | Nη1 | Asp168 | Oδ2 | 2.86 |
| Asn176 | Nδ2 | Ile75 | O | 2.91 |
| Gln48 | O | Lys61 | Cε | 2.96 |
| Lys44 | Nζ | Glu166 | Oε1 | 2.98 |
| Lys61 | Nζ | Ile179 | OT1 | 3.12 |
| Gln49 | Oε1 | Lys61 | Nζ | 3.15 |

¹Amino acid residues correspond to residues in human IL-22, SEQ ID NO: 2.

TABLE 4
Atomic Coordinates of human IL-22 determined as described herein.¹

| | | | | | | | | | | |
|---|---|---|---|---|---|---|---|---|---|---|
| CRYST1 | 55.430 | 61.610 | 73.470 | 90.00 | 90.00 | 90.00 | P | 21 | 21 | 21 |

| | | | | |
|---|---|---|---|---|
| SCALE1 | 0.01804 | 0.00000 | 0.00000 | 0.00000 |
| SCALE2 | 0.00000 | 0.01623 | 0.00000 | 0.00000 |
| SCALE3 | 0.00000 | 0.00000 | 0.01361 | 0.00000 |

| | | | | | | | | | | | |
|---|---|---|---|---|---|---|---|---|---|---|---|
| ATOM | | 1 | CB | SER | A | 38 | 8.633 | 14.375 | 24.449 | 1.00 | 47.79 |
| A | C | | | | | | | | | | |
| ATOM | | 2 | OG | SER | A | 38 | 8.362 | 14.381 | 23.062 | 1.00 | 49.55 |
| A | O | | | | | | | | | | |
| ATOM | | 3 | C | SER | A | 38 | 8.165 | 16.820 | 24.641 | 1.00 | 44.87 |
| A | C | | | | | | | | | | |
| ATOM | | 4 | O | SER | A | 38 | 7.431 | 17.426 | 23.855 | 1.00 | 44.76 |
| A | O | | | | | | | | | | |
| ATOM | | 5 | N | SER | A | 38 | 6.339 | 15.165 | 24.930 | 1.00 | 47.40 |
| A | N | | | | | | | | | | |
| ATOM | | 6 | CA | SER | A | 38 | 7.787 | 15.434 | 25.155 | 1.00 | 46.37 |
| A | C | | | | | | | | | | |
| ATOM | | 7 | N | HIS | A | 39 | 9.311 | 17.315 | 25.099 | 1.00 | 42.59 |
| A | N | | | | | | | | | | |
| ATOM | | 8 | CA | HIS | A | 39 | 9.807 | 18.631 | 24.708 | 1.00 | 40.15 |
| A | C | | | | | | | | | | |
| ATOM | | 9 | CB | HIS | A | 39 | 10.759 | 19.164 | 25.780 | 1.00 | 43.57 |
| A | C | | | | | | | | | | |
| ATOM | | 10 | CG | HIS | A | 39 | 11.980 | 18.320 | 25.978 | 1.00 | 46.37 |
| A | C | | | | | | | | | | |
| ATOM | | 11 | CD2 | HIS | A | 39 | 13.290 | 18.653 | 26.075 | 1.00 | 48.11 |
| A | C | | | | | | | | | | |
| ATOM | | 12 | ND1 | HIS | A | 39 | 11.923 | 16.950 | 26.124 | 1.00 | 48.60 |
| A | N | | | | | | | | | | |
| ATOM | | 13 | CE1 | HIS | A | 39 | 13.143 | 16.476 | 26.302 | 1.00 | 49.04 |
| A | C | | | | | | | | | | |
| ATOM | | 14 | NE2 | HIS | A | 39 | 13.992 | 17.489 | 26.276 | 1.00 | 48.92 |
| A | N | | | | | | | | | | |
| ATOM | | 15 | C | HIS | A | 39 | 10.535 | 18.557 | 23.371 | 1.00 | 36.84 |
| A | C | | | | | | | | | | |
| ATOM | | 16 | O | HIS | A | 39 | 11.370 | 17.680 | 23.157 | 1.00 | 36.46 |
| A | O | | | | | | | | | | |
| ATOM | | 17 | N | CYS | A | 40 | 10.221 | 19.475 | 22.465 | 1.00 | 32.36 |
| A | N | | | | | | | | | | |
| ATOM | | 18 | CA | CYS | A | 40 | 10.875 | 19.461 | 21.169 | 1.00 | 29.17 |
| A | C | | | | | | | | | | |
| ATOM | | 19 | C | CYS | A | 40 | 12.286 | 20.011 | 21.292 | 1.00 | 26.49 |
| A | C | | | | | | | | | | |
| ATOM | | 20 | O | CYS | A | 40 | 12.490 | 21.114 | 21.793 | 1.00 | 24.81 |
| A | O | | | | | | | | | | |
| ATOM | | 21 | CB | CYS | A | 40 | 10.095 | 20.298 | 20.162 | 1.00 | 29.95 |
| A | C | | | | | | | | | | |
| ATOM | | 22 | SG | CYS | A | 40 | 8.366 | 19.818 | 19.887 | 1.00 | 28.05 |
| A | S | | | | | | | | | | |
| ATOM | | 23 | N | ARG | A | 41 | 13.259 | 19.238 | 20.828 | 1.00 | 24.42 |
| A | N | | | | | | | | | | |
| ATOM | | 24 | CA | ARG | A | 41 | 14.648 | 19.657 | 20.890 | 1.00 | 24.30 |
| A | C | | | | | | | | | | |
| ATOM | | 25 | CB | ARG | A | 41 | 15.144 | 19.580 | 22.339 | 1.00 | 28.36 |
| A | C | | | | | | | | | | |
| ATOM | | 26 | CG | ARG | A | 41 | 16.568 | 20.046 | 22.548 | 1.00 | 35.78 |
| A | C | | | | | | | | | | |
| ATOM | | 27 | CD | ARG | A | 41 | 16.733 | 20.673 | 23.927 | 1.00 | 40.37 |
| A | C | | | | | | | | | | |
| ATOM | | 28 | NE | ARG | A | 41 | 15.954 | 21.902 | 24.052 | 1.00 | 43.92 |
| A | N | | | | | | | | | | |
| ATOM | | 29 | CZ | ARG | A | 41 | 15.888 | 22.643 | 25.155 | 1.00 | 47.14 |
| A | C | | | | | | | | | | |
| ATOM | | 30 | NH1 | ARG | A | 41 | 16.558 | 22.282 | 26.244 | 1.00 | 46.80 |
| A | N | | | | | | | | | | |
| ATOM | | 31 | NH2 | ARG | A | 41 | 15.147 | 23.745 | 25.169 | 1.00 | 49.17 |
| A | N | | | | | | | | | | |
| ATOM | | 32 | C | ARG | A | 41 | 15.489 | 18.762 | 19.990 | 1.00 | 22.91 |
| A | C | | | | | | | | | | |
| ATOM | | 33 | O | ARG | A | 41 | 15.087 | 17.650 | 19.668 | 1.00 | 22.24 |
| A | O | | | | | | | | | | |
| ATOM | | 34 | N | LEU | A | 42 | 16.650 | 19.265 | 19.578 | 1.00 | 20.60 |
| A | N | | | | | | | | | | |
| ATOM | | 35 | CA | LEU | A | 42 | 17.568 | 18.528 | 18.715 | 1.00 | 17.57 |
| A | C | | | | | | | | | | |
| ATOM | | 36 | CB | LEU | A | 42 | 17.411 | 18.989 | 17.264 | 1.00 | 17.84 |
| A | C | | | | | | | | | | |
| ATOM | | 37 | CG | LEU | A | 42 | 16.086 | 18.717 | 16.557 | 1.00 | 18.08 |
| A | C | | | | | | | | | | |
| ATOM | | 38 | CD1 | LEU | A | 42 | 16.074 | 19.406 | 15.195 | 1.00 | 17.09 |
| A | C | | | | | | | | | | |
| ATOM | | 39 | CD2 | LEU | A | 42 | 15.902 | 17.218 | 16.405 | 1.00 | 18.48 |
| A | C | | | | | | | | | | |
| ATOM | | 40 | C | LEU | A | 42 | 18.992 | 18.814 | 19.184 | 1.00 | 16.85 |
| A | C | | | | | | | | | | |
| ATOM | | 41 | O | LEU | A | 42 | 19.391 | 19.973 | 19.306 | 1.00 | 18.05 |
| A | O | | | | | | | | | | |
| ATOM | | 42 | N | ASP | A | 43 | 19.760 | 17.766 | 19.455 | 1.00 | 15.30 |
| A | N | | | | | | | | | | |
| ATOM | | 43 | CA | ASP | A | 43 | 21.130 | 17.962 | 19.902 | 1.00 | 13.64 |
| A | C | | | | | | | | | | |
| ATOM | | 44 | CB | ASP | A | 43 | 21.815 | 16.607 | 20.093 | 1.00 | 13.53 |
| A | C | | | | | | | | | | |
| ATOM | | 45 | CG | ASP | A | 43 | 23.177 | 16.732 | 20.753 | 1.00 | 18.06 |
| A | C | | | | | | | | | | |
| ATOM | | 46 | OD1 | ASP | A | 43 | 24.185 | 16.931 | 20.042 | 1.00 | 15.90 |
| A | O | | | | | | | | | | |
| ATOM | | 47 | OD2 | ASP | A | 43 | 23.235 | 16.645 | 21.993 | 1.00 | 20.18 |
| A | O | | | | | | | | | | |
| ATOM | | 48 | C | ASP | A | 43 | 21.869 | 18.802 | 18.855 | 1.00 | 12.61 |
| A | C | | | | | | | | | | |
| ATOM | | 49 | O | ASP | A | 43 | 21.634 | 18.661 | 17.655 | 1.00 | 12.49 |
| A | O | | | | | | | | | | |
| ATOM | | 50 | N | LYS | A | 44 | 22.755 | 19.682 | 19.303 | 1.00 | 11.01 |
| A | N | | | | | | | | | | |
| ATOM | | 51 | CA | LYS | A | 44 | 23.497 | 20.525 | 18.373 | 1.00 | 11.24 |
| A | C | | | | | | | | | | |
| ATOM | | 52 | CB | LYS | A | 44 | 24.368 | 21.536 | 19.129 | 1.00 | 11.63 |
| A | C | | | | | | | | | | |
| ATOM | | 53 | CG | LYS | A | 44 | 24.903 | 22.642 | 18.219 | 1.00 | 14.43 |
| A | C | | | | | | | | | | |
| ATOM | | 54 | CD | LYS | A | 44 | 25.657 | 23.738 | 18.977 | 1.00 | 16.69 |
| A | C | | | | | | | | | | |
| ATOM | | 55 | CE | LYS | A | 44 | 26.076 | 24.856 | 18.021 | 1.00 | 16.93 |
| A | C | | | | | | | | | | |
| ATOM | | 56 | NZ | LYS | A | 44 | 26.812 | 25.974 | 18.696 | 1.00 | 17.12 |
| A | N | | | | | | | | | | |
| ATOM | | 57 | C | LYS | A | 44 | 24.375 | 19.734 | 17.392 | 1.00 | 10.50 |
| A | C | | | | | | | | | | |
| ATOM | | 58 | O | LYS | A | 44 | 24.702 | 20.240 | 16.321 | 1.00 | 10.60 |
| A | O | | | | | | | | | | |
| ATOM | | 59 | N | SER | A | 45 | 24.753 | 18.507 | 17.745 | 1.00 | 8.11 |
| A | N | | | | | | | | | | |
| ATOM | | 60 | CA | SER | A | 45 | 25.585 | 17.699 | 16.844 | 1.00 | 10.59 |
| A | C | | | | | | | | | | |
| ATOM | | 61 | CB | SER | A | 45 | 25.941 | 16.352 | 17.479 | 1.00 | 10.95 |
| A | C | | | | | | | | | | |
| ATOM | | 62 | OG | SER | A | 45 | 24.779 | 15.624 | 17.827 | 1.00 | 12.31 |
| A | O | | | | | | | | | | |
| ATOM | | 63 | C | SER | A | 45 | 24.907 | 17.460 | 15.499 | 1.00 | 11.14 |
| A | C | | | | | | | | | | |
| ATOM | | 64 | O | SER | A | 45 | 25.571 | 17.208 | 14.496 | 1.00 | 10.24 |
| A | O | | | | | | | | | | |
| ATOM | | 65 | N | ASN | A | 46 | 23.582 | 17.533 | 15.477 | 1.00 | 10.77 |
| A | N | | | | | | | | | | |
| ATOM | | 66 | CA | ASN | A | 46 | 22.841 | 17.352 | 14.232 | 1.00 | 10.71 |
| A | C | | | | | | | | | | |
| ATOM | | 67 | CB | ASN | A | 46 | 21.335 | 17.486 | 14.476 | 1.00 | 10.57 |
| A | C | | | | | | | | | | |
| ATOM | | 68 | CG | ASN | A | 46 | 20.737 | 16.244 | 15.094 | 1.00 | 11.94 |
| A | C | | | | | | | | | | |
| ATOM | | 69 | OD1 | ASN | A | 46 | 20.651 | 15.204 | 14.443 | 1.00 | 12.54 |
| A | O | | | | | | | | | | |
| ATOM | | 70 | ND2 | ASN | A | 46 | 20.333 | 16.339 | 16.361 | 1.00 | 8.37 |
| A | N | | | | | | | | | | |
| ATOM | | 71 | C | ASN | A | 46 | 23.231 | 18.402 | 13.207 | 1.00 | 11.00 |
| A | C | | | | | | | | | | |
| ATOM | | 72 | O | ASN | A | 46 | 23.116 | 18.172 | 12.011 | 1.00 | 11.41 |
| A | O | | | | | | | | | | |
| ATOM | | 73 | N | PHE | A | 47 | 23.691 | 19.551 | 13.688 | 1.00 | 11.89 |
| A | N | | | | | | | | | | |
| ATOM | | 74 | CA | PHE | A | 47 | 24.028 | 20.671 | 12.812 | 1.00 | 13.43 |
| A | C | | | | | | | | | | |
| ATOM | | 75 | CB | PHE | A | 47 | 23.217 | 21.890 | 13.254 | 1.00 | 13.84 |
| A | C | | | | | | | | | | |
| ATOM | | 76 | CG | PHE | A | 47 | 21.760 | 21.593 | 13.463 | 1.00 | 13.84 |
| A | C | | | | | | | | | | |
| ATOM | | 77 | CD1 | PHE | A | 47 | 20.886 | 21.510 | 12.377 | 1.00 | 15.85 |
| A | C | | | | | | | | | | |
| ATOM | | 78 | CD2 | PHE | A | 47 | 21.273 | 21.347 | 14.739 | 1.00 | 12.02 |
| A | C | | | | | | | | | | |
| ATOM | | 79 | CE1 | PHE | A | 47 | 19.543 | 21.182 | 12.567 | 1.00 | 15.39 |
| A | C | | | | | | | | | | |
| ATOM | | 80 | CE2 | PHE | A | 47 | 19.937 | 21.019 | 14.940 | 1.00 | 14.91 |
| A | C | | | | | | | | | | |
| ATOM | | 81 | CZ | PHE | A | 47 | 19.068 | 20.935 | 13.855 | 1.00 | 12.76 |
| A | C | | | | | | | | | | |
| ATOM | | 82 | C | PHE | A | 47 | 25.498 | 21.047 | 12.773 | 1.00 | 12.62 |
| A | C | | | | | | | | | | |
| ATOM | | 83 | O | PHE | A | 47 | 25.846 | 22.136 | 12.312 | 1.00 | 13.52 |
| A | O | | | | | | | | | | |
| ATOM | | 84 | N | GLN | A | 48 | 26.361 | 20.152 | 13.243 | 1.00 | 13.34 |
| A | N | | | | | | | | | | |
| ATOM | | 85 | CA | GLN | A | 48 | 27.789 | 20.436 | 13.281 | 1.00 | 12.80 |
| A | C | | | | | | | | | | |
| ATOM | | 86 | CB | GLN | A | 48 | 28.300 | 20.324 | 14.719 | 1.00 | 12.87 |
| A | C | | | | | | | | | | |
| ATOM | | 87 | CG | GLN | A | 48 | 27.678 | 21.326 | 15.686 | 1.00 | 14.56 |
| A | C | | | | | | | | | | |
| ATOM | | 88 | CD | GLN | A | 48 | 28.082 | 21.066 | 17.116 | 1.00 | 16.25 |
| A | C | | | | | | | | | | |
| ATOM | | 89 | OE1 | GLN | A | 48 | 27.892 | 19.965 | 17.634 | 1.00 | 18.12 |
| A | O | | | | | | | | | | |
| ATOM | | 90 | NE2 | GLN | A | 48 | 28.644 | 22.075 | 17.766 | 1.00 | 15.04 |
| A | N | | | | | | | | | | |
| ATOM | | 91 | C | GLN | A | 48 | 28.636 | 19.542 | 12.388 | 1.00 | 14.54 |
| A | C | | | | | | | | | | |
| ATOM | | 92 | O | GLN | A | 48 | 29.860 | 19.568 | 12.486 | 1.00 | 13.57 |
| A | O | | | | | | | | | | |
| ATOM | | 93 | N | GLN | A | 49 | 27.998 | 18.749 | 11.528 | 1.00 | 12.16 |
| A | N | | | | | | | | | | |
| ATOM | | 94 | CA | GLN | A | 49 | 28.742 | 17.858 | 10.640 | 1.00 | 12.52 |
| A | C | | | | | | | | | | |
| ATOM | | 95 | CB | GLN | A | 49 | 27.990 | 16.530 | 10.503 | 1.00 | 12.58 |
| A | C | | | | | | | | | | |
| ATOM | | 96 | CG | GLN | A | 49 | 27.439 | 16.022 | 11.847 | 1.00 | 12.67 |
| A | C | | | | | | | | | | |
| ATOM | | 97 | CD | GLN | A | 49 | 28.483 | 16.019 | 12.961 | 1.00 | 15.57 |
| A | C | | | | | | | | | | |
| ATOM | | 98 | OE1 | GLN | A | 49 | 29.730 | 15.772 | 12.598 | 1.00 | 12.50 |
| A | O | | | | | | | | | | |
| ATOM | | 99 | NE2 | GLN | A | 49 | 28.165 | 16.234 | 14.136 | 1.00 | 14.24 |
| A | N | | | | | | | | | | |
| ATOM | | 100 | C | GLN | A | 49 | 28.941 | 18.568 | 9.295 | 1.00 | 12.85 |
| A | C | | | | | | | | | | |
| ATOM | | 101 | O | GLN | A | 49 | 27.985 | 18.899 | 8.589 | 1.00 | 11.22 |
| A | O | | | | | | | | | | |
| ATOM | | 102 | N | PRO | A | 50 | 30.204 | 18.823 | 8.927 | 1.00 | 13.42 |
| A | N | | | | | | | | | | |
| ATOM | | 103 | CD | PRO | A | 50 | 31.449 | 18.453 | 9.628 | 1.00 | 12.27 |
| A | C | | | | | | | | | | |
| ATOM | | 104 | CA | PRO | A | 50 | 30.492 | 19.514 | 7.666 | 1.00 | 12.60 |
| A | C | | | | | | | | | | |
| ATOM | | 105 | CB | PRO | A | 50 | 32.022 | 19.622 | 7.663 | 1.00 | 11.32 |
| A | C | | | | | | | | | | |
| ATOM | | 106 | CG | PRO | A | 50 | 32.460 | 18.448 | 8.495 | 1.00 | 14.19 |
| A | C | | | | | | | | | | |
| ATOM | | 107 | C | PRO | A | 50 | 29.948 | 18.944 | 6.370 | 1.00 | 11.49 |
| A | C | | | | | | | | | | |
| ATOM | | 108 | O | PRO | A | 50 | 29.464 | 19.697 | 5.520 | 1.00 | 10.14 |
| A | O | | | | | | | | | | |
| ATOM | | 109 | N | TYR | A | 51 | 30.001 | 17.629 | 6.205 | 1.00 | 11.00 |
| A | N | | | | | | | | | | |
| ATOM | | 110 | CA | TYR | A | 51 | 29.530 | 17.056 | 4.955 | 1.00 | 12.96 |
| A | C | | | | | | | | | | |
| ATOM | | 111 | CB | TYR | A | 51 | 29.750 | 15.535 | 4.932 | 1.00 | 14.28 |
| A | C | | | | | | | | | | |
| ATOM | | 112 | CG | TYR | A | 51 | 29.310 | 14.977 | 3.603 | 1.00 | 16.43 |
| A | C | | | | | | | | | | |
| ATOM | | 113 | CD1 | TYR | A | 51 | 30.152 | 15.062 | 2.498 | 1.00 | 16.36 |
| A | C | | | | | | | | | | |
| ATOM | | 114 | CE1 | TYR | A | 51 | 29.742 | 14.584 | 1.244 | 1.00 | 18.58 |
| A | C | | | | | | | | | | |
| ATOM | | 115 | CD2 | TYR | A | 51 | 28.041 | 14.386 | 3.425 | 1.00 | 17.28 |
| A | C | | | | | | | | | | |
| ATOM | | 116 | CE2 | TYR | A | 51 | 27.619 | 13.935 | 2.175 | 1.00 | 17.57 |
| A | C | | | | | | | | | | |
| ATOM | | 117 | CZ | TYR | A | 51 | 28.476 | 14.041 | 1.089 | 1.00 | 19.05 |
| A | C | | | | | | | | | | |
| ATOM | | 118 | OH | TYR | A | 51 | 28.068 | 13.623 | −0.157 | 1.00 | 20.18 |
| A | O | | | | | | | | | | |
| ATOM | | 119 | C | TYR | A | 51 | 28.068 | 17.375 | 4.634 | 1.00 | 11.00 |
| A | C | | | | | | | | | | |
| ATOM | | 120 | O | TYR | A | 51 | 27.773 | 17.999 | 3.612 | 1.00 | 12.39 |
| A | O | | | | | | | | | | |
| ATOM | | 121 | N | ILE | A | 52 | 27.159 | 16.938 | 5.494 | 1.00 | 10.51 |
| A | N | | | | | | | | | | |
| ATOM | | 122 | CA | ILE | A | 52 | 25.746 | 17.175 | 5.222 | 1.00 | 10.19 |
| A | C | | | | | | | | | | |
| ATOM | | 123 | CB | ILE | A | 52 | 24.838 | 16.307 | 6.125 | 1.00 | 7.95 |
| A | C | | | | | | | | | | |
| ATOM | | 124 | CG2 | ILE | A | 52 | 24.850 | 16.814 | 7.558 | 1.00 | 7.43 |
| A | C | | | | | | | | | | |
| ATOM | | 125 | CG1 | ILE | A | 52 | 23.427 | 16.282 | 5.537 | 1.00 | 9.73 |
| A | C | | | | | | | | | | |
| ATOM | | 126 | CD | ILE | A | 52 | 23.341 | 15.576 | 4.193 | 1.00 | 9.58 |
| A | C | | | | | | | | | | |
| ATOM | | 127 | C | ILE | A | 52 | 25.351 | 18.640 | 5.335 | 1.00 | 9.53 |
| A | C | | | | | | | | | | |
| ATOM | | 128 | O | ILE | A | 52 | 24.416 | 19.073 | 4.671 | 1.00 | 10.79 |
| A | O | | | | | | | | | | |
| ATOM | | 129 | N | THR | A | 53 | 26.053 | 19.407 | 6.167 | 1.00 | 10.80 |
| A | N | | | | | | | | | | |
| ATOM | | 130 | CA | THR | A | 53 | 25.739 | 20.828 | 6.293 | 1.00 | 9.84 |
| A | C | | | | | | | | | | |
| ATOM | | 131 | CB | THR | A | 53 | 26.507 | 21.481 | 7.459 | 1.00 | 11.16 |
| A | C | | | | | | | | | | |
| ATOM | | 132 | OG1 | THR | A | 53 | 26.155 | 20.833 | 8.690 | 1.00 | 12.87 |
| A | O | | | | | | | | | | |
| ATOM | | 133 | CG2 | THR | A | 53 | 26.163 | 22.967 | 7.554 | 1.00 | 9.20 |
| A | C | | | | | | | | | | |
| ATOM | | 134 | C | THR | A | 53 | 26.126 | 21.519 | 4.977 | 1.00 | 11.45 |
| A | C | | | | | | | | | | |
| ATOM | | 135 | O | THR | A | 53 | 25.410 | 22.387 | 4.479 | 1.00 | 11.63 |
| A | O | | | | | | | | | | |
| ATOM | | 136 | N | ASN | A | 54 | 27.257 | 21.114 | 4.407 | 1.00 | 13.24 |
| A | N | | | | | | | | | | |
| ATOM | | 137 | CA | ASN | A | 54 | 27.708 | 21.691 | 3.141 | 1.00 | 13.84 |
| A | C | | | | | | | | | | |
| ATOM | | 138 | CB | ASN | A | 54 | 29.105 | 21.170 | 2.785 | 1.00 | 14.48 |
| A | C | | | | | | | | | | |
| ATOM | | 139 | CG | ASN | A | 54 | 29.639 | 21.771 | 1.500 | 1.00 | 14.91 |
| A | C | | | | | | | | | | |
| ATOM | | 140 | OD1 | ASN | A | 54 | 29.687 | 22.986 | 1.348 | 1.00 | 18.44 |
| A | O | | | | | | | | | | |
| ATOM | | 141 | ND2 | ASN | A | 54 | 30.045 | 20.920 | 0.571 | 1.00 | 19.20 |
| A | N | | | | | | | | | | |
| ATOM | | 142 | C | ASN | A | 54 | 26.719 | 21.350 | 2.025 | 1.00 | 12.10 |
| A | C | | | | | | | | | | |
| ATOM | | 143 | O | ASN | A | 54 | 26.380 | 22.200 | 1.205 | 1.00 | 13.81 |
| A | O | | | | | | | | | | |
| ATOM | | 144 | N | ARG | A | 55 | 26.260 | 20.102 | 1.997 | 1.00 | 14.14 |
| A | N | | | | | | | | | | |
| ATOM | | 145 | CA | ARG | A | 55 | 25.290 | 19.664 | 0.994 | 1.00 | 13.41 |
| A | C | | | | | | | | | | |
| ATOM | | 146 | CB | ARG | A | 55 | 24.950 | 18.180 | 1.176 | 1.00 | 15.87 |
| A | C | | | | | | | | | | |
| ATOM | | 147 | CG | ARG | A | 55 | 26.059 | 17.196 | 0.801 | 1.00 | 19.73 |
| A | C | | | | | | | | | | |
| ATOM | | 148 | CD | ARG | A | 55 | 26.505 | 17.386 | −0.642 | 1.00 | 23.68 |
| A | C | | | | | | | | | | |
| ATOM | | 149 | NE | ARG | A | 55 | 27.405 | 16.325 | −1.085 | 1.00 | 25.59 |
| A | N | | | | | | | | | | |
| ATOM | | 150 | CZ | ARG | A | 55 | 28.442 | 16.519 | −1.891 | 1.00 | 27.42 |
| A | C | | | | | | | | | | |
| ATOM | | 151 | NH1 | ARG | A | 55 | 28.710 | 17.733 | −2.343 | 1.00 | 28.76 |
| A | N | | | | | | | | | | |
| ATOM | | 152 | NH2 | ARG | A | 55 | 29.216 | 15.503 | −2.241 | 1.00 | 27.48 |
| A | N | | | | | | | | | | |
| ATOM | | 153 | C | ARG | A | 55 | 24.007 | 20.480 | 1.129 | 1.00 | 11.99 |
| A | C | | | | | | | | | | |
| ATOM | | 154 | O | ARG | A | 55 | 23.412 | 20.893 | 0.134 | 1.00 | 11.77 |
| A | O | | | | | | | | | | |
| ATOM | | 155 | N | THR | A | 56 | 23.575 | 20.701 | 2.367 | 1.00 | 12.12 |
| A | N | | | | | | | | | | |
| ATOM | | 156 | CA | THR | A | 56 | 22.357 | 21.464 | 2.605 | 1.00 | 10.35 |
| A | C | | | | | | | | | | |
| ATOM | | 157 | CB | THR | A | 56 | 22.017 | 21.513 | 4.104 | 1.00 | 12.01 |
| A | C | | | | | | | | | | |
| ATOM | | 158 | OG1 | THR | A | 56 | 21.792 | 20.180 | 4.591 | 1.00 | 7.11 |
| A | O | | | | | | | | | | |
| ATOM | | 159 | CG2 | THR | A | 56 | 20.766 | 22.340 | 4.337 | 1.00 | 8.42 |
| A | C | | | | | | | | | | |
| ATOM | | 160 | C | THR | A | 56 | 22.483 | 22.889 | 2.062 | 1.00 | 12.24 |
| A | C | | | | | | | | | | |
| ATOM | | 161 | O | THR | A | 56 | 21.604 | 23.370 | 1.345 | 1.00 | 8.45 |
| A | O | | | | | | | | | | |
| ATOM | | 162 | N | PHE | A | 57 | 23.571 | 23.573 | 2.399 | 1.00 | 11.55 |
| A | N | | | | | | | | | | |
| ATOM | | 163 | CA | PHE | A | 57 | 23.753 | 24.937 | 1.914 | 1.00 | 12.54 |
| A | C | | | | | | | | | | |
| ATOM | | 164 | CB | PHE | A | 57 | 24.929 | 25.609 | 2.624 | 1.00 | 12.16 |
| A | C | | | | | | | | | | |
| ATOM | | 165 | CG | PHE | A | 57 | 24.568 | 26.181 | 3.960 | 1.00 | 13.36 |
| A | C | | | | | | | | | | |
| ATOM | | 166 | CD1 | PHE | A | 57 | 24.313 | 25.354 | 5.046 | 1.00 | 13.15 |
| A | C | | | | | | | | | | |
| ATOM | | 167 | CD2 | PHE | A | 57 | 24.464 | 27.553 | 4.130 | 1.00 | 14.39 |
| A | C | | | | | | | | | | |
| ATOM | | 168 | CE1 | PHE | A | 57 | 23.961 | 25.887 | 6.282 | 1.00 | 14.05 |
| A | C | | | | | | | | | | |
| ATOM | | 169 | CE2 | PHE | A | 57 | 24.113 | 28.092 | 5.359 | 1.00 | 12.06 |
| A | C | | | | | | | | | | |
| ATOM | | 170 | CZ | PHE | A | 57 | 23.862 | 27.259 | 6.438 | 1.00 | 13.80 |
| A | C | | | | | | | | | | |
| ATOM | | 171 | C | PHE | A | 57 | 23.936 | 25.003 | 0.398 | 1.00 | 13.58 |
| A | C | | | | | | | | | | |
| ATOM | | 172 | O | PHE | A | 57 | 23.483 | 25.952 | −0.244 | 1.00 | 13.09 |
| A | O | | | | | | | | | | |
| ATOM | | 173 | N | MET | A | 58 | 24.579 | 23.992 | −0.177 | 1.00 | 13.90 |
| A | N | | | | | | | | | | |
| ATOM | | 174 | CA | MET | A | 58 | 24.782 | 23.969 | −1.622 | 1.00 | 15.62 |
| A | C | | | | | | | | | | |
| ATOM | | 175 | CB | MET | A | 58 | 25.750 | 22.859 | −2.019 | 1.00 | 16.17 |
| A | C | | | | | | | | | | |
| ATOM | | 176 | CG | MET | A | 58 | 27.164 | 23.047 | −1.506 | 1.00 | 21.09 |
| A | C | | | | | | | | | | |
| ATOM | | 177 | SD | MET | A | 58 | 28.342 | 21.989 | −2.379 | 1.00 | 27.56 |
| A | S | | | | | | | | | | |
| ATOM | | 178 | CE | MET | A | 58 | 27.683 | 20.368 | −2.012 | 1.00 | 26.89 |
| A | C | | | | | | | | | | |
| ATOM | | 179 | C | MET | A | 58 | 23.454 | 23.759 | −2.337 | 1.00 | 13.03 |
| A | C | | | | | | | | | | |
| ATOM | | 180 | O | MET | A | 58 | 23.215 | 24.322 | −3.405 | 1.00 | 11.50 |
| A | O | | | | | | | | | | |
| ATOM | | 181 | N | LEU | A | 59 | 22.589 | 22.940 | −1.749 | 1.00 | 12.01 |
| A | N | | | | | | | | | | |
| ATOM | | 182 | CA | LEU | A | 59 | 21.286 | 22.696 | −2.347 | 1.00 | 12.06 |
| A | C | | | | | | | | | | |
| ATOM | | 183 | CB | LEU | A | 59 | 20.548 | 21.587 | −1.597 | 1.00 | 12.51 |
| A | C | | | | | | | | | | |
| ATOM | | 184 | CG | LEU | A | 59 | 19.085 | 21.390 | −2.012 | 1.00 | 13.80 |
| A | C | | | | | | | | | | |
| ATOM | | 185 | CD1 | LEU | A | 59 | 18.991 | 21.124 | −3.512 | 1.00 | 14.82 |
| A | C | | | | | | | | | | |
| ATOM | | 186 | CD2 | LEU | A | 59 | 18.486 | 20.243 | −1.225 | 1.00 | 10.84 |
| A | C | | | | | | | | | | |
| ATOM | | 187 | C | LEU | A | 59 | 20.474 | 23.990 | −2.307 | 1.00 | 14.49 |
| A | C | | | | | | | | | | |
| ATOM | | 188 | O | LEU | A | 59 | 19.769 | 24.325 | −3.262 | 1.00 | 13.13 |
| A | O | | | | | | | | | | |
| ATOM | | 189 | N | ALA | A | 60 | 20.578 | 24.718 | −1.195 | 1.00 | 14.81 |
| A | N | | | | | | | | | | |
| ATOM | | 190 | CA | ALA | A | 60 | 19.857 | 25.974 | −1.046 | 1.00 | 14.10 |
| A | C | | | | | | | | | | |
| ATOM | | 191 | CB | ALA | A | 60 | 20.077 | 26.545 | 0.344 | 1.00 | 14.30 |
| A | C | | | | | | | | | | |
| ATOM | | 192 | C | ALA | A | 60 | 20.339 | 26.963 | −2.100 | 1.00 | 15.08 |
| A | C | | | | | | | | | | |
| ATOM | | 193 | O | ALA | A | 60 | 19.537 | 27.621 | −2.761 | 1.00 | 12.89 |
| A | O | | | | | | | | | | |
| ATOM | | 194 | N | LYS | A | 61 | 21.654 | 27.065 | −2.263 | 1.00 | 14.92 |
| A | N | | | | | | | | | | |
| ATOM | | 195 | CA | LYS | A | 61 | 22.209 | 27.985 | −3.246 | 1.00 | 14.74 |
| A | C | | | | | | | | | | |
| ATOM | | 196 | CB | LYS | A | 61 | 23.740 | 27.926 | −3.227 | 1.00 | 16.05 |
| A | C | | | | | | | | | | |
| ATOM | | 197 | CG | LYS | A | 61 | 24.421 | 28.890 | −4.194 | 1.00 | 17.02 |
| A | C | | | | | | | | | | |
| ATOM | | 198 | CD | LYS | A | 61 | 24.047 | 30.325 | −3.891 | 1.00 | 19.86 |
| A | C | | | | | | | | | | |
| ATOM | | 199 | CE | LYS | A | 61 | 24.717 | 31.288 | −4.854 | 1.00 | 24.25 |
| A | C | | | | | | | | | | |
| ATOM | | 200 | NZ | LYS | A | 61 | 24.367 | 32.697 | −4.518 | 1.00 | 25.65 |
| A | N | | | | | | | | | | |
| ATOM | | 201 | C | LYS | A | 61 | 21.687 | 27.665 | −4.642 | 1.00 | 15.27 |
| A | C | | | | | | | | | | |
| ATOM | | 202 | O | LYS | A | 61 | 21.161 | 28.537 | −5.328 | 1.00 | 14.82 |
| A | O | | | | | | | | | | |
| ATOM | | 203 | N | GLU | A | 62 | 21.819 | 26.411 | −5.057 | 1.00 | 15.81 |
| A | N | | | | | | | | | | |
| ATOM | | 204 | CA | GLU | A | 62 | 21.353 | 26.001 | −6.373 | 1.00 | 18.42 |
| A | C | | | | | | | | | | |
| ATOM | | 205 | CB | GLU | A | 62 | 21.572 | 24.498 | −6.555 | 1.00 | 20.56 |
| A | C | | | | | | | | | | |
| ATOM | | 206 | CG | GLU | A | 62 | 21.338 | 24.016 | −7.969 | 1.00 | 27.23 |
| A | C | | | | | | | | | | |
| ATOM | | 207 | CD | GLU | A | 62 | 21.791 | 22.573 | −8.214 | 1.00 | 31.47 |
| A | C | | | | | | | | | | |
| ATOM | | 208 | OE1 | GLU | A | 62 | 22.875 | 22.136 | −7.748 | 1.00 | 32.95 |
| A | O | | | | | | | | | | |
| ATOM | | 209 | OE2 | GLU | A | 62 | 21.044 | 21.870 | −8.914 | 1.00 | 33.23 |
| A | O | | | | | | | | | | |
| ATOM | | 210 | C | GLU | A | 62 | 19.879 | 26.348 | −6.592 | 1.00 | 17.89 |
| A | C | | | | | | | | | | |
| ATOM | | 211 | O | GLU | A | 62 | 19.514 | 26.908 | −7.625 | 1.00 | 16.93 |
| A | O | | | | | | | | | | |
| ATOM | | 212 | N | ALA | A | 63 | 19.029 | 26.021 | −5.623 | 1.00 | 17.26 |
| A | N | | | | | | | | | | |
| ATOM | | 213 | CA | ALA | A | 63 | 17.605 | 26.316 | −5.752 | 1.00 | 16.93 |
| A | C | | | | | | | | | | |
| ATOM | | 214 | CB | ALA | A | 63 | 16.832 | 25.692 | −4.598 | 1.00 | 16.38 |
| A | C | | | | | | | | | | |
| ATOM | | 215 | C | ALA | A | 63 | 17.341 | 27.818 | −5.801 | 1.00 | 17.17 |
| A | C | | | | | | | | | | |
| ATOM | | 216 | O | ALA | A | 63 | 16.477 | 28.280 | −6.552 | 1.00 | 16.33 |
| A | O | | | | | | | | | | |
| ATOM | | 217 | N | SER | A | 64 | 18.079 | 28.586 | −5.006 | 1.00 | 15.83 |
| A | N | | | | | | | | | | |
| ATOM | | 218 | CA | SER | A | 64 | 17.876 | 30.026 | −4.992 | 1.00 | 18.51 |
| A | C | | | | | | | | | | |
| ATOM | | 219 | CB | SER | A | 64 | 18.748 | 30.686 | −3.918 | 1.00 | 19.42 |
| A | C | | | | | | | | | | |
| ATOM | | 220 | OG | SER | A | 64 | 20.118 | 30.643 | −4.267 | 1.00 | 21.79 |
| A | O | | | | | | | | | | |
| ATOM | | 221 | C | SER | A | 64 | 18.187 | 30.625 | −6.364 | 1.00 | 19.15 |
| A | C | | | | | | | | | | |
| ATOM | | 222 | O | SER | A | 64 | 17.632 | 31.659 | −6.739 | 1.00 | 18.21 |
| A | O | | | | | | | | | | |
| ATOM | | 223 | N | LEU | A | 65 | 19.067 | 29.966 | −7.116 | 1.00 | 20.27 |
| A | N | | | | | | | | | | |
| ATOM | | 224 | CA | LEU | A | 65 | 19.433 | 30.454 | −8.442 | 1.00 | 19.37 |
| A | C | | | | | | | | | | |
| ATOM | | 225 | CB | LEU | A | 65 | 20.727 | 29.797 | −8.924 | 1.00 | 19.96 |
| A | C | | | | | | | | | | |
| ATOM | | 226 | CG | LEU | A | 65 | 22.002 | 30.321 | −8.250 | 1.00 | 20.32 |
| A | C | | | | | | | | | | |
| ATOM | | 227 | CD1 | LEU | A | 65 | 23.174 | 29.425 | −8.604 | 1.00 | 21.39 |
| A | C | | | | | | | | | | |
| ATOM | | 228 | CD2 | LEU | A | 65 | 22.268 | 31.752 | −8.694 | 1.00 | 19.85 |
| A | C | | | | | | | | | | |
| ATOM | | 229 | C | LEU | A | 65 | 18.317 | 30.182 | −9.429 | 1.00 | 20.47 |
| A | C | | | | | | | | | | |
| ATOM | | 230 | O | LEU | A | 65 | 18.318 | 30.707 | −10.545 | 1.00 | 19.18 |
| A | O | | | | | | | | | | |
| ATOM | | 231 | N | ALA | A | 66 | 17.363 | 29.358 | −9.010 | 1.00 | 18.96 |
| A | N | | | | | | | | | | |
| ATOM | | 232 | CA | ALA | A | 66 | 16.231 | 29.022 | −9.859 | 1.00 | 20.12 |
| A | C | | | | | | | | | | |
| ATOM | | 233 | CB | ALA | A | 66 | 15.983 | 27.521 | −9.822 | 1.00 | 19.46 |
| A | C | | | | | | | | | | |
| ATOM | | 234 | C | ALA | A | 66 | 14.989 | 29.775 | −9.390 | 1.00 | 21.39 |
| A | C | | | | | | | | | | |
| ATOM | | 235 | O | ALA | A | 66 | 13.926 | 29.661 | −9.985 | 1.00 | 20.75 |
| A | O | | | | | | | | | | |
| ATOM | | 236 | N | ASP | A | 67 | 15.141 | 30.560 | −8.331 | 1.00 | 22.23 |
| A | N | | | | | | | | | | |
| ATOM | | 237 | CA | ASP | A | 67 | 14.036 | 31.320 | −7.761 | 1.00 | 24.31 |
| A | C | | | | | | | | | | |
| ATOM | | 238 | CB | ASP | A | 67 | 14.022 | 31.084 | −6.246 | 1.00 | 25.00 |
| A | C | | | | | | | | | | |
| ATOM | | 239 | CG | ASP | A | 67 | 13.134 | 32.055 | −5.497 | 1.00 | 24.66 |
| A | C | | | | | | | | | | |
| ATOM | | 240 | OD1 | ASP | A | 67 | 12.112 | 32.513 | −6.056 | 1.00 | 24.92 |
| A | O | | | | | | | | | | |
| ATOM | | 241 | OD2 | ASP | A | 67 | 13.465 | 32.341 | −4.328 | 1.00 | 21.55 |
| A | O | | | | | | | | | | |
| ATOM | | 242 | C | ASP | A | 67 | 14.116 | 32.813 | −8.083 | 1.00 | 25.59 |
| A | C | | | | | | | | | | |
| ATOM | | 243 | O | ASP | A | 67 | 15.012 | 33.511 | −7.609 | 1.00 | 25.98 |
| A | O | | | | | | | | | | |
| ATOM | | 244 | N | ASN | A | 68 | 13.172 | 33.297 | −8.889 | 1.00 | 27.63 |
| A | N | | | | | | | | | | |
| ATOM | | 245 | CA | ASN | A | 68 | 13.138 | 34.708 | −9.271 | 1.00 | 30.16 |
| A | C | | | | | | | | | | |
| ATOM | | 246 | CB | ASN | A | 68 | 12.696 | 34.857 | −10.731 | 1.00 | 32.04 |
| A | C | | | | | | | | | | |
| ATOM | | 247 | CG | ASN | A | 68 | 13.815 | 34.567 | −11.710 | 1.00 | 35.26 |
| A | C | | | | | | | | | | |
| ATOM | | 248 | OD1 | ASN | A | 68 | 14.891 | 35.160 | −11.627 | 1.00 | 37.18 |
| A | O | | | | | | | | | | |
| ATOM | | 249 | ND2 | ASN | A | 68 | 13.567 | 33.661 | −12.651 | 1.00 | 36.56 |
| A | N | | | | | | | | | | |
| ATOM | | 250 | C | ASN | A | 68 | 12.245 | 35.585 | −8.401 | 1.00 | 31.73 |
| A | C | | | | | | | | | | |
| ATOM | | 251 | O | ASN | A | 68 | 12.134 | 36.785 | −8.649 | 1.00 | 31.89 |
| A | O | | | | | | | | | | |
| ATOM | | 252 | N | ASN | A | 69 | 11.586 | 34.992 | −7.408 | 1.00 | 32.78 |
| A | N | | | | | | | | | | |
| ATOM | | 253 | CA | ASN | A | 69 | 10.698 | 35.744 | −6.519 | 1.00 | 35.19 |
| A | C | | | | | | | | | | |
| ATOM | | 254 | CB | ASN | A | 69 | 9.746 | 34.801 | −5.772 | 1.00 | 36.74 |
| A | C | | | | | | | | | | |
| ATOM | | 255 | CG | ASN | A | 69 | 9.056 | 33.805 | −6.688 | 1.00 | 40.93 |
| A | C | | | | | | | | | | |
| ATOM | | 256 | OD1 | ASN | A | 69 | 8.300 | 34.186 | −7.586 | 1.00 | 41.57 |
| A | O | | | | | | | | | | |
| ATOM | | 257 | ND2 | ASN | A | 69 | 9.311 | 32.515 | −6.461 | 1.00 | 40.68 |
| A | N | | | | | | | | | | |
| ATOM | | 258 | C | ASN | A | 69 | 11.542 | 36.510 | −5.501 | 1.00 | 35.09 |
| A | C | | | | | | | | | | |
| ATOM | | 259 | O | ASN | A | 69 | 11.646 | 36.109 | −4.339 | 1.00 | 32.29 |
| A | O | | | | | | | | | | |
| ATOM | | 260 | N | THR | A | 70 | 12.134 | 37.615 | −5.942 | 1.00 | 35.82 |
| A | N | | | | | | | | | | |
| ATOM | | 261 | CA | THR | A | 70 | 12.979 | 38.430 | −5.078 | 1.00 | 37.87 |
| A | C | | | | | | | | | | |
| ATOM | | 262 | CB | THR | A | 70 | 13.852 | 39.386 | −5.906 | 1.00 | 38.42 |
| A | C | | | | | | | | | | |
| ATOM | | 263 | OG1 | THR | A | 70 | 13.014 | 40.346 | −6.555 | 1.00 | 38.69 |
| A | O | | | | | | | | | | |
| ATOM | | 264 | CG2 | THR | A | 70 | 14.631 | 38.623 | −6.960 | 1.00 | 38.88 |
| A | C | | | | | | | | | | |
| ATOM | | 265 | C | THR | A | 70 | 12.192 | 39.275 | −4.072 | 1.00 | 38.60 |
| A | C | | | | | | | | | | |
| ATOM | | 266 | O | THR | A | 70 | 12.781 | 39.904 | −3.190 | 1.00 | 40.16 |
| A | O | | | | | | | | | | |
| ATOM | | 267 | N | ASP | A | 71 | 10.868 | 39.282 | −4.196 | 1.00 | 39.04 |
| A | N | | | | | | | | | | |
| ATOM | | 268 | CA | ASP | A | 71 | 10.017 | 40.069 | −3.305 | 1.00 | 39.32 |
| A | C | | | | | | | | | | |
| ATOM | | 269 | CB | ASP | A | 71 | 8.838 | 40.662 | −4.078 | 1.00 | 42.61 |
| A | C | | | | | | | | | | |
| ATOM | | 270 | CG | ASP | A | 71 | 9.276 | 41.445 | −5.281 | 1.00 | 47.01 |
| A | C | | | | | | | | | | |
| ATOM | | 271 | OD1 | ASP | A | 71 | 9.915 | 40.831 | −6.152 | 1.00 | 49.64 |
| A | O | | | | | | | | | | |
| ATOM | | 272 | OD2 | ASP | A | 71 | 8.991 | 42.661 | −5.362 | 1.00 | 50.35 |
| A | O | | | | | | | | | | |
| ATOM | | 273 | C | ASP | A | 71 | 9.451 | 39.301 | −2.119 | 1.00 | 37.56 |
| A | C | | | | | | | | | | |
| ATOM | | 274 | O | ASP | A | 71 | 8.886 | 39.906 | −1.206 | 1.00 | 37.36 |
| A | O | | | | | | | | | | |
| ATOM | | 275 | N | VAL | A | 72 | 9.590 | 37.979 | −2.128 | 1.00 | 33.02 |
| A | N | | | | | | | | | | |
| ATOM | | 276 | CA | VAL | A | 72 | 9.040 | 37.171 | −1.048 | 1.00 | 29.68 |
| A | C | | | | | | | | | | |
| ATOM | | 277 | CB | VAL | A | 72 | 8.003 | 36.165 | −1.590 | 1.00 | 31.44 |
| A | C | | | | | | | | | | |
| ATOM | | 278 | CG1 | VAL | A | 72 | 7.361 | 35.408 | −0.437 | 1.00 | 31.76 |
| A | C | | | | | | | | | | |
| ATOM | | 279 | CG2 | VAL | A | 72 | 6.951 | 36.899 | −2.416 | 1.00 | 34.37 |
| A | C | | | | | | | | | | |
| ATOM | | 280 | C | VAL | A | 72 | 10.074 | 36.392 | −0.252 | 1.00 | 26.18 |
| A | C | | | | | | | | | | |
| ATOM | | 281 | O | VAL | A | 72 | 11.011 | 35.825 | −0.814 | 1.00 | 24.53 |
| A | O | | | | | | | | | | |
| ATOM | | 282 | N | ARG | A | 73 | 9.879 | 36.372 | 1.061 | 1.00 | 24.81 |
| A | N | | | | | | | | | | |
| ATOM | | 283 | CA | ARG | A | 73 | 10.744 | 35.656 | 1.996 | 1.00 | 25.04 |
| A | C | | | | | | | | | | |
| ATOM | | 284 | CB | ARG | A | 73 | 11.426 | 36.640 | 2.951 | 1.00 | 27.84 |
| A | C | | | | | | | | | | |
| ATOM | | 285 | CG | ARG | A | 73 | 12.237 | 35.992 | 4.066 | 1.00 | 31.02 |
| A | C | | | | | | | | | | |
| ATOM | | 286 | CD | ARG | A | 73 | 13.488 | 35.315 | 3.527 | 1.00 | 33.29 |
| A | C | | | | | | | | | | |
| ATOM | | 287 | NE | ARG | A | 73 | 14.469 | 36.271 | 3.019 | 1.00 | 37.87 |
| A | N | | | | | | | | | | |
| ATOM | | 288 | CZ | ARG | A | 73 | 15.093 | 37.178 | 3.765 | 1.00 | 39.29 |
| A | C | | | | | | | | | | |
| ATOM | | 289 | NH1 | ARG | A | 73 | 14.847 | 37.266 | 5.066 | 1.00 | 39.10 |
| A | N | | | | | | | | | | |
| ATOM | | 290 | NH2 | ARG | A | 73 | 15.967 | 38.002 | 3.207 | 1.00 | 42.25 |
| A | N | | | | | | | | | | |
| ATOM | | 291 | C | ARG | A | 73 | 9.819 | 34.739 | 2.783 | 1.00 | 23.44 |
| A | C | | | | | | | | | | |
| ATOM | | 292 | O | ARG | A | 73 | 8.797 | 35.189 | 3.303 | 1.00 | 23.25 |
| A | O | | | | | | | | | | |
| ATOM | | 293 | N | LEU | A | 74 | 10.158 | 33.458 | 2.871 | 1.00 | 20.73 |
| A | N | | | | | | | | | | |
| ATOM | | 294 | CA | LEU | A | 74 | 9.311 | 32.522 | 3.602 | 1.00 | 18.25 |
| A | C | | | | | | | | | | |
| ATOM | | 295 | CB | LEU | A | 74 | 9.412 | 31.120 | 2.994 | 1.00 | 19.13 |
| A | C | | | | | | | | | | |
| ATOM | | 296 | CG | LEU | A | 74 | 8.937 | 30.973 | 1.546 | 1.00 | 19.57 |
| A | C | | | | | | | | | | |
| ATOM | | 297 | CD1 | LEU | A | 74 | 9.020 | 29.523 | 1.122 | 1.00 | 19.10 |
| A | C | | | | | | | | | | |
| ATOM | | 298 | CD2 | LEU | A | 74 | 7.504 | 31.462 | 1.420 | 1.00 | 19.94 |
| A | C | | | | | | | | | | |
| ATOM | | 299 | C | LEU | A | 74 | 9.655 | 32.466 | 5.081 | 1.00 | 18.81 |
| A | C | | | | | | | | | | |
| ATOM | | 300 | O | LEU | A | 74 | 8.772 | 32.562 | 5.930 | 1.00 | 17.72 |
| A | O | | | | | | | | | | |
| ATOM | | 301 | N | ILE | A | 75 | 10.940 | 32.307 | 5.391 | 1.00 | 17.09 |
| A | N | | | | | | | | | | |
| ATOM | | 302 | CA | ILE | A | 75 | 11.375 | 32.239 | 6.781 | 1.00 | 16.71 |
| A | C | | | | | | | | | | |
| ATOM | | 303 | CB | ILE | A | 75 | 12.503 | 31.195 | 6.948 | 1.00 | 17.07 |
| A | C | | | | | | | | | | |
| ATOM | | 304 | CG2 | ILE | A | 75 | 13.077 | 31.258 | 8.361 | 1.00 | 17.86 |
| A | C | | | | | | | | | | |
| ATOM | | 305 | CG1 | ILE | A | 75 | 11.950 | 29.798 | 6.642 | 1.00 | 18.78 |
| A | C | | | | | | | | | | |
| ATOM | | 306 | CD | ILE | A | 75 | 12.978 | 28.681 | 6.749 | 1.00 | 18.86 |
| A | C | | | | | | | | | | |
| ATOM | | 307 | C | ILE | A | 75 | 11.848 | 33.612 | 7.263 | 1.00 | 17.51 |
| A | C | | | | | | | | | | |
| ATOM | | 308 | O | ILE | A | 75 | 12.939 | 34.062 | 6.931 | 1.00 | 15.26 |
| A | O | | | | | | | | | | |
| ATOM | | 309 | N | GLY | A | 76 | 11.017 | 34.281 | 8.052 | 1.00 | 18.34 |
| A | N | | | | | | | | | | |
| ATOM | | 310 | CA | GLY | A | 76 | 11.389 | 35.600 | 8.530 | 1.00 | 21.35 |
| A | C | | | | | | | | | | |
| ATOM | | 311 | C | GLY | A | 76 | 10.608 | 36.007 | 9.761 | 1.00 | 23.15 |
| A | C | | | | | | | | | | |
| ATOM | | 312 | O | GLY | A | 76 | 9.916 | 35.187 | 10.357 | 1.00 | 21.36 |
| A | O | | | | | | | | | | |
| ATOM | | 313 | N | GLU | A | 77 | 10.706 | 37.282 | 10.125 | 1.00 | 26.14 |
| A | N | | | | | | | | | | |
| ATOM | | 314 | CA | GLU | A | 77 | 10.043 | 37.817 | 11.313 | 1.00 | 27.91 |
| A | C | | | | | | | | | | |
| ATOM | | 315 | CB | GLU | A | 77 | 10.118 | 39.349 | 11.300 | 1.00 | 31.26 |
| A | C | | | | | | | | | | |
| ATOM | | 316 | CG | GLU | A | 77 | 9.551 | 40.011 | 12.548 | 1.00 | 36.17 |
| A | C | | | | | | | | | | |
| ATOM | | 317 | CD | GLU | A | 77 | 9.807 | 41.508 | 12.584 | 1.00 | 39.76 |
| A | C | | | | | | | | | | |
| ATOM | | 318 | OE1 | GLU | A | 77 | 9.440 | 42.203 | 11.609 | 1.00 | 39.06 |
| A | O | | | | | | | | | | |
| ATOM | | 319 | OE2 | GLU | A | 77 | 10.375 | 41.986 | 13.591 | 1.00 | 41.22 |
| A | O | | | | | | | | | | |
| ATOM | | 320 | C | GLU | A | 77 | 8.595 | 37.370 | 11.517 | 1.00 | 27.70 |
| A | C | | | | | | | | | | |
| ATOM | | 321 | O | GLU | A | 77 | 8.235 | 36.899 | 12.595 | 1.00 | 28.81 |
| A | O | | | | | | | | | | |
| ATOM | | 322 | N | LYS | A | 78 | 7.767 | 37.514 | 10.491 | 1.00 | 27.14 |
| A | N | | | | | | | | | | |
| ATOM | | 323 | CA | LYS | A | 78 | 6.364 | 37.125 | 10.594 | 1.00 | 26.90 |
| A | C | | | | | | | | | | |
| ATOM | | 324 | CB | LYS | A | 78 | 5.652 | 37.351 | 9.254 | 1.00 | 29.37 |
| A | C | | | | | | | | | | |
| ATOM | | 325 | CG | LYS | A | 78 | 6.481 | 36.998 | 8.023 | 1.00 | 34.70 |
| A | C | | | | | | | | | | |
| ATOM | | 326 | CD | LYS | A | 78 | 7.597 | 38.018 | 7.773 | 1.00 | 35.71 |
| A | C | | | | | | | | | | |
| ATOM | | 327 | CE | LYS | A | 78 | 8.457 | 37.619 | 6.584 | 1.00 | 37.67 |
| A | C | | | | | | | | | | |
| ATOM | | 328 | NZ | LYS | A | 78 | 9.458 | 38.670 | 6.251 | 1.00 | 39.35 |
| A | N | | | | | | | | | | |
| ATOM | | 329 | C | LYS | A | 78 | 6.147 | 35.688 | 11.061 | 1.00 | 24.99 |
| A | C | | | | | | | | | | |
| ATOM | | 330 | O | LYS | A | 78 | 5.244 | 35.419 | 11.850 | 1.00 | 24.73 |
| A | O | | | | | | | | | | |
| ATOM | | 331 | N | LEU | A | 79 | 6.974 | 34.765 | 10.580 | 1.00 | 23.10 |
| A | N | | | | | | | | | | |
| ATOM | | 332 | CA | LEU | A | 79 | 6.851 | 33.360 | 10.954 | 1.00 | 21.97 |
| A | C | | | | | | | | | | |
| ATOM | | 333 | CB | LEU | A | 79 | 7.903 | 32.533 | 10.206 | 1.00 | 21.38 |
| A | C | | | | | | | | | | |
| ATOM | | 334 | CG | LEU | A | 79 | 7.963 | 31.034 | 10.510 | 1.00 | 19.79 |
| A | C | | | | | | | | | | |
| ATOM | | 335 | CD1 | LEU | A | 79 | 6.618 | 30.386 | 10.186 | 1.00 | 18.36 |
| A | C | | | | | | | | | | |
| ATOM | | 336 | CD2 | LEU | A | 79 | 9.076 | 30.395 | 9.688 | 1.00 | 18.38 |
| A | C | | | | | | | | | | |
| ATOM | | 337 | C | LEU | A | 79 | 7.005 | 33.126 | 12.457 | 1.00 | 22.48 |
| A | C | | | | | | | | | | |
| ATOM | | 338 | O | LEU | A | 79 | 6.378 | 32.232 | 13.026 | 1.00 | 21.54 |
| A | O | | | | | | | | | | |
| ATOM | | 339 | N | PHE | A | 80 | 7.839 | 33.936 | 13.098 | 1.00 | 24.35 |
| A | N | | | | | | | | | | |
| ATOM | | 340 | CA | PHE | A | 80 | 8.102 | 33.799 | 14.525 | 1.00 | 25.81 |
| A | C | | | | | | | | | | |
| ATOM | | 341 | CB | PHE | A | 80 | 9.599 | 33.972 | 14.768 | 1.00 | 24.41 |
| A | C | | | | | | | | | | |
| ATOM | | 342 | CG | PHE | A | 80 | 10.447 | 33.009 | 13.990 | 1.00 | 24.03 |
| A | C | | | | | | | | | | |
| ATOM | | 343 | CD1 | PHE | A | 80 | 10.510 | 31.667 | 14.351 | 1.00 | 23.62 |
| A | C | | | | | | | | | | |
| ATOM | | 344 | CD2 | PHE | A | 80 | 11.158 | 33.437 | 12.875 | 1.00 | 23.15 |
| A | C | | | | | | | | | | |
| ATOM | | 345 | CE1 | PHE | A | 80 | 11.270 | 30.759 | 13.606 | 1.00 | 23.39 |
| A | C | | | | | | | | | | |
| ATOM | | 346 | CE2 | PHE | A | 80 | 11.917 | 32.543 | 12.125 | 1.00 | 23.09 |
| A | C | | | | | | | | | | |
| ATOM | | 347 | CZ | PHE | A | 80 | 11.974 | 31.200 | 12.491 | 1.00 | 23.28 |
| A | C | | | | | | | | | | |
| ATOM | | 348 | C | PHE | A | 80 | 7.324 | 34.775 | 15.410 | 1.00 | 28.55 |
| A | C | | | | | | | | | | |
| ATOM | | 349 | O | PHE | A | 80 | 7.362 | 34.676 | 16.637 | 1.00 | 28.74 |
| A | O | | | | | | | | | | |
| ATOM | | 350 | N | HIS | A | 81 | 6.618 | 35.711 | 14.787 | 1.00 | 30.56 |
| A | N | | | | | | | | | | |
| ATOM | | 351 | CA | HIS | A | 81 | 5.849 | 36.711 | 15.519 | 1.00 | 32.02 |
| A | C | | | | | | | | | | |
| ATOM | | 352 | CE | HIS | A | 81 | 5.051 | 37.579 | 14.540 | 1.00 | 34.06 |
| A | C | | | | | | | | | | |
| ATOM | | 353 | CG | HIS | A | 81 | 4.321 | 38.711 | 15.193 | 1.00 | 36.89 |
| A | C | | | | | | | | | | |
| ATOM | | 354 | CD2 | HIS | A | 81 | 2.996 | 38.959 | 15.325 | 1.00 | 37.63 |
| A | C | | | | | | | | | | |
| ATOM | | 355 | ND1 | HIS | A | 81 | 4.971 | 39.748 | 15.829 | 1.00 | 38.12 |
| A | N | | | | | | | | | | |
| ATOM | | 356 | CE1 | HIS | A | 81 | 4.078 | 40.585 | 16.325 | 1.00 | 38.01 |
| A | C | | | | | | | | | | |
| ATOM | | 357 | NE2 | HIS | A | 81 | 2.873 | 40.129 | 16.033 | 1.00 | 38.42 |
| A | N | | | | | | | | | | |
| ATOM | | 358 | C | HIS | A | 81 | 4.902 | 36.097 | 16.547 | 1.00 | 30.87 |
| A | C | | | | | | | | | | |
| ATOM | | 359 | O | HIS | A | 81 | 4.060 | 35.268 | 16.211 | 1.00 | 32.80 |
| A | O | | | | | | | | | | |
| ATOM | | 360 | N | GLY | A | 82 | 5.046 | 36.511 | 17.802 | 1.00 | 30.56 |
| A | N | | | | | | | | | | |
| ATOM | | 361 | CA | GLY | A | 82 | 4.192 | 35.997 | 18.859 | 1.00 | 29.32 |
| A | C | | | | | | | | | | |
| ATOM | | 362 | C | GLY | A | 82 | 4.593 | 34.640 | 19.420 | 1.00 | 28.91 |
| A | C | | | | | | | | | | |
| ATOM | | 363 | O | GLY | A | 82 | 3.989 | 34.165 | 20.380 | 1.00 | 29.61 |
| A | O | | | | | | | | | | |
| ATOM | | 364 | N | VAL | A | 83 | 5.609 | 34.011 | 18.835 | 1.00 | 26.58 |
| A | N | | | | | | | | | | |
| ATOM | | 365 | CA | VAL | A | 83 | 6.064 | 32.697 | 19.295 | 1.00 | 24.46 |
| A | C | | | | | | | | | | |
| ATOM | | 366 | CB | VAL | A | 83 | 6.569 | 31.855 | 18.096 | 1.00 | 24.84 |
| A | C | | | | | | | | | | |
| ATOM | | 367 | CG1 | VAL | A | 83 | 6.971 | 30.470 | 18.531 | 1.00 | 24.49 |
| A | C | | | | | | | | | | |
| ATOM | | 368 | CG2 | VAL | A | 83 | 5.480 | 31.749 | 17.066 | 1.00 | 24.95 |
| A | C | | | | | | | | | | |
| ATOM | | 369 | C | VAL | A | 83 | 7.180 | 32.847 | 20.328 | 1.00 | 23.20 |
| A | C | | | | | | | | | | |
| ATOM | | 370 | O | VAL | A | 83 | 8.218 | 33.429 | 20.043 | 1.00 | 20.58 |
| A | O | | | | | | | | | | |
| ATOM | | 371 | N | SER | A | 84 | 6.956 | 32.336 | 21.535 | 1.00 | 22.74 |
| A | N | | | | | | | | | | |
| ATOM | | 372 | CA | SER | A | 84 | 7.961 | 32.427 | 22.586 | 1.00 | 22.59 |
| A | C | | | | | | | | | | |
| ATOM | | 373 | CB | SER | A | 84 | 7.325 | 32.136 | 23.942 | 1.00 | 24.87 |
| A | C | | | | | | | | | | |
| ATOM | | 374 | OG | SER | A | 84 | 6.740 | 30.847 | 23.960 | 1.00 | 28.28 |
| A | O | | | | | | | | | | |
| ATOM | | 375 | C | SER | A | 84 | 9.091 | 31.438 | 22.319 | 1.00 | 22.22 |
| A | C | | | | | | | | | | |
| ATOM | | 376 | O | SER | A | 84 | 8.891 | 30.428 | 21.649 | 1.00 | 19.25 |
| A | O | | | | | | | | | | |
| ATOM | | 377 | N | MET | A | 85 | 10.271 | 31.723 | 22.860 | 1.00 | 22.89 |
| A | N | | | | | | | | | | |
| ATOM | | 378 | CA | MET | A | 85 | 11.438 | 30.864 | 22.661 | 1.00 | 23.51 |
| A | C | | | | | | | | | | |
| ATOM | | 379 | CB | MET | A | 85 | 12.626 | 31.391 | 23.470 | 1.00 | 26.40 |
| A | C | | | | | | | | | | |
| ATOM | | 380 | CG | MET | A | 85 | 12.965 | 32.850 | 23.205 | 1.00 | 32.22 |
| A | C | | | | | | | | | | |
| ATOM | | 381 | SD | MET | A | 85 | 13.130 | 33.234 | 21.443 | 1.00 | 38.24 |
| A | S | | | | | | | | | | |
| ATOM | | 382 | CE | MET | A | 85 | 11.634 | 34.156 | 21.178 | 1.00 | 39.11 |
| A | C | | | | | | | | | | |
| ATOM | | 383 | C | MET | A | 85 | 11.213 | 29.391 | 23.010 | 1.00 | 21.82 |
| A | C | | | | | | | | | | |
| ATOM | | 384 | O | MET | A | 85 | 11.799 | 28.506 | 22.387 | 1.00 | 21.96 |
| A | O | | | | | | | | | | |
| ATOM | | 385 | N | SER | A | 86 | 10.377 | 29.121 | 24.006 | 1.00 | 20.05 |
| A | N | | | | | | | | | | |
| ATOM | | 386 | CA | SER | A | 86 | 10.123 | 27.743 | 24.405 | 1.00 | 20.16 |
| A | C | | | | | | | | | | |
| ATOM | | 387 | CB | SER | A | 86 | 9.504 | 27.701 | 25.801 | 1.00 | 20.31 |
| A | C | | | | | | | | | | |
| ATOM | | 388 | OG | SER | A | 86 | 8.281 | 28.406 | 25.816 | 1.00 | 19.38 |
| A | O | | | | | | | | | | |
| ATOM | | 389 | C | SER | A | 86 | 9.215 | 26.993 | 23.436 | 1.00 | 20.16 |
| A | C | | | | | | | | | | |
| ATOM | | 390 | O | SER | A | 86 | 9.039 | 25.780 | 23.563 | 1.00 | 21.23 |
| A | O | | | | | | | | | | |
| ATOM | | 391 | N | GLU | A | 87 | 8.633 | 27.702 | 22.475 | 1.00 | 18.55 |
| A | N | | | | | | | | | | |
| ATOM | | 392 | CA | GLU | A | 87 | 7.757 | 27.044 | 21.505 | 1.00 | 19.19 |
| A | C | | | | | | | | | | |
| ATOM | | 393 | CB | GLU | A | 87 | 6.381 | 27.717 | 21.480 | 1.00 | 21.48 |
| A | C | | | | | | | | | | |
| ATOM | | 394 | CG | GLU | A | 87 | 5.848 | 28.071 | 22.849 | 1.00 | 27.63 |
| A | C | | | | | | | | | | |
| ATOM | | 395 | CD | GLU | A | 87 | 4.583 | 28.925 | 22.796 | 1.00 | 30.13 |
| A | C | | | | | | | | | | |
| ATOM | | 396 | OE1 | GLU | A | 87 | 4.609 | 30.033 | 22.220 | 1.00 | 30.21 |
| A | O | | | | | | | | | | |
| ATOM | | 397 | OE2 | GLU | A | 87 | 3.553 | 28.497 | 23.344 | 1.00 | 33.94 |
| A | O | | | | | | | | | | |
| ATOM | | 398 | C | GLU | A | 87 | 8.337 | 27.066 | 20.095 | 1.00 | 17.25 |
| A | C | | | | | | | | | | |
| ATOM | | 399 | O | GLU | A | 87 | 7.786 | 26.443 | 19.189 | 1.00 | 15.95 |
| A | O | | | | | | | | | | |
| ATOM | | 400 | N | ARG | A | 88 | 9.447 | 27.778 | 19.910 | 1.00 | 16.02 |
| A | N | | | | | | | | | | |
| ATOM | | 401 | CA | ARG | A | 88 | 10.081 | 27.887 | 18.599 | 1.00 | 15.63 |
| A | C | | | | | | | | | | |
| ATOM | | 402 | CB | ARG | A | 88 | 11.356 | 28.729 | 18.693 | 1.00 | 18.69 |
| A | C | | | | | | | | | | |
| ATOM | | 403 | CG | ARG | A | 88 | 11.146 | 30.190 | 19.067 | 1.00 | 23.67 |
| A | C | | | | | | | | | | |
| ATOM | | 404 | CD | ARG | A | 88 | 10.457 | 30.944 | 17.961 | 1.00 | 26.36 |
| A | C | | | | | | | | | | |
| ATOM | | 405 | NE | ARG | A | 88 | 10.175 | 32.337 | 18.311 | 1.00 | 30.28 |
| A | N | | | | | | | | | | |
| ATOM | | 406 | CZ | ARG | A | 88 | 11.044 | 33.337 | 18.206 | 1.00 | 31.77 |
| A | C | | | | | | | | | | |
| ATOM | | 407 | NH1 | ARG | A | 88 | 12.270 | 33.111 | 17.762 | 1.00 | 34.62 |
| A | N | | | | | | | | | | |
| ATOM | | 408 | NH2 | ARG | A | 88 | 10.678 | 34.574 | 18.518 | 1.00 | 30.69 |
| A | N | | | | | | | | | | |
| ATOM | | 409 | C | ARG | A | 88 | 10.423 | 26.541 | 17.951 | 1.00 | 15.43 |
| A | C | | | | | | | | | | |
| ATOM | | 410 | O | ARG | A | 88 | 10.153 | 26.333 | 16.767 | 1.00 | 15.17 |
| A | O | | | | | | | | | | |
| ATOM | | 411 | N | CYS | A | 89 | 11.025 | 25.628 | 18.708 | 1.00 | 12.14 |
| A | N | | | | | | | | | | |
| ATOM | | 412 | CA | CYS | A | 89 | 11.381 | 24.342 | 18.120 | 1.00 | 13.07 |
| A | C | | | | | | | | | | |
| ATOM | | 413 | C | CYS | A | 89 | 10.133 | 23.605 | 17.611 | 1.00 | 14.78 |
| A | C | | | | | | | | | | |
| ATOM | | 414 | O | CYS | A | 89 | 10.158 | 23.020 | 16.528 | 1.00 | 13.46 |
| A | O | | | | | | | | | | |
| ATOM | | 415 | CB | CYS | A | 89 | 12.169 | 23.472 | 19.107 | 1.00 | 12.57 |
| A | C | | | | | | | | | | |
| ATOM | | 416 | SG | CYS | A | 89 | 12.780 | 21.964 | 18.306 | 1.00 | 15.35 |
| A | S | | | | | | | | | | |
| ATOM | | 417 | N | TYR | A | 90 | 9.044 | 23.653 | 18.378 | 1.00 | 13.52 |
| A | N | | | | | | | | | | |
| ATOM | | 418 | CA | TYR | A | 90 | 7.789 | 23.017 | 17.970 | 1.00 | 14.36 |
| A | C | | | | | | | | | | |
| ATOM | | 419 | CB | TYR | A | 90 | 6.729 | 23.149 | 19.063 | 1.00 | 16.25 |
| A | C | | | | | | | | | | |
| ATOM | | 420 | CG | TYR | A | 90 | 5.386 | 22.598 | 18.640 | 1.00 | 20.73 |
| A | C | | | | | | | | | | |
| ATOM | | 421 | CD1 | TYR | A | 90 | 5.172 | 21.220 | 18.551 | 1.00 | 22.84 |
| A | C | | | | | | | | | | |
| ATOM | | 422 | CE1 | TYR | A | 90 | 3.936 | 20.704 | 18.142 | 1.00 | 25.51 |
| A | C | | | | | | | | | | |
| ATOM | | 423 | CD2 | TYR | A | 90 | 4.334 | 23.453 | 18.308 | 1.00 | 21.97 |
| A | C | | | | | | | | | | |
| ATOM | | 424 | CE2 | TYR | A | 90 | 3.095 | 22.947 | 17.894 | 1.00 | 25.27 |
| A | C | | | | | | | | | | |
| ATOM | | 425 | CZ | TYR | A | 90 | 2.908 | 21.575 | 17.813 | 1.00 | 26.64 |
| A | C | | | | | | | | | | |
| ATOM | | 426 | OH | TYR | A | 90 | 1.698 | 21.073 | 17.393 | 1.00 | 31.95 |
| A | O | | | | | | | | | | |
| ATOM | | 427 | C | TYR | A | 90 | 7.283 | 23.691 | 16.699 | 1.00 | 12.19 |
| A | C | | | | | | | | | | |
| ATOM | | 428 | O | TYR | A | 90 | 6.762 | 23.032 | 15.797 | 1.00 | 13.82 |
| A | O | | | | | | | | | | |
| ATOM | | 429 | N | LEU | A | 91 | 7.430 | 25.011 | 16.630 | 1.00 | 13.87 |
| A | N | | | | | | | | | | |
| ATOM | | 430 | CA | LEU | A | 91 | 7.020 | 25.763 | 15.445 | 1.00 | 14.25 |
| A | C | | | | | | | | | | |
| ATOM | | 431 | CB | LEU | A | 91 | 7.279 | 27.259 | 15.657 | 1.00 | 16.00 |
| A | C | | | | | | | | | | |
| ATOM | | 432 | CG | LEU | A | 91 | 7.389 | 28.125 | 14.403 | 1.00 | 20.38 |
| A | C | | | | | | | | | | |
| ATOM | | 433 | CD1 | LEU | A | 91 | 6.049 | 28.180 | 13.691 | 1.00 | 20.90 |
| A | C | | | | | | | | | | |
| ATOM | | 434 | CD2 | LEU | A | 91 | 7.848 | 29.522 | 14.795 | 1.00 | 23.19 |
| A | C | | | | | | | | | | |
| ATOM | | 435 | C | LEU | A | 91 | 7.841 | 25.271 | 14.250 | 1.00 | 14.26 |
| A | C | | | | | | | | | | |
| ATOM | | 436 | O | LEU | A | 91 | 7.298 | 25.011 | 13.166 | 1.00 | 13.95 |
| A | O | | | | | | | | | | |
| ATOM | | 437 | N | MET | A | 92 | 9.156 | 25.144 | 14.454 | 1.00 | 13.32 |
| A | N | | | | | | | | | | |
| ATOM | | 438 | CA | MET | A | 92 | 10.054 | 24.693 | 13.386 | 1.00 | 12.34 |
| A | C | | | | | | | | | | |
| ATOM | | 439 | CB | MET | A | 92 | 11.520 | 24.855 | 13.791 | 1.00 | 13.46 |
| A | C | | | | | | | | | | |
| ATOM | | 440 | CG | MET | A | 92 | 11.943 | 26.329 | 13.855 | 1.00 | 13.93 |
| A | C | | | | | | | | | | |
| ATOM | | 441 | SD | MET | A | 92 | 11.513 | 27.272 | 12.365 | 1.00 | 19.44 |
| A | S | | | | | | | | | | |
| ATOM | | 442 | CE | MET | A | 92 | 12.398 | 26.314 | 11.091 | 1.00 | 16.94 |
| A | C | | | | | | | | | | |
| ATOM | | 443 | C | MET | A | 92 | 9.763 | 23.272 | 12.941 | 1.00 | 13.49 |
| A | C | | | | | | | | | | |
| ATOM | | 444 | O | MET | A | 92 | 9.975 | 22.924 | 11.777 | 1.00 | 14.17 |
| A | O | | | | | | | | | | |
| ATOM | | 445 | N | LYS | A | 93 | 9.255 | 22.465 | 13.864 | 1.00 | 12.98 |
| A | N | | | | | | | | | | |
| ATOM | | 446 | CA | LYS | A | 93 | 8.889 | 21.097 | 13.557 | 1.00 | 13.82 |
| A | C | | | | | | | | | | |
| ATOM | | 447 | CB | LYS | A | 93 | 8.385 | 20.395 | 14.810 | 1.00 | 13.12 |
| A | C | | | | | | | | | | |
| ATOM | | 448 | CG | LYS | A | 93 | 7.672 | 19.082 | 14.522 | 1.00 | 18.61 |
| A | C | | | | | | | | | | |
| ATOM | | 449 | CD | LYS | A | 93 | 6.889 | 18.604 | 15.736 | 1.00 | 18.28 |
| A | C | | | | | | | | | | |
| ATOM | | 450 | CE | LYS | A | 93 | 6.085 | 17.352 | 15.441 | 1.00 | 20.53 |
| A | C | | | | | | | | | | |
| ATOM | | 451 | NZ | LYS | A | 93 | 5.352 | 16.889 | 16.657 | 1.00 | 19.33 |
| A | N | | | | | | | | | | |
| ATOM | | 452 | C | LYS | A | 93 | 7.770 | 21.124 | 12.526 | 1.00 | 13.87 |
| A | C | | | | | | | | | | |
| ATOM | | 453 | O | LYS | A | 93 | 7.760 | 20.328 | 11.593 | 1.00 | 15.68 |
| A | O | | | | | | | | | | |
| ATOM | | 454 | N | GLN | A | 94 | 6.823 | 22.043 | 12.700 | 1.00 | 15.16 |
| A | N | | | | | | | | | | |
| ATOM | | 455 | CA | GLN | A | 94 | 5.692 | 22.144 | 11.778 | 1.00 | 15.15 |
| A | C | | | | | | | | | | |
| ATOM | | 456 | CB | GLN | A | 94 | 4.630 | 23.089 | 12.344 | 1.00 | 17.59 |
| A | C | | | | | | | | | | |
| ATOM | | 457 | CG | GLN | A | 94 | 4.254 | 22.779 | 13.786 | 1.00 | 23.02 |
| A | C | | | | | | | | | | |
| ATOM | | 458 | CD | GLN | A | 94 | 3.713 | 21.376 | 13.964 | 1.00 | 25.41 |
| A | C | | | | | | | | | | |
| ATOM | | 459 | OE1 | GLN | A | 94 | 3.829 | 20.788 | 15.038 | 1.00 | 31.14 |
| A | O | | | | | | | | | | |
| ATOM | | 460 | NE2 | GLN | A | 94 | 3.110 | 20.836 | 12.917 | 1.00 | 25.01 |
| A | N | | | | | | | | | | |
| ATOM | | 461 | C | GLN | A | 94 | 6.157 | 22.644 | 10.419 | 1.00 | 13.97 |
| A | C | | | | | | | | | | |
| ATOM | | 462 | O | GLN | A | 94 | 5.687 | 22.179 | 9.378 | 1.00 | 13.38 |
| A | O | | | | | | | | | | |
| ATOM | | 463 | N | VAL | A | 95 | 7.082 | 23.599 | 10.429 | 1.00 | 12.42 |
| A | N | | | | | | | | | | |
| ATOM | | 464 | CA | VAL | A | 95 | 7.598 | 24.140 | 9.183 | 1.00 | 11.71 |
| A | C | | | | | | | | | | |
| ATOM | | 465 | CB | VAL | A | 95 | 8.539 | 25.324 | 9.420 | 1.00 | 12.14 |
| A | C | | | | | | | | | | |
| ATOM | | 466 | CG1 | VAL | A | 95 | 9.180 | 25.740 | 8.107 | 1.00 | 13.43 |
| A | C | | | | | | | | | | |
| ATOM | | 467 | CG2 | VAL | A | 95 | 7.767 | 26.483 | 9.997 | 1.00 | 12.64 |
| A | C | | | | | | | | | | |
| ATOM | | 468 | C | VAL | A | 95 | 8.373 | 23.060 | 8.452 | 1.00 | 12.55 |
| A | C | | | | | | | | | | |
| ATOM | | 469 | O | VAL | A | 95 | 8.269 | 22.930 | 7.232 | 1.00 | 10.70 |
| A | O | | | | | | | | | | |
| ATOM | | 470 | N | LEU | A | 96 | 9.150 | 22.287 | 9.210 | 1.00 | 11.35 |
| A | N | | | | | | | | | | |
| ATOM | | 471 | CA | LEU | A | 96 | 9.953 | 21.212 | 8.639 | 1.00 | 9.86 |
| A | C | | | | | | | | | | |
| ATOM | | 472 | CB | LEU | A | 96 | 10.822 | 20.559 | 9.712 | 1.00 | 10.52 |
| A | C | | | | | | | | | | |
| ATOM | | 473 | CG | LEU | A | 96 | 11.572 | 19.302 | 9.248 | 1.00 | 12.76 |
| A | C | | | | | | | | | | |
| ATOM | | 474 | CD1 | LEU | A | 96 | 12.575 | 19.657 | 8.158 | 1.00 | 11.47 |
| A | C | | | | | | | | | | |
| ATOM | | 475 | CD2 | LEU | A | 96 | 12.282 | 18.662 | 10.445 | 1.00 | 11.92 |
| A | C | | | | | | | | | | |
| ATOM | | 476 | C | LEU | A | 96 | 9.088 | 20.145 | 7.994 | 1.00 | 10.33 |
| A | C | | | | | | | | | | |
| ATOM | | 477 | O | LEU | A | 96 | 9.327 | 19.751 | 6.855 | 1.00 | 10.88 |
| A | O | | | | | | | | | | |
| ATOM | | 478 | N | ASN | A | 97 | 8.086 | 19.669 | 8.725 | 1.00 | 11.10 |
| A | N | | | | | | | | | | |
| ATOM | | 479 | CA | ASN | A | 97 | 7.215 | 18.630 | 8.192 | 1.00 | 12.58 |
| A | C | | | | | | | | | | |
| ATOM | | 480 | CB | ASN | A | 97 | 6.270 | 18.130 | 9.290 | 1.00 | 15.76 |
| A | C | | | | | | | | | | |
| ATOM | | 481 | CG | ASN | A | 97 | 7.013 | 17.361 | 10.377 | 1.00 | 16.70 |
| A | C | | | | | | | | | | |
| ATOM | | 482 | OD1 | ASN | A | 97 | 8.146 | 16.920 | 10.168 | 1.00 | 19.35 |
| A | O | | | | | | | | | | |
| ATOM | | 483 | ND2 | ASN | A | 97 | 6.380 | 17.182 | 11.525 | 1.00 | 17.01 |
| A | N | | | | | | | | | | |
| ATOM | | 484 | C | ASN | A | 97 | 6.462 | 19.114 | 6.964 | 1.00 | 11.81 |
| A | C | | | | | | | | | | |
| ATOM | | 485 | O | ASN | A | 97 | 6.281 | 18.361 | 6.006 | 1.00 | 12.10 |
| A | O | | | | | | | | | | |
| ATOM | | 486 | N | PHE | A | 98 | 6.037 | 20.374 | 6.971 | 1.00 | 12.85 |
| A | N | | | | | | | | | | |
| ATOM | | 487 | CA | PHE | A | 98 | 5.347 | 20.917 | 5.806 | 1.00 | 13.45 |
| A | C | | | | | | | | | | |
| ATOM | | 488 | CB | PHE | A | 98 | 4.907 | 22.357 | 6.052 | 1.00 | 14.09 |
| A | C | | | | | | | | | | |
| ATOM | | 489 | CG | PHE | A | 98 | 4.623 | 23.121 | 4.788 | 1.00 | 17.24 |
| A | C | | | | | | | | | | |
| ATOM | | 490 | CD1 | PHE | A | 98 | 3.535 | 22.793 | 3.986 | 1.00 | 18.81 |
| A | C | | | | | | | | | | |
| ATOM | | 491 | CD2 | PHE | A | 98 | 5.472 | 24.141 | 4.378 | 1.00 | 18.95 |
| A | C | | | | | | | | | | |
| ATOM | | 492 | CE1 | PHE | A | 98 | 3.301 | 23.469 | 2.790 | 1.00 | 19.48 |
| A | C | | | | | | | | | | |
| ATOM | | 493 | CE2 | PHE | A | 98 | 5.247 | 24.820 | 3.188 | 1.00 | 18.82 |
| A | C | | | | | | | | | | |
| ATOM | | 494 | CZ | PHE | A | 98 | 4.163 | 24.484 | 2.392 | 1.00 | 18.93 |
| A | C | | | | | | | | | | |
| ATOM | | 495 | C | PHE | A | 98 | 6.316 | 20.906 | 4.629 | 1.00 | 13.98 |
| A | C | | | | | | | | | | |
| ATOM | | 496 | O | PHE | A | 98 | 5.991 | 20.458 | 3.519 | 1.00 | 12.54 |
| A | O | | | | | | | | | | |
| ATOM | | 497 | N | THR | A | 99 | 7.519 | 21.405 | 4.878 | 1.00 | 12.46 |
| A | N | | | | | | | | | | |
| ATOM | | 498 | CA | THR | A | 99 | 8.509 | 21.473 | 3.818 | 1.00 | 12.12 |
| A | C | | | | | | | | | | |
| ATOM | | 499 | CB | THR | A | 99 | 9.789 | 22.174 | 4.297 | 1.00 | 13.25 |
| A | C | | | | | | | | | | |
| ATOM | | 500 | OG1 | THR | A | 99 | 9.459 | 23.469 | 4.825 | 1.00 | 11.71 |
| A | O | | | | | | | | | | |
| ATOM | | 501 | CG2 | THR | A | 99 | 10.745 | 22.345 | 3.137 | 1.00 | 12.11 |
| A | C | | | | | | | | | | |
| ATOM | | 502 | C | THR | A | 99 | 8.852 | 20.099 | 3.260 | 1.00 | 11.68 |
| A | C | | | | | | | | | | |
| ATOM | | 503 | O | THR | A | 99 | 9.081 | 19.950 | 2.061 | 1.00 | 12.57 |
| A | O | | | | | | | | | | |
| ATOM | | 504 | N | LEU | A | 100 | 8.892 | 19.092 | 4.124 | 1.00 | 12.47 |
| A | N | | | | | | | | | | |
| ATOM | | 505 | CA | LEU | A | 100 | 9.189 | 17.741 | 3.668 | 1.00 | 13.58 |
| A | C | | | | | | | | | | |
| ATOM | | 506 | CB | LEU | A | 100 | 9.405 | 16.817 | 4.870 | 1.00 | 13.64 |
| A | C | | | | | | | | | | |
| ATOM | | 507 | CG | LEU | A | 100 | 10.794 | 16.844 | 5.519 | 1.00 | 14.13 |
| A | C | | | | | | | | | | |
| ATOM | | 508 | CD1 | LEU | A | 100 | 10.766 | 16.117 | 6.859 | 1.00 | 15.07 |
| A | C | | | | | | | | | | |
| ATOM | | 509 | CD2 | LEU | A | 100 | 11.788 | 16.191 | 4.583 | 1.00 | 11.06 |
| A | C | | | | | | | | | | |
| ATOM | | 510 | C | LEU | A | 100 | 8.056 | 17.179 | 2.802 | 1.00 | 15.63 |
| A | C | | | | | | | | | | |
| ATOM | | 511 | O | LEU | A | 100 | 8.285 | 16.726 | 1.684 | 1.00 | 14.71 |
| A | O | | | | | | | | | | |
| ATOM | | 512 | N | GLU | A | 101 | 6.838 | 17.223 | 3.332 | 1.00 | 15.43 |
| A | N | | | | | | | | | | |
| ATOM | | 513 | CA | GLU | A | 101 | 5.660 | 16.687 | 2.656 | 1.00 | 17.18 |
| A | C | | | | | | | | | | |
| ATOM | | 514 | CB | GLU | A | 101 | 4.467 | 16.670 | 3.618 | 1.00 | 18.69 |
| A | C | | | | | | | | | | |
| ATOM | | 515 | CG | GLU | A | 101 | 4.530 | 15.627 | 4.730 | 1.00 | 22.02 |
| A | C | | | | | | | | | | |
| ATOM | | 516 | CD | GLU | A | 101 | 4.465 | 14.207 | 4.212 | 1.00 | 26.32 |
| A | C | | | | | | | | | | |
| ATOM | | 517 | OE1 | GLU | A | 101 | 3.657 | 13.940 | 3.299 | 1.00 | 27.27 |
| A | O | | | | | | | | | | |
| ATOM | | 518 | OE2 | GLU | A | 101 | 5.212 | 13.344 | 4.724 | 1.00 | 29.94 |
| A | O | | | | | | | | | | |
| ATOM | | 519 | C | GLU | A | 101 | 5.220 | 17.375 | 1.370 | 1.00 | 17.32 |
| A | C | | | | | | | | | | |
| ATOM | | 520 | O | GLU | A | 101 | 4.913 | 16.706 | 0.378 | 1.00 | 17.41 |
| A | O | | | | | | | | | | |
| ATOM | | 521 | N | GLU | A | 102 | 5.194 | 18.702 | 1.378 | 1.00 | 16.65 |
| A | N | | | | | | | | | | |
| ATOM | | 522 | CA | GLU | A | 102 | 4.725 | 19.442 | 0.212 | 1.00 | 16.41 |
| A | C | | | | | | | | | | |
| ATOM | | 523 | CB | GLU | A | 102 | 3.629 | 20.402 | 0.657 | 1.00 | 19.20 |
| A | C | | | | | | | | | | |
| ATOM | | 524 | CG | GLU | A | 102 | 2.440 | 19.644 | 1.233 | 1.00 | 23.30 |
| A | C | | | | | | | | | | |
| ATOM | | 525 | CD | GLU | A | 102 | 1.369 | 20.554 | 1.771 | 1.00 | 26.38 |
| A | C | | | | | | | | | | |
| ATOM | | 526 | OE1 | GLU | A | 102 | 0.828 | 21.357 | 0.981 | 1.00 | 25.51 |
| A | O | | | | | | | | | | |
| ATOM | | 527 | OE2 | GLU | A | 102 | 1.073 | 20.465 | 2.984 | 1.00 | 27.95 |
| A | O | | | | | | | | | | |
| ATOM | | 528 | C | GLU | A | 102 | 5.745 | 20.170 | −0.650 | 1.00 | 16.28 |
| A | C | | | | | | | | | | |
| ATOM | | 529 | O | GLU | A | 102 | 5.385 | 20.783 | −1.653 | 1.00 | 14.86 |
| A | O | | | | | | | | | | |
| ATOM | | 530 | N | VAL | A | 103 | 7.015 | 20.109 | −0.271 | 1.00 | 12.43 |
| A | N | | | | | | | | | | |
| ATOM | | 531 | CA | VAL | A | 103 | 8.045 | 20.755 | −1.077 | 1.00 | 13.07 |
| A | C | | | | | | | | | | |
| ATOM | | 532 | CB | VAL | A | 103 | 8.742 | 21.907 | −0.320 | 1.00 | 12.76 |
| A | C | | | | | | | | | | |
| ATOM | | 533 | CG1 | VAL | A | 103 | 9.889 | 22.458 | −1.162 | 1.00 | 12.19 |
| A | C | | | | | | | | | | |
| ATOM | | 534 | CG2 | VAL | A | 103 | 7.753 | 23.024 | −0.027 | 1.00 | 13.33 |
| A | C | | | | | | | | | | |
| ATOM | | 535 | C | VAL | A | 103 | 9.122 | 19.763 | −1.509 | 1.00 | 13.14 |
| A | C | | | | | | | | | | |
| ATOM | | 536 | O | VAL | A | 103 | 9.271 | 19.462 | −2.690 | 1.00 | 13.73 |
| A | O | | | | | | | | | | |
| ATOM | | 537 | N | LEU | A | 104 | 9.863 | 19.248 | −0.538 | 1.00 | 12.65 |
| A | N | | | | | | | | | | |
| ATOM | | 538 | CA | LEU | A | 104 | 10.952 | 18.330 | −0.824 | 1.00 | 14.40 |
| A | C | | | | | | | | | | |
| ATOM | | 539 | CB | LEU | A | 104 | 11.785 | 18.113 | 0.441 | 1.00 | 14.50 |
| A | C | | | | | | | | | | |
| ATOM | | 540 | CG | LEU | A | 104 | 12.438 | 19.396 | 0.972 | 1.00 | 14.36 |
| A | C | | | | | | | | | | |
| ATOM | | 541 | CD1 | LEU | A | 104 | 13.126 | 19.115 | 2.295 | 1.00 | 15.72 |
| A | C | | | | | | | | | | |
| ATOM | | 542 | CD2 | LEU | A | 104 | 13.441 | 19.928 | −0.050 | 1.00 | 14.39 |
| A | C | | | | | | | | | | |
| ATOM | | 543 | C | LEU | A | 104 | 10.544 | 16.989 | −1.415 | 1.00 | 15.37 |
| A | C | | | | | | | | | | |
| ATOM | | 544 | O | LEU | A | 104 | 11.215 | 16.480 | −2.313 | 1.00 | 12.81 |
| A | O | | | | | | | | | | |
| ATOM | | 545 | N | PHE | A | 105 | 9.467 | 16.399 | −0.916 | 1.00 | 15.46 |
| A | N | | | | | | | | | | |
| ATOM | | 546 | CA | PHE | A | 105 | 9.054 | 15.115 | −1.466 | 1.00 | 17.77 |
| A | C | | | | | | | | | | |
| ATOM | | 547 | CB | PHE | A | 105 | 7.926 | 14.508 | −0.621 | 1.00 | 18.57 |
| A | C | | | | | | | | | | |
| ATOM | | 548 | CG | PHE | A | 105 | 8.400 | 13.952 | 0.706 | 1.00 | 20.35 |
| A | C | | | | | | | | | | |
| ATOM | | 549 | CD1 | PHE | A | 105 | 9.750 | 14.016 | 1.066 | 1.00 | 22.06 |
| A | C | | | | | | | | | | |
| ATOM | | 550 | CD2 | PHE | A | 105 | 7.505 | 13.366 | 1.590 | 1.00 | 21.24 |
| A | C | | | | | | | | | | |
| ATOM | | 551 | CE1 | PHE | A | 105 | 10.193 | 13.501 | 2.288 | 1.00 | 22.72 |
| A | C | | | | | | | | | | |
| ATOM | | 552 | CE2 | PHE | A | 105 | 7.935 | 12.847 | 2.817 | 1.00 | 21.30 |
| A | C | | | | | | | | | | |
| ATOM | | 553 | CZ | PHE | A | 105 | 9.280 | 12.914 | 3.168 | 1.00 | 24.04 |
| A | C | | | | | | | | | | |
| ATOM | | 554 | C | PHE | A | 105 | 8.658 | 15.282 | −2.935 | 1.00 | 17.82 |
| A | C | | | | | | | | | | |
| ATOM | | 555 | O | PHE | A | 105 | 9.150 | 14.549 | −3.798 | 1.00 | 17.32 |
| A | O | | | | | | | | | | |
| ATOM | | 556 | N | PRO | A | 106 | 7.788 | 16.263 | −3.244 | 1.00 | 19.25 |
| A | N | | | | | | | | | | |
| ATOM | | 557 | CD | PRO | A | 106 | 6.995 | 17.100 | −2.323 | 1.00 | 18.44 |
| A | C | | | | | | | | | | |
| ATOM | | 558 | CA | PRO | A | 106 | 7.372 | 16.487 | −4.634 | 1.00 | 19.95 |
| A | C | | | | | | | | | | |
| ATOM | | 559 | CB | PRO | A | 106 | 6.417 | 17.672 | −4.521 | 1.00 | 20.31 |
| A | C | | | | | | | | | | |
| ATOM | | 560 | CG | PRO | A | 106 | 5.806 | 17.480 | −3.174 | 1.00 | 19.88 |
| A | C | | | | | | | | | | |
| ATOM | | 561 | C | PRO | A | 106 | 8.563 | 16.800 | −5.541 | 1.00 | 20.82 |
| A | C | | | | | | | | | | |
| ATOM | | 562 | O | PRO | A | 106 | 8.553 | 16.483 | −6.734 | 1.00 | 19.57 |
| A | O | | | | | | | | | | |
| ATOM | | 563 | N | GLN | A | 107 | 9.593 | 17.417 | −4.964 | 1.00 | 21.36 |
| A | N | | | | | | | | | | |
| ATOM | | 564 | CA | GLN | A | 107 | 10.791 | 17.800 | −5.714 | 1.00 | 21.96 |
| A | C | | | | | | | | | | |
| ATOM | | 565 | CB | GLN | A | 107 | 11.198 | 19.233 | −5.335 | 1.00 | 22.81 |
| A | C | | | | | | | | | | |
| ATOM | | 566 | CG | GLN | A | 107 | 10.156 | 20.298 | −5.638 | 1.00 | 24.02 |
| A | C | | | | | | | | | | |
| ATOM | | 567 | CD | GLN | A | 107 | 10.184 | 20.735 | −7.086 | 1.00 | 27.15 |
| A | C | | | | | | | | | | |
| ATOM | | 568 | OE1 | GLN | A | 107 | 9.302 | 21.459 | −7.547 | 1.00 | 28.19 |
| A | O | | | | | | | | | | |
| ATOM | | 569 | NE2 | GLN | A | 107 | 11.211 | 20.306 | −7.811 | 1.00 | 27.09 |
| A | N | | | | | | | | | | |
| ATOM | | 570 | C | GLN | A | 107 | 11.968 | 16.872 | −5.438 | 1.00 | 22.05 |
| A | C | | | | | | | | | | |
| ATOM | | 571 | O | GLN | A | 107 | 13.090 | 17.145 | −5.871 | 1.00 | 21.11 |
| A | O | | | | | | | | | | |
| ATOM | | 572 | N | SER | A | 108 | 11.709 | 15.773 | −4.734 | 1.00 | 22.40 |
| A | N | | | | | | | | | | |
| ATOM | | 573 | CA | SER | A | 108 | 12.756 | 14.829 | −4.342 | 1.00 | 24.12 |
| A | C | | | | | | | | | | |
| ATOM | | 574 | CB | SER | A | 108 | 12.142 | 13.665 | −3.559 | 1.00 | 21.76 |
| A | C | | | | | | | | | | |
| ATOM | | 575 | OG | SER | A | 108 | 11.263 | 12.909 | −4.370 | 1.00 | 25.11 |
| A | O | | | | | | | | | | |
| ATOM | | 576 | C | SER | A | 108 | 13.667 | 14.276 | −5.434 | 1.00 | 25.68 |
| A | C | | | | | | | | | | |
| ATOM | | 577 | O | SER | A | 108 | 14.823 | 13.942 | −5.168 | 1.00 | 25.48 |
| A | O | | | | | | | | | | |
| ATOM | | 578 | N | ASP | A | 109 | 13.165 | 14.179 | −6.658 | 1.00 | 28.58 |
| A | N | | | | | | | | | | |
| ATOM | | 579 | CA | ASP | A | 109 | 13.976 | 13.654 | −7.750 | 1.00 | 29.53 |
| A | C | | | | | | | | | | |
| ATOM | | 580 | CB | ASP | A | 109 | 13.131 | 12.719 | −8.623 | 1.00 | 31.30 |
| A | C | | | | | | | | | | |
| ATOM | | 581 | CG | ASP | A | 109 | 11.897 | 13.398 | −9.189 | 1.00 | 33.47 |
| A | C | | | | | | | | | | |
| ATOM | | 582 | OD1 | ASP | A | 109 | 11.371 | 14.328 | −8.542 | 1.00 | 33.88 |
| A | O | | | | | | | | | | |
| ATOM | | 583 | OD2 | ASP | A | 109 | 11.443 | 12.987 | −10.278 | 1.00 | 33.45 |
| A | O | | | | | | | | | | |
| ATOM | | 584 | C | ASP | A | 109 | 14.587 | 14.765 | −8.593 | 1.00 | 30.64 |
| A | C | | | | | | | | | | |
| ATOM | | 585 | O | ASP | A | 109 | 15.058 | 14.527 | −9.706 | 1.00 | 32.81 |
| A | O | | | | | | | | | | |
| ATOM | | 586 | N | ARG | A | 110 | 14.592 | 15.979 | −8.050 | 1.00 | 29.79 |
| A | N | | | | | | | | | | |
| ATOM | | 587 | CA | ARG | A | 110 | 15.145 | 17.134 | −8.748 | 1.00 | 28.46 |
| A | C | | | | | | | | | | |
| ATOM | | 588 | CB | ARG | A | 110 | 14.107 | 18.251 | −8.819 | 1.00 | 31.87 |
| A | C | | | | | | | | | | |
| ATOM | | 589 | CG | ARG | A | 110 | 14.497 | 19.382 | −9.753 | 1.00 | 37.18 |
| A | C | | | | | | | | | | |
| ATOM | | 590 | CD | ARG | A | 110 | 13.655 | 19.374 | −11.020 | 1.00 | 41.24 |
| A | C | | | | | | | | | | |
| ATOM | | 591 | NE | ARG | A | 110 | 12.340 | 19.974 | −10.803 | 1.00 | 43.98 |
| A | N | | | | | | | | | | |
| ATOM | | 592 | CZ | ARG | A | 110 | 12.146 | 21.255 | −10.497 | 1.00 | 44.48 |
| A | C | | | | | | | | | | |
| ATOM | | 593 | NH1 | ARG | A | 110 | 13.180 | 22.074 | −10.373 | 1.00 | 44.65 |
| A | N | | | | | | | | | | |
| ATOM | | 594 | NH2 | ARG | A | 110 | 10.916 | 21.718 | −10.315 | 1.00 | 45.63 |
| A | N | | | | | | | | | | |
| ATOM | | 595 | C | ARG | A | 110 | 16.384 | 17.629 | −8.008 | 1.00 | 25.45 |
| A | C | | | | | | | | | | |
| ATOM | | 596 | O | ARG | A | 110 | 16.715 | 17.118 | −6.942 | 1.00 | 25.04 |
| A | O | | | | | | | | | | |
| ATOM | | 597 | N | PHE | A | 111 | 17.070 | 18.616 | −8.576 | 1.00 | 22.98 |
| A | N | | | | | | | | | | |
| ATOM | | 598 | CA | PHE | A | 111 | 18.278 | 19.163 | −7.965 | 1.00 | 22.26 |
| A | C | | | | | | | | | | |
| ATOM | | 599 | CB | PHE | A | 111 | 17.900 | 20.022 | −6.749 | 1.00 | 21.90 |
| A | C | | | | | | | | | | |
| ATOM | | 600 | CG | PHE | A | 111 | 17.148 | 21.274 | −7.111 | 1.00 | 18.30 |
| A | C | | | | | | | | | | |
| ATOM | | 601 | CD1 | PHE | A | 111 | 17.795 | 22.325 | −7.747 | 1.00 | 20.61 |
| A | C | | | | | | | | | | |
| ATOM | | 602 | CD2 | PHE | A | 111 | 15.793 | 21.381 | −6.866 | 1.00 | 19.71 |
| A | C | | | | | | | | | | |
| ATOM | | 603 | CE1 | PHE | A | 111 | 17.101 | 23.461 | −8.135 | 1.00 | 20.07 |
| A | C | | | | | | | | | | |
| ATOM | | 604 | CE2 | PHE | A | 111 | 15.085 | 22.520 | −7.254 | 1.00 | 20.77 |
| A | C | | | | | | | | | | |
| ATOM | | 605 | CZ | PHE | A | 111 | 15.742 | 23.557 | −7.890 | 1.00 | 22.06 |
| A | C | | | | | | | | | | |
| ATOM | | 606 | C | PHE | A | 111 | 19.281 | 18.070 | −7.561 | 1.00 | 23.53 |
| A | C | | | | | | | | | | |
| ATOM | | 607 | O | PHE | A | 111 | 19.819 | 18.082 | −6.453 | 1.00 | 21.50 |
| A | O | | | | | | | | | | |
| ATOM | | 608 | N | GLN | A | 112 | 19.528 | 17.125 | −8.468 | 1.00 | 24.20 |
| A | N | | | | | | | | | | |
| ATOM | | 609 | CA | GLN | A | 112 | 20.486 | 16.042 | −8.215 | 1.00 | 24.96 |
| A | C | | | | | | | | | | |
| ATOM | | 610 | CB | GLN | A | 112 | 20.397 | 14.962 | −9.304 | 1.00 | 27.91 |
| A | C | | | | | | | | | | |
| ATOM | | 611 | CG | GLN | A | 112 | 19.033 | 14.309 | −9.375 | 1.00 | 32.20 |
| A | C | | | | | | | | | | |
| ATOM | | 612 | CD | GLN | A | 112 | 18.866 | 13.316 | −10.503 | 1.00 | 36.00 |
| A | C | | | | | | | | | | |
| ATOM | | 613 | OE1 | GLN | A | 112 | 17.772 | 12.806 | −10.719 | 1.00 | 37.02 |
| A | O | | | | | | | | | | |
| ATOM | | 614 | NE2 | GLN | A | 112 | 19.946 | 13.033 | −11.225 | 1.00 | 37.13 |
| A | N | | | | | | | | | | |
| ATOM | | 615 | C | GLN | A | 112 | 21.897 | 16.630 | −8.196 | 1.00 | 26.21 |
| A | C | | | | | | | | | | |
| ATOM | | 616 | O | GLN | A | 112 | 22.184 | 17.597 | −8.908 | 1.00 | 27.42 |
| A | O | | | | | | | | | | |
| ATOM | | 617 | N | PRO | A | 113 | 22.806 | 16.047 | −7.392 | 1.00 | 25.53 |
| A | N | | | | | | | | | | |
| ATOM | | 618 | CD | PRO | A | 113 | 24.251 | 16.272 | −7.577 | 1.00 | 25.70 |
| A | C | | | | | | | | | | |
| ATOM | | 619 | CA | PRO | A | 113 | 22.577 | 14.900 | −6.506 | 1.00 | 25.89 |
| A | C | | | | | | | | | | |
| ATOM | | 620 | CB | PRO | A | 113 | 23.853 | 14.098 | −6.695 | 1.00 | 26.16 |
| A | C | | | | | | | | | | |
| ATOM | | 621 | CG | PRO | A | 113 | 24.876 | 15.203 | −6.687 | 1.00 | 25.44 |
| A | C | | | | | | | | | | |
| ATOM | | 622 | C | PRO | A | 113 | 22.385 | 15.287 | −5.032 | 1.00 | 25.87 |
| A | C | | | | | | | | | | |
| ATOM | | 623 | O | PRO | A | 113 | 22.095 | 14.434 | −4.195 | 1.00 | 25.05 |
| A | O | | | | | | | | | | |
| ATOM | | 624 | N | TYR | A | 114 | 22.541 | 16.571 | −4.717 | 1.00 | 26.68 |
| A | N | | | | | | | | | | |
| ATOM | | 625 | CA | TYR | A | 114 | 22.430 | 17.029 | −3.331 | 1.00 | 27.34 |
| A | C | | | | | | | | | | |
| ATOM | | 626 | CB | TYR | A | 114 | 22.838 | 18.510 | −3.228 | 1.00 | 30.74 |
| A | C | | | | | | | | | | |
| ATOM | | 627 | CG | TYR | A | 114 | 24.167 | 18.813 | −3.898 | 1.00 | 33.96 |
| A | C | | | | | | | | | | |
| ATOM | | 628 | CD1 | TYR | A | 114 | 25.228 | 17.916 | −3.804 | 1.00 | 36.01 |
| A | C | | | | | | | | | | |
| ATOM | | 629 | CE1 | TYR | A | 114 | 26.435 | 18.161 | −4.438 | 1.00 | 38.72 |
| A | C | | | | | | | | | | |
| ATOM | | 630 | CD2 | TYR | A | 114 | 24.354 | 19.979 | −4.648 | 1.00 | 35.86 |
| A | C | | | | | | | | | | |
| ATOM | | 631 | CE2 | TYR | A | 114 | 25.568 | 20.238 | −5.290 | 1.00 | 37.49 |
| A | C | | | | | | | | | | |
| ATOM | | 632 | CZ | TYR | A | 114 | 26.601 | 19.321 | −5.178 | 1.00 | 39.49 |
| A | C | | | | | | | | | | |
| ATOM | | 633 | OH | TYR | A | 114 | 27.809 | 19.560 | −5.789 | 1.00 | 40.81 |
| A | O | | | | | | | | | | |
| ATOM | | 634 | C | TYR | A | 114 | 21.069 | 16.810 | −2.677 | 1.00 | 26.15 |
| A | C | | | | | | | | | | |
| ATOM | | 635 | O | TYR | A | 114 | 20.987 | 16.549 | −1.476 | 1.00 | 24.90 |
| A | O | | | | | | | | | | |
| ATOM | | 636 | N | MET | A | 115 | 20.006 | 16.906 | −3.467 | 1.00 | 25.38 |
| A | N | | | | | | | | | | |
| ATOM | | 637 | CA | MET | A | 115 | 18.662 | 16.721 | −2.948 | 1.00 | 24.05 |
| A | C | | | | | | | | | | |
| ATOM | | 638 | CB | MET | A | 115 | 17.646 | 16.865 | −4.084 | 1.00 | 24.96 |
| A | C | | | | | | | | | | |
| ATOM | | 639 | CG | MET | A | 115 | 16.206 | 16.603 | −3.708 | 1.00 | 24.12 |
| A | C | | | | | | | | | | |
| ATOM | | 640 | SD | MET | A | 115 | 15.563 | 17.820 | −2.574 | 1.00 | 23.41 |
| A | S | | | | | | | | | | |
| ATOM | | 641 | CE | MET | A | 115 | 15.122 | 19.162 | −3.708 | 1.00 | 23.60 |
| A | C | | | | | | | | | | |
| ATOM | | 642 | C | MET | A | 115 | 18.535 | 15.348 | −2.300 | 1.00 | 24.70 |
| A | C | | | | | | | | | | |
| ATOM | | 643 | O | MET | A | 115 | 17.980 | 15.225 | −1.218 | 1.00 | 22.93 |
| A | O | | | | | | | | | | |
| ATOM | | 644 | N | GLN | A | 116 | 19.085 | 14.327 | −2.951 | 1.00 | 23.45 |
| A | N | | | | | | | | | | |
| ATOM | | 645 | CA | GLN | A | 116 | 19.005 | 12.962 | −2.453 | 1.00 | 23.38 |
| A | C | | | | | | | | | | |
| ATOM | | 646 | CB | GLN | A | 116 | 19.439 | 12.001 | −3.561 | 1.00 | 25.27 |
| A | C | | | | | | | | | | |
| ATOM | | 647 | CG | GLN | A | 116 | 18.491 | 12.034 | −4.765 | 1.00 | 28.21 |
| A | C | | | | | | | | | | |
| ATOM | | 648 | CD | GLN | A | 116 | 18.550 | 13.344 | −5.550 | 1.00 | 29.58 |
| A | C | | | | | | | | | | |
| ATOM | | 649 | OE1 | GLN | A | 116 | 17.549 | 13.786 | −6.116 | 1.00 | 32.33 |
| A | O | | | | | | | | | | |
| ATOM | | 650 | NE2 | GLN | A | 116 | 19.727 | 13.956 | −5.600 | 1.00 | 27.60 |
| A | N | | | | | | | | | | |
| ATOM | | 651 | C | GLN | A | 116 | 19.766 | 12.683 | −1.160 | 1.00 | 21.18 |
| A | C | | | | | | | | | | |
| ATOM | | 652 | O | GLN | A | 116 | 19.570 | 11.650 | −0.527 | 1.00 | 21.75 |
| A | O | | | | | | | | | | |
| ATOM | | 653 | N | GLU | A | 117 | 20.632 | 13.603 | −0.761 | 1.00 | 19.23 |
| A | N | | | | | | | | | | |
| ATOM | | 654 | CA | GLU | A | 117 | 21.383 | 13.426 | 0.472 | 1.00 | 18.78 |
| A | C | | | | | | | | | | |
| ATOM | | 655 | CB | GLU | A | 117 | 22.834 | 13.851 | 0.272 | 1.00 | 22.01 |
| A | C | | | | | | | | | | |
| ATOM | | 656 | CG | GLU | A | 117 | 23.574 | 13.015 | −0.751 | 1.00 | 26.21 |
| A | C | | | | | | | | | | |
| ATOM | | 657 | CD | GLU | A | 117 | 25.011 | 13.446 | −0.915 | 1.00 | 28.09 |
| A | C | | | | | | | | | | |
| ATOM | | 658 | OE1 | GLU | A | 117 | 25.245 | 14.584 | −1.373 | 1.00 | 31.04 |
| A | O | | | | | | | | | | |
| ATOM | | 659 | OE2 | GLU | A | 117 | 25.907 | 12.643 | −0.583 | 1.00 | 29.29 |
| A | O | | | | | | | | | | |
| ATOM | | 660 | C | GLU | A | 117 | 20.765 | 14.252 | 1.592 | 1.00 | 15.64 |
| A | C | | | | | | | | | | |
| ATOM | | 661 | O | GLU | A | 117 | 20.748 | 13.834 | 2.742 | 1.00 | 12.05 |
| A | O | | | | | | | | | | |
| ATOM | | 662 | N | VAL | A | 118 | 20.247 | 15.423 | 1.233 | 1.00 | 12.54 |
| A | N | | | | | | | | | | |
| ATOM | | 663 | CA | VAL | A | 118 | 19.643 | 16.340 | 2.190 | 1.00 | 10.94 |
| A | C | | | | | | | | | | |
| ATOM | | 664 | CB | VAL | A | 118 | 19.521 | 17.752 | 1.571 | 1.00 | 13.38 |
| A | C | | | | | | | | | | |
| ATOM | | 665 | CG1 | VAL | A | 118 | 18.818 | 18.689 | 2.529 | 1.00 | 11.25 |
| A | C | | | | | | | | | | |
| ATOM | | 666 | CG2 | VAL | A | 118 | 20.913 | 18.285 | 1.227 | 1.00 | 11.27 |
| A | C | | | | | | | | | | |
| ATOM | | 667 | C | VAL | A | 118 | 18.266 | 15.887 | 2.686 | 1.00 | 12.41 |
| A | C | | | | | | | | | | |
| ATOM | | 668 | O | VAL | A | 118 | 17.971 | 15.999 | 3.872 | 1.00 | 12.25 |
| A | O | | | | | | | | | | |
| ATOM | | 669 | N | VAL | A | 119 | 17.427 | 15.372 | 1.793 | 1.00 | 11.42 |
| A | N | | | | | | | | | | |
| ATOM | | 670 | CA | VAL | A | 119 | 16.097 | 14.942 | 2.208 | 1.00 | 13.06 |
| A | C | | | | | | | | | | |
| ATOM | | 671 | CB | VAL | A | 119 | 15.275 | 14.419 | 1.007 | 1.00 | 14.89 |
| A | C | | | | | | | | | | |
| ATOM | | 672 | CG1 | VAL | A | 119 | 14.001 | 13.738 | 1.495 | 1.00 | 13.96 |
| A | C | | | | | | | | | | |
| ATOM | | 673 | CG2 | VAL | A | 119 | 14.937 | 15.586 | 0.072 | 1.00 | 12.52 |
| A | C | | | | | | | | | | |
| ATOM | | 674 | C | VAL | A | 119 | 16.123 | 13.902 | 3.337 | 1.00 | 12.86 |
| A | C | | | | | | | | | | |
| ATOM | | 675 | O | VAL | A | 119 | 15.425 | 14.069 | 4.335 | 1.00 | 13.99 |
| A | O | | | | | | | | | | |
| ATOM | | 676 | N | PRO | A | 120 | 16.933 | 12.832 | 3.208 | 1.00 | 13.22 |
| A | N | | | | | | | | | | |
| ATOM | | 677 | CD | PRO | A | 120 | 17.731 | 12.385 | 2.052 | 1.00 | 13.49 |
| A | C | | | | | | | | | | |
| ATOM | | 678 | CA | PRO | A | 120 | 16.979 | 11.825 | 4.274 | 1.00 | 13.95 |
| A | C | | | | | | | | | | |
| ATOM | | 679 | CB | PRO | A | 120 | 17.979 | 10.798 | 3.743 | 1.00 | 13.50 |
| A | C | | | | | | | | | | |
| ATOM | | 680 | CG | PRO | A | 120 | 17.776 | 10.882 | 2.264 | 1.00 | 16.77 |
| A | C | | | | | | | | | | |
| ATOM | | 681 | C | PRO | A | 120 | 17.427 | 12.439 | 5.590 | 1.00 | 13.93 |
| A | C | | | | | | | | | | |
| ATOM | | 682 | O | PRO | A | 120 | 16.951 | 12.058 | 6.652 | 1.00 | 13.50 |
| A | O | | | | | | | | | | |
| ATOM | | 683 | N | PHE | A | 121 | 18.357 | 13.385 | 5.508 | 1.00 | 11.40 |
| A | N | | | | | | | | | | |
| ATOM | | 684 | CA | PHE | A | 121 | 18.860 | 14.078 | 6.692 | 1.00 | 11.01 |
| A | C | | | | | | | | | | |
| ATOM | | 685 | CB | PHE | A | 121 | 19.978 | 15.048 | 6.270 | 1.00 | 10.39 |
| A | C | | | | | | | | | | |
| ATOM | | 686 | CG | PHE | A | 121 | 20.327 | 16.085 | 7.309 | 1.00 | 12.29 |
| A | C | | | | | | | | | | |
| ATOM | | 687 | CD1 | PHE | A | 121 | 20.796 | 15.713 | 8.561 | 1.00 | 10.86 |
| A | C | | | | | | | | | | |
| ATOM | | 688 | CD2 | PHE | A | 121 | 20.220 | 17.440 | 7.011 | 1.00 | 12.84 |
| A | C | | | | | | | | | | |
| ATOM | | 689 | CE1 | PHE | A | 121 | 21.153 | 16.678 | 9.506 | 1.00 | 11.52 |
| A | C | | | | | | | | | | |
| ATOM | | 690 | CE2 | PHE | A | 121 | 20.573 | 18.416 | 7.951 | 1.00 | 13.64 |
| A | C | | | | | | | | | | |
| ATOM | | 691 | CZ | PHE | A | 121 | 21.042 | 18.034 | 9.195 | 1.00 | 11.27 |
| A | C | | | | | | | | | | |
| ATOM | | 692 | C | PHE | A | 121 | 17.715 | 14.837 | 7.383 | 1.00 | 9.92 |
| A | C | | | | | | | | | | |
| ATOM | | 693 | O | PHE | A | 121 | 17.474 | 14.658 | 8.575 | 1.00 | 10.41 |
| A | O | | | | | | | | | | |
| ATOM | | 694 | N | LEU | A | 122 | 17.003 | 15.673 | 6.631 | 1.00 | 8.04 |
| A | N | | | | | | | | | | |
| ATOM | | 695 | CA | LEU | A | 122 | 15.895 | 16.445 | 7.202 | 1.00 | 9.54 |
| A | C | | | | | | | | | | |
| ATOM | | 696 | CB | LEU | A | 122 | 15.364 | 17.439 | 6.167 | 1.00 | 9.32 |
| A | C | | | | | | | | | | |
| ATOM | | 697 | CG | LEU | A | 122 | 16.417 | 18.482 | 5.780 | 1.00 | 10.24 |
| A | C | | | | | | | | | | |
| ATOM | | 698 | CD1 | LEU | A | 122 | 15.888 | 19.366 | 4.658 | 1.00 | 12.89 |
| A | C | | | | | | | | | | |
| ATOM | | 699 | CD2 | LEU | A | 122 | 16.779 | 19.325 | 6.997 | 1.00 | 10.42 |
| A | C | | | | | | | | | | |
| ATOM | | 700 | C | LEU | A | 122 | 14.765 | 15.551 | 7.714 | 1.00 | 9.19 |
| A | C | | | | | | | | | | |
| ATOM | | 701 | O | LEU | A | 122 | 14.133 | 15.853 | 8.729 | 1.00 | 11.17 |
| A | O | | | | | | | | | | |
| ATOM | | 702 | N | ALA | A | 123 | 14.508 | 14.454 | 7.012 | 1.00 | 10.07 |
| A | N | | | | | | | | | | |
| ATOM | | 703 | CA | ALA | A | 123 | 13.473 | 13.518 | 7.432 | 1.00 | 11.76 |
| A | C | | | | | | | | | | |
| ATOM | | 704 | CB | ALA | A | 123 | 13.307 | 12.415 | 6.385 | 1.00 | 11.58 |
| A | C | | | | | | | | | | |
| ATOM | | 705 | C | ALA | A | 123 | 13.844 | 12.901 | 8.785 | 1.00 | 12.42 |
| A | C | | | | | | | | | | |
| ATOM | | 706 | O | ALA | A | 123 | 12.972 | 12.651 | 9.629 | 1.00 | 10.87 |
| A | O | | | | | | | | | | |
| ATOM | | 707 | N | ARG | A | 124 | 15.129 | 12.637 | 8.996 | 1.00 | 13.52 |
| A | N | | | | | | | | | | |
| ATOM | | 708 | CA | ARG | A | 124 | 15.537 | 12.057 | 10.268 | 1.00 | 15.12 |
| A | C | | | | | | | | | | |
| ATOM | | 709 | CB | ARG | A | 124 | 16.998 | 11.599 | 10.230 | 1.00 | 16.39 |
| A | C | | | | | | | | | | |
| ATOM | | 710 | CG | ARG | A | 124 | 17.222 | 10.361 | 9.361 | 1.00 | 18.81 |
| A | C | | | | | | | | | | |
| ATOM | | 711 | CD | ARG | A | 124 | 18.514 | 9.644 | 9.730 | 1.00 | 19.88 |
| A | C | | | | | | | | | | |
| ATOM | | 712 | NE | ARG | A | 124 | 19.684 | 10.503 | 9.592 | 1.00 | 21.22 |
| A | N | | | | | | | | | | |
| ATOM | | 713 | CZ | ARG | A | 124 | 20.241 | 10.818 | 8.429 | 1.00 | 21.14 |
| A | C | | | | | | | | | | |
| ATOM | | 714 | NH1 | ARG | A | 124 | 19.739 | 10.339 | 7.300 | 1.00 | 21.19 |
| A | N | | | | | | | | | | |
| ATOM | | 715 | NH2 | ARG | A | 124 | 21.292 | 11.620 | 8.398 | 1.00 | 21.84 |
| A | N | | | | | | | | | | |
| ATOM | | 716 | C | ARG | A | 124 | 15.319 | 13.052 | 11.396 | 1.00 | 14.67 |
| A | C | | | | | | | | | | |
| ATOM | | 717 | O | ARG | A | 124 | 14.968 | 12.666 | 12.509 | 1.00 | 14.02 |
| A | O | | | | | | | | | | |
| ATOM | | 718 | N | LEU | A | 125 | 15.512 | 14.336 | 11.113 | 1.00 | 12.38 |
| A | N | | | | | | | | | | |
| ATOM | | 719 | CA | LEU | A | 125 | 15.305 | 15.348 | 12.142 | 1.00 | 12.66 |
| A | C | | | | | | | | | | |
| ATOM | | 720 | CB | LEU | A | 125 | 15.757 | 16.724 | 11.655 | 1.00 | 11.19 |
| A | C | | | | | | | | | | |
| ATOM | | 721 | CG | LEU | A | 125 | 17.257 | 16.863 | 11.400 | 1.00 | 12.07 |
| A | C | | | | | | | | | | |
| ATOM | | 722 | CD1 | LEU | A | 125 | 17.571 | 18.262 | 10.880 | 1.00 | 11.21 |
| A | C | | | | | | | | | | |
| ATOM | | 723 | CD2 | LEU | A | 125 | 18.015 | 16.567 | 12.691 | 1.00 | 10.90 |
| A | C | | | | | | | | | | |
| ATOM | | 724 | C | LEU | A | 125 | 13.824 | 15.384 | 12.457 | 1.00 | 12.64 |
| A | C | | | | | | | | | | |
| ATOM | | 725 | O | LEU | A | 125 | 13.424 | 15.502 | 13.615 | 1.00 | 13.64 |
| A | O | | | | | | | | | | |
| ATOM | | 726 | N | SER | A | 126 | 13.010 | 15.289 | 11.412 | 1.00 | 11.87 |
| A | N | | | | | | | | | | |
| ATOM | | 727 | CA | SER | A | 126 | 11.567 | 15.301 | 11.584 | 1.00 | 12.10 |
| A | C | | | | | | | | | | |
| ATOM | | 728 | CB | SER | A | 126 | 10.869 | 15.199 | 10.219 | 1.00 | 12.32 |
| A | C | | | | | | | | | | |
| ATOM | | 729 | OG | SER | A | 126 | 9.511 | 14.794 | 10.350 | 1.00 | 12.93 |
| A | O | | | | | | | | | | |
| ATOM | | 730 | C | SER | A | 126 | 11.174 | 14.124 | 12.466 | 1.00 | 14.19 |
| A | C | | | | | | | | | | |
| ATOM | | 731 | O | SER | A | 126 | 10.379 | 14.267 | 13.397 | 1.00 | 14.24 |
| A | O | | | | | | | | | | |
| ATOM | | 732 | N | ASN | A | 127 | 11.751 | 12.960 | 12.182 | 1.00 | 15.34 |
| A | N | | | | | | | | | | |
| ATOM | | 733 | CA | ASN | A | 127 | 11.441 | 11.761 | 12.953 | 1.00 | 19.77 |
| A | C | | | | | | | | | | |
| ATOM | | 734 | CB | ASN | A | 127 | 12.143 | 10.532 | 12.363 | 1.00 | 22.15 |
| A | C | | | | | | | | | | |
| ATOM | | 735 | CG | ASN | A | 127 | 11.687 | 9.236 | 13.020 | 1.00 | 27.13 |
| A | C | | | | | | | | | | |
| ATOM | | 736 | OD1 | ASN | A | 127 | 10.492 | 8.925 | 13.039 | 1.00 | 29.33 |
| A | O | | | | | | | | | | |
| ATOM | | 737 | ND2 | ASN | A | 127 | 12.634 | 8.479 | 13.564 | 1.00 | 27.25 |
| A | N | | | | | | | | | | |
| ATOM | | 738 | C | ASN | A | 127 | 11.839 | 11.922 | 14.416 | 1.00 | 19.60 |
| A | C | | | | | | | | | | |
| ATOM | | 739 | O | ASN | A | 127 | 11.162 | 11.412 | 15.305 | 1.00 | 19.90 |
| A | O | | | | | | | | | | |
| ATOM | | 740 | N | ARG | A | 128 | 12.934 | 12.629 | 14.672 | 1.00 | 19.27 |
| A | N | | | | | | | | | | |
| ATOM | | 741 | CA | ARG | A | 128 | 13.396 | 12.855 | 16.043 | 1.00 | 20.04 |
| A | C | | | | | | | | | | |
| ATOM | | 742 | CB | ARG | A | 128 | 14.783 | 13.534 | 15.992 | 1.00 | 20.58 |
| A | C | | | | | | | | | | |
| ATOM | | 743 | CG | ARG | A | 128 | 15.659 | 13.507 | 17.262 | 1.00 | 24.13 |
| A | C | | | | | | | | | | |
| ATOM | | 744 | CD | ARG | A | 128 | 15.120 | 14.392 | 18.371 | 1.00 | 27.44 |
| A | C | | | | | | | | | | |
| ATOM | | 745 | NE | ARG | A | 128 | 16.165 | 14.909 | 19.249 | 1.00 | 27.58 |
| A | N | | | | | | | | | | |
| ATOM | | 746 | CZ | ARG | A | 128 | 15.991 | 15.179 | 20.539 | 1.00 | 29.81 |
| A | C | | | | | | | | | | |
| ATOM | | 747 | NH1 | ARG | A | 128 | 14.809 | 14.970 | 21.107 | 1.00 | 29.81 |
| A | N | | | | | | | | | | |
| ATOM | | 748 | NH2 | ARG | A | 128 | 16.991 | 15.671 | 21.256 | 1.00 | 27.09 |
| A | N | | | | | | | | | | |
| ATOM | | 749 | C | ARG | A | 128 | 12.373 | 13.743 | 16.788 | 1.00 | 20.54 |
| A | C | | | | | | | | | | |
| ATOM | | 750 | O | ARG | A | 128 | 12.069 | 13.519 | 17.967 | 1.00 | 18.67 |
| A | O | | | | | | | | | | |
| ATOM | | 751 | N | LEU | A | 129 | 11.850 | 14.753 | 16.098 | 1.00 | 19.98 |
| A | N | | | | | | | | | | |
| ATOM | | 752 | CA | LEU | A | 129 | 10.876 | 15.669 | 16.695 | 1.00 | 21.90 |
| A | C | | | | | | | | | | |
| ATOM | | 753 | CB | LEU | A | 129 | 10.813 | 16.965 | 15.886 | 1.00 | 19.92 |
| A | C | | | | | | | | | | |
| ATOM | | 754 | CG | LEU | A | 129 | 12.044 | 17.872 | 15.908 | 1.00 | 19.27 |
| A | C | | | | | | | | | | |
| ATOM | | 755 | CD1 | LEU | A | 129 | 11.925 | 18.930 | 14.824 | 1.00 | 19.33 |
| A | C | | | | | | | | | | |
| ATOM | | 756 | CD2 | LEU | A | 129 | 12.188 | 18.504 | 17.287 | 1.00 | 16.10 |
| A | C | | | | | | | | | | |
| ATOM | | 757 | C | LEU | A | 129 | 9.474 | 15.075 | 16.750 | 1.00 | 25.34 |
| A | C | | | | | | | | | | |
| ATOM | | 758 | O | LEU | A | 129 | 8.592 | 15.579 | 17.452 | 1.00 | 25.92 |
| A | O | | | | | | | | | | |
| ATOM | | 759 | N | SER | A | 130 | 9.270 | 14.007 | 15.994 | 1.00 | 28.06 |
| A | N | | | | | | | | | | |
| ATOM | | 760 | CA | SER | A | 130 | 7.969 | 13.364 | 15.910 | 1.00 | 33.42 |
| A | C | | | | | | | | | | |
| ATOM | | 761 | CB | SER | A | 130 | 8.095 | 11.979 | 15.288 | 1.00 | 32.53 |
| A | C | | | | | | | | | | |
| ATOM | | 762 | OG | SER | A | 130 | 8.750 | 11.096 | 16.177 | 1.00 | 35.12 |
| A | O | | | | | | | | | | |
| ATOM | | 763 | C | SER | A | 130 | 7.200 | 13.233 | 17.210 | 1.00 | 36.11 |
| A | C | | | | | | | | | | |
| ATOM | | 764 | O | SER | A | 130 | 7.681 | 12.658 | 18.190 | 1.00 | 38.33 |
| A | O | | | | | | | | | | |
| ATOM | | 765 | N | THR | A | 131 | 5.993 | 13.785 | 17.200 | 1.00 | 39.23 |
| A | N | | | | | | | | | | |
| ATOM | | 766 | CA | THR | A | 131 | 5.098 | 13.700 | 18.340 | 1.00 | 42.38 |
| A | C | | | | | | | | | | |
| ATOM | | 767 | CB | THR | A | 131 | 4.968 | 12.214 | 18.733 | 1.00 | 44.32 |
| A | C | | | | | | | | | | |
| ATOM | | 768 | OG1 | THR | A | 131 | 4.843 | 11.445 | 17.527 | 1.00 | 47.57 |
| A | O | | | | | | | | | | |
| ATOM | | 769 | CG2 | THR | A | 131 | 3.725 | 11.944 | 19.561 | 1.00 | 45.19 |
| A | C | | | | | | | | | | |
| ATOM | | 770 | C | THR | A | 131 | 5.448 | 14.576 | 19.558 | 1.00 | 42.64 |
| A | C | | | | | | | | | | |
| ATOM | | 771 | O | THR | A | 131 | 4.710 | 14.560 | 20.551 | 1.00 | 43.84 |
| A | O | | | | | | | | | | |
| ATOM | | 772 | N | CYS | A | 132 | 6.559 | 15.325 | 19.496 | 1.00 | 41.52 |
| A | N | | | | | | | | | | |
| ATOM | | 773 | CA | CYS | A | 132 | 6.930 | 16.219 | 20.603 | 1.00 | 39.84 |
| A | C | | | | | | | | | | |
| ATOM | | 774 | C | CYS | A | 132 | 5.899 | 17.299 | 20.541 | 1.00 | 40.10 |
| A | C | | | | | | | | | | |
| ATOM | | 775 | O | CYS | A | 132 | 5.358 | 17.570 | 19.471 | 1.00 | 40.22 |
| A | O | | | | | | | | | | |
| ATOM | | 776 | CB | CYS | A | 132 | 8.309 | 16.869 | 20.413 | 1.00 | 36.93 |
| A | C | | | | | | | | | | |
| ATOM | | 777 | SG | CYS | A | 132 | 8.480 | 18.000 | 18.999 | 1.00 | 32.53 |
| A | S | | | | | | | | | | |
| ATOM | | 778 | N | HIS | A | 133 | 5.624 | 17.927 | 21.673 | 1.00 | 40.74 |
| A | N | | | | | | | | | | |
| ATOM | | 779 | CA | HIS | A | 133 | 4.629 | 18.979 | 21.689 | 1.00 | 41.87 |
| A | C | | | | | | | | | | |
| ATOM | | 780 | CB | HIS | A | 133 | 3.251 | 18.373 | 21.933 | 1.00 | 44.51 |
| A | C | | | | | | | | | | |
| ATOM | | 781 | CG | HIS | A | 133 | 3.122 | 17.694 | 23.260 | 1.00 | 47.49 |
| A | C | | | | | | | | | | |
| ATOM | | 782 | CD2 | HIS | A | 133 | 2.913 | 16.396 | 23.583 | 1.00 | 48.42 |
| A | C | | | | | | | | | | |
| ATOM | | 783 | ND1 | HIS | A | 133 | 3.241 | 18.371 | 24.455 | 1.00 | 48.25 |
| A | N | | | | | | | | | | |
| ATOM | | 784 | CE1 | HIS | A | 133 | 3.114 | 17.518 | 25.457 | 1.00 | 48.41 |
| A | C | | | | | | | | | | |
| ATOM | | 785 | NE2 | HIS | A | 133 | 2.915 | 16.313 | 24.955 | 1.00 | 48.80 |
| A | N | | | | | | | | | | |
| ATOM | | 786 | C | HIS | A | 133 | 4.916 | 20.023 | 22.750 | 1.00 | 41.49 |
| A | C | | | | | | | | | | |
| ATOM | | 787 | O | HIS | A | 133 | 5.897 | 19.924 | 23.488 | 1.00 | 40.33 |
| A | O | | | | | | | | | | |
| ATOM | | 788 | N | ILE | A | 134 | 4.050 | 21.030 | 22.811 | 1.00 | 41.32 |
| A | N | | | | | | | | | | |
| ATOM | | 789 | CA | ILE | A | 134 | 4.176 | 22.099 | 23.794 | 1.00 | 42.67 |
| A | C | | | | | | | | | | |
| ATOM | | 790 | CB | ILE | A | 134 | 4.176 | 23.493 | 23.120 | 1.00 | 42.74 |
| A | C | | | | | | | | | | |
| ATOM | | 791 | CG2 | ILE | A | 134 | 5.446 | 23.663 | 22.301 | 1.00 | 42.04 |
| A | C | | | | | | | | | | |
| ATOM | | 792 | CG1 | ILE | A | 134 | 2.918 | 23.649 | 22.255 | 1.00 | 41.70 |
| A | C | | | | | | | | | | |
| ATOM | | 793 | CD | ILE | A | 134 | 2.780 | 24.957 | 21.499 | 1.00 | 41.81 |
| A | C | | | | | | | | | | |
| ATOM | | 794 | C | ILE | A | 134 | 3.000 | 21.999 | 24.763 | 1.00 | 43.64 |
| A | C | | | | | | | | | | |
| ATOM | | 795 | O | ILE | A | 134 | 1.906 | 21.571 | 24.387 | 1.00 | 43.52 |
| A | O | | | | | | | | | | |
| ATOM | | 796 | N | GLU | A | 135 | 3.229 | 22.391 | 26.011 | 1.00 | 44.33 |
| A | N | | | | | | | | | | |
| ATOM | | 797 | CA | GLU | A | 135 | 2.189 | 22.319 | 27.031 | 1.00 | 45.59 |
| A | C | | | | | | | | | | |
| ATOM | | 798 | CB | GLU | A | 135 | 2.832 | 22.272 | 28.431 | 1.00 | 47.61 |
| A | C | | | | | | | | | | |
| ATOM | | 799 | CG | GLU | A | 135 | 3.765 | 23.448 | 28.742 | 1.00 | 50.52 |
| A | C | | | | | | | | | | |
| ATOM | | 800 | CD | GLU | A | 135 | 4.589 | 23.260 | 30.002 | 1.00 | 51.87 |
| A | C | | | | | | | | | | |
| ATOM | | 801 | OE1 | GLU | A | 135 | 5.538 | 22.454 | 29.942 | 1.00 | 51.72 |
| A | O | | | | | | | | | | |
| ATOM | | 802 | OE2 | GLU | A | 135 | 4.301 | 23.912 | 31.037 | 1.00 | 52.55 |
| A | O | | | | | | | | | | |
| ATOM | | 803 | C | GLU | A | 135 | 1.191 | 23.474 | 26.938 | 1.00 | 45.27 |
| A | C | | | | | | | | | | |
| ATOM | | 804 | O | GLU | A | 135 | 0.204 | 23.512 | 27.674 | 1.00 | 45.54 |
| A | O | | | | | | | | | | |
| ATOM | | 805 | N | GLY | A | 136 | 1.447 | 24.415 | 26.033 | 1.00 | 44.63 |
| A | N | | | | | | | | | | |
| ATOM | | 806 | CA | GLY | A | 136 | 0.557 | 25.553 | 25.878 | 1.00 | 43.02 |
| A | C | | | | | | | | | | |
| ATOM | | 807 | C | GLY | A | 136 | −0.268 | 25.466 | 24.612 | 1.00 | 42.19 |
| A | C | | | | | | | | | | |
| ATOM | | 808 | O | GLY | A | 136 | −0.205 | 24.469 | 23.894 | 1.00 | 42.03 |
| A | O | | | | | | | | | | |
| ATOM | | 809 | N | ASP | A | 137 | −1.048 | 26.507 | 24.341 | 1.00 | 41.98 |
| A | N | | | | | | | | | | |
| ATOM | | 810 | CA | ASP | A | 137 | −1.881 | 26.544 | 23.146 | 1.00 | 41.03 |
| A | C | | | | | | | | | | |
| ATOM | | 811 | CB | ASP | A | 137 | −2.996 | 27.576 | 23.310 | 1.00 | 43.41 |
| A | C | | | | | | | | | | |
| ATOM | | 812 | CG | ASP | A | 137 | −3.844 | 27.710 | 22.064 | 1.00 | 45.06 |
| A | C | | | | | | | | | | |
| ATOM | | 813 | OD1 | ASP | A | 137 | −4.458 | 26.704 | 21.647 | 1.00 | 47.37 |
| A | O | | | | | | | | | | |
| ATOM | | 814 | OD2 | ASP | A | 137 | −3.892 | 28.821 | 21.499 | 1.00 | 45.71 |
| A | O | | | | | | | | | | |
| ATOM | | 815 | C | ASP | A | 137 | −1.016 | 26.908 | 21.944 | 1.00 | 39.51 |
| A | C | | | | | | | | | | |
| ATOM | | 816 | O | ASP | A | 137 | −0.162 | 27.783 | 22.036 | 1.00 | 39.46 |
| A | O | | | | | | | | | | |
| ATOM | | 817 | N | ASP | A | 138 | −1.241 | 26.244 | 20.814 | 1.00 | 38.39 |
| A | N | | | | | | | | | | |
| ATOM | | 818 | CA | ASP | A | 138 | −0.439 | 26.511 | 19.621 | 1.00 | 37.99 |
| A | C | | | | | | | | | | |
| ATOM | | 819 | CB | ASP | A | 138 | 0.168 | 25.208 | 19.102 | 1.00 | 39.01 |
| A | C | | | | | | | | | | |
| ATOM | | 820 | CG | ASP | A | 138 | −0.874 | 24.264 | 18.548 | 1.00 | 39.32 |
| A | C | | | | | | | | | | |
| ATOM | | 821 | OD1 | ASP | A | 138 | −2.070 | 24.475 | 18.826 | 1.00 | 39.65 |
| A | O | | | | | | | | | | |
| ATOM | | 822 | OD2 | ASP | A | 138 | −0.500 | 23.307 | 17.842 | 1.00 | 41.75 |
| A | O | | | | | | | | | | |
| ATOM | | 823 | C | ASP | A | 138 | −1.219 | 27.186 | 18.501 | 1.00 | 36.81 |
| A | C | | | | | | | | | | |
| ATOM | | 824 | O | ASP | A | 138 | −0.832 | 27.116 | 17.335 | 1.00 | 35.87 |
| A | O | | | | | | | | | | |
| ATOM | | 825 | N | LEU | A | 139 | −2.315 | 27.849 | 18.857 | 1.00 | 34.57 |
| A | N | | | | | | | | | | |
| ATOM | | 826 | CA | LEU | A | 139 | −3.139 | 28.532 | 17.870 | 1.00 | 33.64 |
| A | C | | | | | | | | | | |
| ATOM | | 827 | CB | LEU | A | 139 | −4.321 | 29.219 | 18.563 | 1.00 | 34.78 |
| A | C | | | | | | | | | | |
| ATOM | | 828 | CG | LEU | A | 139 | −5.618 | 29.352 | 17.757 | 1.00 | 36.95 |
| A | C | | | | | | | | | | |
| ATOM | | 829 | CD1 | LEU | A | 139 | −6.663 | 30.071 | 18.602 | 1.00 | 36.17 |
| A | C | | | | | | | | | | |
| ATOM | | 830 | CD2 | LEU | A | 139 | −5.364 | 30.112 | 16.464 | 1.00 | 36.43 |
| A | C | | | | | | | | | | |
| ATOM | | 831 | C | LEU | A | 139 | −2.326 | 29.561 | 17.079 | 1.00 | 31.14 |
| A | C | | | | | | | | | | |
| ATOM | | 832 | O | LEU | A | 139 | −2.461 | 29.666 | 15.862 | 1.00 | 30.79 |
| A | O | | | | | | | | | | |
| ATOM | | 833 | N | HIS | A | 140 | −1.483 | 30.318 | 17.769 | 1.00 | 29.61 |
| A | N | | | | | | | | | | |
| ATOM | | 834 | CA | HIS | A | 140 | −0.671 | 31.329 | 17.103 | 1.00 | 29.77 |
| A | C | | | | | | | | | | |
| ATOM | | 835 | CB | HIS | A | 140 | 0.039 | 32.210 | 18.138 | 1.00 | 32.63 |
| A | C | | | | | | | | | | |
| ATOM | | 836 | CG | HIS | A | 140 | 0.926 | 31.453 | 19.078 | 1.00 | 35.82 |
| A | C | | | | | | | | | | |
| ATOM | | 837 | CD2 | HIS | A | 140 | 2.183 | 31.706 | 19.515 | 1.00 | 36.65 |
| A | C | | | | | | | | | | |
| ATOM | | 838 | ND1 | HIS | A | 140 | 0.524 | 30.297 | 19.713 | 1.00 | 37.28 |
| A | N | | | | | | | | | | |
| ATOM | | 839 | CE1 | HIS | A | 140 | 1.496 | 29.870 | 20.500 | 1.00 | 38.18 |
| A | C | | | | | | | | | | |
| ATOM | | 840 | NE2 | HIS | A | 140 | 2.514 | 30.707 | 20.399 | 1.00 | 36.65 |
| A | N | | | | | | | | | | |
| ATOM | | 841 | C | HIS | A | 140 | 0.342 | 30.664 | 16.180 | 1.00 | 28.45 |
| A | C | | | | | | | | | | |
| ATOM | | 842 | O | HIS | A | 140 | 0.639 | 31.171 | 15.102 | 1.00 | 28.05 |
| A | O | | | | | | | | | | |
| ATOM | | 843 | N | ILE | A | 141 | 0.857 | 29.514 | 16.597 | 1.00 | 27.82 |
| A | N | | | | | | | | | | |
| ATOM | | 844 | CA | ILE | A | 141 | 1.825 | 28.784 | 15.790 | 1.00 | 26.79 |
| A | C | | | | | | | | | | |
| ATOM | | 845 | CB | ILE | A | 141 | 2.424 | 27.616 | 16.595 | 1.00 | 26.52 |
| A | C | | | | | | | | | | |
| ATOM | | 846 | CG2 | ILE | A | 141 | 3.057 | 26.598 | 15.667 | 1.00 | 24.81 |
| A | C | | | | | | | | | | |
| ATOM | | 847 | CG1 | ILE | A | 141 | 3.434 | 28.174 | 17.605 | 1.00 | 26.50 |
| A | C | | | | | | | | | | |
| ATOM | | 848 | CD | ILE | A | 141 | 3.981 | 27.146 | 18.563 | 1.00 | 28.45 |
| A | C | | | | | | | | | | |
| ATOM | | 849 | C | ILE | A | 141 | 1.168 | 28.269 | 14.516 | 1.00 | 27.29 |
| A | C | | | | | | | | | | |
| ATOM | | 850 | O | ILE | A | 141 | 1.749 | 28.344 | 13.434 | 1.00 | 26.99 |
| A | O | | | | | | | | | | |
| ATOM | | 851 | N | GLN | A | 142 | −0.053 | 27.760 | 14.647 | 1.00 | 28.53 |
| A | N | | | | | | | | | | |
| ATOM | | 852 | CA | GLN | A | 142 | −0.788 | 27.249 | 13.497 | 1.00 | 27.65 |
| A | C | | | | | | | | | | |
| ATOM | | 853 | CB | GLN | A | 142 | −2.128 | 26.667 | 13.940 | 1.00 | 29.20 |
| A | C | | | | | | | | | | |
| ATOM | | 854 | CG | GLN | A | 142 | −2.026 | 25.340 | 14.674 | 1.00 | 32.99 |
| A | C | | | | | | | | | | |
| ATOM | | 855 | CD | GLN | A | 142 | −3.388 | 24.786 | 15.049 | 1.00 | 35.51 |
| A | C | | | | | | | | | | |
| ATOM | | 856 | OE1 | GLN | A | 142 | −4.111 | 25.378 | 15.850 | 1.00 | 40.18 |
| A | O | | | | | | | | | | |
| ATOM | | 857 | NE2 | GLN | A | 142 | −3.748 | 23.651 | 14.464 | 1.00 | 36.87 |
| A | N | | | | | | | | | | |
| ATOM | | 858 | C | GLN | A | 142 | −1.025 | 28.354 | 12.473 | 1.00 | 26.74 |
| A | C | | | | | | | | | | |
| ATOM | | 859 | O | GLN | A | 142 | −0.895 | 28.132 | 11.272 | 1.00 | 26.24 |
| A | O | | | | | | | | | | |
| ATOM | | 860 | N | ARG | A | 143 | −1.376 | 29.542 | 12.953 | 1.00 | 27.29 |
| A | N | | | | | | | | | | |
| ATOM | | 861 | CA | ARG | A | 143 | −1.622 | 30.679 | 12.071 | 1.00 | 27.82 |
| A | C | | | | | | | | | | |
| ATOM | | 862 | CB | ARG | A | 143 | −2.106 | 31.886 | 12.878 | 1.00 | 29.63 |
| A | C | | | | | | | | | | |
| ATOM | | 863 | CG | ARG | A | 143 | −3.526 | 31.758 | 13.405 | 1.00 | 34.47 |
| A | C | | | | | | | | | | |
| ATOM | | 864 | CD | ARG | A | 143 | −3.788 | 32.781 | 14.507 | 1.00 | 39.86 |
| A | C | | | | | | | | | | |
| ATOM | | 865 | NE | ARG | A | 143 | −5.170 | 32.741 | 14.977 | 1.00 | 44.13 |
| A | N | | | | | | | | | | |
| ATOM | | 866 | CZ | ARG | A | 143 | −5.575 | 33.213 | 16.152 | 1.00 | 46.89 |
| A | C | | | | | | | | | | |
| ATOM | | 867 | NH1 | ARG | A | 143 | −4.701 | 33.762 | 16.987 | 1.00 | 47.65 |
| A | N | | | | | | | | | | |
| ATOM | | 868 | NH2 | ARG | A | 143 | −6.856 | 33.132 | 16.493 | 1.00 | 48.27 |
| A | N | | | | | | | | | | |
| ATOM | | 869 | C | ARG | A | 143 | −0.363 | 31.064 | 11.301 | 1.00 | 26.24 |
| A | C | | | | | | | | | | |
| ATOM | | 870 | O | ARG | A | 143 | −0.410 | 31.289 | 10.094 | 1.00 | 24.10 |
| A | O | | | | | | | | | | |
| ATOM | | 871 | N | ASN | A | 144 | 0.761 | 31.136 | 12.004 | 1.00 | 25.57 |
| A | N | | | | | | | | | | |
| ATOM | | 872 | CA | ASN | A | 144 | 2.024 | 31.511 | 11.377 | 1.00 | 25.34 |
| A | C | | | | | | | | | | |
| ATOM | | 873 | CB | ASN | A | 144 | 3.113 | 31.670 | 12.442 | 1.00 | 25.23 |
| A | C | | | | | | | | | | |
| ATOM | | 874 | CG | ASN | A | 144 | 2.901 | 32.901 | 13.310 | 1.00 | 27.47 |
| A | C | | | | | | | | | | |
| ATOM | | 875 | OD1 | ASN | A | 144 | 1.855 | 33.552 | 13.238 | 1.00 | 25.19 |
| A | O | | | | | | | | | | |
| ATOM | | 876 | ND2 | ASN | A | 144 | 3.894 | 33.225 | 14.137 | 1.00 | 26.15 |
| A | N | | | | | | | | | | |
| ATOM | | 877 | C | ASN | A | 144 | 2.460 | 30.508 | 10.319 | 1.00 | 24.78 |
| A | C | | | | | | | | | | |
| ATOM | | 878 | O | ASN | A | 144 | 2.963 | 30.896 | 9.259 | 1.00 | 24.90 |
| A | O | | | | | | | | | | |
| ATOM | | 879 | N | VAL | A | 145 | 2.262 | 29.222 | 10.599 | 1.00 | 23.95 |
| A | N | | | | | | | | | | |
| ATOM | | 880 | CA | VAL | A | 145 | 2.638 | 28.177 | 9.650 | 1.00 | 22.12 |
| A | C | | | | | | | | | | |
| ATOM | | 881 | CB | VAL | A | 145 | 2.578 | 26.771 | 10.298 | 1.00 | 22.24 |
| A | C | | | | | | | | | | |
| ATOM | | 882 | CG1 | VAL | A | 145 | 2.893 | 25.703 | 9.265 | 1.00 | 22.51 |
| A | C | | | | | | | | | | |
| ATOM | | 883 | CG2 | VAL | A | 145 | 3.571 | 26.688 | 11.442 | 1.00 | 19.36 |
| A | C | | | | | | | | | | |
| ATOM | | 884 | C | VAL | A | 145 | 1.705 | 28.219 | 8.445 | 1.00 | 23.02 |
| A | C | | | | | | | | | | |
| ATOM | | 885 | O | VAL | A | 145 | 2.143 | 28.075 | 7.304 | 1.00 | 20.64 |
| A | O | | | | | | | | | | |
| ATOM | | 886 | N | GLN | A | 146 | 0.417 | 28.430 | 8.703 | 1.00 | 24.15 |
| A | N | | | | | | | | | | |
| ATOM | | 887 | CA | GLN | A | 146 | −0.566 | 28.491 | 7.630 | 1.00 | 23.03 |
| A | C | | | | | | | | | | |
| ATOM | | 888 | CB | GLN | A | 146 | −1.962 | 28.746 | 8.197 | 1.00 | 25.85 |
| A | C | | | | | | | | | | |
| ATOM | | 889 | CG | GLN | A | 146 | −3.056 | 28.631 | 7.153 | 1.00 | 27.55 |
| A | C | | | | | | | | | | |
| ATOM | | 890 | CD | GLN | A | 146 | −3.028 | 27.291 | 6.443 | 1.00 | 27.72 |
| A | C | | | | | | | | | | |
| ATOM | | 891 | OE1 | GLN | A | 146 | −2.920 | 27.228 | 5.221 | 1.00 | 28.53 |
| A | O | | | | | | | | | | |
| ATOM | | 892 | NE2 | GLN | A | 146 | −3.125 | 26.210 | 7.210 | 1.00 | 29.98 |
| A | N | | | | | | | | | | |
| ATOM | | 893 | C | GLN | A | 146 | −0.227 | 29.586 | 6.625 | 1.00 | 23.12 |
| A | C | | | | | | | | | | |
| ATOM | | 894 | O | GLN | A | 146 | −0.409 | 29.411 | 5.422 | 1.00 | 22.07 |
| A | O | | | | | | | | | | |
| ATOM | | 895 | N | LYS | A | 147 | 0.253 | 30.720 | 7.120 | 1.00 | 21.60 |
| A | N | | | | | | | | | | |
| ATOM | | 896 | CA | LYS | A | 147 | 0.609 | 31.826 | 6.242 | 1.00 | 23.99 |
| A | C | | | | | | | | | | |
| ATOM | | 897 | CB | LYS | A | 147 | 0.982 | 33.069 | 7.054 | 1.00 | 27.26 |
| A | C | | | | | | | | | | |
| ATOM | | 898 | CG | LYS | A | 147 | 1.356 | 34.266 | 6.183 | 1.00 | 33.40 |
| A | C | | | | | | | | | | |
| ATOM | | 899 | CD | LYS | A | 147 | 0.223 | 34.614 | 5.211 | 1.00 | 37.04 |
| A | C | | | | | | | | | | |
| ATOM | | 900 | CE | LYS | A | 147 | 0.599 | 35.760 | 4.278 | 1.00 | 38.65 |
| A | C | | | | | | | | | | |
| ATOM | | 901 | NZ | LYS | A | 147 | 0.882 | 37.019 | 5.027 | 1.00 | 40.65 |
| A | N | | | | | | | | | | |
| ATOM | | 902 | C | LYS | A | 147 | 1.778 | 31.441 | 5.349 | 1.00 | 24.34 |
| A | C | | | | | | | | | | |
| ATOM | | 903 | O | LYS | A | 147 | 1.860 | 31.861 | 4.193 | 1.00 | 21.81 |
| A | O | | | | | | | | | | |
| ATOM | | 904 | N | LEU | A | 148 | 2.696 | 30.650 | 5.894 | 1.00 | 23.83 |
| A | N | | | | | | | | | | |
| ATOM | | 905 | CA | LEU | A | 148 | 3.844 | 30.216 | 5.120 | 1.00 | 23.73 |
| A | C | | | | | | | | | | |
| ATOM | | 906 | CB | LEU | A | 148 | 4.864 | 29.516 | 6.029 | 1.00 | 24.60 |
| A | C | | | | | | | | | | |
| ATOM | | 907 | CG | LEU | A | 148 | 6.213 | 29.108 | 5.425 | 1.00 | 25.23 |
| A | C | | | | | | | | | | |
| ATOM | | 908 | CD1 | LEU | A | 148 | 7.213 | 28.802 | 6.539 | 1.00 | 27.40 |
| A | C | | | | | | | | | | |
| ATOM | | 909 | CD2 | LEU | A | 148 | 6.023 | 27.897 | 4.535 | 1.00 | 25.60 |
| A | C | | | | | | | | | | |
| ATOM | | 910 | C | LEU | A | 148 | 3.315 | 29.272 | 4.043 | 1.00 | 21.03 |
| A | C | | | | | | | | | | |
| ATOM | | 911 | O | LEU | A | 148 | 3.706 | 29.364 | 2.885 | 1.00 | 22.18 |
| A | O | | | | | | | | | | |
| ATOM | | 912 | N | LYS | A | 149 | 2.411 | 28.376 | 4.422 | 1.00 | 21.82 |
| A | N | | | | | | | | | | |
| ATOM | | 913 | CA | LYS | A | 149 | 1.834 | 27.439 | 3.461 | 1.00 | 22.95 |
| A | C | | | | | | | | | | |
| ATOM | | 914 | CB | LYS | A | 149 | 0.852 | 26.495 | 4.148 | 1.00 | 24.40 |
| A | C | | | | | | | | | | |
| ATOM | | 915 | CG | LYS | A | 149 | 1.462 | 25.605 | 5.209 | 1.00 | 26.03 |
| A | C | | | | | | | | | | |
| ATOM | | 916 | CD | LYS | A | 149 | 0.433 | 24.591 | 5.683 | 1.00 | 29.36 |
| A | C | | | | | | | | | | |
| ATOM | | 917 | CE | LYS | A | 149 | 1.026 | 23.603 | 6.667 | 1.00 | 31.50 |
| A | C | | | | | | | | | | |
| ATOM | | 918 | NZ | LYS | A | 149 | 0.039 | 22.535 | 7.000 | 1.00 | 33.82 |
| A | N | | | | | | | | | | |
| ATOM | | 919 | C | LYS | A | 149 | 1.098 | 28.204 | 2.366 | 1.00 | 23.21 |
| A | C | | | | | | | | | | |
| ATOM | | 920 | O | LYS | A | 149 | 1.239 | 27.899 | 1.177 | 1.00 | 21.94 |
| A | O | | | | | | | | | | |
| ATOM | | 921 | N | ASP | A | 150 | 0.319 | 29.203 | 2.771 | 1.00 | 22.28 |
| A | N | | | | | | | | | | |
| ATOM | | 922 | CA | ASP | A | 150 | −0.441 | 30.003 | 1.814 | 1.00 | 23.20 |
| A | C | | | | | | | | | | |
| ATOM | | 923 | CB | ASP | A | 150 | −1.303 | 31.054 | 2.526 | 1.00 | 25.06 |
| A | C | | | | | | | | | | |
| ATOM | | 924 | CG | ASP | A | 150 | −2.359 | 30.444 | 3.430 | 1.00 | 27.96 |
| A | C | | | | | | | | | | |
| ATOM | | 925 | OD1 | ASP | A | 150 | −2.832 | 29.327 | 3.140 | 1.00 | 29.25 |
| A | O | | | | | | | | | | |
| ATOM | | 926 | OD2 | ASP | A | 150 | −2.731 | 31.100 | 4.428 | 1.00 | 30.84 |
| A | O | | | | | | | | | | |
| ATOM | | 927 | C | ASP | A | 150 | 0.487 | 30.716 | 0.844 | 1.00 | 22.09 |
| A | C | | | | | | | | | | |
| ATOM | | 928 | O | ASP | A | 150 | 0.201 | 30.804 | −0.349 | 1.00 | 22.07 |
| A | O | | | | | | | | | | |
| ATOM | | 929 | N | THR | A | 151 | 1.599 | 31.228 | 1.360 | 1.00 | 20.13 |
| A | N | | | | | | | | | | |
| ATOM | | 930 | CA | THR | A | 151 | 2.557 | 31.949 | 0.533 | 1.00 | 20.21 |
| A | C | | | | | | | | | | |
| ATOM | | 931 | CB | THR | A | 151 | 3.676 | 32.578 | 1.390 | 1.00 | 21.32 |
| A | C | | | | | | | | | | |
| ATOM | | 932 | OG1 | THR | A | 151 | 3.097 | 33.422 | 2.392 | 1.00 | 22.82 |
| A | O | | | | | | | | | | |
| ATOM | | 933 | CG2 | THR | A | 151 | 4.602 | 33.410 | 0.520 | 1.00 | 23.30 |
| A | C | | | | | | | | | | |
| ATOM | | 934 | C | THR | A | 151 | 3.184 | 31.045 | −0.521 | 1.00 | 19.34 |
| A | C | | | | | | | | | | |
| ATOM | | 935 | O | THR | A | 151 | 3.403 | 31.466 | −1.659 | 1.00 | 16.62 |
| A | O | | | | | | | | | | |
| ATOM | | 936 | N | VAL | A | 152 | 3.482 | 29.809 | −0.134 | 1.00 | 17.83 |
| A | N | | | | | | | | | | |
| ATOM | | 937 | CA | VAL | A | 152 | 4.071 | 28.849 | −1.056 | 1.00 | 20.82 |
| A | C | | | | | | | | | | |
| ATOM | | 938 | CB | VAL | A | 152 | 4.465 | 27.545 | −0.322 | 1.00 | 19.02 |
| A | C | | | | | | | | | | |
| ATOM | | 939 | CG1 | VAL | A | 152 | 4.739 | 26.433 | −1.323 | 1.00 | 21.92 |
| A | C | | | | | | | | | | |
| ATOM | | 940 | CG2 | VAL | A | 152 | 5.716 | 27.782 | 0.507 | 1.00 | 20.80 |
| A | C | | | | | | | | | | |
| ATOM | | 941 | C | VAL | A | 152 | 3.087 | 28.535 | −2.183 | 1.00 | 22.16 |
| A | C | | | | | | | | | | |
| ATOM | | 942 | O | VAL | A | 152 | 3.479 | 28.449 | −3.346 | 1.00 | 23.43 |
| A | O | | | | | | | | | | |
| ATOM | | 943 | N | LYS | A | 153 | 1.810 | 28.379 | −1.843 | 1.00 | 24.38 |
| A | N | | | | | | | | | | |
| ATOM | | 944 | CA | LYS | A | 153 | 0.789 | 28.087 | −2.849 | 1.00 | 27.82 |
| A | C | | | | | | | | | | |
| ATOM | | 945 | CB | LYS | A | 153 | −0.534 | 27.732 | −2.176 | 1.00 | 31.58 |
| A | C | | | | | | | | | | |
| ATOM | | 946 | CG | LYS | A | 153 | −0.441 | 26.583 | −1.209 | 1.00 | 35.04 |
| A | C | | | | | | | | | | |
| ATOM | | 947 | CD | LYS | A | 153 | −1.778 | 26.401 | −0.548 | 1.00 | 40.47 |
| A | C | | | | | | | | | | |
| ATOM | | 948 | CE | LYS | A | 153 | −1.689 | 25.379 | 0.560 | 1.00 | 43.02 |
| A | C | | | | | | | | | | |
| ATOM | | 949 | NZ | LYS | A | 153 | −2.952 | 25.190 | 1.344 | 1.00 | 44.36 |
| A | N | | | | | | | | | | |
| ATOM | | 950 | C | LYS | A | 153 | 0.585 | 29.279 | −3.784 | 1.00 | 29.16 |
| A | C | | | | | | | | | | |
| ATOM | | 951 | O | LYS | A | 153 | 0.437 | 29.108 | −4.996 | 1.00 | 28.68 |
| A | O | | | | | | | | | | |
| ATOM | | 952 | N | LYS | A | 154 | 0.580 | 30.484 | −3.218 | 1.00 | 28.50 |
| A | N | | | | | | | | | | |
| ATOM | | 953 | CA | LYS | A | 154 | 0.402 | 31.699 | −4.005 | 1.00 | 29.69 |
| A | C | | | | | | | | | | |
| ATOM | | 954 | CB | LYS | A | 154 | 0.479 | 32.942 | −3.106 | 1.00 | 32.52 |
| A | C | | | | | | | | | | |
| ATOM | | 955 | CG | LYS | A | 154 | −0.616 | 33.048 | −2.053 | 1.00 | 37.41 |
| A | C | | | | | | | | | | |
| ATOM | | 956 | CD | LYS | A | 154 | −0.339 | 34.206 | −1.095 | 1.00 | 40.74 |
| A | C | | | | | | | | | | |
| ATOM | | 957 | CE | LYS | A | 154 | −1.385 | 34.290 | 0.009 | 1.00 | 42.32 |
| A | C | | | | | | | | | | |
| ATOM | | 958 | NZ | LYS | A | 154 | −1.038 | 35.319 | 1.032 | 1.00 | 42.89 |
| A | N | | | | | | | | | | |
| ATOM | | 959 | C | LYS | A | 154 | 1.496 | 31.788 | −5.064 | 1.00 | 28.78 |
| A | C | | | | | | | | | | |
| ATOM | | 960 | O | LYS | A | 154 | 1.291 | 32.351 | −6.144 | 1.00 | 28.74 |
| A | O | | | | | | | | | | |
| ATOM | | 961 | N | LEU | A | 155 | 2.661 | 31.232 | −4.738 | 1.00 | 26.21 |
| A | N | | | | | | | | | | |
| ATOM | | 962 | CA | LEU | A | 155 | 3.805 | 31.253 | −5.640 | 1.00 | 24.24 |
| A | C | | | | | | | | | | |
| ATOM | | 963 | CB | LEU | A | 155 | 5.118 | 31.313 | −4.847 | 1.00 | 22.63 |
| A | C | | | | | | | | | | |
| ATOM | | 964 | CG | LEU | A | 155 | 5.392 | 32.561 | −3.979 | 1.00 | 24.03 |
| A | C | | | | | | | | | | |
| ATOM | | 965 | CD1 | LEU | A | 155 | 6.695 | 32.374 | −3.263 | 1.00 | 25.32 |
| A | C | | | | | | | | | | |
| ATOM | | 966 | CD2 | LEU | A | 155 | 5.518 | 33.828 | −4.793 | 1.00 | 23.74 |
| A | C | | | | | | | | | | |
| ATOM | | 967 | C | LEU | A | 155 | 3.807 | 30.058 | −6.576 | 1.00 | 22.47 |
| A | C | | | | | | | | | | |
| ATOM | | 968 | O | LEU | A | 155 | 4.504 | 30.059 | −7.590 | 1.00 | 22.33 |
| A | O | | | | | | | | | | |
| ATOM | | 969 | N | GLY | A | 156 | 3.009 | 29.051 | −6.240 | 1.00 | 23.16 |
| A | N | | | | | | | | | | |
| ATOM | | 970 | CA | GLY | A | 156 | 2.927 | 27.864 | −7.068 | 1.00 | 22.02 |
| A | C | | | | | | | | | | |
| ATOM | | 971 | C | GLY | A | 156 | 4.208 | 27.063 | −7.063 | 1.00 | 21.25 |
| A | C | | | | | | | | | | |
| ATOM | | 972 | O | GLY | A | 156 | 4.829 | 26.876 | −6.020 | 1.00 | 21.24 |
| A | O | | | | | | | | | | |
| ATOM | | 973 | N | GLU | A | 157 | 4.604 | 26.585 | −8.236 | 1.00 | 20.06 |
| A | N | | | | | | | | | | |
| ATOM | | 974 | CA | GLU | A | 157 | 5.816 | 25.788 | −8.371 | 1.00 | 21.41 |
| A | C | | | | | | | | | | |
| ATOM | | 975 | CB | GLU | A | 157 | 6.055 | 25.449 | −9.849 | 1.00 | 23.31 |
| A | C | | | | | | | | | | |
| ATOM | | 976 | CG | GLU | A | 157 | 7.319 | 24.650 | −10.107 | 1.00 | 28.64 |
| A | C | | | | | | | | | | |
| ATOM | | 977 | CD | GLU | A | 157 | 7.147 | 23.189 | −9.770 | 1.00 | 31.67 |
| A | C | | | | | | | | | | |
| ATOM | | 978 | OE1 | GLU | A | 157 | 6.397 | 22.891 | −8.824 | 1.00 | 34.33 |
| A | O | | | | | | | | | | |
| ATOM | | 979 | OE2 | GLU | A | 157 | 7.765 | 22.334 | −10.439 | 1.00 | 33.96 |
| A | O | | | | | | | | | | |
| ATOM | | 980 | C | GLU | A | 157 | 7.029 | 26.539 | −7.817 | 1.00 | 19.72 |
| A | C | | | | | | | | | | |
| ATOM | | 981 | O | GLU | A | 157 | 7.860 | 25.971 | −7.105 | 1.00 | 19.07 |
| A | O | | | | | | | | | | |
| ATOM | | 982 | N | SER | A | 158 | 7.129 | 27.821 | −8.147 | 1.00 | 18.69 |
| A | N | | | | | | | | | | |
| ATOM | | 983 | CA | SER | A | 158 | 8.249 | 28.624 | −7.680 | 1.00 | 19.07 |
| A | C | | | | | | | | | | |
| ATOM | | 984 | CB | SER | A | 158 | 8.218 | 30.006 | −8.337 | 1.00 | 20.27 |
| A | C | | | | | | | | | | |
| ATOM | | 985 | OG | SER | A | 158 | 7.058 | 30.717 | −7.957 | 1.00 | 24.37 |
| A | O | | | | | | | | | | |
| ATOM | | 986 | C | SER | A | 158 | 8.225 | 28.761 | −6.158 | 1.00 | 17.90 |
| A | C | | | | | | | | | | |
| ATOM | | 987 | O | SER | A | 158 | 9.214 | 29.166 | −5.549 | 1.00 | 16.77 |
| A | O | | | | | | | | | | |
| ATOM | | 988 | N | GLY | A | 159 | 7.088 | 28.433 | −5.552 | 1.00 | 15.07 |
| A | N | | | | | | | | | | |
| ATOM | | 989 | CA | GLY | A | 159 | 6.975 | 28.500 | −4.108 | 1.00 | 13.85 |
| A | C | | | | | | | | | | |
| ATOM | | 990 | C | GLY | A | 159 | 7.806 | 27.375 | −3.524 | 1.00 | 12.96 |
| A | C | | | | | | | | | | |
| ATOM | | 991 | O | GLY | A | 159 | 8.496 | 27.545 | −2.521 | 1.00 | 11.66 |
| A | O | | | | | | | | | | |
| ATOM | | 992 | N | GLU | A | 160 | 7.729 | 26.215 | −4.163 | 1.00 | 13.13 |
| A | N | | | | | | | | | | |
| ATOM | | 993 | CA | GLU | A | 160 | 8.486 | 25.046 | −3.741 | 1.00 | 14.60 |
| A | C | | | | | | | | | | |
| ATOM | | 994 | CB | GLU | A | 160 | 8.017 | 23.820 | −4.522 | 1.00 | 16.03 |
| A | C | | | | | | | | | | |
| ATOM | | 995 | CG | GLU | A | 160 | 6.665 | 23.291 | −4.073 | 1.00 | 17.68 |
| A | C | | | | | | | | | | |
| ATOM | | 996 | CD | GLU | A | 160 | 6.076 | 22.326 | −5.082 | 1.00 | 19.55 |
| A | C | | | | | | | | | | |
| ATOM | | 997 | OE1 | GLU | A | 160 | 6.849 | 21.556 | −5.687 | 1.00 | 19.93 |
| A | O | | | | | | | | | | |
| ATOM | | 998 | OE2 | GLU | A | 160 | 4.843 | 22.341 | −5.266 | 1.00 | 24.36 |
| A | O | | | | | | | | | | |
| ATOM | | 999 | C | GLU | A | 160 | 9.972 | 25.293 | −3.988 | 1.00 | 13.84 |
| A | C | | | | | | | | | | |
| ATOM | | 1000 | O | GLU | A | 160 | 10.819 | 24.908 | −3.180 | 1.00 | 13.51 |
| A | O | | | | | | | | | | |
| ATOM | | 1001 | N | ILE | A | 161 | 10.278 | 25.941 | −5.109 | 1.00 | 13.97 |
| A | N | | | | | | | | | | |
| ATOM | | 1002 | CA | ILE | A | 161 | 11.659 | 26.262 | −5.457 | 1.00 | 15.01 |
| A | C | | | | | | | | | | |
| ATOM | | 1003 | CB | ILE | A | 161 | 11.752 | 26.949 | −6.853 | 1.00 | 16.72 |
| A | C | | | | | | | | | | |
| ATOM | | 1004 | CG2 | ILE | A | 161 | 13.191 | 27.359 | −7.146 | 1.00 | 15.56 |
| A | C | | | | | | | | | | |
| ATOM | | 1005 | CG1 | ILE | A | 161 | 11.225 | 26.012 | −7.947 | 1.00 | 16.62 |
| A | C | | | | | | | | | | |
| ATOM | | 1006 | CD | ILE | A | 161 | 11.853 | 24.641 | −7.954 | 1.00 | 19.54 |
| A | C | | | | | | | | | | |
| ATOM | | 1007 | C | ILE | A | 161 | 12.220 | 27.219 | −4.399 | 1.00 | 14.73 |
| A | C | | | | | | | | | | |
| ATOM | | 1008 | O | ILE | A | 161 | 13.351 | 27.053 | −3.927 | 1.00 | 15.29 |
| A | O | | | | | | | | | | |
| ATOM | | 1009 | N | LYS | A | 162 | 11.424 | 28.216 | −4.024 | 1.00 | 11.62 |
| A | N | | | | | | | | | | |
| ATOM | | 1010 | CA | LYS | A | 162 | 11.852 | 29.185 | −3.021 | 1.00 | 12.12 |
| A | C | | | | | | | | | | |
| ATOM | | 1011 | CB | LYS | A | 162 | 10.787 | 30.269 | −2.829 | 1.00 | 12.70 |
| A | C | | | | | | | | | | |
| ATOM | | 1012 | CG | LYS | A | 162 | 11.098 | 31.245 | −1.697 | 1.00 | 11.50 |
| A | C | | | | | | | | | | |
| ATOM | | 1013 | CD | LYS | A | 162 | 10.128 | 32.416 | −1.676 | 1.00 | 15.16 |
| A | C | | | | | | | | | | |
| ATOM | | 1014 | CE | LYS | A | 162 | 10.361 | 33.352 | −2.853 | 1.00 | 15.54 |
| A | C | | | | | | | | | | |
| ATOM | | 1015 | NZ | LYS | A | 162 | 11.753 | 33.881 | −2.894 | 1.00 | 17.14 |
| A | N | | | | | | | | | | |
| ATOM | | 1016 | C | LYS | A | 162 | 12.146 | 28.503 | −1.686 | 1.00 | 13.23 |
| A | C | | | | | | | | | | |
| ATOM | | 1017 | O | LYS | A | 162 | 13.160 | 28.792 | −1.050 | 1.00 | 13.30 |
| A | O | | | | | | | | | | |
| ATOM | | 1018 | N | ALA | A | 163 | 11.260 | 27.603 | −1.266 | 1.00 | 12.38 |
| A | N | | | | | | | | | | |
| ATOM | | 1019 | CA | ALA | A | 163 | 11.438 | 26.880 | −0.009 | 1.00 | 12.47 |
| A | C | | | | | | | | | | |
| ATOM | | 1020 | CB | ALA | A | 163 | 10.286 | 25.913 | 0.211 | 1.00 | 12.98 |
| A | C | | | | | | | | | | |
| ATOM | | 1021 | C | ALA | A | 163 | 12.756 | 26.117 | −0.011 | 1.00 | 13.14 |
| A | C | | | | | | | | | | |
| ATOM | | 1022 | O | ALA | A | 163 | 13.452 | 26.062 | 1.008 | 1.00 | 12.48 |
| A | O | | | | | | | | | | |
| ATOM | | 1023 | N | ILE | A | 164 | 13.095 | 25.516 | −1.149 | 1.00 | 12.42 |
| A | N | | | | | | | | | | |
| ATOM | | 1024 | CA | ILE | A | 164 | 14.346 | 24.780 | −1.260 | 1.00 | 11.86 |
| A | C | | | | | | | | | | |
| ATOM | | 1025 | CB | ILE | A | 164 | 14.435 | 23.993 | −2.589 | 1.00 | 12.07 |
| A | C | | | | | | | | | | |
| ATOM | | 1026 | CG2 | ILE | A | 164 | 15.731 | 23.202 | −2.635 | 1.00 | 10.78 |
| A | C | | | | | | | | | | |
| ATOM | | 1027 | CG1 | ILE | A | 164 | 13.262 | 23.017 | −2.718 | 1.00 | 12.17 |
| A | C | | | | | | | | | | |
| ATOM | | 1028 | CD | ILE | A | 164 | 13.278 | 22.229 | −4.022 | 1.00 | 12.86 |
| A | C | | | | | | | | | | |
| ATOM | | 1029 | C | ILE | A | 164 | 15.491 | 25.800 | −1.204 | 1.00 | 12.22 |
| A | C | | | | | | | | | | |
| ATOM | | 1030 | O | ILE | A | 164 | 16.541 | 25.545 | −0.610 | 1.00 | 11.10 |
| A | O | | | | | | | | | | |
| ATOM | | 1031 | N | GLY | A | 165 | 15.281 | 26.955 | −1.826 | 1.00 | 10.67 |
| A | N | | | | | | | | | | |
| ATOM | | 1032 | CA | GLY | A | 165 | 16.303 | 27.989 | −1.813 | 1.00 | 12.02 |
| A | C | | | | | | | | | | |
| ATOM | | 1033 | C | GLY | A | 165 | 16.550 | 28.534 | −0.417 | 1.00 | 13.62 |
| A | C | | | | | | | | | | |
| ATOM | | 1034 | O | GLY | A | 165 | 17.609 | 29.107 | −0.140 | 1.00 | 12.29 |
| A | O | | | | | | | | | | |
| ATOM | | 1035 | N | GLU | A | 166 | 15.571 | 28.365 | 0.468 | 1.00 | 12.40 |
| A | N | | | | | | | | | | |
| ATOM | | 1036 | CA | GLU | A | 166 | 15.698 | 28.837 | 1.846 | 1.00 | 13.51 |
| A | C | | | | | | | | | | |
| ATOM | | 1037 | CB | GLU | A | 166 | 14.395 | 29.508 | 2.309 | 1.00 | 13.61 |
| A | C | | | | | | | | | | |
| ATOM | | 1038 | CG | GLU | A | 166 | 14.217 | 30.919 | 1.757 | 1.00 | 16.87 |
| A | C | | | | | | | | | | |
| ATOM | | 1039 | CD | GLU | A | 166 | 13.013 | 31.651 | 2.334 | 1.00 | 18.65 |
| A | C | | | | | | | | | | |
| ATOM | | 1040 | OE1 | GLU | A | 166 | 12.660 | 31.392 | 3.504 | 1.00 | 15.68 |
| A | O | | | | | | | | | | |
| ATOM | | 1041 | OE2 | GLU | A | 166 | 12.435 | 32.507 | 1.618 | 1.00 | 18.26 |
| A | O | | | | | | | | | | |
| ATOM | | 1042 | C | GLU | A | 166 | 16.068 | 27.720 | 2.821 | 1.00 | 13.52 |
| A | C | | | | | | | | | | |
| ATOM | | 1043 | O | GLU | A | 166 | 15.908 | 27.877 | 4.028 | 1.00 | 12.78 |
| A | O | | | | | | | | | | |
| ATOM | | 1044 | N | LEU | A | 167 | 16.563 | 26.594 | 2.310 | 1.00 | 12.05 |
| A | N | | | | | | | | | | |
| ATOM | | 1045 | CA | LEU | A | 167 | 16.937 | 25.505 | 3.203 | 1.00 | 11.58 |
| A | C | | | | | | | | | | |
| ATOM | | 1046 | CB | LEU | A | 167 | 17.366 | 24.264 | 2.417 | 1.00 | 13.77 |
| A | C | | | | | | | | | | |
| ATOM | | 1047 | CG | LEU | A | 167 | 16.223 | 23.441 | 1.818 | 1.00 | 14.22 |
| A | C | | | | | | | | | | |
| ATOM | | 1048 | CD1 | LEU | A | 167 | 16.778 | 22.130 | 1.246 | 1.00 | 16.24 |
| A | C | | | | | | | | | | |
| ATOM | | 1049 | CD2 | LEU | A | 167 | 15.179 | 23.155 | 2.895 | 1.00 | 16.98 |
| A | C | | | | | | | | | | |
| ATOM | | 1050 | C | LEU | A | 167 | 18.048 | 25.922 | 4.158 | 1.00 | 12.19 |
| A | C | | | | | | | | | | |
| ATOM | | 1051 | O | LEU | A | 167 | 18.206 | 25.334 | 5.233 | 1.00 | 11.69 |
| A | O | | | | | | | | | | |
| ATOM | | 1052 | N | ASP | A | 168 | 18.816 | 26.937 | 3.770 | 1.00 | 12.76 |
| A | N | | | | | | | | | | |
| ATOM | | 1053 | CA | ASP | A | 168 | 19.887 | 27.419 | 4.625 | 1.00 | 13.15 |
| A | C | | | | | | | | | | |
| ATOM | | 1054 | CB | ASP | A | 168 | 20.843 | 28.342 | 3.848 | 1.00 | 16.25 |
| A | C | | | | | | | | | | |
| ATOM | | 1055 | CG | ASP | A | 168 | 20.139 | 29.497 | 3.160 | 1.00 | 19.91 |
| A | C | | | | | | | | | | |
| ATOM | | 1056 | OD1 | ASP | A | 168 | 18.915 | 29.421 | 2.926 | 1.00 | 18.66 |
| A | O | | | | | | | | | | |
| ATOM | | 1057 | OD2 | ASP | A | 168 | 20.827 | 30.487 | 2.832 | 1.00 | 20.94 |
| A | O | | | | | | | | | | |
| ATOM | | 1058 | C | ASP | A | 168 | 19.279 | 28.116 | 5.836 | 1.00 | 13.72 |
| A | C | | | | | | | | | | |
| ATOM | | 1059 | O | ASP | A | 168 | 19.777 | 27.975 | 6.953 | 1.00 | 15.78 |
| A | O | | | | | | | | | | |
| ATOM | | 1060 | N | LEU | A | 169 | 18.190 | 28.851 | 5.622 | 1.00 | 13.06 |
| A | N | | | | | | | | | | |
| ATOM | | 1061 | CA | LEU | A | 169 | 17.499 | 29.523 | 6.717 | 1.00 | 13.54 |
| A | C | | | | | | | | | | |
| ATOM | | 1062 | CB | LEU | A | 169 | 16.525 | 30.577 | 6.173 | 1.00 | 14.93 |
| A | C | | | | | | | | | | |
| ATOM | | 1063 | CG | LEU | A | 169 | 17.254 | 31.653 | 5.365 | 1.00 | 16.86 |
| A | C | | | | | | | | | | |
| ATOM | | 1064 | CD1 | LEU | A | 169 | 16.306 | 32.688 | 4.873 | 1.00 | 17.37 |
| A | C | | | | | | | | | | |
| ATOM | | 1065 | CD2 | LEU | A | 169 | 18.260 | 32.337 | 6.229 | 1.00 | 19.91 |
| A | C | | | | | | | | | | |
| ATOM | | 1066 | C | LEU | A | 169 | 16.738 | 28.499 | 7.556 | 1.00 | 12.68 |
| A | C | | | | | | | | | | |
| ATOM | | 1067 | O | LEU | A | 169 | 16.595 | 28.659 | 8.770 | 1.00 | 13.63 |
| A | O | | | | | | | | | | |
| ATOM | | 1068 | N | LEU | A | 170 | 16.251 | 27.446 | 6.905 | 1.00 | 12.87 |
| A | N | | | | | | | | | | |
| ATOM | | 1069 | CA | LEU | A | 170 | 15.526 | 26.397 | 7.604 | 1.00 | 12.73 |
| A | C | | | | | | | | | | |
| ATOM | | 1070 | CB | LEU | A | 170 | 14.937 | 25.387 | 6.612 | 1.00 | 13.33 |
| A | C | | | | | | | | | | |
| ATOM | | 1071 | CG | LEU | A | 170 | 14.185 | 24.231 | 7.280 | 1.00 | 14.18 |
| A | C | | | | | | | | | | |
| ATOM | | 1072 | CD1 | LEU | A | 170 | 13.048 | 24.789 | 8.111 | 1.00 | 15.94 |
| A | C | | | | | | | | | | |
| ATOM | | 1073 | CD2 | LEU | A | 170 | 13.649 | 23.275 | 6.225 | 1.00 | 15.77 |
| A | C | | | | | | | | | | |
| ATOM | | 1074 | C | LEU | A | 170 | 16.514 | 25.677 | 8.506 | 1.00 | 11.78 |
| A | C | | | | | | | | | | |
| ATOM | | 1075 | O | LEU | A | 170 | 16.230 | 25.398 | 9.671 | 1.00 | 10.15 |
| A | O | | | | | | | | | | |
| ATOM | | 1076 | N | PHE | A | 171 | 17.676 | 25.371 | 7.947 | 1.00 | 12.13 |
| A | N | | | | | | | | | | |
| ATOM | | 1077 | CA | PHE | A | 171 | 18.716 | 24.684 | 8.690 | 1.00 | 11.64 |
| A | C | | | | | | | | | | |
| ATOM | | 1078 | CB | PHE | A | 171 | 19.938 | 24.478 | 7.787 | 1.00 | 12.34 |
| A | C | | | | | | | | | | |
| ATOM | | 1079 | CG | PHE | A | 171 | 21.100 | 23.800 | 8.465 | 1.00 | 13.38 |
| A | C | | | | | | | | | | |
| ATOM | | 1080 | CD1 | PHE | A | 171 | 21.980 | 24.529 | 9.262 | 1.00 | 12.95 |
| A | C | | | | | | | | | | |
| ATOM | | 1081 | CD2 | PHE | A | 171 | 21.330 | 22.438 | 8.282 | 1.00 | 12.74 |
| A | C | | | | | | | | | | |
| ATOM | | 1082 | CE1 | PHE | A | 171 | 23.077 | 23.911 | 9.862 | 1.00 | 12.19 |
| A | C | | | | | | | | | | |
| ATOM | | 1083 | CE2 | PHE | A | 171 | 22.426 | 21.808 | 8.878 | 1.00 | 13.42 |
| A | C | | | | | | | | | | |
| ATOM | | 1084 | CZ | PHE | A | 171 | 23.303 | 22.549 | 9.670 | 1.00 | 9.66 |
| A | C | | | | | | | | | | |
| ATOM | | 1085 | C | PHE | A | 171 | 19.097 | 25.487 | 9.933 | 1.00 | 12.85 |
| A | C | | | | | | | | | | |
| ATOM | | 1086 | O | PHE | A | 171 | 19.112 | 24.956 | 11.047 | 1.00 | 13.16 |
| A | O | | | | | | | | | | |
| ATOM | | 1087 | N | MET | A | 172 | 19.367 | 26.773 | 9.739 | 1.00 | 12.41 |
| A | N | | | | | | | | | | |
| ATOM | | 1088 | CA | MET | A | 172 | 19.770 | 27.633 | 10.845 | 1.00 | 13.98 |
| A | C | | | | | | | | | | |
| ATOM | | 1089 | CB | AMET | A | 172 | 20.339 | 28.952 | 10.310 | 0.80 | 15.64 |
| A | C | | | | | | | | | | |
| ATOM | | 1090 | CB | BMET | A | 172 | 20.370 | 28.959 | 10.355 | 0.20 | 14.94 |
| A | C | | | | | | | | | | |
| ATOM | | 1091 | CG | AMET | A | 172 | 21.571 | 28.732 | 9.435 | 0.80 | 16.09 |
| A | C | | | | | | | | | | |
| ATOM | | 1092 | CG | BMET | A | 172 | 19.527 | 29.804 | 9.448 | 0.20 | 15.64 |
| A | C | | | | | | | | | | |
| ATOM | | 1093 | SD | AMET | A | 172 | 22.552 | 30.211 | 9.177 | 0.80 | 21.52 |
| A | S | | | | | | | | | | |
| ATOM | | 1094 | SD | BMET | A | 172 | 20.472 | 31.295 | 9.068 | 0.20 | 14.45 |
| A | S | | | | | | | | | | |
| ATOM | | 1095 | CE | AMET | A | 172 | 21.697 | 30.932 | 7.776 | 0.80 | 17.67 |
| A | C | | | | | | | | | | |
| ATOM | | 1096 | CE | BMET | A | 172 | 21.541 | 30.699 | 7.752 | 0.20 | 18.36 |
| A | C | | | | | | | | | | |
| ATOM | | 1097 | C | MET | A | 172 | 18.662 | 27.902 | 11.848 | 1.00 | 13.07 |
| A | C | | | | | | | | | | |
| ATOM | | 1098 | O | MET | A | 172 | 18.937 | 28.048 | 13.039 | 1.00 | 13.04 |
| A | O | | | | | | | | | | |
| ATOM | | 1099 | N | SER | A | 173 | 17.418 | 27.947 | 11.379 | 1.00 | 13.08 |
| A | N | | | | | | | | | | |
| ATOM | | 1100 | CA | SER | A | 173 | 16.284 | 28.199 | 12.261 | 1.00 | 11.55 |
| A | C | | | | | | | | | | |
| ATOM | | 1101 | CB | SER | A | 173 | 15.044 | 28.557 | 11.440 | 1.00 | 11.95 |
| A | C | | | | | | | | | | |
| ATOM | | 1102 | OG | SER | A | 173 | 15.174 | 29.828 | 10.818 | 1.00 | 14.62 |
| A | O | | | | | | | | | | |
| ATOM | | 1103 | C | SER | A | 173 | 16.019 | 26.963 | 13.127 | 1.00 | 11.91 |
| A | C | | | | | | | | | | |
| ATOM | | 1104 | O | SER | A | 173 | 15.713 | 27.082 | 14.315 | 1.00 | 14.96 |
| A | O | | | | | | | | | | |
| ATOM | | 1105 | N | LEU | A | 174 | 16.121 | 25.777 | 12.529 | 1.00 | 11.90 |
| A | N | | | | | | | | | | |
| ATOM | | 1106 | CA | LEU | A | 174 | 15.927 | 24.539 | 13.279 | 1.00 | 12.23 |
| A | C | | | | | | | | | | |
| ATOM | | 1107 | CB | LEU | A | 174 | 16.053 | 23.319 | 12.351 | 1.00 | 13.54 |
| A | C | | | | | | | | | | |
| ATOM | | 1108 | CG | LEU | A | 174 | 14.780 | 22.851 | 11.634 | 1.00 | 15.89 |
| A | C | | | | | | | | | | |
| ATOM | | 1109 | CD1 | LEU | A | 174 | 15.132 | 21.898 | 10.491 | 1.00 | 14.93 |
| A | C | | | | | | | | | | |
| ATOM | | 1110 | CD2 | LEU | A | 174 | 13.845 | 22.183 | 12.637 | 1.00 | 12.22 |
| A | C | | | | | | | | | | |
| ATOM | | 1111 | C | LEU | A | 174 | 16.999 | 24.465 | 14.371 | 1.00 | 12.75 |
| A | C | | | | | | | | | | |
| ATOM | | 1112 | O | LEU | A | 174 | 16.710 | 24.160 | 15.521 | 1.00 | 13.46 |
| A | O | | | | | | | | | | |
| ATOM | | 1113 | N | ARG | A | 175 | 18.243 | 24.746 | 13.993 | 1.00 | 11.68 |
| A | N | | | | | | | | | | |
| ATOM | | 1114 | CA | ARG | A | 175 | 19.360 | 24.709 | 14.930 | 1.00 | 14.76 |
| A | C | | | | | | | | | | |
| ATOM | | 1115 | CB | ARG | A | 175 | 20.673 | 24.990 | 14.177 | 1.00 | 12.95 |
| A | C | | | | | | | | | | |
| ATOM | | 1116 | CG | ARG | A | 175 | 21.888 | 25.301 | 15.040 | 1.00 | 15.09 |
| A | C | | | | | | | | | | |
| ATOM | | 1117 | CD | ARG | A | 175 | 23.156 | 25.325 | 14.175 | 1.00 | 13.01 |
| A | C | | | | | | | | | | |
| ATOM | | 1118 | NE | ARG | A | 175 | 24.292 | 25.954 | 14.847 | 1.00 | 16.67 |
| A | N | | | | | | | | | | |
| ATOM | | 1119 | CZ | ARG | A | 175 | 25.566 | 25.771 | 14.505 | 1.00 | 15.77 |
| A | C | | | | | | | | | | |
| ATOM | | 1120 | NH1 | ARG | A | 175 | 25.888 | 24.962 | 13.502 | 1.00 | 11.71 |
| A | N | | | | | | | | | | |
| ATOM | | 1121 | NH2 | ARG | A | 175 | 26.525 | 26.429 | 15.144 | 1.00 | 16.45 |
| A | N | | | | | | | | | | |
| ATOM | | 1122 | C | ARG | A | 175 | 19.146 | 25.733 | 16.051 | 1.00 | 14.85 |
| A | C | | | | | | | | | | |
| ATOM | | 1123 | O | ARG | A | 175 | 19.163 | 25.392 | 17.235 | 1.00 | 16.14 |
| A | O | | | | | | | | | | |
| ATOM | | 1124 | N | ASN | A | 176 | 18.898 | 26.978 | 15.678 | 1.00 | 12.31 |
| A | N | | | | | | | | | | |
| ATOM | | 1125 | CA | ASN | A | 176 | 18.708 | 28.021 | 16.678 | 1.00 | 13.85 |
| A | C | | | | | | | | | | |
| ATOM | | 1126 | CB | ASN | A | 176 | 18.582 | 29.387 | 16.000 | 1.00 | 11.88 |
| A | C | | | | | | | | | | |
| ATOM | | 1127 | CG | ASN | A | 176 | 19.870 | 29.825 | 15.332 | 1.00 | 15.20 |
| A | C | | | | | | | | | | |
| ATOM | | 1128 | OD1 | ASN | A | 176 | 20.956 | 29.382 | 15.705 | 1.00 | 14.68 |
| A | O | | | | | | | | | | |
| ATOM | | 1129 | ND2 | ASN | A | 176 | 19.759 | 30.709 | 14.350 | 1.00 | 13.94 |
| A | N | | | | | | | | | | |
| ATOM | | 1130 | C | ASN | A | 176 | 17.519 | 27.799 | 17.612 | 1.00 | 13.45 |
| A | C | | | | | | | | | | |
| ATOM | | 1131 | O | ASN | A | 176 | 17.609 | 28.060 | 18.812 | 1.00 | 13.27 |
| A | O | | | | | | | | | | |
| ATOM | | 1132 | N | ALA | A | 177 | 16.410 | 27.303 | 17.079 | 1.00 | 12.70 |
| A | N | | | | | | | | | | |
| ATOM | | 1133 | CA | ALA | A | 177 | 15.233 | 27.092 | 17.913 | 1.00 | 13.82 |
| A | C | | | | | | | | | | |
| ATOM | | 1134 | CB | ALA | A | 177 | 13.961 | 27.151 | 17.049 | 1.00 | 12.99 |
| A | C | | | | | | | | | | |
| ATOM | | 1135 | C | ALA | A | 177 | 15.240 | 25.793 | 18.701 | 1.00 | 13.69 |
| A | C | | | | | | | | | | |
| ATOM | | 1136 | O | ALA | A | 177 | 14.581 | 25.693 | 19.735 | 1.00 | 13.56 |
| A | O | | | | | | | | | | |
| ATOM | | 1137 | N | CYS | A | 178 | 16.013 | 24.808 | 18.255 | 1.00 | 12.34 |
| A | N | | | | | | | | | | |
| ATOM | | 1138 | CA | CYS | A | 178 | 15.954 | 23.505 | 18.917 | 1.00 | 12.68 |
| A | C | | | | | | | | | | |
| ATOM | | 1139 | C | CYS | A | 178 | 17.144 | 23.035 | 19.788 | 1.00 | 12.67 |
| A | C | | | | | | | | | | |
| ATOM | | 1140 | O | CYS | A | 178 | 16.965 | 22.126 | 20.591 | 1.00 | 12.42 |
| A | O | | | | | | | | | | |
| ATOM | | 1141 | CB | CYS | A | 178 | 15.703 | 22.419 | 17.872 | 1.00 | 12.70 |
| A | C | | | | | | | | | | |
| ATOM | | 1142 | SG | CYS | A | 178 | 14.143 | 22.611 | 16.953 | 1.00 | 13.36 |
| A | S | | | | | | | | | | |
| ATOM | | 1143 | N | ILE | A | 179 | 18.323 | 23.620 | 19.637 | 1.00 | 15.23 |
| A | N | | | | | | | | | | |
| ATOM | | 1144 | CA | ILE | A | 179 | 19.456 | 23.173 | 20.438 | 1.00 | 18.51 |
| A | C | | | | | | | | | | |
| ATOM | | 1145 | CB | ILE | A | 179 | 20.789 | 23.611 | 19.808 | 1.00 | 17.80 |
| A | C | | | | | | | | | | |
| ATOM | | 1146 | CG2 | ILE | A | 179 | 20.887 | 23.039 | 18.390 | 1.00 | 15.56 |
| A | C | | | | | | | | | | |
| ATOM | | 1147 | CG1 | ILE | A | 179 | 20.891 | 25.138 | 19.793 | 1.00 | 17.43 |
| A | C | | | | | | | | | | |
| ATOM | | 1148 | CD | ILE | A | 179 | 22.185 | 25.660 | 19.200 | 1.00 | 18.12 |
| A | C | | | | | | | | | | |
| ATOM | | 1149 | C | ILE | A | 179 | 19.384 | 23.657 | 21.886 | 1.00 | 22.43 |
| A | C | | | | | | | | | | |
| ATOM | | 1150 | OT1 | ILE | A | 179 | 19.937 | 22.965 | 22.766 | 1.00 | 24.68 |
| A | O | | | | | | | | | | |
| ATOM | | 1151 | OT2 | ILE | A | 179 | 18.783 | 24.728 | 22.123 | 1.00 | 25.46 |
| A | O | | | | | | | | | | |
| ATOM | | 1152 | CB | HIS | B | 39 | 27.892 | 44.470 | −6.060 | 1.00 | 45.22 |
| B | C | | | | | | | | | | |
| ATOM | | 1153 | CG | HIS | B | 39 | 28.041 | 43.710 | −7.341 | 1.00 | 48.50 |
| B | C | | | | | | | | | | |
| ATOM | | 1154 | CD2 | HIS | B | 39 | 27.587 | 42.488 | −7.704 | 1.00 | 50.10 |
| B | C | | | | | | | | | | |
| ATOM | | 1155 | ND1 | HIS | B | 39 | 28.700 | 44.216 | −8.442 | 1.00 | 50.41 |
| B | N | | | | | | | | | | |
| ATOM | | 1156 | CE1 | HIS | B | 39 | 28.644 | 43.338 | −9.428 | 1.00 | 50.37 |
| B | C | | | | | | | | | | |
| ATOM | | 1157 | NE2 | HIS | B | 39 | 27.974 | 42.281 | −9.007 | 1.00 | 50.98 |
| B | N | | | | | | | | | | |
| ATOM | | 1158 | C | HIS | B | 39 | 29.987 | 43.757 | −4.873 | 1.00 | 43.85 |
| B | C | | | | | | | | | | |
| ATOM | | 1159 | O | HIS | B | 39 | 31.015 | 43.319 | −5.402 | 1.00 | 43.28 |
| B | O | | | | | | | | | | |
| ATOM | | 1160 | N | HIS | B | 39 | 29.995 | 45.653 | −6.496 | 1.00 | 43.51 |
| B | N | | | | | | | | | | |
| ATOM | | 1161 | CA | HIS | B | 39 | 29.215 | 44.936 | −5.463 | 1.00 | 44.10 |
| B | C | | | | | | | | | | |
| ATOM | | 1162 | N | CYS | B | 40 | 29.484 | 43.231 | −3.770 | 1.00 | 42.96 |
| B | N | | | | | | | | | | |
| ATOM | | 1163 | CA | CYS | B | 40 | 30.174 | 42.138 | −3.125 | 1.00 | 42.42 |
| B | C | | | | | | | | | | |
| ATOM | | 1164 | C | CYS | B | 40 | 29.968 | 40.767 | −3.775 | 1.00 | 42.46 |
| B | C | | | | | | | | | | |
| ATOM | | 1165 | O | CYS | B | 40 | 28.844 | 40.378 | −4.077 | 1.00 | 42.43 |
| B | O | | | | | | | | | | |
| ATOM | | 1166 | CB | CYS | B | 40 | 29.771 | 42.107 | −1.658 | 1.00 | 41.23 |
| B | C | | | | | | | | | | |
| ATOM | | 1167 | SG | CYS | B | 40 | 30.160 | 43.644 | −0.756 | 1.00 | 38.48 |
| B | S | | | | | | | | | | |
| ATOM | | 1168 | N | ARG | B | 41 | 31.068 | 40.041 | −3.980 | 1.00 | 42.65 |
| B | N | | | | | | | | | | |
| ATOM | | 1169 | CA | ARG | B | 41 | 31.038 | 38.705 | −4.584 | 1.00 | 43.37 |
| B | C | | | | | | | | | | |
| ATOM | | 1170 | CB | ARG | B | 41 | 30.698 | 38.804 | −6.076 | 1.00 | 44.07 |
| B | C | | | | | | | | | | |
| ATOM | | 1171 | CG | ARG | B | 41 | 30.800 | 37.482 | −6.831 | 1.00 | 45.44 |
| B | C | | | | | | | | | | |
| ATOM | | 1172 | CD | ARG | B | 41 | 30.385 | 37.641 | −8.290 | 1.00 | 46.08 |
| B | C | | | | | | | | | | |
| ATOM | | 1173 | NE | ARG | B | 41 | 30.473 | 36.388 | −9.038 | 1.00 | 47.65 |
| B | N | | | | | | | | | | |
| ATOM | | 1174 | CZ | ARG | B | 41 | 31.612 | 35.781 | −9.356 | 1.00 | 47.69 |
| B | C | | | | | | | | | | |
| ATOM | | 1175 | NH1 | ARG | B | 41 | 32.774 | 36.307 | −8.992 | 1.00 | 49.10 |
| B | N | | | | | | | | | | |
| ATOM | | 1176 | NH2 | ARG | B | 41 | 31.590 | 34.646 | −10.040 | 1.00 | 47.59 |
| B | N | | | | | | | | | | |
| ATOM | | 1177 | C | ARG | B | 41 | 32.390 | 38.014 | −4.407 | 1.00 | 43.67 |
| B | C | | | | | | | | | | |
| ATOM | | 1178 | O | ARG | B | 41 | 33.417 | 38.683 | −4.295 | 1.00 | 43.52 |
| B | O | | | | | | | | | | |
| ATOM | | 1179 | N | LEU | B | 42 | 32.381 | 36.682 | −4.382 | 1.00 | 43.47 |
| B | N | | | | | | | | | | |
| ATOM | | 1180 | CA | LEU | B | 42 | 33.600 | 35.902 | −4.210 | 1.00 | 44.42 |
| B | C | | | | | | | | | | |
| ATOM | | 1181 | CB | LEU | B | 42 | 33.695 | 35.356 | −2.774 | 1.00 | 42.98 |
| B | C | | | | | | | | | | |
| ATOM | | 1182 | CG | LEU | B | 42 | 33.903 | 36.365 | −1.643 | 1.00 | 41.64 |
| B | C | | | | | | | | | | |
| ATOM | | 1183 | CD1 | LEU | B | 42 | 33.743 | 35.674 | −0.299 | 1.00 | 41.04 |
| B | C | | | | | | | | | | |
| ATOM | | 1184 | CD2 | LEU | B | 42 | 35.283 | 36.981 | −1.768 | 1.00 | 40.35 |
| B | C | | | | | | | | | | |
| ATOM | | 1185 | C | LEU | B | 42 | 33.647 | 34.744 | −5.195 | 1.00 | 45.72 |
| B | C | | | | | | | | | | |
| ATOM | | 1186 | O | LEU | B | 42 | 32.635 | 34.098 | −5.470 | 1.00 | 45.97 |
| B | O | | | | | | | | | | |
| ATOM | | 1187 | N | ASP | B | 43 | 34.834 | 34.483 | −5.735 | 1.00 | 46.61 |
| B | N | | | | | | | | | | |
| ATOM | | 1188 | CA | ASP | B | 43 | 34.998 | 33.394 | −6.693 | 1.00 | 47.21 |
| B | C | | | | | | | | | | |
| ATOM | | 1189 | CB | AASP | B | 43 | 36.396 | 33.446 | −7.314 | 0.50 | 48.39 |
| B | C | | | | | | | | | | |
| ATOM | | 1190 | CB | BASP | B | 43 | 36.405 | 33.416 | −7.276 | 0.50 | 48.20 |
| B | C | | | | | | | | | | |
| ATOM | | 1191 | CG | AASP | B | 43 | 36.566 | 32.454 | −8.457 | 0.50 | 49.57 |
| B | C | | | | | | | | | | |
| ATOM | | 1192 | CG | BASP | B | 43 | 36.845 | 34.805 | −7.676 | 0.50 | 49.13 |
| B | C | | | | | | | | | | |
| ATOM | | 1193 | OD1 | AASP | B | 43 | 35.827 | 32.564 | −9.459 | 0.50 | 50.63 |
| B | O | | | | | | | | | | |
| ATOM | | 1194 | OD1 | BASP | B | 43 | 37.622 | 35.414 | −6.914 | 0.50 | 50.04 |
| B | O | | | | | | | | | | |
| ATOM | | 1195 | OD2 | AASP | B | 43 | 37.432 | 31.557 | −8.358 | 0.50 | 49.95 |
| B | O | | | | | | | | | | |
| ATOM | | 1196 | OD2 | BASP | B | 43 | 36.422 | 35.282 | −8.749 | 0.50 | 49.61 |
| B | O | | | | | | | | | | |
| ATOM | | 1197 | C | ASP | B | 43 | 34.785 | 32.037 | −6.023 | 1.00 | 47.20 |
| B | C | | | | | | | | | | |
| ATOM | | 1198 | O | ASP | B | 43 | 35.203 | 31.836 | −4.884 | 1.00 | 46.97 |
| B | O | | | | | | | | | | |
| ATOM | | 1199 | N | LYS | B | 44 | 34.151 | 31.108 | −6.736 | 1.00 | 47.14 |
| B | N | | | | | | | | | | |
| ATOM | | 1200 | CA | LYS | B | 44 | 33.869 | 29.771 | −6.212 | 1.00 | 46.50 |
| B | C | | | | | | | | | | |
| ATOM | | 1201 | CB | LYS | B | 44 | 33.244 | 28.904 | −7.310 | 1.00 | 48.36 |
| B | C | | | | | | | | | | |
| ATOM | | 1202 | CG | LYS | B | 44 | 31.876 | 29.408 | −7.787 | 1.00 | 50.69 |
| B | C | | | | | | | | | | |
| ATOM | | 1203 | CD | LYS | B | 44 | 31.231 | 28.403 | −8.735 | 1.00 | 52.34 |
| B | C | | | | | | | | | | |
| ATOM | | 1204 | CE | LYS | B | 44 | 29.832 | 28.829 | −9.150 | 1.00 | 53.13 |
| B | C | | | | | | | | | | |
| ATOM | | 1205 | NZ | LYS | B | 44 | 29.175 | 27.806 | −10.015 | 1.00 | 54.70 |
| B | N | | | | | | | | | | |
| ATOM | | 1206 | C | LYS | B | 44 | 35.121 | 29.094 | −5.665 | 1.00 | 45.46 |
| B | C | | | | | | | | | | |
| ATOM | | 1207 | O | LYS | B | 44 | 35.083 | 28.353 | −4.677 | 1.00 | 45.04 |
| B | O | | | | | | | | | | |
| ATOM | | 1208 | N | SER | B | 45 | 36.246 | 29.362 | −6.314 | 1.00 | 44.73 |
| B | N | | | | | | | | | | |
| ATOM | | 1209 | CA | SER | B | 45 | 37.514 | 28.777 | −5.899 | 1.00 | 44.17 |
| B | C | | | | | | | | | | |
| ATOM | | 1210 | CB | ASER | B | 45 | 38.659 | 29.317 | −6.765 | 0.50 | 44.61 |
| B | C | | | | | | | | | | |
| ATOM | | 1211 | CB | BSER | B | 45 | 38.645 | 29.351 | −6.767 | 0.50 | 44.45 |
| B | C | | | | | | | | | | |
| ATOM | | 1212 | OG | ASER | B | 45 | 39.915 | 28.799 | −6.349 | 0.50 | 45.01 |
| B | O | | | | | | | | | | |
| ATOM | | 1213 | OG | BSER | B | 45 | 38.341 | 29.256 | −8.145 | 0.50 | 44.34 |
| B | O | | | | | | | | | | |
| ATOM | | 1214 | C | SER | B | 45 | 37.828 | 29.037 | −4.430 | 1.00 | 43.68 |
| B | C | | | | | | | | | | |
| ATOM | | 1215 | O | SER | B | 45 | 38.358 | 28.164 | −3.739 | 1.00 | 43.94 |
| B | O | | | | | | | | | | |
| ATOM | | 1216 | N | ASN | B | 46 | 37.504 | 30.237 | −3.958 | 1.00 | 43.08 |
| B | N | | | | | | | | | | |
| ATOM | | 1217 | CA | ASN | B | 46 | 37.758 | 30.613 | −2.573 | 1.00 | 41.72 |
| B | C | | | | | | | | | | |
| ATOM | | 1218 | CB | ASN | B | 46 | 37.229 | 32.023 | −2.313 | 1.00 | 42.60 |
| B | C | | | | | | | | | | |
| ATOM | | 1219 | CG | ASN | B | 46 | 37.938 | 33.069 | −3.134 | 1.00 | 42.66 |
| B | C | | | | | | | | | | |
| ATOM | | 1220 | OD1 | ASN | B | 46 | 37.794 | 33.119 | −4.353 | 1.00 | 43.85 |
| B | O | | | | | | | | | | |
| ATOM | | 1221 | ND2 | ASN | B | 46 | 38.726 | 33.906 | −2.471 | 1.00 | 43.52 |
| B | N | | | | | | | | | | |
| ATOM | | 1222 | C | ASN | B | 46 | 37.118 | 29.655 | −1.569 | 1.00 | 40.56 |
| B | C | | | | | | | | | | |
| ATOM | | 1223 | O | ASN | B | 46 | 37.615 | 29.501 | −0.457 | 1.00 | 40.87 |
| B | O | | | | | | | | | | |
| ATOM | | 1224 | N | PHE | B | 47 | 36.012 | 29.020 | −1.949 | 1.00 | 39.67 |
| B | N | | | | | | | | | | |
| ATOM | | 1225 | CA | PHE | B | 47 | 35.319 | 28.103 | −1.050 | 1.00 | 37.86 |
| B | C | | | | | | | | | | |
| ATOM | | 1226 | CB | PHE | B | 47 | 33.832 | 28.458 | −1.007 | 1.00 | 37.18 |
| B | C | | | | | | | | | | |
| ATOM | | 1227 | CG | PHE | B | 47 | 33.549 | 29.753 | −0.297 | 1.00 | 38.02 |
| B | C | | | | | | | | | | |
| ATOM | | 1228 | CD1 | PHE | B | 47 | 33.520 | 29.807 | 1.093 | 1.00 | 37.75 |
| B | C | | | | | | | | | | |
| ATOM | | 1229 | CD2 | PHE | B | 47 | 33.346 | 30.928 | −1.017 | 1.00 | 37.55 |
| B | C | | | | | | | | | | |
| ATOM | | 1230 | CE1 | PHE | B | 47 | 33.293 | 31.013 | 1.755 | 1.00 | 37.90 |
| B | C | | | | | | | | | | |
| ATOM | | 1231 | CE2 | PHE | B | 47 | 33.120 | 32.137 | −0.364 | 1.00 | 37.70 |
| B | C | | | | | | | | | | |
| ATOM | | 1232 | CZ | PHE | B | 47 | 33.094 | 32.179 | 1.024 | 1.00 | 37.15 |
| B | C | | | | | | | | | | |
| ATOM | | 1233 | C | PHE | B | 47 | 35.505 | 26.620 | −1.365 | 1.00 | 38.38 |
| B | C | | | | | | | | | | |
| ATOM | | 1234 | O | PHE | B | 47 | 34.863 | 25.764 | −0.758 | 1.00 | 39.24 |
| B | O | | | | | | | | | | |
| ATOM | | 1235 | N | GLN | B | 48 | 36.379 | 26.309 | −2.313 | 1.00 | 37.58 |
| B | N | | | | | | | | | | |
| ATOM | | 1236 | CA | GLN | B | 48 | 36.642 | 24.915 | −2.636 | 1.00 | 37.04 |
| B | C | | | | | | | | | | |
| ATOM | | 1237 | CB | GLN | B | 48 | 36.928 | 24.757 | −4.129 | 1.00 | 38.28 |
| B | C | | | | | | | | | | |
| ATOM | | 1238 | CG | GLN | B | 48 | 35.687 | 24.947 | −4.988 | 1.00 | 40.62 |
| B | C | | | | | | | | | | |
| ATOM | | 1239 | CD | GLN | B | 48 | 35.964 | 24.815 | −6.471 | 1.00 | 41.52 |
| B | C | | | | | | | | | | |
| ATOM | | 1240 | OE1 | GLN | B | 48 | 35.040 | 24.827 | −7.284 | 1.00 | 42.61 |
| B | O | | | | | | | | | | |
| ATOM | | 1241 | NE2 | GLN | B | 48 | 37.238 | 24.692 | −6.832 | 1.00 | 42.47 |
| B | N | | | | | | | | | | |
| ATOM | | 1242 | C | GLN | B | 48 | 37.835 | 24.476 | −1.793 | 1.00 | 35.19 |
| B | C | | | | | | | | | | |
| ATOM | | 1243 | O | GLN | B | 48 | 38.900 | 24.139 | −2.307 | 1.00 | 35.18 |
| B | O | | | | | | | | | | |
| ATOM | | 1244 | N | GLN | B | 49 | 37.635 | 24.504 | −0.480 | 1.00 | 33.48 |
| B | N | | | | | | | | | | |
| ATOM | | 1245 | CA | GLN | B | 49 | 38.661 | 24.131 | 0.484 | 1.00 | 32.10 |
| B | C | | | | | | | | | | |
| ATOM | | 1246 | CB | GLN | B | 49 | 39.186 | 25.377 | 1.211 | 1.00 | 34.97 |
| B | C | | | | | | | | | | |
| ATOM | | 1247 | CG | GLN | B | 49 | 40.433 | 26.012 | 0.597 | 1.00 | 39.74 |
| B | C | | | | | | | | | | |
| ATOM | | 1248 | CD | GLN | B | 49 | 40.157 | 26.729 | −0.707 | 1.00 | 42.56 |
| B | C | | | | | | | | | | |
| ATOM | | 1249 | OE1 | GLN | B | 49 | 40.844 | 26.316 | −1.768 | 1.00 | 44.68 |
| B | O | | | | | | | | | | |
| ATOM | | 1250 | NE2 | GLN | B | 49 | 39.341 | 27.649 | −0.760 | 1.00 | 44.50 |
| B | N | | | | | | | | | | |
| ATOM | | 1251 | C | GLN | B | 49 | 38.077 | 23.157 | 1.503 | 1.00 | 29.66 |
| B | C | | | | | | | | | | |
| ATOM | | 1252 | O | GLN | B | 49 | 37.583 | 23.569 | 2.556 | 1.00 | 28.91 |
| B | O | | | | | | | | | | |
| ATOM | | 1253 | N | PRO | B | 50 | 38.114 | 21.850 | 1.199 | 1.00 | 26.63 |
| B | N | | | | | | | | | | |
| ATOM | | 1254 | CD | PRO | B | 50 | 38.639 | 21.200 | −0.013 | 1.00 | 26.53 |
| B | C | | | | | | | | | | |
| ATOM | | 1255 | CA | PRO | B | 50 | 37.570 | 20.860 | 2.134 | 1.00 | 24.12 |
| B | C | | | | | | | | | | |
| ATOM | | 1256 | CB | PRO | B | 50 | 37.720 | 19.541 | 1.375 | 1.00 | 26.35 |
| B | C | | | | | | | | | | |
| ATOM | | 1257 | CG | PRO | B | 50 | 38.902 | 19.796 | 0.468 | 1.00 | 26.74 |
| B | C | | | | | | | | | | |
| ATOM | | 1258 | C | PRO | B | 50 | 38.287 | 20.846 | 3.476 | 1.00 | 20.94 |
| B | C | | | | | | | | | | |
| ATOM | | 1259 | O | PRO | B | 50 | 37.652 | 20.728 | 4.522 | 1.00 | 20.16 |
| B | O | | | | | | | | | | |
| ATOM | | 1260 | N | TYR | B | 51 | 39.608 | 20.978 | 3.451 | 1.00 | 18.35 |
| B | N | | | | | | | | | | |
| ATOM | | 1261 | CA | TYR | B | 51 | 40.384 | 20.964 | 4.688 | 1.00 | 17.88 |
| B | C | | | | | | | | | | |
| ATOM | | 1262 | CB | TYR | B | 51 | 41.877 | 21.137 | 4.394 | 1.00 | 19.37 |
| B | C | | | | | | | | | | |
| ATOM | | 1263 | CG | TYR | B | 51 | 42.737 | 21.177 | 5.638 | 1.00 | 18.71 |
| B | C | | | | | | | | | | |
| ATOM | | 1264 | CD1 | TYR | B | 51 | 43.015 | 20.014 | 6.352 | 1.00 | 19.74 |
| B | C | | | | | | | | | | |
| ATOM | | 1265 | CE1 | TYR | B | 51 | 43.777 | 20.043 | 7.512 | 1.00 | 20.59 |
| B | C | | | | | | | | | | |
| ATOM | | 1266 | CD2 | TYR | B | 51 | 43.250 | 22.385 | 6.118 | 1.00 | 20.02 |
| B | C | | | | | | | | | | |
| ATOM | | 1267 | CE2 | TYR | B | 51 | 44.018 | 22.428 | 7.282 | 1.00 | 21.12 |
| B | C | | | | | | | | | | |
| ATOM | | 1268 | CZ | TYR | B | 51 | 44.274 | 21.253 | 7.973 | 1.00 | 22.10 |
| B | C | | | | | | | | | | |
| ATOM | | 1269 | OH | TYR | B | 51 | 45.008 | 21.287 | 9.137 | 1.00 | 24.61 |
| B | O | | | | | | | | | | |
| ATOM | | 1270 | C | TYR | B | 51 | 39.933 | 22.065 | 5.637 | 1.00 | 17.24 |
| B | C | | | | | | | | | | |
| ATOM | | 1271 | O | TYR | B | 51 | 39.560 | 21.793 | 6.773 | 1.00 | 17.29 |
| B | O | | | | | | | | | | |
| ATOM | | 1272 | N | ILE | B | 52 | 39.969 | 23.304 | 5.160 | 1.00 | 17.58 |
| B | N | | | | | | | | | | |
| ATOM | | 1273 | CA | ILE | B | 52 | 39.571 | 24.448 | 5.973 | 1.00 | 21.11 |
| B | C | | | | | | | | | | |
| ATOM | | 1274 | CB | ILE | B | 52 | 39.827 | 25.760 | 5.210 | 1.00 | 24.48 |
| B | C | | | | | | | | | | |
| ATOM | | 1275 | CG2 | ILE | B | 52 | 39.180 | 26.934 | 5.937 | 1.00 | 26.45 |
| B | C | | | | | | | | | | |
| ATOM | | 1276 | CG1 | ILE | B | 52 | 41.342 | 25.956 | 5.062 | 1.00 | 26.38 |
| B | C | | | | | | | | | | |
| ATOM | | 1277 | CD | ILE | B | 52 | 41.748 | 27.215 | 4.340 | 1.00 | 31.77 |
| B | C | | | | | | | | | | |
| ATOM | | 1278 | C | ILE | B | 52 | 38.114 | 24.378 | 6.426 | 1.00 | 19.79 |
| B | C | | | | | | | | | | |
| ATOM | | 1279 | O | ILE | B | 52 | 37.782 | 24.812 | 7.530 | 1.00 | 20.54 |
| B | O | | | | | | | | | | |
| ATOM | | 1280 | N | THR | B | 53 | 37.246 | 23.834 | 5.579 | 1.00 | 18.58 |
| B | N | | | | | | | | | | |
| ATOM | | 1281 | CA | THR | B | 53 | 35.837 | 23.701 | 5.932 | 1.00 | 17.34 |
| B | C | | | | | | | | | | |
| ATOM | | 1282 | CB | THR | B | 53 | 35.032 | 23.103 | 4.765 | 1.00 | 19.54 |
| B | C | | | | | | | | | | |
| ATOM | | 1283 | OG1 | THR | B | 53 | 35.186 | 23.939 | 3.610 | 1.00 | 21.25 |
| B | O | | | | | | | | | | |
| ATOM | | 1284 | CG2 | THR | B | 53 | 33.558 | 23.017 | 5.121 | 1.00 | 18.98 |
| B | C | | | | | | | | | | |
| ATOM | | 1285 | C | THR | B | 53 | 35.732 | 22.782 | 7.146 | 1.00 | 16.46 |
| B | C | | | | | | | | | | |
| ATOM | | 1286 | O | THR | B | 53 | 35.092 | 23.116 | 8.139 | 1.00 | 17.44 |
| B | O | | | | | | | | | | |
| ATOM | | 1287 | N | ASN | B | 54 | 36.393 | 21.632 | 7.074 | 1.00 | 16.45 |
| B | N | | | | | | | | | | |
| ATOM | | 1288 | CA | ASN | B | 54 | 36.376 | 20.675 | 8.178 | 1.00 | 15.65 |
| B | C | | | | | | | | | | |
| ATOM | | 1289 | CB | ASN | B | 54 | 37.164 | 19.413 | 7.806 | 1.00 | 17.11 |
| B | C | | | | | | | | | | |
| ATOM | | 1290 | CG | ASN | B | 54 | 36.359 | 18.455 | 6.942 | 1.00 | 19.54 |
| B | C | | | | | | | | | | |
| ATOM | | 1291 | OD1 | ASN | B | 54 | 35.337 | 18.827 | 6.372 | 1.00 | 19.10 |
| B | O | | | | | | | | | | |
| ATOM | | 1292 | ND2 | ASN | B | 54 | 36.826 | 17.221 | 6.835 | 1.00 | 20.54 |
| B | N | | | | | | | | | | |
| ATOM | | 1293 | C | ASN | B | 54 | 36.969 | 21.288 | 9.441 | 1.00 | 16.14 |
| B | C | | | | | | | | | | |
| ATOM | | 1294 | O | ASN | B | 54 | 36.422 | 21.134 | 10.535 | 1.00 | 16.51 |
| B | O | | | | | | | | | | |
| ATOM | | 1295 | N | ARG | B | 55 | 38.087 | 21.990 | 9.284 | 1.00 | 16.11 |
| B | N | | | | | | | | | | |
| ATOM | | 1296 | CA | ARG | B | 55 | 38.748 | 22.618 | 10.425 | 1.00 | 16.50 |
| B | C | | | | | | | | | | |
| ATOM | | 1297 | CB | AARG | B | 55 | 40.100 | 23.214 | 10.006 | 0.50 | 18.63 |
| B | C | | | | | | | | | | |
| ATOM | | 1298 | CB | BARG | B | 55 | 40.069 | 23.223 | 10.002 | 0.50 | 18.75 |
| B | C | | | | | | | | | | |
| ATOM | | 1299 | CG | AARG | B | 55 | 41.043 | 22.199 | 9.379 | 0.50 | 22.49 |
| B | C | | | | | | | | | | |
| ATOM | | 1300 | CG | BARG | B | 55 | 41.066 | 22.263 | 9.348 | 0.50 | 22.67 |
| B | C | | | | | | | | | | |
| ATOM | | 1301 | CD | AARG | B | 55 | 41.300 | 21.017 | 10.307 | 0.50 | 25.57 |
| B | C | | | | | | | | | | |
| ATOM | | 1302 | CD | BARG | B | 55 | 41.866 | 21.447 | 10.363 | 0.50 | 26.03 |
| B | C | | | | | | | | | | |
| ATOM | | 1303 | NE | AARG | B | 55 | 42.403 | 21.236 | 11.238 | 0.50 | 28.75 |
| B | N | | | | | | | | | | |
| ATOM | | 1304 | NE | BARG | B | 55 | 41.176 | 20.260 | 10.872 | 0.50 | 28.52 |
| B | N | | | | | | | | | | |
| ATOM | | 1305 | CZ | AARG | B | 55 | 42.799 | 20.343 | 12.141 | 0.50 | 30.20 |
| B | C | | | | | | | | | | |
| ATOM | | 1306 | CZ | BARG | B | 55 | 41.716 | 19.416 | 11.751 | 0.50 | 30.40 |
| B | C | | | | | | | | | | |
| ATOM | | 1307 | NH1 | AARG | B | 55 | 42.180 | 19.173 | 12.241 | 0.50 | 32.50 |
| B | N | | | | | | | | | | |
| ATOM | | 1308 | NH1 | BARG | B | 55 | 42.935 | 19.643 | 12.215 | 0.50 | 31.14 |
| B | N | | | | | | | | | | |
| ATOM | | 1309 | NH2 | AARG | B | 55 | 43.825 | 20.611 | 12.935 | 0.50 | 31.35 |
| B | N | | | | | | | | | | |
| ATOM | | 1310 | NH2 | BARG | B | 55 | 41.066 | 18.325 | 12.142 | 0.50 | 30.17 |
| B | N | | | | | | | | | | |
| ATOM | | 1311 | C | ARG | B | 55 | 37.883 | 23.712 | 11.041 | 1.00 | 13.93 |
| B | C | | | | | | | | | | |
| ATOM | | 1312 | O | ARG | B | 55 | 37.924 | 23.939 | 12.252 | 1.00 | 13.25 |
| B | O | | | | | | | | | | |
| ATOM | | 1313 | N | THR | B | 56 | 37.100 | 24.392 | 10.208 | 1.00 | 14.26 |
| B | N | | | | | | | | | | |
| ATOM | | 1314 | CA | THR | B | 56 | 36.230 | 25.452 | 10.693 | 1.00 | 11.34 |
| B | C | | | | | | | | | | |
| ATOM | | 1315 | CB | THR | B | 56 | 35.585 | 26.237 | 9.525 | 1.00 | 14.38 |
| B | C | | | | | | | | | | |
| ATOM | | 1316 | OG1 | THR | B | 56 | 36.614 | 26.908 | 8.779 | 1.00 | 14.44 |
| B | O | | | | | | | | | | |
| ATOM | | 1317 | CG2 | THR | B | 56 | 34.587 | 27.266 | 10.052 | 1.00 | 11.15 |
| B | C | | | | | | | | | | |
| ATOM | | 1318 | C | THR | B | 56 | 35.149 | 24.817 | 11.556 | 1.00 | 13.75 |
| B | C | | | | | | | | | | |
| ATOM | | 1319 | O | THR | B | 56 | 34.817 | 25.319 | 12.631 | 1.00 | 14.25 |
| B | O | | | | | | | | | | |
| ATOM | | 1320 | N | PHE | B | 57 | 34.598 | 23.700 | 11.089 | 1.00 | 13.72 |
| B | N | | | | | | | | | | |
| ATOM | | 1321 | CA | PHE | B | 57 | 33.565 | 23.018 | 11.851 | 1.00 | 13.15 |
| B | C | | | | | | | | | | |
| ATOM | | 1322 | CB | PHE | B | 57 | 32.899 | 21.932 | 11.001 | 1.00 | 13.02 |
| B | C | | | | | | | | | | |
| ATOM | | 1323 | CG | PHE | B | 57 | 31.799 | 22.453 | 10.117 | 1.00 | 11.26 |
| B | C | | | | | | | | | | |
| ATOM | | 1324 | CD1 | PHE | B | 57 | 32.085 | 23.289 | 9.044 | 1.00 | 12.61 |
| B | C | | | | | | | | | | |
| ATOM | | 1325 | CD2 | PHE | B | 57 | 30.472 | 22.125 | 10.377 | 1.00 | 11.56 |
| B | C | | | | | | | | | | |
| ATOM | | 1326 | CE1 | PHE | B | 57 | 31.069 | 23.792 | 8.241 | 1.00 | 14.35 |
| B | C | | | | | | | | | | |
| ATOM | | 1327 | CE2 | PHE | B | 57 | 29.443 | 22.621 | 9.582 | 1.00 | 11.35 |
| B | C | | | | | | | | | | |
| ATOM | | 1328 | CZ | PHE | B | 57 | 29.741 | 23.458 | 8.509 | 1.00 | 11.81 |
| B | C | | | | | | | | | | |
| ATOM | | 1329 | C | PHE | B | 57 | 34.138 | 22.431 | 13.142 | 1.00 | 13.86 |
| B | C | | | | | | | | | | |
| ATOM | | 1330 | O | PHE | B | 57 | 33.444 | 22.354 | 14.153 | 1.00 | 14.13 |
| B | O | | | | | | | | | | |
| ATOM | | 1331 | N | MET | B | 58 | 35.405 | 22.029 | 13.124 | 1.00 | 15.45 |
| B | N | | | | | | | | | | |
| ATOM | | 1332 | CA | MET | B | 58 | 36.004 | 21.481 | 14.335 | 1.00 | 16.62 |
| B | C | | | | | | | | | | |
| ATOM | | 1333 | CB | MET | B | 58 | 37.325 | 20.792 | 14.017 | 1.00 | 21.38 |
| B | C | | | | | | | | | | |
| ATOM | | 1334 | CG | MET | B | 58 | 37.130 | 19.435 | 13.368 | 1.00 | 27.49 |
| B | C | | | | | | | | | | |
| ATOM | | 1335 | SD | MET | B | 58 | 36.220 | 18.220 | 14.333 | 1.00 | 33.41 |
| B | S | | | | | | | | | | |
| ATOM | | 1336 | CE | MET | B | 58 | 37.378 | 17.916 | 15.662 | 1.00 | 29.95 |
| B | C | | | | | | | | | | |
| ATOM | | 1337 | C | MET | B | 58 | 36.221 | 22.601 | 15.343 | 1.00 | 15.87 |
| B | C | | | | | | | | | | |
| ATOM | | 1338 | O | MET | B | 58 | 36.000 | 22.429 | 16.538 | 1.00 | 14.52 |
| B | O | | | | | | | | | | |
| ATOM | | 1339 | N | LEU | B | 59 | 36.652 | 23.755 | 14.852 | 1.00 | 14.79 |
| B | N | | | | | | | | | | |
| ATOM | | 1340 | CA | LEU | B | 59 | 36.871 | 24.898 | 15.719 | 1.00 | 13.60 |
| B | C | | | | | | | | | | |
| ATOM | | 1341 | CB | LEU | B | 59 | 37.416 | 26.076 | 14.910 | 1.00 | 12.59 |
| B | C | | | | | | | | | | |
| ATOM | | 1342 | CG | LEU | B | 59 | 37.506 | 27.417 | 15.643 | 1.00 | 12.48 |
| B | C | | | | | | | | | | |
| ATOM | | 1343 | CD1 | LEU | B | 59 | 38.352 | 27.260 | 16.896 | 1.00 | 13.58 |
| B | C | | | | | | | | | | |
| ATOM | | 1344 | CD2 | LEU | B | 59 | 38.105 | 28.476 | 14.710 | 1.00 | 11.97 |
| B | C | | | | | | | | | | |
| ATOM | | 1345 | C | LEU | B | 59 | 35.540 | 25.283 | 16.364 | 1.00 | 13.10 |
| B | C | | | | | | | | | | |
| ATOM | | 1346 | O | LEU | B | 59 | 35.480 | 25.538 | 17.566 | 1.00 | 14.28 |
| B | O | | | | | | | | | | |
| ATOM | | 1347 | N | ALA | B | 60 | 34.477 | 25.325 | 15.558 | 1.00 | 11.11 |
| B | N | | | | | | | | | | |
| ATOM | | 1348 | CA | ALA | B | 60 | 33.145 | 25.670 | 16.055 | 1.00 | 12.18 |
| B | C | | | | | | | | | | |
| ATOM | | 1349 | CB | ALA | B | 60 | 32.148 | 25.744 | 14.879 | 1.00 | 11.09 |
| B | C | | | | | | | | | | |
| ATOM | | 1350 | C | ALA | B | 60 | 32.665 | 24.659 | 17.103 | 1.00 | 12.00 |
| B | C | | | | | | | | | | |
| ATOM | | 1351 | O | ALA | B | 60 | 32.070 | 25.031 | 18.119 | 1.00 | 9.58 |
| B | O | | | | | | | | | | |
| ATOM | | 1352 | N | LYS | B | 61 | 32.915 | 23.376 | 16.863 | 1.00 | 12.27 |
| B | N | | | | | | | | | | |
| ATOM | | 1353 | CA | LYS | B | 61 | 32.515 | 22.351 | 17.818 | 1.00 | 13.95 |
| B | C | | | | | | | | | | |
| ATOM | | 1354 | CB | LYS | B | 61 | 32.842 | 20.952 | 17.294 | 1.00 | 14.14 |
| B | C | | | | | | | | | | |
| ATOM | | 1355 | CG | LYS | B | 61 | 31.862 | 20.412 | 16.282 | 1.00 | 19.93 |
| B | C | | | | | | | | | | |
| ATOM | | 1356 | CD | LYS | B | 61 | 32.281 | 19.014 | 15.841 | 1.00 | 19.80 |
| B | C | | | | | | | | | | |
| ATOM | | 1357 | CE | LYS | B | 61 | 31.339 | 18.454 | 14.799 | 1.00 | 23.77 |
| B | C | | | | | | | | | | |
| ATOM | | 1358 | NZ | LYS | B | 61 | 32.028 | 17.403 | 14.007 | 1.00 | 22.83 |
| B | N | | | | | | | | | | |
| ATOM | | 1359 | C | LYS | B | 61 | 33.228 | 22.534 | 19.143 | 1.00 | 13.07 |
| B | C | | | | | | | | | | |
| ATOM | | 1360 | O | LYS | B | 61 | 32.611 | 22.446 | 20.203 | 1.00 | 13.89 |
| B | O | | | | | | | | | | |
| ATOM | | 1361 | N | GLU | B | 62 | 34.532 | 22.782 | 19.079 | 1.00 | 12.95 |
| B | N | | | | | | | | | | |
| ATOM | | 1362 | CA | GLU | B | 62 | 35.335 | 22.955 | 20.289 | 1.00 | 13.90 |
| B | C | | | | | | | | | | |
| ATOM | | 1363 | CB | GLU | B | 62 | 36.804 | 23.164 | 19.906 | 1.00 | 15.80 |
| B | C | | | | | | | | | | |
| ATOM | | 1364 | CG | GLU | B | 62 | 37.756 | 23.426 | 21.071 | 1.00 | 19.60 |
| B | C | | | | | | | | | | |
| ATOM | | 1365 | CD | GLU | B | 62 | 37.749 | 22.321 | 22.122 | 1.00 | 23.26 |
| B | C | | | | | | | | | | |
| ATOM | | 1366 | OE1 | GLU | B | 62 | 37.759 | 21.127 | 21.747 | 1.00 | 23.30 |
| B | O | | | | | | | | | | |
| ATOM | | 1367 | OE2 | GLU | B | 62 | 37.746 | 22.654 | 23.332 | 1.00 | 25.11 |
| B | O | | | | | | | | | | |
| ATOM | | 1368 | C | GLU | B | 62 | 34.841 | 24.116 | 21.137 | 1.00 | 13.12 |
| B | C | | | | | | | | | | |
| ATOM | | 1369 | O | GLU | B | 62 | 34.654 | 23.974 | 22.346 | 1.00 | 15.86 |
| B | O | | | | | | | | | | |
| ATOM | | 1370 | N | ALA | B | 63 | 34.638 | 25.269 | 20.509 | 1.00 | 12.37 |
| B | N | | | | | | | | | | |
| ATOM | | 1371 | CA | ALA | B | 63 | 34.164 | 26.444 | 21.228 | 1.00 | 11.98 |
| B | C | | | | | | | | | | |
| ATOM | | 1372 | CB | ALA | B | 63 | 34.214 | 27.674 | 20.322 | 1.00 | 10.58 |
| B | C | | | | | | | | | | |
| ATOM | | 1373 | C | ALA | B | 63 | 32.743 | 26.220 | 21.732 | 1.00 | 12.40 |
| B | C | | | | | | | | | | |
| ATOM | | 1374 | O | ALA | B | 63 | 32.383 | 26.695 | 22.802 | 1.00 | 14.78 |
| B | O | | | | | | | | | | |
| ATOM | | 1375 | N | SER | B | 64 | 31.938 | 25.487 | 20.967 | 1.00 | 13.59 |
| B | N | | | | | | | | | | |
| ATOM | | 1376 | CA | SER | B | 64 | 30.557 | 25.225 | 21.361 | 1.00 | 14.14 |
| B | C | | | | | | | | | | |
| ATOM | | 1377 | CB | SER | B | 64 | 29.825 | 24.450 | 20.266 | 1.00 | 13.79 |
| B | C | | | | | | | | | | |
| ATOM | | 1378 | OG | SER | B | 64 | 29.537 | 25.295 | 19.166 | 1.00 | 15.71 |
| B | O | | | | | | | | | | |
| ATOM | | 1379 | C | SER | B | 64 | 30.464 | 24.468 | 22.680 | 1.00 | 16.02 |
| B | C | | | | | | | | | | |
| ATOM | | 1380 | O | SER | B | 64 | 29.456 | 24.576 | 23.389 | 1.00 | 14.81 |
| B | O | | | | | | | | | | |
| ATOM | | 1381 | N | LEU | B | 65 | 31.516 | 23.715 | 23.009 | 1.00 | 16.10 |
| B | N | | | | | | | | | | |
| ATOM | | 1382 | CA | LEU | B | 65 | 31.546 | 22.951 | 24.258 | 1.00 | 18.41 |
| B | C | | | | | | | | | | |
| ATOM | | 1383 | CB | LEU | B | 65 | 32.829 | 22.124 | 24.362 | 1.00 | 19.27 |
| B | C | | | | | | | | | | |
| ATOM | | 1384 | CG | LEU | B | 65 | 33.084 | 21.082 | 23.277 | 1.00 | 19.94 |
| B | C | | | | | | | | | | |
| ATOM | | 1385 | CD1 | LEU | B | 65 | 34.420 | 20.401 | 23.556 | 1.00 | 21.12 |
| B | C | | | | | | | | | | |
| ATOM | | 1386 | CD2 | LEU | B | 65 | 31.941 | 20.069 | 23.233 | 1.00 | 19.85 |
| B | C | | | | | | | | | | |
| ATOM | | 1387 | C | LEU | B | 65 | 31.461 | 23.873 | 25.472 | 1.00 | 21.28 |
| B | C | | | | | | | | | | |
| ATOM | | 1388 | O | LEU | B | 65 | 30.913 | 23.496 | 26.508 | 1.00 | 20.85 |
| B | O | | | | | | | | | | |
| ATOM | | 1389 | N | ALA | B | 66 | 31.993 | 25.084 | 25.337 | 1.00 | 22.77 |
| B | N | | | | | | | | | | |
| ATOM | | 1390 | CA | ALA | B | 66 | 31.991 | 26.041 | 26.440 | 1.00 | 23.05 |
| B | C | | | | | | | | | | |
| ATOM | | 1391 | CB | ALA | B | 66 | 33.340 | 26.759 | 26.505 | 1.00 | 23.03 |
| B | C | | | | | | | | | | |
| ATOM | | 1392 | C | ALA | B | 66 | 30.864 | 27.063 | 26.349 | 1.00 | 25.33 |
| B | C | | | | | | | | | | |
| ATOM | | 1393 | O | ALA | B | 66 | 30.794 | 27.989 | 27.160 | 1.00 | 24.62 |
| B | O | | | | | | | | | | |
| ATOM | | 1394 | N | ASP | B | 67 | 29.984 | 26.895 | 25.366 | 1.00 | 24.71 |
| B | N | | | | | | | | | | |
| ATOM | | 1395 | CA | ASP | B | 67 | 28.859 | 27.805 | 25.169 | 1.00 | 24.59 |
| B | C | | | | | | | | | | |
| ATOM | | 1396 | CB | ASP | B | 67 | 28.843 | 28.293 | 23.715 | 1.00 | 21.80 |
| B | C | | | | | | | | | | |
| ATOM | | 1397 | CG | ASP | B | 67 | 27.658 | 29.193 | 23.409 | 1.00 | 23.11 |
| B | C | | | | | | | | | | |
| ATOM | | 1398 | OD1 | ASP | B | 67 | 27.492 | 30.220 | 24.101 | 1.00 | 21.79 |
| B | O | | | | | | | | | | |
| ATOM | | 1399 | OD2 | ASP | B | 67 | 26.894 | 28.881 | 22.468 | 1.00 | 22.86 |
| B | O | | | | | | | | | | |
| ATOM | | 1400 | C | ASP | B | 67 | 27.539 | 27.105 | 25.484 | 1.00 | 24.11 |
| B | C | | | | | | | | | | |
| ATOM | | 1401 | O | ASP | B | 67 | 27.339 | 25.961 | 25.090 | 1.00 | 24.85 |
| B | O | | | | | | | | | | |
| ATOM | | 1402 | N | ASN | B | 68 | 26.643 | 27.781 | 26.199 | 1.00 | 23.66 |
| B | N | | | | | | | | | | |
| ATOM | | 1403 | CA | ASN | B | 68 | 25.341 | 27.190 | 26.505 | 1.00 | 26.10 |
| B | C | | | | | | | | | | |
| ATOM | | 1404 | CB | ASN | B | 68 | 24.606 | 27.991 | 27.584 | 1.00 | 26.12 |
| B | C | | | | | | | | | | |
| ATOM | | 1405 | CG | ASN | B | 68 | 23.178 | 27.507 | 27.791 | 1.00 | 28.10 |
| B | C | | | | | | | | | | |
| ATOM | | 1406 | OD1 | ASN | B | 68 | 22.902 | 26.309 | 27.704 | 1.00 | 30.31 |
| B | O | | | | | | | | | | |
| ATOM | | 1407 | ND2 | ASN | B | 68 | 22.267 | 28.432 | 28.075 | 1.00 | 29.58 |
| B | N | | | | | | | | | | |
| ATOM | | 1408 | C | ASN | B | 68 | 24.514 | 27.191 | 25.223 | 1.00 | 26.09 |
| B | C | | | | | | | | | | |
| ATOM | | 1409 | O | ASN | B | 68 | 24.122 | 28.252 | 24.733 | 1.00 | 25.01 |
| B | O | | | | | | | | | | |
| ATOM | | 1410 | N | ASN | B | 69 | 24.245 | 26.006 | 24.682 | 1.00 | 26.55 |
| B | N | | | | | | | | | | |
| ATOM | | 1411 | CA | ASN | B | 69 | 23.488 | 25.905 | 23.435 | 1.00 | 28.23 |
| B | C | | | | | | | | | | |
| ATOM | | 1412 | CB | ASN | B | 69 | 23.321 | 24.443 | 23.029 | 1.00 | 29.69 |
| B | C | | | | | | | | | | |
| ATOM | | 1413 | CG | ASN | B | 69 | 24.642 | 23.743 | 22.885 | 1.00 | 32.95 |
| B | C | | | | | | | | | | |
| ATOM | | 1414 | OD1 | ASN | B | 69 | 25.616 | 24.339 | 22.428 | 1.00 | 33.35 |
| B | O | | | | | | | | | | |
| ATOM | | 1415 | ND2 | ASN | B | 69 | 24.690 | 22.470 | 23.268 | 1.00 | 35.41 |
| B | N | | | | | | | | | | |
| ATOM | | 1416 | C | ASN | B | 69 | 22.129 | 26.575 | 23.462 | 1.00 | 27.38 |
| B | C | | | | | | | | | | |
| ATOM | | 1417 | O | ASN | B | 69 | 21.754 | 27.280 | 22.527 | 1.00 | 26.91 |
| B | O | | | | | | | | | | |
| ATOM | | 1418 | N | THR | B | 70 | 21.386 | 26.367 | 24.533 | 1.00 | 27.43 |
| B | N | | | | | | | | | | |
| ATOM | | 1419 | CA | THR | B | 70 | 20.063 | 26.963 | 24.620 | 1.00 | 31.14 |
| B | C | | | | | | | | | | |
| ATOM | | 1420 | CB | THR | B | 70 | 19.290 | 26.312 | 25.724 | 1.00 | 33.84 |
| B | C | | | | | | | | | | |
| ATOM | | 1421 | OG1 | THR | B | 70 | 20.017 | 26.483 | 26.949 | 1.00 | 38.79 |
| B | O | | | | | | | | | | |
| ATOM | | 1422 | CG2 | THR | B | 70 | 19.099 | 24.825 | 25.414 | 1.00 | 35.38 |
| B | C | | | | | | | | | | |
| ATOM | | 1423 | C | THR | B | 70 | 20.084 | 28.472 | 24.850 | 1.00 | 29.99 |
| B | C | | | | | | | | | | |
| ATOM | | 1424 | O | THR | B | 70 | 19.032 | 29.100 | 24.983 | 1.00 | 31.80 |
| B | O | | | | | | | | | | |
| ATOM | | 1425 | N | ASP | B | 71 | 21.280 | 29.048 | 24.900 | 1.00 | 28.37 |
| B | N | | | | | | | | | | |
| ATOM | | 1426 | CA | ASP | B | 71 | 21.420 | 30.481 | 25.102 | 1.00 | 25.20 |
| B | C | | | | | | | | | | |
| ATOM | | 1427 | CB | ASP | B | 71 | 22.838 | 30.798 | 25.589 | 1.00 | 24.99 |
| B | C | | | | | | | | | | |
| ATOM | | 1428 | CG | ASP | B | 71 | 22.995 | 32.241 | 26.019 | 1.00 | 25.37 |
| B | C | | | | | | | | | | |
| ATOM | | 1429 | OD1 | ASP | B | 71 | 21.991 | 32.842 | 26.457 | 1.00 | 24.56 |
| B | O | | | | | | | | | | |
| ATOM | | 1430 | OD2 | ASP | B | 71 | 24.120 | 32.775 | 25.929 | 1.00 | 24.43 |
| B | O | | | | | | | | | | |
| ATOM | | 1431 | C | ASP | B | 71 | 21.124 | 31.210 | 23.789 | 1.00 | 24.70 |
| B | C | | | | | | | | | | |
| ATOM | | 1432 | O | ASP | B | 71 | 20.958 | 30.579 | 22.741 | 1.00 | 25.80 |
| B | O | | | | | | | | | | |
| ATOM | | 1433 | N | VAL | B | 72 | 21.051 | 32.536 | 23.852 | 1.00 | 22.24 |
| B | N | | | | | | | | | | |
| ATOM | | 1434 | CA | VAL | B | 72 | 20.769 | 33.352 | 22.674 | 1.00 | 21.58 |
| B | C | | | | | | | | | | |
| ATOM | | 1435 | CB | VAL | B | 72 | 20.887 | 34.856 | 23.015 | 1.00 | 21.53 |
| B | C | | | | | | | | | | |
| ATOM | | 1436 | CG1 | VAL | B | 72 | 22.310 | 35.177 | 23.443 | 1.00 | 21.59 |
| B | C | | | | | | | | | | |
| ATOM | | 1437 | CG2 | VAL | B | 72 | 20.468 | 35.711 | 21.818 | 1.00 | 24.37 |
| B | C | | | | | | | | | | |
| ATOM | | 1438 | C | VAL | B | 72 | 21.729 | 33.005 | 21.534 | 1.00 | 21.11 |
| B | C | | | | | | | | | | |
| ATOM | | 1439 | O | VAL | B | 72 | 22.864 | 32.600 | 21.773 | 1.00 | 17.71 |
| B | O | | | | | | | | | | |
| ATOM | | 1440 | N | ARG | B | 73 | 21.259 | 33.162 | 20.300 | 1.00 | 19.03 |
| B | N | | | | | | | | | | |
| ATOM | | 1441 | CA | ARG | B | 73 | 22.062 | 32.861 | 19.120 | 1.00 | 21.00 |
| B | C | | | | | | | | | | |
| ATOM | | 1442 | CB | ARG | B | 73 | 21.242 | 32.005 | 18.145 | 1.00 | 20.05 |
| B | C | | | | | | | | | | |
| ATOM | | 1443 | CG | ARG | B | 73 | 20.914 | 30.607 | 18.658 | 1.00 | 22.93 |
| B | C | | | | | | | | | | |
| ATOM | | 1444 | CD | ARG | B | 73 | 22.184 | 29.788 | 18.848 | 1.00 | 23.32 |
| B | C | | | | | | | | | | |
| ATOM | | 1445 | NE | ARG | B | 73 | 22.541 | 29.607 | 20.253 | 1.00 | 26.02 |
| B | N | | | | | | | | | | |
| ATOM | | 1446 | CZ | ARG | B | 73 | 23.792 | 29.534 | 20.701 | 1.00 | 28.62 |
| B | C | | | | | | | | | | |
| ATOM | | 1447 | NH1 | ARG | B | 73 | 24.810 | 29.643 | 19.850 | 1.00 | 27.83 |
| B | N | | | | | | | | | | |
| ATOM | | 1448 | NH2 | ARG | B | 73 | 24.032 | 29.318 | 21.991 | 1.00 | 24.28 |
| B | N | | | | | | | | | | |
| ATOM | | 1449 | C | ARG | B | 73 | 22.562 | 34.130 | 18.422 | 1.00 | 19.92 |
| B | C | | | | | | | | | | |
| ATOM | | 1450 | O | ARG | B | 73 | 22.231 | 35.242 | 18.823 | 1.00 | 23.35 |
| B | O | | | | | | | | | | |
| ATOM | | 1451 | N | LEU | B | 74 | 23.361 | 33.956 | 17.375 | 1.00 | 18.84 |
| B | N | | | | | | | | | | |
| ATOM | | 1452 | CA | LEU | B | 74 | 23.928 | 35.091 | 16.644 | 1.00 | 18.10 |
| B | C | | | | | | | | | | |
| ATOM | | 1453 | CB | LEU | B | 74 | 25.452 | 35.071 | 16.764 | 1.00 | 17.88 |
| B | C | | | | | | | | | | |
| ATOM | | 1454 | CG | LEU | B | 74 | 26.021 | 35.213 | 18.178 | 1.00 | 17.88 |
| B | C | | | | | | | | | | |
| ATOM | | 1455 | CD1 | LEU | B | 74 | 27.514 | 34.966 | 18.146 | 1.00 | 19.62 |
| B | C | | | | | | | | | | |
| ATOM | | 1456 | CD2 | LEU | B | 74 | 25.711 | 36.602 | 18.727 | 1.00 | 16.68 |
| B | C | | | | | | | | | | |
| ATOM | | 1457 | C | LEU | B | 74 | 23.550 | 35.113 | 15.172 | 1.00 | 18.69 |
| B | C | | | | | | | | | | |
| ATOM | | 1458 | O | LEU | B | 74 | 22.990 | 36.094 | 14.678 | 1.00 | 17.25 |
| B | O | | | | | | | | | | |
| ATOM | | 1459 | N | ILE | B | 75 | 23.864 | 34.026 | 14.478 | 1.00 | 18.86 |
| B | N | | | | | | | | | | |
| ATOM | | 1460 | CA | ILE | B | 75 | 23.565 | 33.900 | 13.055 | 1.00 | 19.94 |
| B | C | | | | | | | | | | |
| ATOM | | 1461 | CB | AILE | B | 75 | 24.683 | 33.133 | 12.315 | 0.50 | 19.70 |
| B | C | | | | | | | | | | |
| ATOM | | 1462 | CB | BILE | B | 75 | 24.685 | 33.159 | 12.306 | 0.50 | 19.21 |
| B | C | | | | | | | | | | |
| ATOM | | 1463 | CG2 | AILE | B | 75 | 24.354 | 33.044 | 10.830 | 0.50 | 20.21 |
| B | C | | | | | | | | | | |
| ATOM | | 1464 | CG2 | BILE | B | 75 | 24.280 | 32.922 | 10.857 | 0.50 | 19.75 |
| B | C | | | | | | | | | | |
| ATOM | | 1465 | CG1 | AILE | B | 75 | 26.032 | 33.829 | 12.533 | 0.50 | 21.96 |
| B | C | | | | | | | | | | |
| ATOM | | 1466 | CG1 | BILE | B | 75 | 25.976 | 33.983 | 12.354 | 0.50 | 20.78 |
| B | C | | | | | | | | | | |
| ATOM | | 1467 | CD | AILE | B | 75 | 26.075 | 35.259 | 12.036 | 0.50 | 21.49 |
| B | C | | | | | | | | | | |
| ATOM | | 1468 | CD | BILE | B | 75 | 27.167 | 33.330 | 11.684 | 0.50 | 19.44 |
| B | C | | | | | | | | | | |
| ATOM | | 1469 | C | ILE | B | 75 | 22.250 | 33.159 | 12.850 | 1.00 | 19.75 |
| B | C | | | | | | | | | | |
| ATOM | | 1470 | O | ILE | B | 75 | 22.118 | 32.001 | 13.241 | 1.00 | 19.53 |
| B | O | | | | | | | | | | |
| ATOM | | 1471 | N | GLY | B | 76 | 21.279 | 33.834 | 12.244 | 1.00 | 20.72 |
| B | N | | | | | | | | | | |
| ATOM | | 1472 | CA | GLY | B | 76 | 19.991 | 33.215 | 12.002 | 1.00 | 21.85 |
| B | C | | | | | | | | | | |
| ATOM | | 1473 | C | GLY | B | 76 | 19.144 | 34.080 | 11.090 | 1.00 | 23.94 |
| B | C | | | | | | | | | | |
| ATOM | | 1474 | O | GLY | B | 76 | 19.583 | 35.137 | 10.625 | 1.00 | 21.15 |
| B | O | | | | | | | | | | |
| ATOM | | 1475 | N | GLU | B | 77 | 17.915 | 33.637 | 10.849 | 1.00 | 24.76 |
| B | N | | | | | | | | | | |
| ATOM | | 1476 | CA | GLU | B | 77 | 16.994 | 34.355 | 9.981 | 1.00 | 25.59 |
| B | C | | | | | | | | | | |
| ATOM | | 1477 | CB | GLU | B | 77 | 15.622 | 33.683 | 10.024 | 1.00 | 28.23 |
| B | C | | | | | | | | | | |
| ATOM | | 1478 | CG | GLU | B | 77 | 14.939 | 33.702 | 11.385 | 1.00 | 29.97 |
| B | C | | | | | | | | | | |
| ATOM | | 1479 | CD | GLU | B | 77 | 14.300 | 35.043 | 11.702 | 1.00 | 31.49 |
| B | C | | | | | | | | | | |
| ATOM | | 1480 | OE1 | GLU | B | 77 | 13.646 | 35.618 | 10.805 | 1.00 | 31.82 |
| B | O | | | | | | | | | | |
| ATOM | | 1481 | OE2 | GLU | B | 77 | 14.438 | 35.516 | 12.851 | 1.00 | 32.75 |
| B | O | | | | | | | | | | |
| ATOM | | 1482 | C | GLU | B | 77 | 16.863 | 35.826 | 10.340 | 1.00 | 26.42 |
| B | C | | | | | | | | | | |
| ATOM | | 1483 | O | GLU | B | 77 | 16.733 | 36.667 | 9.454 | 1.00 | 25.64 |
| B | O | | | | | | | | | | |
| ATOM | | 1484 | N | LYS | B | 78 | 16.903 | 36.141 | 11.632 | 1.00 | 26.48 |
| B | N | | | | | | | | | | |
| ATOM | | 1485 | CA | LYS | B | 78 | 16.769 | 37.529 | 12.071 | 1.00 | 26.66 |
| B | C | | | | | | | | | | |
| ATOM | | 1486 | CB | LYS | B | 78 | 16.769 | 37.614 | 13.607 | 1.00 | 29.51 |
| B | C | | | | | | | | | | |
| ATOM | | 1487 | CG | LYS | B | 78 | 17.949 | 36.911 | 14.306 | 1.00 | 34.38 |
| B | C | | | | | | | | | | |
| ATOM | | 1488 | CD | LYS | B | 78 | 17.866 | 35.363 | 14.172 | 1.00 | 34.31 |
| B | C | | | | | | | | | | |
| ATOM | | 1489 | CE | LYS | B | 78 | 19.033 | 34.584 | 14.838 | 1.00 | 35.41 |
| B | C | | | | | | | | | | |
| ATOM | | 1490 | NZ | LYS | B | 78 | 19.360 | 35.086 | 16.206 | 1.00 | 33.27 |
| B | N | | | | | | | | | | |
| ATOM | | 1491 | C | LYS | B | 78 | 17.845 | 38.439 | 11.488 | 1.00 | 25.68 |
| B | C | | | | | | | | | | |
| ATOM | | 1492 | O | LYS | B | 78 | 17.585 | 39.607 | 11.182 | 1.00 | 25.09 |
| B | O | | | | | | | | | | |
| ATOM | | 1493 | N | LEU | B | 79 | 19.049 | 37.903 | 11.322 | 1.00 | 22.46 |
| B | N | | | | | | | | | | |
| ATOM | | 1494 | CA | LEU | B | 79 | 20.158 | 38.663 | 10.759 | 1.00 | 22.51 |
| B | C | | | | | | | | | | |
| ATOM | | 1495 | CB | LEU | B | 79 | 21.421 | 37.790 | 10.744 | 1.00 | 21.91 |
| B | C | | | | | | | | | | |
| ATOM | | 1496 | CG | LEU | B | 79 | 22.765 | 38.360 | 10.272 | 1.00 | 21.91 |
| B | C | | | | | | | | | | |
| ATOM | | 1497 | CD1 | LEU | B | 79 | 23.199 | 39.526 | 11.164 | 1.00 | 20.83 |
| B | C | | | | | | | | | | |
| ATOM | | 1498 | CD2 | LEU | B | 79 | 23.819 | 37.255 | 10.317 | 1.00 | 18.82 |
| B | C | | | | | | | | | | |
| ATOM | | 1499 | C | LEU | B | 79 | 19.833 | 39.125 | 9.332 | 1.00 | 23.76 |
| B | C | | | | | | | | | | |
| ATOM | | 1500 | O | LEU | B | 79 | 20.355 | 40.139 | 8.858 | 1.00 | 21.25 |
| B | O | | | | | | | | | | |
| ATOM | | 1501 | N | PHE | B | 80 | 18.957 | 38.389 | 8.655 | 1.00 | 23.14 |
| B | N | | | | | | | | | | |
| ATOM | | 1502 | CA | PHE | B | 80 | 18.605 | 38.721 | 7.283 | 1.00 | 24.04 |
| B | C | | | | | | | | | | |
| ATOM | | 1503 | CB | PHE | B | 80 | 18.576 | 37.448 | 6.434 | 1.00 | 22.67 |
| B | C | | | | | | | | | | |
| ATOM | | 1504 | CG | PHE | B | 80 | 19.852 | 36.665 | 6.484 | 1.00 | 22.24 |
| B | C | | | | | | | | | | |
| ATOM | | 1505 | CD1 | PHE | B | 80 | 20.086 | 35.754 | 7.508 | 1.00 | 22.35 |
| B | C | | | | | | | | | | |
| ATOM | | 1506 | CD2 | PHE | B | 80 | 20.837 | 36.863 | 5.527 | 1.00 | 22.73 |
| B | C | | | | | | | | | | |
| ATOM | | 1507 | CE1 | PHE | B | 80 | 21.286 | 35.051 | 7.578 | 1.00 | 23.09 |
| B | C | | | | | | | | | | |
| ATOM | | 1508 | CE2 | PHE | B | 80 | 22.039 | 36.164 | 5.591 | 1.00 | 24.92 |
| B | C | | | | | | | | | | |
| ATOM | | 1509 | CZ | PHE | B | 80 | 22.262 | 35.255 | 6.622 | 1.00 | 23.28 |
| B | C | | | | | | | | | | |
| ATOM | | 1510 | C | PHE | B | 80 | 17.289 | 39.474 | 7.113 | 1.00 | 25.53 |
| B | C | | | | | | | | | | |
| ATOM | | 1511 | O | PHE | B | 80 | 16.817 | 39.650 | 5.989 | 1.00 | 25.25 |
| B | O | | | | | | | | | | |
| ATOM | | 1512 | N | HIS | B | 81 | 16.705 | 39.928 | 8.218 | 1.00 | 26.38 |
| B | N | | | | | | | | | | |
| ATOM | | 1513 | CA | HIS | B | 81 | 15.444 | 40.662 | 8.192 | 1.00 | 27.94 |
| B | C | | | | | | | | | | |
| ATOM | | 1514 | CB | AHIS | B | 81 | 15.100 | 41.146 | 9.593 | 0.50 | 31.29 |
| B | C | | | | | | | | | | |
| ATOM | | 1515 | CB | BHIS | B | 81 | 15.009 | 41.082 | 9.560 | 0.50 | 31.04 |
| B | C | | | | | | | | | | |
| ATOM | | 1516 | CG | AHIS | B | 81 | 13.748 | 41.770 | 9.701 | 0.50 | 34.69 |
| B | C | | | | | | | | | | |
| ATOM | | 1517 | CG | BHIS | B | 81 | 14.361 | 40.000 | 10.367 | 0.50 | 33.94 |
| B | C | | | | | | | | | | |
| ATOM | | 1518 | CD2 | AHIS | B | 81 | 12.617 | 41.581 | 8.966 | 0.50 | 35.93 |
| B | C | | | | | | | | | | |
| ATOM | | 1519 | CD2 | BHIS | B | 81 | 14.072 | 38.713 | 10.063 | 0.50 | 35.25 |
| B | C | | | | | | | | | | |
| ATOM | | 1520 | ND1 | AHIS | B | 81 | 13.422 | 42.706 | 10.655 | 0.50 | 36.16 |
| B | N | | | | | | | | | | |
| ATOM | | 1521 | ND1 | BHIS | B | 81 | 13.923 | 40.196 | 11.660 | 0.50 | 35.74 |
| B | N | | | | | | | | | | |
| ATOM | | 1522 | CE1 | AHIS | B | 81 | 12.158 | 43.070 | 10.511 | 0.50 | 37.27 |
| B | C | | | | | | | | | | |
| ATOM | | 1523 | CE1 | BHIS | B | 81 | 13.393 | 39.075 | 12.117 | 0.50 | 36.86 |
| B | C | | | | | | | | | | |
| ATOM | | 1524 | NE2 | AHIS | B | 81 | 11.653 | 42.400 | 9.493 | 0.50 | 37.17 |
| B | N | | | | | | | | | | |
| ATOM | | 1525 | NE2 | BHIS | B | 81 | 13.470 | 38.160 | 11.168 | 0.50 | 36.06 |
| B | N | | | | | | | | | | |
| ATOM | | 1526 | C | HIS | B | 81 | 15.507 | 41.870 | 7.260 | 1.00 | 27.52 |
| B | C | | | | | | | | | | |
| ATOM | | 1527 | O | HIS | B | 81 | 16.309 | 42.781 | 7.473 | 1.00 | 27.00 |
| B | O | | | | | | | | | | |
| ATOM | | 1528 | N | GLY | B | 82 | 14.685 | 41.857 | 6.213 | 1.00 | 26.26 |
| B | N | | | | | | | | | | |
| ATOM | | 1529 | CA | GLY | B | 82 | 14.667 | 42.960 | 5.270 | 1.00 | 24.51 |
| B | C | | | | | | | | | | |
| ATOM | | 1530 | C | GLY | B | 82 | 15.874 | 43.081 | 4.366 | 1.00 | 24.60 |
| B | C | | | | | | | | | | |
| ATOM | | 1531 | O | GLY | B | 82 | 16.060 | 44.108 | 3.722 | 1.00 | 25.03 |
| B | O | | | | | | | | | | |
| ATOM | | 1532 | N | VAL | B | 83 | 16.702 | 42.044 | 4.317 | 1.00 | 26.22 |
| B | N | | | | | | | | | | |
| ATOM | | 1533 | CA | VAL | B | 83 | 17.894 | 42.058 | 3.474 | 1.00 | 24.22 |
| B | C | | | | | | | | | | |
| ATOM | | 1534 | CB | VAL | B | 83 | 19.099 | 41.422 | 4.198 | 1.00 | 23.59 |
| B | C | | | | | | | | | | |
| ATOM | | 1535 | CG1 | VAL | B | 83 | 20.338 | 41.501 | 3.315 | 1.00 | 21.40 |
| B | C | | | | | | | | | | |
| ATOM | | 1536 | CG2 | VAL | B | 83 | 19.332 | 42.121 | 5.526 | 1.00 | 20.96 |
| B | C | | | | | | | | | | |
| ATOM | | 1537 | C | VAL | B | 83 | 17.635 | 41.282 | 2.188 | 1.00 | 26.23 |
| B | C | | | | | | | | | | |
| ATOM | | 1538 | O | VAL | B | 83 | 17.400 | 40.078 | 2.220 | 1.00 | 26.03 |
| B | O | | | | | | | | | | |
| ATOM | | 1539 | N | SER | B | 84 | 17.671 | 41.974 | 1.055 | 1.00 | 29.00 |
| B | N | | | | | | | | | | |
| ATOM | | 1540 | CA | SER | B | 84 | 17.438 | 41.317 | −0.226 | 1.00 | 31.27 |
| B | C | | | | | | | | | | |
| ATOM | | 1541 | CB | SER | B | 84 | 17.307 | 42.360 | −1.341 | 1.00 | 31.72 |
| B | C | | | | | | | | | | |
| ATOM | | 1542 | OG | SER | B | 84 | 18.503 | 43.103 | −1.500 | 1.00 | 34.12 |
| B | O | | | | | | | | | | |
| ATOM | | 1543 | C | SER | B | 84 | 18.599 | 40.374 | −0.522 | 1.00 | 32.20 |
| B | C | | | | | | | | | | |
| ATOM | | 1544 | O | SER | B | 84 | 19.659 | 40.476 | 0.099 | 1.00 | 31.68 |
| B | O | | | | | | | | | | |
| ATOM | | 1545 | N | MET | B | 85 | 18.396 | 39.454 | −1.462 | 1.00 | 32.77 |
| B | N | | | | | | | | | | |
| ATOM | | 1546 | CA | MET | B | 85 | 19.434 | 38.492 | −1.837 | 1.00 | 33.78 |
| B | C | | | | | | | | | | |
| ATOM | | 1547 | CB | MET | B | 85 | 18.916 | 37.542 | −2.925 | 1.00 | 36.68 |
| B | C | | | | | | | | | | |
| ATOM | | 1548 | CG | MET | B | 85 | 17.883 | 36.545 | −2.439 | 1.00 | 40.28 |
| B | C | | | | | | | | | | |
| ATOM | | 1549 | SD | MET | B | 85 | 18.469 | 35.551 | −1.056 | 1.00 | 48.72 |
| B | S | | | | | | | | | | |
| ATOM | | 1550 | CE | MET | B | 85 | 19.962 | 34.852 | −1.742 | 1.00 | 44.99 |
| B | C | | | | | | | | | | |
| ATOM | | 1551 | C | MET | B | 85 | 20.704 | 39.167 | −2.342 | 1.00 | 32.27 |
| B | C | | | | | | | | | | |
| ATOM | | 1552 | O | MET | B | 85 | 21.814 | 38.650 | −2.165 | 1.00 | 33.11 |
| B | O | | | | | | | | | | |
| ATOM | | 1553 | N | SER | B | 86 | 20.538 | 40.323 | −2.971 | 1.00 | 30.58 |
| B | N | | | | | | | | | | |
| ATOM | | 1554 | CA | SER | B | 86 | 21.669 | 41.053 | −3.521 | 1.00 | 31.46 |
| B | C | | | | | | | | | | |
| ATOM | | 1555 | CB | SER | B | 86 | 21.187 | 42.065 | −4.573 | 1.00 | 32.63 |
| B | C | | | | | | | | | | |
| ATOM | | 1556 | OG | SER | B | 86 | 20.319 | 43.052 | −4.031 | 1.00 | 39.50 |
| B | O | | | | | | | | | | |
| ATOM | | 1557 | C | SER | B | 86 | 22.492 | 41.774 | −2.471 | 1.00 | 29.04 |
| B | C | | | | | | | | | | |
| ATOM | | 1558 | O | SER | B | 86 | 23.608 | 42.227 | −2.746 | 1.00 | 28.91 |
| B | O | | | | | | | | | | |
| ATOM | | 1559 | N | GLU | B | 87 | 21.941 | 41.863 | −1.265 | 1.00 | 26.59 |
| B | N | | | | | | | | | | |
| ATOM | | 1560 | CA | GLU | B | 87 | 22.608 | 42.539 | −0.162 | 1.00 | 24.76 |
| B | C | | | | | | | | | | |
| ATOM | | 1561 | CB | GLU | B | 87 | 21.601 | 43.420 | 0.581 | 1.00 | 25.49 |
| B | C | | | | | | | | | | |
| ATOM | | 1562 | CG | GLU | B | 87 | 20.847 | 44.392 | −0.313 | 1.00 | 29.17 |
| B | C | | | | | | | | | | |
| ATOM | | 1563 | CD | GLU | B | 87 | 19.853 | 45.248 | 0.449 | 1.00 | 30.87 |
| B | C | | | | | | | | | | |
| ATOM | | 1564 | OE1 | GLU | B | 87 | 19.028 | 44.689 | 1.206 | 1.00 | 30.33 |
| B | O | | | | | | | | | | |
| ATOM | | 1565 | OE2 | GLU | B | 87 | 19.891 | 46.484 | 0.283 | 1.00 | 35.00 |
| B | O | | | | | | | | | | |
| ATOM | | 1566 | C | GLU | B | 87 | 23.257 | 41.563 | 0.821 | 1.00 | 23.73 |
| B | C | | | | | | | | | | |
| ATOM | | 1567 | O | GLU | B | 87 | 24.029 | 41.972 | 1.685 | 1.00 | 21.32 |
| B | O | | | | | | | | | | |
| ATOM | | 1568 | N | ARG | B | 88 | 22.957 | 40.276 | 0.676 | 1.00 | 22.70 |
| B | N | | | | | | | | | | |
| ATOM | | 1569 | CA | ARG | B | 88 | 23.499 | 39.261 | 1.578 | 1.00 | 24.54 |
| B | C | | | | | | | | | | |
| ATOM | | 1570 | CB | ARG | B | 88 | 22.967 | 37.881 | 1.194 | 1.00 | 25.66 |
| B | C | | | | | | | | | | |
| ATOM | | 1571 | CG | ARG | B | 88 | 21.509 | 37.681 | 1.537 | 1.00 | 27.47 |
| B | C | | | | | | | | | | |
| ATOM | | 1572 | CD | ARG | B | 88 | 21.118 | 36.224 | 1.391 | 1.00 | 29.90 |
| B | C | | | | | | | | | | |
| ATOM | | 1573 | NE | ARG | B | 88 | 19.755 | 35.977 | 1.853 | 1.00 | 33.50 |
| B | N | | | | | | | | | | |
| ATOM | | 1574 | CZ | ARG | B | 88 | 19.197 | 34.772 | 1.926 | 1.00 | 34.03 |
| B | C | | | | | | | | | | |
| ATOM | | 1575 | NH1 | ARG | B | 88 | 19.888 | 33.699 | 1.569 | 1.00 | 34.95 |
| B | N | | | | | | | | | | |
| ATOM | | 1576 | NH2 | ARG | B | 88 | 17.947 | 34.642 | 2.350 | 1.00 | 35.52 |
| B | N | | | | | | | | | | |
| ATOM | | 1577 | C | ARG | B | 88 | 25.018 | 39.187 | 1.727 | 1.00 | 23.31 |
| B | C | | | | | | | | | | |
| ATOM | | 1578 | O | ARG | B | 88 | 25.522 | 39.058 | 2.841 | 1.00 | 23.06 |
| B | O | | | | | | | | | | |
| ATOM | | 1579 | N | CYS | B | 89 | 25.756 | 39.256 | 0.626 | 1.00 | 23.32 |
| B | N | | | | | | | | | | |
| ATOM | | 1580 | CA | CYS | B | 89 | 27.208 | 39.170 | 0.739 | 1.00 | 22.87 |
| B | C | | | | | | | | | | |
| ATOM | | 1581 | C | CYS | B | 89 | 27.778 | 40.378 | 1.486 | 1.00 | 22.34 |
| B | C | | | | | | | | | | |
| ATOM | | 1582 | O | CYS | B | 89 | 28.706 | 40.238 | 2.287 | 1.00 | 20.71 |
| B | O | | | | | | | | | | |
| ATOM | | 1583 | CB | CYS | B | 89 | 27.865 | 39.037 | −0.639 | 1.00 | 22.30 |
| B | C | | | | | | | | | | |
| ATOM | | 1584 | SG | CYS | B | 89 | 29.668 | 38.836 | −0.507 | 1.00 | 24.58 |
| B | S | | | | | | | | | | |
| ATOM | | 1585 | N | TYR | B | 90 | 27.207 | 41.555 | 1.232 | 1.00 | 22.12 |
| B | N | | | | | | | | | | |
| ATOM | | 1586 | CA | TYR | B | 90 | 27.636 | 42.786 | 1.891 | 1.00 | 21.33 |
| B | C | | | | | | | | | | |
| ATOM | | 1587 | CB | TYR | B | 90 | 26.896 | 43.990 | 1.307 | 1.00 | 24.19 |
| B | C | | | | | | | | | | |
| ATOM | | 1588 | CG | TYR | B | 90 | 27.227 | 45.299 | 1.989 | 1.00 | 25.69 |
| B | C | | | | | | | | | | |
| ATOM | | 1589 | CD1 | TYR | B | 90 | 28.484 | 45.897 | 1.838 | 1.00 | 26.69 |
| B | C | | | | | | | | | | |
| ATOM | | 1590 | CE1 | TYR | B | 90 | 28.795 | 47.101 | 2.483 | 1.00 | 26.25 |
| B | C | | | | | | | | | | |
| ATOM | | 1591 | CD2 | TYR | B | 90 | 26.290 | 45.933 | 2.803 | 1.00 | 26.17 |
| B | C | | | | | | | | | | |
| ATOM | | 1592 | CE2 | TYR | B | 90 | 26.591 | 47.130 | 3.452 | 1.00 | 27.02 |
| B | C | | | | | | | | | | |
| ATOM | | 1593 | CZ | TYR | B | 90 | 27.842 | 47.707 | 3.288 | 1.00 | 26.75 |
| B | C | | | | | | | | | | |
| ATOM | | 1594 | OH | TYR | B | 90 | 28.129 | 48.891 | 3.925 | 1.00 | 28.36 |
| B | O | | | | | | | | | | |
| ATOM | | 1595 | C | TYR | B | 90 | 27.310 | 42.652 | 3.375 | 1.00 | 20.94 |
| B | C | | | | | | | | | | |
| ATOM | | 1596 | O | TYR | B | 90 | 28.081 | 43.075 | 4.239 | 1.00 | 21.09 |
| B | O | | | | | | | | | | |
| ATOM | | 1597 | N | LEU | B | 91 | 26.156 | 42.065 | 3.658 | 1.00 | 17.63 |
| B | N | | | | | | | | | | |
| ATOM | | 1598 | CA | LEU | B | 91 | 25.724 | 41.828 | 5.031 | 1.00 | 18.40 |
| B | C | | | | | | | | | | |
| ATOM | | 1599 | CB | LEU | B | 91 | 24.349 | 41.158 | 5.025 | 1.00 | 19.21 |
| B | C | | | | | | | | | | |
| ATOM | | 1600 | CG | LEU | B | 91 | 23.874 | 40.595 | 6.361 | 1.00 | 22.33 |
| B | C | | | | | | | | | | |
| ATOM | | 1601 | CD1 | LEU | B | 91 | 23.538 | 41.736 | 7.296 | 1.00 | 19.22 |
| B | C | | | | | | | | | | |
| ATOM | | 1602 | CD2 | LEU | B | 91 | 22.657 | 39.700 | 6.137 | 1.00 | 23.86 |
| B | C | | | | | | | | | | |
| ATOM | | 1603 | C | LEU | B | 91 | 26.734 | 40.912 | 5.741 | 1.00 | 17.20 |
| B | C | | | | | | | | | | |
| ATOM | | 1604 | O | LEU | B | 91 | 27.210 | 41.211 | 6.841 | 1.00 | 17.10 |
| B | O | | | | | | | | | | |
| ATOM | | 1605 | N | MET | B | 92 | 27.056 | 39.791 | 5.107 | 1.00 | 16.43 |
| B | N | | | | | | | | | | |
| ATOM | | 1606 | CA | MET | B | 92 | 27.997 | 38.847 | 5.685 | 1.00 | 17.07 |
| B | C | | | | | | | | | | |
| ATOM | | 1607 | CB | MET | B | 92 | 28.002 | 37.552 | 4.871 | 1.00 | 16.48 |
| B | C | | | | | | | | | | |
| ATOM | | 1608 | CG | MET | B | 92 | 26.703 | 36.752 | 4.979 | 1.00 | 17.63 |
| B | C | | | | | | | | | | |
| ATOM | | 1609 | SD | MET | B | 92 | 26.128 | 36.605 | 6.690 | 1.00 | 17.99 |
| B | S | | | | | | | | | | |
| ATOM | | 1610 | CE | MET | B | 92 | 27.492 | 35.700 | 7.425 | 1.00 | 14.17 |
| B | C | | | | | | | | | | |
| ATOM | | 1611 | C | MET | B | 92 | 29.410 | 39.430 | 5.790 | 1.00 | 18.87 |
| B | C | | | | | | | | | | |
| ATOM | | 1612 | O | MET | B | 92 | 30.187 | 39.048 | 6.670 | 1.00 | 17.61 |
| B | O | | | | | | | | | | |
| ATOM | | 1613 | N | LYS | B | 93 | 29.747 | 40.350 | 4.891 | 1.00 | 20.69 |
| B | N | | | | | | | | | | |
| ATOM | | 1614 | CA | LYS | B | 93 | 31.060 | 40.988 | 4.936 | 1.00 | 20.45 |
| B | C | | | | | | | | | | |
| ATOM | | 1615 | CB | LYS | B | 93 | 31.219 | 41.984 | 3.791 | 1.00 | 21.79 |
| B | C | | | | | | | | | | |
| ATOM | | 1616 | CG | LYS | B | 93 | 32.527 | 42.755 | 3.838 | 1.00 | 22.89 |
| B | C | | | | | | | | | | |
| ATOM | | 1617 | CD | LYS | B | 93 | 32.490 | 43.949 | 2.892 | 1.00 | 27.03 |
| B | C | | | | | | | | | | |
| ATOM | | 1618 | CE | LYS | B | 93 | 33.765 | 44.778 | 2.995 | 1.00 | 28.50 |
| B | C | | | | | | | | | | |
| ATOM | | 1619 | NZ | LYS | B | 93 | 33.700 | 46.001 | 2.138 | 1.00 | 30.45 |
| B | N | | | | | | | | | | |
| ATOM | | 1620 | C | LYS | B | 93 | 31.181 | 41.732 | 6.267 | 1.00 | 19.66 |
| B | C | | | | | | | | | | |
| ATOM | | 1621 | O | LYS | B | 93 | 32.200 | 41.649 | 6.946 | 1.00 | 19.17 |
| B | O | | | | | | | | | | |
| ATOM | | 1622 | N | GLN | B | 94 | 30.131 | 42.456 | 6.637 | 1.00 | 19.14 |
| B | N | | | | | | | | | | |
| ATOM | | 1623 | CA | GLN | B | 94 | 30.136 | 43.201 | 7.890 | 1.00 | 19.70 |
| B | C | | | | | | | | | | |
| ATOM | | 1624 | CB | GLN | B | 94 | 28.814 | 43.956 | 8.061 | 1.00 | 21.16 |
| B | C | | | | | | | | | | |
| ATOM | | 1625 | CG | GLN | B | 94 | 28.392 | 44.765 | 6.844 | 1.00 | 22.73 |
| B | C | | | | | | | | | | |
| ATOM | | 1626 | CD | GLN | B | 94 | 29.451 | 45.752 | 6.397 | 1.00 | 22.05 |
| B | C | | | | | | | | | | |
| ATOM | | 1627 | OE1 | GLN | B | 94 | 29.802 | 45.806 | 5.217 | 1.00 | 26.54 |
| B | O | | | | | | | | | | |
| ATOM | | 1628 | NE2 | GLN | B | 94 | 29.963 | 46.541 | 7.333 | 1.00 | 18.51 |
| B | N | | | | | | | | | | |
| ATOM | | 1629 | C | GLN | B | 94 | 30.329 | 42.244 | 9.071 | 1.00 | 18.12 |
| B | C | | | | | | | | | | |
| ATOM | | 1630 | O | GLN | B | 94 | 31.121 | 42.510 | 9.980 | 1.00 | 17.93 |
| B | O | | | | | | | | | | |
| ATOM | | 1631 | N | VAL | B | 95 | 29.592 | 41.138 | 9.055 | 1.00 | 14.83 |
| B | N | | | | | | | | | | |
| ATOM | | 1632 | CA | VAL | B | 95 | 29.679 | 40.140 | 10.117 | 1.00 | 13.76 |
| B | C | | | | | | | | | | |
| ATOM | | 1633 | CB | VAL | B | 95 | 28.635 | 39.025 | 9.906 | 1.00 | 13.80 |
| B | C | | | | | | | | | | |
| ATOM | | 1634 | CG1 | VAL | B | 95 | 28.812 | 37.932 | 10.944 | 1.00 | 12.84 |
| B | C | | | | | | | | | | |
| ATOM | | 1635 | CG2 | VAL | B | 95 | 27.246 | 39.611 | 9.997 | 1.00 | 11.25 |
| B | C | | | | | | | | | | |
| ATOM | | 1636 | C | VAL | B | 95 | 31.079 | 39.529 | 10.149 | 1.00 | 13.82 |
| B | C | | | | | | | | | | |
| ATOM | | 1637 | O | VAL | B | 95 | 31.690 | 39.410 | 11.211 | 1.00 | 13.76 |
| B | O | | | | | | | | | | |
| ATOM | | 1638 | N | LEU | B | 96 | 31.583 | 39.149 | 8.980 | 1.00 | 14.12 |
| B | N | | | | | | | | | | |
| ATOM | | 1639 | CA | LEU | B | 96 | 32.918 | 38.570 | 8.870 | 1.00 | 15.35 |
| B | C | | | | | | | | | | |
| ATOM | | 1640 | CB | LEU | B | 96 | 33.251 | 38.298 | 7.403 | 1.00 | 15.65 |
| B | C | | | | | | | | | | |
| ATOM | | 1641 | CG | LEU | B | 96 | 34.721 | 37.969 | 7.126 | 1.00 | 16.49 |
| B | C | | | | | | | | | | |
| ATOM | | 1642 | CD1 | LEU | B | 96 | 35.100 | 36.677 | 7.833 | 1.00 | 13.79 |
| B | C | | | | | | | | | | |
| ATOM | | 1643 | CD2 | LEU | B | 96 | 34.947 | 37.856 | 5.628 | 1.00 | 15.98 |
| B | C | | | | | | | | | | |
| ATOM | | 1644 | C | LEU | B | 96 | 34.010 | 39.471 | 9.465 | 1.00 | 16.84 |
| B | C | | | | | | | | | | |
| ATOM | | 1645 | O | LEU | B | 96 | 34.856 | 39.007 | 10.238 | 1.00 | 16.28 |
| B | O | | | | | | | | | | |
| ATOM | | 1646 | N | ASN | B | 97 | 33.994 | 40.752 | 9.097 | 1.00 | 15.44 |
| B | N | | | | | | | | | | |
| ATOM | | 1647 | CA | ASN | B | 97 | 34.997 | 41.699 | 9.590 | 1.00 | 16.09 |
| B | C | | | | | | | | | | |
| ATOM | | 1648 | CB | ASN | B | 97 | 34.901 | 43.021 | 8.821 | 1.00 | 17.20 |
| B | C | | | | | | | | | | |
| ATOM | | 1649 | CG | ASN | B | 97 | 35.362 | 42.873 | 7.385 | 1.00 | 18.71 |
| B | C | | | | | | | | | | |
| ATOM | | 1650 | OD1 | ASN | B | 97 | 36.125 | 41.961 | 7.081 | 1.00 | 18.61 |
| B | O | | | | | | | | | | |
| ATOM | | 1651 | ND2 | ASN | B | 97 | 34.910 | 43.761 | 6.499 | 1.00 | 17.57 |
| B | N | | | | | | | | | | |
| ATOM | | 1652 | C | ASN | B | 97 | 34.896 | 41.940 | 11.085 | 1.00 | 15.40 |
| B | C | | | | | | | | | | |
| ATOM | | 1653 | O | ASN | B | 97 | 35.914 | 42.005 | 11.777 | 1.00 | 16.22 |
| B | O | | | | | | | | | | |
| ATOM | | 1654 | N | PHE | B | 98 | 33.673 | 42.066 | 11.588 | 1.00 | 14.94 |
| B | N | | | | | | | | | | |
| ATOM | | 1655 | CA | PHE | B | 98 | 33.476 | 42.266 | 13.017 | 1.00 | 14.55 |
| B | C | | | | | | | | | | |
| ATOM | | 1656 | CB | PHE | B | 98 | 31.986 | 42.369 | 13.356 | 1.00 | 13.40 |
| B | C | | | | | | | | | | |
| ATOM | | 1657 | CG | PHE | B | 98 | 31.700 | 42.315 | 14.834 | 1.00 | 15.06 |
| B | C | | | | | | | | | | |
| ATOM | | 1658 | CD1 | PHE | B | 98 | 31.958 | 43.415 | 15.649 | 1.00 | 14.98 |
| B | C | | | | | | | | | | |
| ATOM | | 1659 | CD2 | PHE | B | 98 | 31.200 | 41.150 | 15.418 | 1.00 | 14.58 |
| B | C | | | | | | | | | | |
| ATOM | | 1660 | CE1 | PHE | B | 98 | 31.720 | 43.360 | 17.032 | 1.00 | 14.56 |
| B | C | | | | | | | | | | |
| ATOM | | 1661 | CE2 | PHE | B | 98 | 30.957 | 41.077 | 16.799 | 1.00 | 13.20 |
| B | C | | | | | | | | | | |
| ATOM | | 1662 | CZ | PHE | B | 98 | 31.218 | 42.188 | 17.608 | 1.00 | 12.63 |
| B | C | | | | | | | | | | |
| ATOM | | 1663 | C | PHE | B | 98 | 34.055 | 41.074 | 13.764 | 1.00 | 14.69 |
| B | C | | | | | | | | | | |
| ATOM | | 1664 | O | PHE | B | 98 | 34.793 | 41.227 | 14.735 | 1.00 | 14.16 |
| B | O | | | | | | | | | | |
| ATOM | | 1665 | N | THR | B | 99 | 33.705 | 39.880 | 13.300 | 1.00 | 14.39 |
| B | N | | | | | | | | | | |
| ATOM | | 1666 | CA | THR | B | 99 | 34.154 | 38.651 | 13.932 | 1.00 | 13.57 |
| B | C | | | | | | | | | | |
| ATOM | | 1667 | CB | THR | B | 99 | 33.490 | 37.433 | 13.257 | 1.00 | 12.94 |
| B | C | | | | | | | | | | |
| ATOM | | 1668 | OG1 | THR | B | 99 | 32.059 | 37.576 | 13.316 | 1.00 | 12.53 |
| B | O | | | | | | | | | | |
| ATOM | | 1669 | CG2 | THR | B | 99 | 33.896 | 36.143 | 13.958 | 1.00 | 12.70 |
| B | C | | | | | | | | | | |
| ATOM | | 1670 | C | THR | B | 99 | 35.677 | 38.516 | 13.893 | 1.00 | 13.81 |
| B | C | | | | | | | | | | |
| ATOM | | 1671 | O | THR | B | 99 | 36.295 | 38.142 | 14.884 | 1.00 | 12.81 |
| B | O | | | | | | | | | | |
| ATOM | | 1672 | N | LEU | B | 100 | 36.285 | 38.830 | 12.754 | 1.00 | 14.39 |
| B | N | | | | | | | | | | |
| ATOM | | 1673 | CA | LEU | B | 100 | 37.735 | 38.732 | 12.645 | 1.00 | 15.17 |
| B | C | | | | | | | | | | |
| ATOM | | 1674 | CB | LEU | B | 100 | 38.181 | 39.035 | 11.213 | 1.00 | 17.01 |
| B | C | | | | | | | | | | |
| ATOM | | 1675 | CG | LEU | B | 100 | 38.047 | 37.902 | 10.187 | 1.00 | 19.42 |
| B | C | | | | | | | | | | |
| ATOM | | 1676 | CD1 | LEU | B | 100 | 38.195 | 38.470 | 8.778 | 1.00 | 20.62 |
| B | C | | | | | | | | | | |
| ATOM | | 1677 | CD2 | LEU | B | 100 | 39.105 | 36.830 | 10.460 | 1.00 | 19.19 |
| B | C | | | | | | | | | | |
| ATOM | | 1678 | C | LEU | B | 100 | 38.439 | 39.694 | 13.608 | 1.00 | 16.32 |
| B | C | | | | | | | | | | |
| ATOM | | 1679 | O | LEU | B | 100 | 39.276 | 39.289 | 14.412 | 1.00 | 15.91 |
| B | O | | | | | | | | | | |
| ATOM | | 1680 | N | GLU | B | 101 | 38.079 | 40.968 | 13.533 | 1.00 | 15.77 |
| B | N | | | | | | | | | | |
| ATOM | | 1681 | CA | GLU | B | 101 | 38.717 | 41.982 | 14.365 | 1.00 | 17.05 |
| B | C | | | | | | | | | | |
| ATOM | | 1682 | CB | GLU | B | 101 | 38.439 | 43.370 | 13.781 | 1.00 | 20.26 |
| B | C | | | | | | | | | | |
| ATOM | | 1683 | CG | GLU | B | 101 | 39.123 | 43.627 | 12.449 | 1.00 | 25.68 |
| B | C | | | | | | | | | | |
| ATOM | | 1684 | CD | GLU | B | 101 | 38.898 | 45.038 | 11.941 | 1.00 | 29.94 |
| B | C | | | | | | | | | | |
| ATOM | | 1685 | OE1 | GLU | B | 101 | 39.087 | 45.992 | 12.730 | 1.00 | 31.04 |
| B | O | | | | | | | | | | |
| ATOM | | 1686 | OE2 | GLU | B | 101 | 38.541 | 45.192 | 10.752 | 1.00 | 31.88 |
| B | O | | | | | | | | | | |
| ATOM | | 1687 | C | GLU | B | 101 | 38.366 | 41.992 | 15.846 | 1.00 | 15.48 |
| B | C | | | | | | | | | | |
| ATOM | | 1688 | O | GLU | B | 101 | 39.232 | 42.214 | 16.684 | 1.00 | 14.15 |
| B | O | | | | | | | | | | |
| ATOM | | 1689 | N | GLU | B | 102 | 37.103 | 41.749 | 16.169 | 1.00 | 14.59 |
| B | N | | | | | | | | | | |
| ATOM | | 1690 | CA | GLU | B | 102 | 36.664 | 41.792 | 17.558 | 1.00 | 16.25 |
| B | C | | | | | | | | | | |
| ATOM | | 1691 | CB | GLU | B | 102 | 35.292 | 42.462 | 17.624 | 1.00 | 15.08 |
| B | C | | | | | | | | | | |
| ATOM | | 1692 | CG | GLU | B | 102 | 35.257 | 43.884 | 17.055 | 1.00 | 16.02 |
| B | C | | | | | | | | | | |
| ATOM | | 1693 | CD | GLU | B | 102 | 35.992 | 44.893 | 17.928 | 1.00 | 15.88 |
| B | C | | | | | | | | | | |
| ATOM | | 1694 | OE1 | GLU | B | 102 | 35.953 | 44.745 | 19.168 | 1.00 | 15.08 |
| B | O | | | | | | | | | | |
| ATOM | | 1695 | OE2 | GLU | B | 102 | 36.595 | 45.846 | 17.383 | 1.00 | 15.59 |
| B | O | | | | | | | | | | |
| ATOM | | 1696 | C | GLU | B | 102 | 36.611 | 40.451 | 18.283 | 1.00 | 17.63 |
| B | C | | | | | | | | | | |
| ATOM | | 1697 | O | GLU | B | 102 | 36.475 | 40.408 | 19.510 | 1.00 | 16.52 |
| B | O | | | | | | | | | | |
| ATOM | | 1698 | N | VAL | B | 103 | 36.721 | 39.357 | 17.539 | 1.00 | 16.71 |
| B | N | | | | | | | | | | |
| ATOM | | 1699 | CA | VAL | B | 103 | 36.660 | 38.039 | 18.151 | 1.00 | 16.91 |
| B | C | | | | | | | | | | |
| ATOM | | 1700 | CB | VAL | B | 103 | 35.392 | 37.286 | 17.709 | 1.00 | 16.05 |
| B | C | | | | | | | | | | |
| ATOM | | 1701 | CG1 | VAL | B | 103 | 35.394 | 35.881 | 18.310 | 1.00 | 19.19 |
| B | C | | | | | | | | | | |
| ATOM | | 1702 | CG2 | VAL | B | 103 | 34.157 | 38.054 | 18.144 | 1.00 | 18.67 |
| B | C | | | | | | | | | | |
| ATOM | | 1703 | C | VAL | B | 103 | 37.845 | 37.128 | 17.862 | 1.00 | 17.70 |
| B | C | | | | | | | | | | |
| ATOM | | 1704 | O | VAL | B | 103 | 38.540 | 36.680 | 18.784 | 1.00 | 16.69 |
| B | O | | | | | | | | | | |
| ATOM | | 1705 | N | LEU | B | 104 | 38.067 | 36.855 | 16.581 | 1.00 | 16.37 |
| B | N | | | | | | | | | | |
| ATOM | | 1706 | CA | LEU | B | 104 | 39.137 | 35.960 | 16.168 | 1.00 | 18.90 |
| B | C | | | | | | | | | | |
| ATOM | | 1707 | CB | LEU | B | 104 | 38.984 | 35.614 | 14.687 | 1.00 | 15.60 |
| B | C | | | | | | | | | | |
| ATOM | | 1708 | CG | LEU | B | 104 | 37.636 | 34.955 | 14.362 | 1.00 | 16.30 |
| B | C | | | | | | | | | | |
| ATOM | | 1709 | CD1 | LEU | B | 104 | 37.541 | 34.663 | 12.874 | 1.00 | 14.16 |
| B | C | | | | | | | | | | |
| ATOM | | 1710 | CD2 | LEU | B | 104 | 37.485 | 33.671 | 15.162 | 1.00 | 16.44 |
| B | C | | | | | | | | | | |
| ATOM | | 1711 | C | LEU | B | 104 | 40.539 | 36.471 | 16.447 | 1.00 | 20.44 |
| B | C | | | | | | | | | | |
| ATOM | | 1712 | O | LEU | B | 104 | 41.326 | 35.780 | 17.086 | 1.00 | 20.16 |
| B | O | | | | | | | | | | |
| ATOM | | 1713 | N | PHE | B | 105 | 40.870 | 37.666 | 15.979 | 1.00 | 21.99 |
| B | N | | | | | | | | | | |
| ATOM | | 1714 | CA | PHE | B | 105 | 42.213 | 38.166 | 16.239 | 1.00 | 27.13 |
| B | C | | | | | | | | | | |
| ATOM | | 1715 | CB | PHE | B | 105 | 42.387 | 39.579 | 15.675 | 1.00 | 28.81 |
| B | C | | | | | | | | | | |
| ATOM | | 1716 | CG | PHE | B | 105 | 42.367 | 39.634 | 14.168 | 1.00 | 31.64 |
| B | C | | | | | | | | | | |
| ATOM | | 1717 | CD1 | PHE | B | 105 | 42.837 | 38.563 | 13.411 | 1.00 | 33.85 |
| B | C | | | | | | | | | | |
| ATOM | | 1718 | CD2 | PHE | B | 105 | 41.888 | 40.757 | 13.506 | 1.00 | 34.49 |
| B | C | | | | | | | | | | |
| ATOM | | 1719 | CE1 | PHE | B | 105 | 42.831 | 38.611 | 12.015 | 1.00 | 34.86 |
| B | C | | | | | | | | | | |
| ATOM | | 1720 | CE2 | PHE | B | 105 | 41.879 | 40.816 | 12.110 | 1.00 | 35.62 |
| B | C | | | | | | | | | | |
| ATOM | | 1721 | CZ | PHE | B | 105 | 42.351 | 39.739 | 11.365 | 1.00 | 35.69 |
| B | C | | | | | | | | | | |
| ATOM | | 1722 | C | PHE | B | 105 | 42.538 | 38.122 | 17.734 | 1.00 | 27.95 |
| B | C | | | | | | | | | | |
| ATOM | | 1723 | O | PHE | B | 105 | 43.607 | 37.656 | 18.121 | 1.00 | 28.77 |
| B | O | | | | | | | | | | |
| ATOM | | 1724 | N | PRO | B | 106 | 41.613 | 38.587 | 18.594 | 1.00 | 28.70 |
| B | N | | | | | | | | | | |
| ATOM | | 1725 | CD | PRO | B | 106 | 40.390 | 39.341 | 18.266 | 1.00 | 28.83 |
| B | C | | | | | | | | | | |
| ATOM | | 1726 | CA | PRO | B | 106 | 41.836 | 38.578 | 20.045 | 1.00 | 27.96 |
| B | C | | | | | | | | | | |
| ATOM | | 1727 | CB | PRO | B | 106 | 40.590 | 39.280 | 20.589 | 1.00 | 28.83 |
| B | C | | | | | | | | | | |
| ATOM | | 1728 | CG | PRO | B | 106 | 40.223 | 40.214 | 19.481 | 1.00 | 28.82 |
| B | C | | | | | | | | | | |
| ATOM | | 1729 | C | PRO | B | 106 | 41.991 | 37.166 | 20.622 | 1.00 | 27.58 |
| B | C | | | | | | | | | | |
| ATOM | | 1730 | O | PRO | B | 106 | 42.607 | 36.983 | 21.671 | 1.00 | 25.86 |
| B | O | | | | | | | | | | |
| ATOM | | 1731 | N | GLN | B | 107 | 41.419 | 36.174 | 19.945 | 1.00 | 26.57 |
| B | N | | | | | | | | | | |
| ATOM | | 1732 | CA | GLN | B | 107 | 41.499 | 34.784 | 20.394 | 1.00 | 27.34 |
| B | C | | | | | | | | | | |
| ATOM | | 1733 | CB | GLN | B | 107 | 40.119 | 34.123 | 20.321 | 1.00 | 25.90 |
| B | C | | | | | | | | | | |
| ATOM | | 1734 | CG | GLN | B | 107 | 39.027 | 34.737 | 21.181 | 1.00 | 27.05 |
| B | C | | | | | | | | | | |
| ATOM | | 1735 | CD | GLN | B | 107 | 39.054 | 34.228 | 22.601 | 1.00 | 27.78 |
| B | C | | | | | | | | | | |
| ATOM | | 1736 | OE1 | GLN | B | 107 | 39.453 | 33.093 | 22.852 | 1.00 | 29.49 |
| B | O | | | | | | | | | | |
| ATOM | | 1737 | NE2 | GLN | B | 107 | 38.613 | 35.056 | 23.539 | 1.00 | 24.76 |
| B | N | | | | | | | | | | |
| ATOM | | 1738 | C | GLN | B | 107 | 42.457 | 33.994 | 19.497 | 1.00 | 28.04 |
| B | C | | | | | | | | | | |
| ATOM | | 1739 | O | GLN | B | 107 | 42.514 | 32.769 | 19.572 | 1.00 | 28.67 |
| B | O | | | | | | | | | | |
| ATOM | | 1740 | N | SER | B | 108 | 43.206 | 34.697 | 18.654 | 1.00 | 29.80 |
| B | N | | | | | | | | | | |
| ATOM | | 1741 | CA | SER | B | 108 | 44.125 | 34.059 | 17.710 | 1.00 | 31.32 |
| B | C | | | | | | | | | | |
| ATOM | | 1742 | CB | SER | B | 108 | 44.873 | 35.129 | 16.917 | 1.00 | 31.54 |
| B | C | | | | | | | | | | |
| ATOM | | 1743 | OG | SER | B | 108 | 45.612 | 35.971 | 17.784 | 1.00 | 33.99 |
| B | O | | | | | | | | | | |
| ATOM | | 1744 | C | SER | B | 108 | 45.131 | 33.064 | 18.291 | 1.00 | 32.91 |
| B | C | | | | | | | | | | |
| ATOM | | 1745 | O | SER | B | 108 | 45.661 | 32.220 | 17.562 | 1.00 | 32.09 |
| B | O | | | | | | | | | | |
| ATOM | | 1746 | N | ASP | B | 109 | 45.399 | 33.156 | 19.589 | 1.00 | 33.94 |
| B | N | | | | | | | | | | |
| ATOM | | 1747 | CA | ASP | B | 109 | 46.346 | 32.243 | 20.221 | 1.00 | 36.43 |
| B | C | | | | | | | | | | |
| ATOM | | 1748 | CB | ASP | B | 109 | 47.268 | 33.002 | 21.178 | 1.00 | 38.99 |
| B | C | | | | | | | | | | |
| ATOM | | 1749 | CG | ASP | B | 109 | 48.429 | 33.660 | 20.464 | 1.00 | 41.54 |
| B | C | | | | | | | | | | |
| ATOM | | 1750 | OD1 | ASP | B | 109 | 48.195 | 34.463 | 19.536 | 1.00 | 43.90 |
| B | O | | | | | | | | | | |
| ATOM | | 1751 | OD2 | ASP | B | 109 | 49.585 | 33.370 | 20.834 | 1.00 | 45.38 |
| B | O | | | | | | | | | | |
| ATOM | | 1752 | C | ASP | B | 109 | 45.669 | 31.103 | 20.965 | 1.00 | 35.97 |
| B | C | | | | | | | | | | |
| ATOM | | 1753 | O | ASP | B | 109 | 46.340 | 30.261 | 21.558 | 1.00 | 37.38 |
| B | O | | | | | | | | | | |
| ATOM | | 1754 | N | ARG | B | 110 | 44.342 | 31.070 | 20.929 | 1.00 | 34.07 |
| B | N | | | | | | | | | | |
| ATOM | | 1755 | CA | ARG | B | 110 | 43.595 | 30.019 | 21.606 | 1.00 | 32.11 |
| B | C | | | | | | | | | | |
| ATOM | | 1756 | CB | ARG | B | 110 | 42.483 | 30.628 | 22.457 | 1.00 | 34.24 |
| B | C | | | | | | | | | | |
| ATOM | | 1757 | CG | ARG | B | 110 | 42.974 | 31.587 | 23.523 | 1.00 | 38.78 |
| B | C | | | | | | | | | | |
| ATOM | | 1758 | CD | ARG | B | 110 | 41.799 | 32.210 | 24.253 | 1.00 | 42.57 |
| B | C | | | | | | | | | | |
| ATOM | | 1759 | NE | ARG | B | 110 | 42.213 | 33.153 | 25.285 | 1.00 | 46.77 |
| B | N | | | | | | | | | | |
| ATOM | | 1760 | CZ | ARG | B | 110 | 42.872 | 32.815 | 26.389 | 1.00 | 48.68 |
| B | C | | | | | | | | | | |
| ATOM | | 1761 | NH1 | ARG | B | 110 | 43.198 | 31.545 | 26.610 | 1.00 | 48.55 |
| B | N | | | | | | | | | | |
| ATOM | | 1762 | NH2 | ARG | B | 110 | 43.197 | 33.750 | 27.274 | 1.00 | 49.15 |
| B | N | | | | | | | | | | |
| ATOM | | 1763 | C | ARG | B | 110 | 42.989 | 29.047 | 20.601 | 1.00 | 29.74 |
| B | C | | | | | | | | | | |
| ATOM | | 1764 | O | ARG | B | 110 | 43.132 | 29.216 | 19.391 | 1.00 | 28.95 |
| B | O | | | | | | | | | | |
| ATOM | | 1765 | N | PHE | B | 111 | 42.314 | 28.025 | 21.119 | 1.00 | 28.23 |
| B | N | | | | | | | | | | |
| ATOM | | 1766 | CA | PHE | B | 111 | 41.671 | 27.018 | 20.289 | 1.00 | 25.68 |
| B | C | | | | | | | | | | |
| ATOM | | 1767 | CB | PHE | B | 111 | 40.429 | 27.619 | 19.627 | 1.00 | 25.02 |
| B | C | | | | | | | | | | |
| ATOM | | 1768 | CG | PHE | B | 111 | 39.351 | 28.002 | 20.604 | 1.00 | 21.00 |
| B | C | | | | | | | | | | |
| ATOM | | 1769 | CD1 | PHE | B | 111 | 38.563 | 27.029 | 21.206 | 1.00 | 22.14 |
| B | C | | | | | | | | | | |
| ATOM | | 1770 | CD2 | PHE | B | 111 | 39.151 | 29.331 | 20.954 | 1.00 | 21.84 |
| B | C | | | | | | | | | | |
| ATOM | | 1771 | CE1 | PHE | B | 111 | 37.586 | 27.374 | 22.146 | 1.00 | 21.32 |
| B | C | | | | | | | | | | |
| ATOM | | 1772 | CE2 | PHE | B | 111 | 38.178 | 29.687 | 21.891 | 1.00 | 22.27 |
| B | C | | | | | | | | | | |
| ATOM | | 1773 | CZ | PHE | B | 111 | 37.397 | 28.704 | 22.487 | 1.00 | 20.90 |
| B | C | | | | | | | | | | |
| ATOM | | 1774 | C | PHE | B | 111 | 42.610 | 26.436 | 19.239 | 1.00 | 25.72 |
| B | C | | | | | | | | | | |
| ATOM | | 1775 | O | PHE | B | 111 | 42.240 | 26.272 | 18.080 | 1.00 | 23.01 |
| B | O | | | | | | | | | | |
| ATOM | | 1776 | N | GLN | B | 112 | 43.829 | 26.119 | 19.659 | 1.00 | 26.79 |
| B | N | | | | | | | | | | |
| ATOM | | 1777 | CA | GLN | B | 112 | 44.820 | 25.530 | 18.764 | 1.00 | 28.39 |
| B | C | | | | | | | | | | |
| ATOM | | 1778 | CE | GLN | B | 112 | 46.211 | 25.622 | 19.392 | 1.00 | 29.98 |
| B | C | | | | | | | | | | |
| ATOM | | 1779 | CG | GLN | B | 112 | 46.709 | 27.043 | 19.587 | 1.00 | 33.82 |
| B | C | | | | | | | | | | |
| ATOM | | 1780 | CD | GLN | B | 112 | 48.085 | 27.100 | 20.227 | 1.00 | 36.64 |
| B | C | | | | | | | | | | |
| ATOM | | 1781 | OE1 | GLN | B | 112 | 49.036 | 26.482 | 19.742 | 1.00 | 39.25 |
| B | O | | | | | | | | | | |
| ATOM | | 1782 | NE2 | GLN | B | 112 | 48.199 | 27.847 | 21.320 | 1.00 | 36.49 |
| B | N | | | | | | | | | | |
| ATOM | | 1783 | C | GLN | B | 112 | 44.470 | 24.063 | 18.501 | 1.00 | 28.37 |
| B | C | | | | | | | | | | |
| ATOM | | 1784 | O | GLN | B | 112 | 43.903 | 23.389 | 19.358 | 1.00 | 27.65 |
| B | O | | | | | | | | | | |
| ATOM | | 1785 | N | PRO | B | 113 | 44.820 | 23.545 | 17.313 | 1.00 | 28.65 |
| B | N | | | | | | | | | | |
| ATOM | | 1786 | CD | PRO | B | 113 | 44.778 | 22.094 | 17.054 | 1.00 | 28.84 |
| B | C | | | | | | | | | | |
| ATOM | | 1787 | CA | PRO | B | 113 | 45.511 | 24.236 | 16.220 | 1.00 | 28.88 |
| B | C | | | | | | | | | | |
| ATOM | | 1788 | CB | PRO | B | 113 | 46.398 | 23.144 | 15.658 | 1.00 | 29.67 |
| B | C | | | | | | | | | | |
| ATOM | | 1789 | CG | PRO | B | 113 | 45.455 | 21.976 | 15.683 | 1.00 | 28.06 |
| B | C | | | | | | | | | | |
| ATOM | | 1790 | C | PRO | B | 113 | 44.550 | 24.757 | 15.160 | 1.00 | 29.20 |
| B | C | | | | | | | | | | |
| ATOM | | 1791 | O | PRO | B | 113 | 44.981 | 25.265 | 14.127 | 1.00 | 31.40 |
| B | O | | | | | | | | | | |
| ATOM | | 1792 | N | TYR | B | 114 | 43.253 | 24.624 | 15.414 | 1.00 | 29.48 |
| B | N | | | | | | | | | | |
| ATOM | | 1793 | CA | TYR | B | 114 | 42.232 | 25.047 | 14.459 | 1.00 | 29.46 |
| B | C | | | | | | | | | | |
| ATOM | | 1794 | CB | TYR | B | 114 | 40.839 | 24.695 | 14.992 | 1.00 | 30.23 |
| B | C | | | | | | | | | | |
| ATOM | | 1795 | CG | TYR | B | 114 | 40.717 | 23.288 | 15.537 | 1.00 | 30.05 |
| B | C | | | | | | | | | | |
| ATOM | | 1796 | CD1 | TYR | B | 114 | 40.943 | 22.177 | 14.724 | 1.00 | 32.21 |
| B | C | | | | | | | | | | |
| ATOM | | 1797 | CE1 | TYR | B | 114 | 40.840 | 20.881 | 15.230 | 1.00 | 32.84 |
| B | C | | | | | | | | | | |
| ATOM | | 1798 | CD2 | TYR | B | 114 | 40.383 | 23.069 | 16.871 | 1.00 | 31.34 |
| B | C | | | | | | | | | | |
| ATOM | | 1799 | CE2 | TYR | B | 114 | 40.278 | 21.784 | 17.385 | 1.00 | 32.91 |
| B | C | | | | | | | | | | |
| ATOM | | 1800 | CZ | TYR | B | 114 | 40.508 | 20.695 | 16.562 | 1.00 | 33.51 |
| B | C | | | | | | | | | | |
| ATOM | | 1801 | OH | TYR | B | 114 | 40.413 | 19.424 | 17.081 | 1.00 | 35.76 |
| B | O | | | | | | | | | | |
| ATOM | | 1802 | C | TYR | B | 114 | 42.271 | 26.530 | 14.117 | 1.00 | 28.68 |
| B | C | | | | | | | | | | |
| ATOM | | 1803 | O | TYR | B | 114 | 42.347 | 26.909 | 12.948 | 1.00 | 29.12 |
| B | O | | | | | | | | | | |
| ATOM | | 1804 | N | MET | B | 115 | 42.213 | 27.366 | 15.146 | 1.00 | 28.56 |
| B | N | | | | | | | | | | |
| ATOM | | 1805 | CA | MET | B | 115 | 42.214 | 28.816 | 14.973 | 1.00 | 27.58 |
| B | C | | | | | | | | | | |
| ATOM | | 1806 | CB | MET | B | 115 | 42.343 | 29.488 | 16.344 | 1.00 | 27.94 |
| B | C | | | | | | | | | | |
| ATOM | | 1807 | CG | MET | B | 115 | 42.184 | 30.992 | 16.341 | 1.00 | 26.96 |
| B | C | | | | | | | | | | |
| ATOM | | 1808 | SD | MET | B | 115 | 40.483 | 31.474 | 16.055 | 1.00 | 27.58 |
| B | S | | | | | | | | | | |
| ATOM | | 1809 | CE | MET | B | 115 | 39.757 | 31.233 | 17.663 | 1.00 | 25.68 |
| B | C | | | | | | | | | | |
| ATOM | | 1810 | C | MET | B | 115 | 43.322 | 29.326 | 14.050 | 1.00 | 27.41 |
| B | C | | | | | | | | | | |
| ATOM | | 1811 | O | MET | B | 115 | 43.087 | 30.163 | 13.181 | 1.00 | 27.38 |
| B | O | | | | | | | | | | |
| ATOM | | 1812 | N | GLN | B | 116 | 44.530 | 28.814 | 14.240 | 1.00 | 28.20 |
| B | N | | | | | | | | | | |
| ATOM | | 1813 | CA | GLN | B | 116 | 45.679 | 29.243 | 13.458 | 1.00 | 29.73 |
| B | C | | | | | | | | | | |
| ATOM | | 1814 | CB | GLN | B | 116 | 46.935 | 28.565 | 13.997 | 1.00 | 31.41 |
| B | C | | | | | | | | | | |
| ATOM | | 1815 | CG | GLN | B | 116 | 47.200 | 28.852 | 15.474 | 1.00 | 34.08 |
| B | C | | | | | | | | | | |
| ATOM | | 1816 | CD | GLN | B | 116 | 46.074 | 28.388 | 16.390 | 1.00 | 35.14 |
| B | C | | | | | | | | | | |
| ATOM | | 1817 | OE1 | GLN | B | 116 | 45.649 | 27.235 | 16.338 | 1.00 | 34.39 |
| B | O | | | | | | | | | | |
| ATOM | | 1818 | NE2 | GLN | B | 116 | 45.590 | 29.292 | 17.239 | 1.00 | 37.03 |
| B | N | | | | | | | | | | |
| ATOM | | 1819 | C | GLN | B | 116 | 45.579 | 29.037 | 11.948 | 1.00 | 30.97 |
| B | C | | | | | | | | | | |
| ATOM | | 1820 | O | GLN | B | 116 | 46.279 | 29.697 | 11.184 | 1.00 | 31.99 |
| B | O | | | | | | | | | | |
| ATOM | | 1821 | N | GLU | B | 117 | 44.714 | 28.129 | 11.513 | 1.00 | 31.07 |
| B | N | | | | | | | | | | |
| ATOM | | 1822 | CA | GLU | B | 117 | 44.549 | 27.875 | 10.086 | 1.00 | 30.90 |
| B | C | | | | | | | | | | |
| ATOM | | 1823 | CB | GLU | B | 117 | 44.409 | 26.375 | 9.839 | 1.00 | 33.41 |
| B | C | | | | | | | | | | |
| ATOM | | 1824 | CG | GLU | B | 117 | 45.624 | 25.581 | 10.273 | 1.00 | 37.00 |
| B | C | | | | | | | | | | |
| ATOM | | 1825 | CD | GLU | B | 117 | 45.416 | 24.089 | 10.155 | 1.00 | 39.15 |
| B | C | | | | | | | | | | |
| ATOM | | 1826 | OE1 | GLU | B | 117 | 44.547 | 23.549 | 10.877 | 1.00 | 41.69 |
| B | O | | | | | | | | | | |
| ATOM | | 1827 | OE2 | GLU | B | 117 | 46.123 | 23.460 | 9.339 | 1.00 | 40.83 |
| B | O | | | | | | | | | | |
| ATOM | | 1828 | C | GLU | B | 117 | 43.328 | 28.601 | 9.532 | 1.00 | 28.96 |
| B | C | | | | | | | | | | |
| ATOM | | 1829 | O | GLU | B | 117 | 43.359 | 29.151 | 8.428 | 1.00 | 29.54 |
| B | O | | | | | | | | | | |
| ATOM | | 1830 | N | VAL | B | 118 | 42.256 | 28.613 | 10.314 | 1.00 | 25.88 |
| B | N | | | | | | | | | | |
| ATOM | | 1831 | CA | VAL | B | 118 | 41.021 | 29.256 | 9.898 | 1.00 | 23.47 |
| B | C | | | | | | | | | | |
| ATOM | | 1832 | CB | VAL | B | 118 | 39.869 | 28.897 | 10.868 | 1.00 | 23.29 |
| B | C | | | | | | | | | | |
| ATOM | | 1833 | CG1 | VAL | B | 118 | 38.603 | 29.663 | 10.491 | 1.00 | 21.92 |
| B | C | | | | | | | | | | |
| ATOM | | 1834 | CG2 | VAL | B | 118 | 39.618 | 27.391 | 10.829 | 1.00 | 22.44 |
| B | C | | | | | | | | | | |
| ATOM | | 1835 | C | VAL | B | 118 | 41.115 | 30.773 | 9.772 | 1.00 | 22.53 |
| B | C | | | | | | | | | | |
| ATOM | | 1836 | O | VAL | B | 118 | 40.622 | 31.347 | 8.804 | 1.00 | 21.65 |
| B | O | | | | | | | | | | |
| ATOM | | 1837 | N | VAL | B | 119 | 41.748 | 31.429 | 10.739 | 1.00 | 23.10 |
| B | N | | | | | | | | | | |
| ATOM | | 1838 | CA | VAL | B | 119 | 41.856 | 32.885 | 10.696 | 1.00 | 23.11 |
| B | C | | | | | | | | | | |
| ATOM | | 1839 | CB | VAL | B | 119 | 42.573 | 33.432 | 11.941 | 1.00 | 23.48 |
| B | C | | | | | | | | | | |
| ATOM | | 1840 | CG1 | VAL | B | 119 | 42.727 | 34.937 | 11.830 | 1.00 | 24.03 |
| B | C | | | | | | | | | | |
| ATOM | | 1841 | CG2 | VAL | B | 119 | 41.771 | 33.094 | 13.182 | 1.00 | 25.07 |
| B | C | | | | | | | | | | |
| ATOM | | 1842 | C | VAL | B | 119 | 42.537 | 33.427 | 9.438 | 1.00 | 22.58 |
| B | C | | | | | | | | | | |
| ATOM | | 1843 | O | VAL | B | 119 | 42.025 | 34.350 | 8.805 | 1.00 | 21.65 |
| B | O | | | | | | | | | | |
| ATOM | | 1844 | N | PRO | B | 120 | 43.705 | 32.874 | 9.063 | 1.00 | 23.16 |
| B | N | | | | | | | | | | |
| ATOM | | 1845 | CD | PRO | B | 120 | 44.529 | 31.844 | 9.721 | 1.00 | 23.03 |
| B | C | | | | | | | | | | |
| ATOM | | 1846 | CA | PRO | B | 120 | 44.371 | 33.378 | 7.856 | 1.00 | 22.66 |
| B | C | | | | | | | | | | |
| ATOM | | 1847 | CB | PRO | B | 120 | 45.574 | 32.452 | 7.715 | 1.00 | 23.90 |
| B | C | | | | | | | | | | |
| ATOM | | 1848 | CG | PRO | B | 120 | 45.899 | 32.114 | 9.133 | 1.00 | 23.90 |
| B | C | | | | | | | | | | |
| ATOM | | 1849 | C | PRO | B | 120 | 43.434 | 33.294 | 6.654 | 1.00 | 23.50 |
| B | C | | | | | | | | | | |
| ATOM | | 1850 | O | PRO | B | 120 | 43.346 | 34.226 | 5.846 | 1.00 | 21.52 |
| B | O | | | | | | | | | | |
| ATOM | | 1851 | N | PHE | B | 121 | 42.727 | 32.169 | 6.555 | 1.00 | 23.36 |
| B | N | | | | | | | | | | |
| ATOM | | 1852 | CA | PHE | B | 121 | 41.786 | 31.932 | 5.466 | 1.00 | 22.84 |
| B | C | | | | | | | | | | |
| ATOM | | 1853 | CB | PHE | B | 121 | 41.168 | 30.541 | 5.585 | 1.00 | 23.11 |
| B | C | | | | | | | | | | |
| ATOM | | 1854 | CG | PHE | B | 121 | 40.089 | 30.278 | 4.579 | 1.00 | 24.84 |
| B | C | | | | | | | | | | |
| ATOM | | 1855 | CD1 | PHE | B | 121 | 40.400 | 30.074 | 3.241 | 1.00 | 25.82 |
| B | C | | | | | | | | | | |
| ATOM | | 1856 | CD2 | PHE | B | 121 | 38.755 | 30.260 | 4.966 | 1.00 | 25.84 |
| B | C | | | | | | | | | | |
| ATOM | | 1857 | CE1 | PHE | B | 121 | 39.396 | 29.856 | 2.302 | 1.00 | 25.59 |
| B | C | | | | | | | | | | |
| ATOM | | 1858 | CE2 | PHE | B | 121 | 37.744 | 30.043 | 4.033 | 1.00 | 27.74 |
| B | C | | | | | | | | | | |
| ATOM | | 1859 | CZ | PHE | B | 121 | 38.067 | 29.841 | 2.699 | 1.00 | 26.97 |
| B | C | | | | | | | | | | |
| ATOM | | 1860 | C | PHE | B | 121 | 40.679 | 32.974 | 5.474 | 1.00 | 22.35 |
| B | C | | | | | | | | | | |
| ATOM | | 1861 | O | PHE | B | 121 | 40.382 | 33.572 | 4.443 | 1.00 | 22.39 |
| B | O | | | | | | | | | | |
| ATOM | | 1862 | N | LEU | B | 122 | 40.059 | 33.180 | 6.633 | 1.00 | 22.63 |
| B | N | | | | | | | | | | |
| ATOM | | 1863 | CA | LEU | B | 122 | 39.003 | 34.181 | 6.732 | 1.00 | 21.52 |
| B | C | | | | | | | | | | |
| ATOM | | 1864 | CB | LEU | B | 122 | 38.321 | 34.102 | 8.109 | 1.00 | 20.83 |
| B | C | | | | | | | | | | |
| ATOM | | 1865 | CG | LEU | B | 122 | 37.578 | 32.795 | 8.420 | 1.00 | 20.51 |
| B | C | | | | | | | | | | |
| ATOM | | 1866 | CD1 | LEU | B | 122 | 36.959 | 32.858 | 9.813 | 1.00 | 19.20 |
| B | C | | | | | | | | | | |
| ATOM | | 1867 | CD2 | LEU | B | 122 | 36.501 | 32.566 | 7.375 | 1.00 | 19.61 |
| B | C | | | | | | | | | | |
| ATOM | | 1868 | C | LEU | B | 122 | 39.527 | 35.600 | 6.460 | 1.00 | 21.22 |
| B | C | | | | | | | | | | |
| ATOM | | 1869 | O | LEU | B | 122 | 38.805 | 36.409 | 5.890 | 1.00 | 20.75 |
| B | O | | | | | | | | | | |
| ATOM | | 1870 | N | ALA | B | 123 | 40.771 | 35.878 | 6.853 | 1.00 | 22.05 |
| B | N | | | | | | | | | | |
| ATOM | | 1871 | CA | ALA | B | 123 | 41.379 | 37.190 | 6.637 | 1.00 | 24.52 |
| B | C | | | | | | | | | | |
| ATOM | | 1872 | CB | ALA | B | 123 | 42.804 | 37.224 | 7.280 | 1.00 | 24.63 |
| B | C | | | | | | | | | | |
| ATOM | | 1873 | C | ALA | B | 123 | 41.489 | 37.419 | 5.129 | 1.00 | 26.44 |
| B | C | | | | | | | | | | |
| ATOM | | 1874 | O | ALA | B | 123 | 41.225 | 38.505 | 4.618 | 1.00 | 25.74 |
| B | O | | | | | | | | | | |
| ATOM | | 1875 | N | ARG | B | 124 | 41.941 | 36.397 | 4.381 | 1.00 | 27.56 |
| B | N | | | | | | | | | | |
| ATOM | | 1876 | CA | ARG | B | 124 | 42.092 | 36.492 | 2.915 | 1.00 | 28.25 |
| B | C | | | | | | | | | | |
| ATOM | | 1877 | CB | AARG | B | 124 | 42.562 | 35.166 | 2.361 | 0.50 | 31.53 |
| B | C | | | | | | | | | | |
| ATOM | | 1878 | CB | BARG | B | 124 | 42.543 | 35.160 | 2.316 | 0.50 | 31.52 |
| B | C | | | | | | | | | | |
| ATOM | | 1879 | CG | AARG | B | 124 | 43.971 | 34.730 | 2.819 | 0.50 | 34.27 |
| B | C | | | | | | | | | | |
| ATOM | | 1880 | CG | BARG | B | 124 | 43.953 | 34.764 | 2.660 | 0.50 | 34.62 |
| B | C | | | | | | | | | | |
| ATOM | | 1881 | CD | AARG | B | 124 | 44.500 | 33.566 | 1.961 | 0.50 | 36.72 |
| B | C | | | | | | | | | | |
| ATOM | | 1882 | CD | BARG | B | 124 | 44.485 | 33.793 | 1.624 | 0.50 | 36.96 |
| B | C | | | | | | | | | | |
| ATOM | | 1883 | NE | AARG | B | 124 | 44.367 | 32.250 | 2.587 | 0.50 | 38.21 |
| B | N | | | | | | | | | | |
| ATOM | | 1884 | NE | BARG | B | 124 | 43.633 | 32.620 | 1.416 | 0.50 | 39.16 |
| B | N | | | | | | | | | | |
| ATOM | | 1885 | CZ | AARG | B | 124 | 45.302 | 31.685 | 3.346 | 0.50 | 39.41 |
| B | C | | | | | | | | | | |
| ATOM | | 1886 | CZ | BARG | B | 124 | 42.571 | 32.573 | 0.613 | 0.50 | 39.85 |
| B | C | | | | | | | | | | |
| ATOM | | 1887 | NH1 | AARG | B | 124 | 46.444 | 32.318 | 3.574 | 0.50 | 39.45 |
| B | N | | | | | | | | | | |
| ATOM | | 1888 | NH1 | BARG | B | 124 | 42.196 | 33.638 | −0.084 | 0.50 | 39.88 |
| B | N | | | | | | | | | | |
| ATOM | | 1889 | NH2 | AARG | B | 124 | 45.097 | 30.487 | 3.874 | 0.50 | 40.70 |
| B | N | | | | | | | | | | |
| ATOM | | 1890 | NH2 | BARG | B | 124 | 41.878 | 31.449 | 0.504 | 0.50 | 40.63 |
| B | N | | | | | | | | | | |
| ATOM | | 1891 | C | ARG | B | 124 | 40.755 | 36.861 | 2.270 | 1.00 | 28.59 |
| B | C | | | | | | | | | | |
| ATOM | | 1892 | O | ARG | B | 124 | 40.699 | 37.698 | 1.347 | 1.00 | 28.10 |
| B | O | | | | | | | | | | |
| ATOM | | 1893 | N | LEU | B | 125 | 39.685 | 36.193 | 2.734 | 1.00 | 28.01 |
| B | N | | | | | | | | | | |
| ATOM | | 1894 | CA | LEU | B | 125 | 38.367 | 36.466 | 2.203 | 1.00 | 25.98 |
| B | C | | | | | | | | | | |
| ATOM | | 1895 | CB | LEU | B | 125 | 37.333 | 35.525 | 2.822 | 1.00 | 27.46 |
| B | C | | | | | | | | | | |
| ATOM | | 1896 | CG | LEU | B | 125 | 37.385 | 34.019 | 2.530 | 1.00 | 27.82 |
| B | C | | | | | | | | | | |
| ATOM | | 1897 | CD1 | LEU | B | 125 | 36.107 | 33.354 | 3.052 | 1.00 | 26.92 |
| B | C | | | | | | | | | | |
| ATOM | | 1898 | CD2 | LEU | B | 125 | 37.519 | 33.771 | 1.039 | 1.00 | 27.76 |
| B | C | | | | | | | | | | |
| ATOM | | 1899 | C | LEU | B | 125 | 37.940 | 37.905 | 2.469 | 1.00 | 25.43 |
| B | C | | | | | | | | | | |
| ATOM | | 1900 | O | LEU | B | 125 | 37.301 | 38.547 | 1.629 | 1.00 | 25.94 |
| B | O | | | | | | | | | | |
| ATOM | | 1901 | N | SER | B | 126 | 38.262 | 38.392 | 3.660 | 1.00 | 24.59 |
| B | N | | | | | | | | | | |
| ATOM | | 1902 | CA | SER | B | 126 | 37.937 | 39.757 | 4.040 | 1.00 | 25.45 |
| B | C | | | | | | | | | | |
| ATOM | | 1903 | CB | SER | B | 126 | 38.427 | 40.030 | 5.463 | 1.00 | 25.70 |
| B | C | | | | | | | | | | |
| ATOM | | 1904 | OG | SER | B | 126 | 38.216 | 41.382 | 5.819 | 1.00 | 24.65 |
| B | O | | | | | | | | | | |
| ATOM | | 1905 | C | SER | B | 126 | 38.671 | 40.668 | 3.067 | 1.00 | 26.58 |
| B | C | | | | | | | | | | |
| ATOM | | 1906 | O | SER | B | 126 | 38.126 | 41.664 | 2.584 | 1.00 | 25.56 |
| B | O | | | | | | | | | | |
| ATOM | | 1907 | N | ASN | B | 127 | 39.920 | 40.311 | 2.789 | 1.00 | 28.53 |
| B | N | | | | | | | | | | |
| ATOM | | 1908 | CA | ASN | B | 127 | 40.766 | 41.069 | 1.875 | 1.00 | 31.42 |
| B | C | | | | | | | | | | |
| ATOM | | 1909 | CB | ASN | B | 127 | 42.149 | 40.415 | 1.810 | 1.00 | 33.56 |
| B | C | | | | | | | | | | |
| ATOM | | 1910 | CG | ASN | B | 127 | 43.145 | 41.230 | 1.017 | 1.00 | 37.76 |
| B | C | | | | | | | | | | |
| ATOM | | 1911 | OD1 | ASN | B | 127 | 43.392 | 42.396 | 1.326 | 1.00 | 41.52 |
| B | O | | | | | | | | | | |
| ATOM | | 1912 | ND2 | ASN | B | 127 | 43.734 | 40.617 | −0.008 | 1.00 | 37.74 |
| B | N | | | | | | | | | | |
| ATOM | | 1913 | C | ASN | B | 127 | 40.114 | 41.110 | 0.492 | 1.00 | 31.83 |
| B | C | | | | | | | | | | |
| ATOM | | 1914 | O | ASN | B | 127 | 40.081 | 42.154 | −0.160 | 1.00 | 32.67 |
| B | O | | | | | | | | | | |
| ATOM | | 1915 | N | ARG | B | 128 | 39.584 | 39.971 | 0.057 | 1.00 | 33.16 |
| B | N | | | | | | | | | | |
| ATOM | | 1916 | CA | ARG | B | 128 | 38.919 | 39.872 | −1.239 | 1.00 | 35.03 |
| B | C | | | | | | | | | | |
| ATOM | | 1917 | CB | ARG | B | 128 | 38.477 | 38.429 | −1.499 | 1.00 | 37.42 |
| B | C | | | | | | | | | | |
| ATOM | | 1918 | CG | ARG | B | 128 | 39.572 | 37.384 | −1.393 | 1.00 | 40.66 |
| B | C | | | | | | | | | | |
| ATOM | | 1919 | CD | ARG | B | 128 | 40.484 | 37.388 | −2.602 | 1.00 | 44.62 |
| B | C | | | | | | | | | | |
| ATOM | | 1920 | NE | ARG | B | 128 | 41.399 | 36.248 | −2.596 | 1.00 | 47.25 |
| B | N | | | | | | | | | | |
| ATOM | | 1921 | CZ | ARG | B | 128 | 42.370 | 36.069 | −1.706 | 1.00 | 49.09 |
| B | C | | | | | | | | | | |
| ATOM | | 1922 | NH1 | ARG | B | 128 | 42.562 | 36.957 | −0.737 | 1.00 | 49.49 |
| B | N | | | | | | | | | | |
| ATOM | | 1923 | NH2 | ARG | B | 128 | 43.155 | 35.002 | −1.787 | 1.00 | 49.90 |
| B | N | | | | | | | | | | |
| ATOM | | 1924 | C | ARG | B | 128 | 37.686 | 40.780 | −1.311 | 1.00 | 35.69 |
| B | C | | | | | | | | | | |
| ATOM | | 1925 | O | ARG | B | 128 | 37.401 | 41.370 | −2.352 | 1.00 | 35.80 |
| B | O | | | | | | | | | | |
| ATOM | | 1926 | N | LEU | B | 129 | 36.955 | 40.886 | −0.206 | 1.00 | 35.75 |
| B | N | | | | | | | | | | |
| ATOM | | 1927 | CA | LEU | B | 129 | 35.746 | 41.709 | −0.161 | 1.00 | 36.71 |
| B | C | | | | | | | | | | |
| ATOM | | 1928 | CB | LEU | B | 129 | 34.816 | 41.213 | 0.952 | 1.00 | 35.61 |
| B | C | | | | | | | | | | |
| ATOM | | 1929 | CG | LEU | B | 129 | 34.227 | 39.810 | 0.784 | 1.00 | 36.13 |
| B | C | | | | | | | | | | |
| ATOM | | 1930 | CD1 | LEU | B | 129 | 33.424 | 39.445 | 2.017 | 1.00 | 35.68 |
| B | C | | | | | | | | | | |
| ATOM | | 1931 | CD2 | LEU | B | 129 | 33.344 | 39.761 | −0.454 | 1.00 | 34.32 |
| B | C | | | | | | | | | | |
| ATOM | | 1932 | C | LEU | B | 129 | 36.018 | 43.200 | 0.043 | 1.00 | 37.24 |
| B | C | | | | | | | | | | |
| ATOM | | 1933 | O | LEU | B | 129 | 35.086 | 43.989 | 0.178 | 1.00 | 37.30 |
| B | O | | | | | | | | | | |
| ATOM | | 1934 | N | SER | B | 130 | 37.291 | 43.577 | 0.059 | 1.00 | 39.20 |
| B | N | | | | | | | | | | |
| ATOM | | 1935 | CA | SER | B | 130 | 37.693 | 44.969 | 0.268 | 1.00 | 41.51 |
| B | C | | | | | | | | | | |
| ATOM | | 1936 | CB | SER | B | 130 | 39.122 | 45.191 | −0.238 | 1.00 | 41.64 |
| B | C | | | | | | | | | | |
| ATOM | | 1937 | OG | SER | B | 130 | 40.062 | 44.614 | 0.651 | 1.00 | 44.96 |
| B | O | | | | | | | | | | |
| ATOM | | 1938 | C | SER | B | 130 | 36.789 | 46.030 | −0.345 | 1.00 | 41.71 |
| B | C | | | | | | | | | | |
| ATOM | | 1939 | O | SER | B | 130 | 36.579 | 46.056 | −1.550 | 1.00 | 43.01 |
| B | O | | | | | | | | | | |
| ATOM | | 1940 | N | THR | B | 131 | 36.259 | 46.900 | 0.508 | 1.00 | 42.65 |
| B | N | | | | | | | | | | |
| ATOM | | 1941 | CA | THR | B | 131 | 35.399 | 48.012 | 0.103 | 1.00 | 43.65 |
| B | C | | | | | | | | | | |
| ATOM | | 1942 | CB | THR | B | 131 | 36.223 | 49.110 | −0.617 | 1.00 | 44.32 |
| B | C | | | | | | | | | | |
| ATOM | | 1943 | OG1 | THR | B | 131 | 36.682 | 48.620 | −1.885 | 1.00 | 45.77 |
| B | O | | | | | | | | | | |
| ATOM | | 1944 | CG2 | THR | B | 131 | 37.428 | 49.511 | 0.236 | 1.00 | 44.02 |
| B | C | | | | | | | | | | |
| ATOM | | 1945 | C | THR | B | 131 | 34.161 | 47.723 | −0.747 | 1.00 | 43.82 |
| B | C | | | | | | | | | | |
| ATOM | | 1946 | O | THR | B | 131 | 33.401 | 48.649 | −1.041 | 1.00 | 43.74 |
| B | O | | | | | | | | | | |
| ATOM | | 1947 | N | CYS | B | 132 | 33.945 | 46.470 | −1.146 | 1.00 | 43.43 |
| B | N | | | | | | | | | | |
| ATOM | | 1948 | CA | CYS | B | 132 | 32.762 | 46.157 | −1.949 | 1.00 | 42.56 |
| B | C | | | | | | | | | | |
| ATOM | | 1949 | C | CYS | B | 132 | 31.564 | 46.639 | −1.146 | 1.00 | 42.53 |
| B | C | | | | | | | | | | |
| ATOM | | 1950 | O | CYS | B | 132 | 31.515 | 46.452 | 0.068 | 1.00 | 42.41 |
| B | O | | | | | | | | | | |
| ATOM | | 1951 | CB | CYS | B | 132 | 32.644 | 44.651 | −2.205 | 1.00 | 41.08 |
| B | C | | | | | | | | | | |
| ATOM | | 1952 | SG | CYS | B | 132 | 32.191 | 43.674 | −0.738 | 1.00 | 40.07 |
| B | S | | | | | | | | | | |
| ATOM | | 1953 | N | HIS | B | 133 | 30.608 | 47.277 | −1.813 | 1.00 | 44.03 |
| B | N | | | | | | | | | | |
| ATOM | | 1954 | CA | HIS | B | 133 | 29.434 | 47.790 | −1.120 | 1.00 | 45.92 |
| B | C | | | | | | | | | | |
| ATOM | | 1955 | CB | HIS | B | 133 | 29.661 | 49.247 | −0.723 | 1.00 | 48.01 |
| B | C | | | | | | | | | | |
| ATOM | | 1956 | CG | HIS | B | 133 | 29.999 | 50.136 | −1.877 | 1.00 | 50.95 |
| B | C | | | | | | | | | | |
| ATOM | | 1957 | CD2 | HIS | B | 133 | 29.344 | 51.195 | −2.408 | 1.00 | 51.52 |
| B | C | | | | | | | | | | |
| ATOM | | 1958 | ND1 | HIS | B | 133 | 31.132 | 49.961 | −2.642 | 1.00 | 52.14 |
| B | N | | | | | | | | | | |
| ATOM | | 1959 | CE1 | HIS | B | 133 | 31.161 | 50.875 | −3.596 | 1.00 | 52.81 |
| B | C | | | | | | | | | | |
| ATOM | | 1960 | NE2 | HIS | B | 133 | 30.088 | 51.636 | −3.477 | 1.00 | 52.91 |
| B | N | | | | | | | | | | |
| ATOM | | 1961 | C | HIS | B | 133 | 28.158 | 47.680 | −1.940 | 1.00 | 45.88 |
| B | C | | | | | | | | | | |
| ATOM | | 1962 | O | HIS | B | 133 | 28.143 | 47.067 | −3.005 | 1.00 | 45.95 |
| B | O | | | | | | | | | | |
| ATOM | | 1963 | N | ILE | B | 134 | 27.086 | 48.281 | −1.433 | 1.00 | 46.79 |
| B | N | | | | | | | | | | |
| ATOM | | 1964 | CA | ILE | B | 134 | 25.798 | 48.245 | −2.110 | 1.00 | 48.22 |
| B | C | | | | | | | | | | |
| ATOM | | 1965 | CB | ILE | B | 134 | 24.686 | 47.725 | −1.172 | 1.00 | 47.47 |
| B | C | | | | | | | | | | |
| ATOM | | 1966 | CG2 | ILE | B | 134 | 25.035 | 46.328 | −0.684 | 1.00 | 47.42 |
| B | C | | | | | | | | | | |
| ATOM | | 1967 | CG1 | ILE | B | 134 | 24.515 | 48.672 | 0.016 | 1.00 | 46.57 |
| B | C | | | | | | | | | | |
| ATOM | | 1968 | CD | ILE | B | 134 | 23.426 | 48.248 | 0.977 | 1.00 | 47.17 |
| B | C | | | | | | | | | | |
| ATOM | | 1969 | C | ILE | B | 134 | 25.385 | 49.614 | −2.644 | 1.00 | 49.84 |
| B | C | | | | | | | | | | |
| ATOM | | 1970 | O | ILE | B | 134 | 26.079 | 50.611 | −2.439 | 1.00 | 49.80 |
| B | O | | | | | | | | | | |
| ATOM | | 1971 | N | GLU | B | 135 | 24.243 | 49.642 | −3.326 | 1.00 | 50.44 |
| B | N | | | | | | | | | | |
| ATOM | | 1972 | CA | GLU | B | 135 | 23.700 | 50.860 | −3.921 | 1.00 | 51.87 |
| B | C | | | | | | | | | | |
| ATOM | | 1973 | CB | GLU | B | 135 | 22.405 | 50.531 | −4.671 | 1.00 | 53.19 |
| B | C | | | | | | | | | | |
| ATOM | | 1974 | CG | GLU | B | 135 | 22.597 | 49.598 | −5.859 | 1.00 | 55.61 |
| B | C | | | | | | | | | | |
| ATOM | | 1975 | CD | GLU | B | 135 | 23.283 | 48.298 | −5.475 | 1.00 | 57.90 |
| B | C | | | | | | | | | | |
| ATOM | | 1976 | OE1 | GLU | B | 135 | 22.787 | 47.613 | −4.554 | 1.00 | 58.58 |
| B | O | | | | | | | | | | |
| ATOM | | 1977 | OE2 | GLU | B | 135 | 24.317 | 47.963 | −6.093 | 1.00 | 59.79 |
| B | O | | | | | | | | | | |
| ATOM | | 1978 | C | GLU | B | 135 | 23.445 | 51.989 | −2.918 | 1.00 | 51.36 |
| B | C | | | | | | | | | | |
| ATOM | | 1979 | O | GLU | B | 135 | 24.324 | 52.820 | −2.677 | 1.00 | 51.02 |
| B | O | | | | | | | | | | |
| ATOM | | 1980 | N | GLY | B | 136 | 22.245 | 52.022 | −2.341 | 1.00 | 50.44 |
| B | N | | | | | | | | | | |
| ATOM | | 1981 | CA | GLY | B | 136 | 21.930 | 53.074 | −1.393 | 1.00 | 49.30 |
| B | C | | | | | | | | | | |
| ATOM | | 1982 | C | GLY | B | 136 | 21.197 | 52.672 | −0.128 | 1.00 | 47.53 |
| B | C | | | | | | | | | | |
| ATOM | | 1983 | O | GLY | B | 136 | 20.289 | 51.839 | −0.147 | 1.00 | 46.93 |
| B | O | | | | | | | | | | |
| ATOM | | 1984 | N | ASP | B | 137 | 21.614 | 53.284 | 0.976 | 1.00 | 46.58 |
| B | N | | | | | | | | | | |
| ATOM | | 1985 | CA | ASP | B | 137 | 21.027 | 53.064 | 2.293 | 1.00 | 45.28 |
| B | C | | | | | | | | | | |
| ATOM | | 1986 | CB | ASP | B | 137 | 19.559 | 53.486 | 2.277 | 1.00 | 47.28 |
| B | C | | | | | | | | | | |
| ATOM | | 1987 | CG | ASP | B | 137 | 18.981 | 53.623 | 3.661 | 1.00 | 48.41 |
| B | C | | | | | | | | | | |
| ATOM | | 1988 | OD1 | ASP | B | 137 | 19.131 | 52.681 | 4.465 | 1.00 | 48.28 |
| B | O | | | | | | | | | | |
| ATOM | | 1989 | OD2 | ASP | B | 137 | 18.378 | 54.675 | 3.947 | 1.00 | 51.40 |
| B | O | | | | | | | | | | |
| ATOM | | 1990 | C | ASP | B | 137 | 21.133 | 51.650 | 2.844 | 1.00 | 43.55 |
| B | C | | | | | | | | | | |
| ATOM | | 1991 | O | ASP | B | 137 | 20.277 | 50.803 | 2.590 | 1.00 | 43.30 |
| B | O | | | | | | | | | | |
| ATOM | | 1992 | N | ASP | B | 138 | 22.184 | 51.416 | 3.620 | 1.00 | 40.92 |
| B | N | | | | | | | | | | |
| ATOM | | 1993 | CA | ASP | B | 138 | 22.419 | 50.118 | 4.237 | 1.00 | 38.17 |
| B | C | | | | | | | | | | |
| ATOM | | 1994 | CB | ASP | B | 138 | 23.899 | 49.774 | 4.155 | 1.00 | 37.09 |
| B | C | | | | | | | | | | |
| ATOM | | 1995 | CG | ASP | B | 138 | 24.751 | 50.703 | 4.992 | 1.00 | 38.19 |
| B | C | | | | | | | | | | |
| ATOM | | 1996 | OD1 | ASP | B | 138 | 24.292 | 51.828 | 5.283 | 1.00 | 38.67 |
| B | O | | | | | | | | | | |
| ATOM | | 1997 | OD2 | ASP | B | 138 | 25.882 | 50.319 | 5.354 | 1.00 | 39.46 |
| B | O | | | | | | | | | | |
| ATOM | | 1998 | C | ASP | B | 138 | 21.988 | 50.124 | 5.712 | 1.00 | 36.48 |
| B | C | | | | | | | | | | |
| ATOM | | 1999 | O | ASP | B | 138 | 22.428 | 49.286 | 6.501 | 1.00 | 36.23 |
| B | O | | | | | | | | | | |
| ATOM | | 2000 | N | LEU | B | 139 | 21.124 | 51.065 | 6.076 | 1.00 | 35.08 |
| B | N | | | | | | | | | | |
| ATOM | | 2001 | CA | LEU | B | 139 | 20.650 | 51.196 | 7.450 | 1.00 | 33.13 |
| B | C | | | | | | | | | | |
| ATOM | | 2002 | CB | LEU | B | 139 | 19.592 | 52.298 | 7.533 | 1.00 | 36.06 |
| B | C | | | | | | | | | | |
| ATOM | | 2003 | CG | LEU | B | 139 | 19.099 | 52.661 | 8.934 | 1.00 | 37.59 |
| B | C | | | | | | | | | | |
| ATOM | | 2004 | CD1 | LEU | B | 139 | 20.224 | 53.321 | 9.706 | 1.00 | 39.27 |
| B | C | | | | | | | | | | |
| ATOM | | 2005 | CD2 | LEU | B | 139 | 17.909 | 53.599 | 8.840 | 1.00 | 40.35 |
| B | C | | | | | | | | | | |
| ATOM | | 2006 | C | LEU | B | 139 | 20.087 | 49.903 | 8.030 | 1.00 | 31.75 |
| B | C | | | | | | | | | | |
| ATOM | | 2007 | O | LEU | B | 139 | 20.423 | 49.524 | 9.153 | 1.00 | 31.56 |
| B | O | | | | | | | | | | |
| ATOM | | 2008 | N | HIS | B | 140 | 19.227 | 49.226 | 7.275 | 1.00 | 29.29 |
| B | N | | | | | | | | | | |
| ATOM | | 2009 | CA | HIS | B | 140 | 18.634 | 47.975 | 7.748 | 1.00 | 27.87 |
| B | C | | | | | | | | | | |
| ATOM | | 2010 | CB | HIS | B | 140 | 17.598 | 47.465 | 6.738 | 1.00 | 29.34 |
| B | C | | | | | | | | | | |
| ATOM | | 2011 | CG | HIS | B | 140 | 18.158 | 47.209 | 5.374 | 1.00 | 30.92 |
| B | C | | | | | | | | | | |
| ATOM | | 2012 | CD2 | HIS | B | 140 | 18.179 | 46.088 | 4.614 | 1.00 | 32.52 |
| B | C | | | | | | | | | | |
| ATOM | | 2013 | ND1 | HIS | B | 140 | 18.796 | 48.183 | 4.636 | 1.00 | 33.09 |
| B | N | | | | | | | | | | |
| ATOM | | 2014 | CE1 | HIS | B | 140 | 19.187 | 47.672 | 3.481 | 1.00 | 33.64 |
| B | C | | | | | | | | | | |
| ATOM | | 2015 | NE2 | HIS | B | 140 | 18.826 | 46.403 | 3.442 | 1.00 | 33.59 |
| B | N | | | | | | | | | | |
| ATOM | | 2016 | C | HIS | B | 140 | 19.714 | 46.912 | 7.977 | 1.00 | 24.95 |
| B | C | | | | | | | | | | |
| ATOM | | 2017 | O | HIS | B | 140 | 19.614 | 46.083 | 8.878 | 1.00 | 24.47 |
| B | O | | | | | | | | | | |
| ATOM | | 2018 | N | ILE | B | 141 | 20.752 | 46.946 | 7.156 | 1.00 | 23.58 |
| B | N | | | | | | | | | | |
| ATOM | | 2019 | CA | ILE | B | 141 | 21.840 | 45.991 | 7.289 | 1.00 | 22.50 |
| B | C | | | | | | | | | | |
| ATOM | | 2020 | CB | ILE | B | 141 | 22.765 | 46.043 | 6.059 | 1.00 | 23.20 |
| B | C | | | | | | | | | | |
| ATOM | | 2021 | CG2 | ILE | B | 141 | 23.982 | 45.171 | 6.283 | 1.00 | 22.62 |
| B | C | | | | | | | | | | |
| ATOM | | 2022 | CG1 | ILE | B | 141 | 21.983 | 45.592 | 4.818 | 1.00 | 24.14 |
| B | C | | | | | | | | | | |
| ATOM | | 2023 | CD | ILE | B | 141 | 22.783 | 45.609 | 3.541 | 1.00 | 26.45 |
| B | C | | | | | | | | | | |
| ATOM | | 2024 | C | ILE | B | 141 | 22.641 | 46.287 | 8.556 | 1.00 | 22.44 |
| B | C | | | | | | | | | | |
| ATOM | | 2025 | O | ILE | B | 141 | 22.899 | 45.390 | 9.361 | 1.00 | 19.54 |
| B | O | | | | | | | | | | |
| ATOM | | 2026 | N | GLN | B | 142 | 23.020 | 47.548 | 8.738 | 1.00 | 21.44 |
| B | N | | | | | | | | | | |
| ATOM | | 2027 | CA | GLN | B | 142 | 23.792 | 47.932 | 9.913 | 1.00 | 20.93 |
| B | C | | | | | | | | | | |
| ATOM | | 2028 | CB | GLN | B | 142 | 24.152 | 49.418 | 9.858 | 1.00 | 23.51 |
| B | C | | | | | | | | | | |
| ATOM | | 2029 | CG | GLN | B | 142 | 25.108 | 49.807 | 8.734 | 1.00 | 27.67 |
| B | C | | | | | | | | | | |
| ATOM | | 2030 | CD | GLN | B | 142 | 26.345 | 48.920 | 8.666 | 1.00 | 31.25 |
| B | C | | | | | | | | | | |
| ATOM | | 2031 | OE1 | GLN | B | 142 | 26.874 | 48.488 | 9.692 | 1.00 | 33.98 |
| B | O | | | | | | | | | | |
| ATOM | | 2032 | NE2 | GLN | B | 142 | 26.819 | 48.659 | 7.451 | 1.00 | 31.45 |
| B | N | | | | | | | | | | |
| ATOM | | 2033 | C | GLN | B | 142 | 23.031 | 47.634 | 11.202 | 1.00 | 19.58 |
| B | C | | | | | | | | | | |
| ATOM | | 2034 | O | GLN | B | 142 | 23.621 | 47.216 | 12.195 | 1.00 | 19.52 |
| B | O | | | | | | | | | | |
| ATOM | | 2035 | N | ARG | B | 143 | 21.718 | 47.847 | 11.182 | 1.00 | 19.46 |
| B | N | | | | | | | | | | |
| ATOM | | 2036 | CA | ARG | B | 143 | 20.891 | 47.598 | 12.355 | 1.00 | 18.97 |
| B | C | | | | | | | | | | |
| ATOM | | 2037 | CB | ARG | B | 143 | 19.454 | 48.084 | 12.117 | 1.00 | 19.80 |
| B | C | | | | | | | | | | |
| ATOM | | 2038 | CG | ARG | B | 143 | 18.572 | 47.982 | 13.361 | 1.00 | 20.29 |
| B | C | | | | | | | | | | |
| ATOM | | 2039 | CD | ARG | B | 143 | 17.151 | 48.457 | 13.118 | 1.00 | 24.78 |
| B | C | | | | | | | | | | |
| ATOM | | 2040 | NE | ARG | B | 143 | 17.081 | 49.859 | 12.703 | 1.00 | 27.22 |
| B | N | | | | | | | | | | |
| ATOM | | 2041 | CZ | ARG | B | 143 | 16.831 | 50.261 | 11.459 | 1.00 | 29.43 |
| B | C | | | | | | | | | | |
| ATOM | | 2042 | NH1 | ARG | B | 143 | 16.625 | 49.367 | 10.499 | 1.00 | 28.10 |
| B | N | | | | | | | | | | |
| ATOM | | 2043 | NH2 | ARG | B | 143 | 16.785 | 51.557 | 11.172 | 1.00 | 29.11 |
| B | N | | | | | | | | | | |
| ATOM | | 2044 | C | ARG | B | 143 | 20.879 | 46.116 | 12.716 | 1.00 | 19.02 |
| B | C | | | | | | | | | | |
| ATOM | | 2045 | O | ARG | B | 143 | 20.936 | 45.751 | 13.896 | 1.00 | 18.36 |
| B | O | | | | | | | | | | |
| ATOM | | 2046 | N | ASN | B | 144 | 20.795 | 45.259 | 11.703 | 1.00 | 17.05 |
| B | N | | | | | | | | | | |
| ATOM | | 2047 | CA | ASN | B | 144 | 20.783 | 43.822 | 11.944 | 1.00 | 17.50 |
| B | C | | | | | | | | | | |
| ATOM | | 2048 | CB | ASN | B | 144 | 20.492 | 43.053 | 10.649 | 1.00 | 16.62 |
| B | C | | | | | | | | | | |
| ATOM | | 2049 | CG | ASN | B | 144 | 19.062 | 43.240 | 10.169 | 1.00 | 18.56 |
| B | C | | | | | | | | | | |
| ATOM | | 2050 | OD1 | ASN | B | 144 | 18.280 | 43.963 | 10.781 | 1.00 | 15.87 |
| B | O | | | | | | | | | | |
| ATOM | | 2051 | ND2 | ASN | B | 144 | 18.714 | 42.582 | 9.070 | 1.00 | 18.04 |
| B | N | | | | | | | | | | |
| ATOM | | 2052 | C | ASN | B | 144 | 22.125 | 43.381 | 12.503 | 1.00 | 16.92 |
| B | C | | | | | | | | | | |
| ATOM | | 2053 | O | ASN | B | 144 | 22.181 | 42.547 | 13.400 | 1.00 | 16.92 |
| B | O | | | | | | | | | | |
| ATOM | | 2054 | N | VAL | B | 145 | 23.205 | 43.943 | 11.967 | 1.00 | 15.33 |
| B | N | | | | | | | | | | |
| ATOM | | 2055 | CA | VAL | B | 145 | 24.544 | 43.598 | 12.423 | 1.00 | 15.98 |
| B | C | | | | | | | | | | |
| ATOM | | 2056 | CB | VAL | B | 145 | 25.615 | 44.191 | 11.469 | 1.00 | 17.14 |
| B | C | | | | | | | | | | |
| ATOM | | 2057 | CG1 | VAL | B | 145 | 27.020 | 43.920 | 12.007 | 1.00 | 17.91 |
| B | C | | | | | | | | | | |
| ATOM | | 2058 | CG2 | VAL | B | 145 | 25.456 | 43.584 | 10.080 | 1.00 | 18.98 |
| B | C | | | | | | | | | | |
| ATOM | | 2059 | C | VAL | B | 145 | 24.760 | 44.118 | 13.846 | 1.00 | 16.15 |
| B | C | | | | | | | | | | |
| ATOM | | 2060 | O | VAL | B | 145 | 25.396 | 43.454 | 14.672 | 1.00 | 13.69 |
| B | O | | | | | | | | | | |
| ATOM | | 2061 | N | GLN | B | 146 | 24.221 | 45.302 | 14.134 | 1.00 | 14.53 |
| B | N | | | | | | | | | | |
| ATOM | | 2062 | CA | GLN | B | 146 | 24.359 | 45.893 | 15.464 | 1.00 | 12.59 |
| B | C | | | | | | | | | | |
| ATOM | | 2063 | CB | GLN | B | 146 | 23.598 | 47.222 | 15.546 | 1.00 | 12.98 |
| B | C | | | | | | | | | | |
| ATOM | | 2064 | CG | GLN | B | 146 | 23.739 | 47.970 | 16.885 | 1.00 | 15.51 |
| B | C | | | | | | | | | | |
| ATOM | | 2065 | CD | GLN | B | 146 | 25.100 | 48.641 | 17.058 | 1.00 | 18.34 |
| B | C | | | | | | | | | | |
| ATOM | | 2066 | OE1 | GLN | B | 146 | 25.593 | 49.306 | 16.148 | 1.00 | 17.29 |
| B | O | | | | | | | | | | |
| ATOM | | 2067 | NE2 | GLN | B | 146 | 25.700 | 48.483 | 18.233 | 1.00 | 19.22 |
| B | N | | | | | | | | | | |
| ATOM | | 2068 | C | GLN | B | 146 | 23.814 | 44.926 | 16.516 | 1.00 | 13.75 |
| B | C | | | | | | | | | | |
| ATOM | | 2069 | O | GLN | B | 146 | 24.388 | 44.778 | 17.592 | 1.00 | 11.09 |
| B | O | | | | | | | | | | |
| ATOM | | 2070 | N | LYS | B | 147 | 22.703 | 44.267 | 16.205 | 1.00 | 14.74 |
| B | N | | | | | | | | | | |
| ATOM | | 2071 | CA | LYS | B | 147 | 22.112 | 43.326 | 17.147 | 1.00 | 15.33 |
| B | C | | | | | | | | | | |
| ATOM | | 2072 | CB | LYS | B | 147 | 20.820 | 42.737 | 16.575 | 1.00 | 19.41 |
| B | C | | | | | | | | | | |
| ATOM | | 2073 | CG | LYS | B | 147 | 19.632 | 43.681 | 16.657 | 1.00 | 24.48 |
| B | C | | | | | | | | | | |
| ATOM | | 2074 | CD | LYS | B | 147 | 18.351 | 43.003 | 16.173 | 1.00 | 27.59 |
| B | C | | | | | | | | | | |
| ATOM | | 2075 | CE | LYS | B | 147 | 17.113 | 43.791 | 16.583 | 1.00 | 30.10 |
| B | C | | | | | | | | | | |
| ATOM | | 2076 | NZ | LYS | B | 147 | 17.197 | 45.226 | 16.199 | 1.00 | 32.34 |
| B | N | | | | | | | | | | |
| ATOM | | 2077 | C | LYS | B | 147 | 23.075 | 42.202 | 17.488 | 1.00 | 14.99 |
| B | C | | | | | | | | | | |
| ATOM | | 2078 | O | LYS | B | 147 | 23.242 | 41.851 | 18.656 | 1.00 | 13.88 |
| B | O | | | | | | | | | | |
| ATOM | | 2079 | N | LEU | B | 148 | 23.698 | 41.630 | 16.463 | 1.00 | 13.47 |
| B | N | | | | | | | | | | |
| ATOM | | 2080 | CA | LEU | B | 148 | 24.646 | 40.546 | 16.664 | 1.00 | 11.96 |
| B | C | | | | | | | | | | |
| ATOM | | 2081 | CB | LEU | B | 148 | 25.128 | 40.020 | 15.309 | 1.00 | 11.28 |
| B | C | | | | | | | | | | |
| ATOM | | 2082 | CG | LEU | B | 148 | 26.174 | 38.901 | 15.273 | 1.00 | 13.54 |
| B | C | | | | | | | | | | |
| ATOM | | 2083 | CD1 | LEU | B | 148 | 26.101 | 38.200 | 13.936 | 1.00 | 12.19 |
| B | C | | | | | | | | | | |
| ATOM | | 2084 | CD2 | LEU | B | 148 | 27.561 | 39.466 | 15.516 | 1.00 | 12.71 |
| B | C | | | | | | | | | | |
| ATOM | | 2085 | C | LEU | B | 148 | 25.818 | 41.072 | 17.486 | 1.00 | 12.75 |
| B | C | | | | | | | | | | |
| ATOM | | 2086 | O | LEU | B | 148 | 26.256 | 40.428 | 18.434 | 1.00 | 13.22 |
| B | O | | | | | | | | | | |
| ATOM | | 2087 | N | LYS | B | 149 | 26.309 | 42.254 | 17.121 | 1.00 | 12.62 |
| B | N | | | | | | | | | | |
| ATOM | | 2088 | CA | LYS | B | 149 | 27.423 | 42.886 | 17.818 | 1.00 | 13.95 |
| B | C | | | | | | | | | | |
| ATOM | | 2089 | CB | LYS | B | 149 | 27.794 | 44.203 | 17.124 | 1.00 | 13.88 |
| B | C | | | | | | | | | | |
| ATOM | | 2090 | CG | LYS | B | 149 | 28.555 | 43.999 | 15.828 | 1.00 | 17.34 |
| B | C | | | | | | | | | | |
| ATOM | | 2091 | CD | LYS | B | 149 | 28.430 | 45.170 | 14.872 | 1.00 | 22.47 |
| B | C | | | | | | | | | | |
| ATOM | | 2092 | CE | LYS | B | 149 | 28.881 | 46.482 | 15.472 | 1.00 | 25.42 |
| B | C | | | | | | | | | | |
| ATOM | | 2093 | NZ | LYS | B | 149 | 29.064 | 47.513 | 14.402 | 1.00 | 24.53 |
| B | N | | | | | | | | | | |
| ATOM | | 2094 | C | LYS | B | 149 | 27.122 | 43.137 | 19.293 | 1.00 | 14.22 |
| B | C | | | | | | | | | | |
| ATOM | | 2095 | O | LYS | B | 149 | 27.956 | 42.850 | 20.151 | 1.00 | 15.40 |
| B | O | | | | | | | | | | |
| ATOM | | 2096 | N | ASP | B | 150 | 25.939 | 43.663 | 19.593 | 1.00 | 13.64 |
| B | N | | | | | | | | | | |
| ATOM | | 2097 | CA | ASP | B | 150 | 25.576 | 43.928 | 20.988 | 1.00 | 13.95 |
| B | C | | | | | | | | | | |
| ATOM | | 2098 | CB | ASP | B | 150 | 24.198 | 44.589 | 21.080 | 1.00 | 15.50 |
| B | C | | | | | | | | | | |
| ATOM | | 2099 | CG | ASP | B | 150 | 24.172 | 45.985 | 20.493 | 1.00 | 16.66 |
| B | C | | | | | | | | | | |
| ATOM | | 2100 | OD1 | ASP | B | 150 | 25.253 | 46.586 | 20.324 | 1.00 | 16.66 |
| B | O | | | | | | | | | | |
| ATOM | | 2101 | OD2 | ASP | B | 150 | 23.055 | 46.480 | 20.212 | 1.00 | 19.61 |
| B | O | | | | | | | | | | |
| ATOM | | 2102 | C | ASP | B | 150 | 25.554 | 42.661 | 21.843 | 1.00 | 13.62 |
| B | C | | | | | | | | | | |
| ATOM | | 2103 | O | ASP | B | 150 | 25.986 | 42.655 | 23.002 | 1.00 | 12.88 |
| B | O | | | | | | | | | | |
| ATOM | | 2104 | N | THR | B | 151 | 25.017 | 41.591 | 21.276 | 1.00 | 12.91 |
| B | N | | | | | | | | | | |
| ATOM | | 2105 | CA | THR | B | 151 | 24.926 | 40.326 | 21.988 | 1.00 | 14.27 |
| B | C | | | | | | | | | | |
| ATOM | | 2106 | CB | THR | B | 151 | 24.211 | 39.278 | 21.127 | 1.00 | 16.02 |
| B | C | | | | | | | | | | |
| ATOM | | 2107 | OG1 | THR | B | 151 | 22.904 | 39.763 | 20.802 | 1.00 | 15.49 |
| B | O | | | | | | | | | | |
| ATOM | | 2108 | CG2 | THR | B | 151 | 24.101 | 37.951 | 21.862 | 1.00 | 15.10 |
| B | C | | | | | | | | | | |
| ATOM | | 2109 | C | THR | B | 151 | 26.313 | 39.830 | 22.340 | 1.00 | 14.06 |
| B | C | | | | | | | | | | |
| ATOM | | 2110 | O | THR | B | 151 | 26.554 | 39.381 | 23.460 | 1.00 | 15.38 |
| B | O | | | | | | | | | | |
| ATOM | | 2111 | N | VAL | B | 152 | 27.231 | 39.915 | 21.383 | 1.00 | 12.61 |
| B | N | | | | | | | | | | |
| ATOM | | 2112 | CA | VAL | B | 152 | 28.594 | 39.469 | 21.621 | 1.00 | 13.27 |
| B | C | | | | | | | | | | |
| ATOM | | 2113 | CB | VAL | B | 152 | 29.429 | 39.499 | 20.321 | 1.00 | 13.23 |
| B | C | | | | | | | | | | |
| ATOM | | 2114 | CG1 | VAL | B | 152 | 30.900 | 39.371 | 20.647 | 1.00 | 12.74 |
| B | C | | | | | | | | | | |
| ATOM | | 2115 | CG2 | VAL | B | 152 | 29.001 | 38.354 | 19.409 | 1.00 | 13.59 |
| B | C | | | | | | | | | | |
| ATOM | | 2116 | C | VAL | B | 152 | 29.283 | 40.335 | 22.672 | 1.00 | 12.43 |
| B | C | | | | | | | | | | |
| ATOM | | 2117 | O | VAL | B | 152 | 29.916 | 39.817 | 23.587 | 1.00 | 13.68 |
| B | O | | | | | | | | | | |
| ATOM | | 2118 | N | LYS | B | 153 | 29.141 | 41.653 | 22.549 | 1.00 | 11.31 |
| B | N | | | | | | | | | | |
| ATOM | | 2119 | CA | LYS | B | 153 | 29.786 | 42.570 | 23.489 | 1.00 | 9.49 |
| B | C | | | | | | | | | | |
| ATOM | | 2120 | CB | LYS | B | 153 | 29.720 | 44.011 | 22.965 | 1.00 | 9.07 |
| B | C | | | | | | | | | | |
| ATOM | | 2121 | CG | LYS | B | 153 | 30.559 | 44.253 | 21.708 | 1.00 | 7.62 |
| B | C | | | | | | | | | | |
| ATOM | | 2122 | CD | LYS | B | 153 | 32.027 | 43.864 | 21.936 | 1.00 | 9.41 |
| B | C | | | | | | | | | | |
| ATOM | | 2123 | CE | LYS | B | 153 | 32.891 | 44.041 | 20.688 | 1.00 | 9.65 |
| B | C | | | | | | | | | | |
| ATOM | | 2124 | NZ | LYS | B | 153 | 34.308 | 43.621 | 20.957 | 1.00 | 8.41 |
| B | N | | | | | | | | | | |
| ATOM | | 2125 | C | LYS | B | 153 | 29.231 | 42.505 | 24.902 | 1.00 | 9.60 |
| B | C | | | | | | | | | | |
| ATOM | | 2126 | O | LYS | B | 153 | 29.990 | 42.550 | 25.872 | 1.00 | 10.58 |
| B | O | | | | | | | | | | |
| ATOM | | 2127 | N | LYS | B | 154 | 27.917 | 42.398 | 25.034 | 1.00 | 10.82 |
| B | N | | | | | | | | | | |
| ATOM | | 2128 | CA | LYS | B | 154 | 27.318 | 42.330 | 26.362 | 1.00 | 12.31 |
| B | C | | | | | | | | | | |
| ATOM | | 2129 | CB | LYS | B | 154 | 25.787 | 42.296 | 26.255 | 1.00 | 14.53 |
| B | C | | | | | | | | | | |
| ATOM | | 2130 | CG | LYS | B | 154 | 25.109 | 41.913 | 27.561 | 1.00 | 20.06 |
| B | C | | | | | | | | | | |
| ATOM | | 2131 | CD | LYS | B | 154 | 23.589 | 42.030 | 27.501 | 1.00 | 24.39 |
| B | C | | | | | | | | | | |
| ATOM | | 2132 | CE | LYS | B | 154 | 22.991 | 41.812 | 28.891 | 1.00 | 24.64 |
| B | C | | | | | | | | | | |
| ATOM | | 2133 | NZ | LYS | B | 154 | 21.499 | 41.697 | 28.874 | 1.00 | 27.27 |
| B | N | | | | | | | | | | |
| ATOM | | 2134 | C | LYS | B | 154 | 27.808 | 41.105 | 27.138 | 1.00 | 14.73 |
| B | C | | | | | | | | | | |
| ATOM | | 2135 | O | LYS | B | 154 | 27.940 | 41.141 | 28.366 | 1.00 | 13.45 |
| B | O | | | | | | | | | | |
| ATOM | | 2136 | N | LEU | B | 155 | 28.090 | 40.026 | 26.412 | 1.00 | 14.59 |
| B | N | | | | | | | | | | |
| ATOM | | 2137 | CA | LEU | B | 155 | 28.546 | 38.783 | 27.024 | 1.00 | 15.48 |
| B | C | | | | | | | | | | |
| ATOM | | 2138 | CB | LEU | B | 155 | 28.120 | 37.596 | 26.156 | 1.00 | 14.97 |
| B | C | | | | | | | | | | |
| ATOM | | 2139 | CG | LEU | B | 155 | 26.614 | 37.319 | 26.219 | 1.00 | 16.78 |
| B | C | | | | | | | | | | |
| ATOM | | 2140 | CD1 | LEU | B | 155 | 26.251 | 36.184 | 25.283 | 1.00 | 17.88 |
| B | C | | | | | | | | | | |
| ATOM | | 2141 | CD2 | LEU | B | 155 | 26.233 | 36.976 | 27.659 | 1.00 | 18.88 |
| B | C | | | | | | | | | | |
| ATOM | | 2142 | C | LEU | B | 155 | 30.043 | 38.710 | 27.305 | 1.00 | 16.52 |
| B | C | | | | | | | | | | |
| ATOM | | 2143 | O | LEU | B | 155 | 30.522 | 37.749 | 27.904 | 1.00 | 16.70 |
| B | O | | | | | | | | | | |
| ATOM | | 2144 | N | GLY | B | 156 | 30.788 | 39.723 | 26.878 | 1.00 | 16.38 |
| B | N | | | | | | | | | | |
| ATOM | | 2145 | CA | GLY | B | 156 | 32.213 | 39.716 | 27.146 | 1.00 | 17.98 |
| B | C | | | | | | | | | | |
| ATOM | | 2146 | C | GLY | B | 156 | 32.918 | 38.521 | 26.542 | 1.00 | 18.96 |
| B | C | | | | | | | | | | |
| ATOM | | 2147 | O | GLY | B | 156 | 32.658 | 38.159 | 25.398 | 1.00 | 19.62 |
| B | O | | | | | | | | | | |
| ATOM | | 2148 | N | GLU | B | 157 | 33.807 | 37.897 | 27.305 | 1.00 | 20.01 |
| B | N | | | | | | | | | | |
| ATOM | | 2149 | CA | GLU | B | 157 | 34.547 | 36.751 | 26.790 | 1.00 | 22.65 |
| B | C | | | | | | | | | | |
| ATOM | | 2150 | CB | GLU | B | 157 | 35.504 | 36.198 | 27.844 | 1.00 | 26.57 |
| B | C | | | | | | | | | | |
| ATOM | | 2151 | CG | GLU | B | 157 | 36.377 | 35.060 | 27.323 | 1.00 | 32.56 |
| B | C | | | | | | | | | | |
| ATOM | | 2152 | CD | GLU | B | 157 | 37.622 | 34.860 | 28.160 | 1.00 | 36.42 |
| B | C | | | | | | | | | | |
| ATOM | | 2153 | OE1 | GLU | B | 157 | 37.487 | 34.500 | 29.348 | 1.00 | 39.12 |
| B | O | | | | | | | | | | |
| ATOM | | 2154 | OE2 | GLU | B | 157 | 38.738 | 35.071 | 27.632 | 1.00 | 40.09 |
| B | O | | | | | | | | | | |
| ATOM | | 2155 | C | GLU | B | 157 | 33.642 | 35.628 | 26.304 | 1.00 | 21.05 |
| B | C | | | | | | | | | | |
| ATOM | | 2156 | O | GLU | B | 157 | 33.940 | 34.972 | 25.309 | 1.00 | 19.94 |
| B | O | | | | | | | | | | |
| ATOM | | 2157 | N | SER | B | 158 | 32.551 | 35.395 | 27.025 | 1.00 | 20.79 |
| B | N | | | | | | | | | | |
| ATOM | | 2158 | CA | SER | B | 158 | 31.602 | 34.357 | 26.650 | 1.00 | 20.47 |
| B | C | | | | | | | | | | |
| ATOM | | 2159 | CB | SER | B | 158 | 30.481 | 34.264 | 27.684 | 1.00 | 21.91 |
| B | C | | | | | | | | | | |
| ATOM | | 2160 | OG | SER | B | 158 | 31.000 | 33.902 | 28.948 | 1.00 | 26.50 |
| B | O | | | | | | | | | | |
| ATOM | | 2161 | C | SER | B | 158 | 31.006 | 34.696 | 25.291 | 1.00 | 19.88 |
| B | C | | | | | | | | | | |
| ATOM | | 2162 | O | SER | B | 158 | 30.649 | 33.803 | 24.519 | 1.00 | 20.24 |
| B | O | | | | | | | | | | |
| ATOM | | 2163 | N | GLY | B | 159 | 30.890 | 35.991 | 25.010 | 1.00 | 18.62 |
| B | N | | | | | | | | | | |
| ATOM | | 2164 | CA | GLY | B | 159 | 30.343 | 36.426 | 23.733 | 1.00 | 17.19 |
| B | C | | | | | | | | | | |
| ATOM | | 2165 | C | GLY | B | 159 | 31.272 | 36.008 | 22.611 | 1.00 | 15.21 |
| B | C | | | | | | | | | | |
| ATOM | | 2166 | O | GLY | B | 159 | 30.828 | 35.574 | 21.536 | 1.00 | 13.42 |
| B | O | | | | | | | | | | |
| ATOM | | 2167 | N | GLU | B | 160 | 32.569 | 36.138 | 22.862 | 1.00 | 12.99 |
| B | N | | | | | | | | | | |
| ATOM | | 2168 | CA | GLU | B | 160 | 33.571 | 35.759 | 21.880 | 1.00 | 15.94 |
| B | C | | | | | | | | | | |
| ATOM | | 2169 | CB | GLU | B | 160 | 34.963 | 36.197 | 22.349 | 1.00 | 18.02 |
| B | C | | | | | | | | | | |
| ATOM | | 2170 | CG | GLU | B | 160 | 35.143 | 37.708 | 22.329 | 1.00 | 21.23 |
| B | C | | | | | | | | | | |
| ATOM | | 2171 | CD | GLU | B | 160 | 36.471 | 38.151 | 22.913 | 1.00 | 25.05 |
| B | C | | | | | | | | | | |
| ATOM | | 2172 | OE1 | GLU | B | 160 | 37.519 | 37.597 | 22.522 | 1.00 | 26.71 |
| B | O | | | | | | | | | | |
| ATOM | | 2173 | OE2 | GLU | B | 160 | 36.467 | 39.065 | 23.758 | 1.00 | 26.22 |
| B | O | | | | | | | | | | |
| ATOM | | 2174 | C | GLU | B | 160 | 33.533 | 34.252 | 21.646 | 1.00 | 16.05 |
| B | C | | | | | | | | | | |
| ATOM | | 2175 | O | GLU | B | 160 | 33.584 | 33.791 | 20.503 | 1.00 | 16.68 |
| B | O | | | | | | | | | | |
| ATOM | | 2176 | N | ILE | B | 161 | 33.430 | 33.489 | 22.728 | 1.00 | 15.72 |
| B | N | | | | | | | | | | |
| ATOM | | 2177 | CA | ILE | B | 161 | 33.380 | 32.033 | 22.628 | 1.00 | 15.75 |
| B | C | | | | | | | | | | |
| ATOM | | 2178 | CB | AILE | B | 161 | 33.334 | 31.371 | 24.025 | 0.50 | 17.43 |
| B | C | | | | | | | | | | |
| ATOM | | 2179 | CB | BILE | B | 161 | 33.345 | 31.389 | 24.043 | 0.50 | 17.65 |
| B | C | | | | | | | | | | |
| ATOM | | 2180 | CG2 | AILE | B | 161 | 33.225 | 29.860 | 23.887 | 0.50 | 18.33 |
| B | C | | | | | | | | | | |
| ATOM | | 2181 | CG2 | BILE | B | 161 | 33.204 | 29.879 | 23.924 | 0.50 | 18.35 |
| B | C | | | | | | | | | | |
| ATOM | | 2182 | CG1 | AILE | B | 161 | 34.588 | 31.753 | 24.820 | 0.50 | 18.68 |
| B | C | | | | | | | | | | |
| ATOM | | 2183 | CG1 | BILE | B | 161 | 34.627 | 31.750 | 24.803 | 0.50 | 18.95 |
| B | C | | | | | | | | | | |
| ATOM | | 2184 | CD | AILE | B | 161 | 35.890 | 31.386 | 24.139 | 0.50 | 18.08 |
| B | C | | | | | | | | | | |
| ATOM | | 2185 | CD | BILE | B | 161 | 34.640 | 31.314 | 26.252 | 0.50 | 19.38 |
| B | C | | | | | | | | | | |
| ATOM | | 2186 | C | ILE | B | 161 | 32.151 | 31.616 | 21.828 | 1.00 | 15.04 |
| B | C | | | | | | | | | | |
| ATOM | | 2187 | O | ILE | B | 161 | 32.233 | 30.745 | 20.960 | 1.00 | 13.38 |
| B | O | | | | | | | | | | |
| ATOM | | 2188 | N | LYS | B | 162 | 31.017 | 32.251 | 22.110 | 1.00 | 14.57 |
| B | N | | | | | | | | | | |
| ATOM | | 2189 | CA | LYS | B | 162 | 29.782 | 31.938 | 21.401 | 1.00 | 14.05 |
| B | C | | | | | | | | | | |
| ATOM | | 2190 | CB | LYS | B | 162 | 28.621 | 32.771 | 21.954 | 1.00 | 14.23 |
| B | C | | | | | | | | | | |
| ATOM | | 2191 | CG | LYS | B | 162 | 27.299 | 32.559 | 21.206 | 1.00 | 15.87 |
| B | C | | | | | | | | | | |
| ATOM | | 2192 | CD | LYS | B | 162 | 26.211 | 33.520 | 21.678 | 1.00 | 16.15 |
| B | C | | | | | | | | | | |
| ATOM | | 2193 | CE | LYS | B | 162 | 25.913 | 33.359 | 23.163 | 1.00 | 18.43 |
| B | C | | | | | | | | | | |
| ATOM | | 2194 | NZ | LYS | B | 162 | 25.341 | 32.018 | 23.490 | 1.00 | 22.02 |
| B | N | | | | | | | | | | |
| ATOM | | 2195 | C | LYS | B | 162 | 29.933 | 32.208 | 19.910 | 1.00 | 12.51 |
| B | C | | | | | | | | | | |
| ATOM | | 2196 | O | LYS | B | 162 | 29.445 | 31.442 | 19.078 | 1.00 | 13.34 |
| B | O | | | | | | | | | | |
| ATOM | | 2197 | N | ALA | B | 163 | 30.611 | 33.297 | 19.574 | 1.00 | 12.30 |
| B | N | | | | | | | | | | |
| ATOM | | 2198 | CA | ALA | B | 163 | 30.822 | 33.656 | 18.177 | 1.00 | 12.59 |
| B | C | | | | | | | | | | |
| ATOM | | 2199 | CB | ALA | B | 163 | 31.474 | 35.036 | 18.089 | 1.00 | 12.94 |
| B | C | | | | | | | | | | |
| ATOM | | 2200 | C | ALA | B | 163 | 31.684 | 32.617 | 17.454 | 1.00 | 12.63 |
| B | C | | | | | | | | | | |
| ATOM | | 2201 | O | ALA | B | 163 | 31.435 | 32.281 | 16.293 | 1.00 | 13.56 |
| B | O | | | | | | | | | | |
| ATOM | | 2202 | N | ILE | B | 164 | 32.704 | 32.111 | 18.135 | 1.00 | 13.23 |
| B | N | | | | | | | | | | |
| ATOM | | 2203 | CA | ILE | B | 164 | 33.578 | 31.107 | 17.532 | 1.00 | 10.59 |
| B | C | | | | | | | | | | |
| ATOM | | 2204 | CB | ILE | B | 164 | 34.824 | 30.853 | 18.403 | 1.00 | 12.38 |
| B | C | | | | | | | | | | |
| ATOM | | 2205 | CG2 | ILE | B | 164 | 35.701 | 29.767 | 17.768 | 1.00 | 8.98 |
| B | C | | | | | | | | | | |
| ATOM | | 2206 | CG1 | ILE | B | 164 | 35.617 | 32.155 | 18.566 | 1.00 | 9.24 |
| B | C | | | | | | | | | | |
| ATOM | | 2207 | CD | ILE | B | 164 | 36.746 | 32.058 | 19.590 | 1.00 | 11.68 |
| B | C | | | | | | | | | | |
| ATOM | | 2208 | C | ILE | B | 164 | 32.793 | 29.804 | 17.379 | 1.00 | 12.14 |
| B | C | | | | | | | | | | |
| ATOM | | 2209 | O | ILE | B | 164 | 33.009 | 29.049 | 16.431 | 1.00 | 12.85 |
| B | O | | | | | | | | | | |
| ATOM | | 2210 | N | GLY | B | 165 | 31.868 | 29.558 | 18.305 | 1.00 | 10.88 |
| B | N | | | | | | | | | | |
| ATOM | | 2211 | CA | GLY | B | 165 | 31.063 | 28.345 | 18.252 | 1.00 | 13.41 |
| B | C | | | | | | | | | | |
| ATOM | | 2212 | C | GLY | B | 165 | 30.109 | 28.337 | 17.072 | 1.00 | 14.68 |
| B | C | | | | | | | | | | |
| ATOM | | 2213 | O | GLY | B | 165 | 29.583 | 27.290 | 16.686 | 1.00 | 14.73 |
| B | O | | | | | | | | | | |
| ATOM | | 2214 | N | GLU | B | 166 | 29.890 | 29.512 | 16.495 | 1.00 | 13.01 |
| B | N | | | | | | | | | | |
| ATOM | | 2215 | CA | GLU | B | 166 | 29.011 | 29.656 | 15.349 | 1.00 | 14.06 |
| B | C | | | | | | | | | | |
| ATOM | | 2216 | CB | GLU | B | 166 | 28.031 | 30.773 | 15.621 | 1.00 | 16.63 |
| B | C | | | | | | | | | | |
| ATOM | | 2217 | CG | GLU | B | 166 | 27.344 | 30.594 | 16.942 | 1.00 | 21.35 |
| B | C | | | | | | | | | | |
| ATOM | | 2218 | CD | GLU | B | 166 | 26.278 | 29.510 | 16.908 | 1.00 | 25.81 |
| B | C | | | | | | | | | | |
| ATOM | | 2219 | OE1 | GLU | B | 166 | 26.648 | 28.326 | 16.873 | 1.00 | 31.72 |
| B | O | | | | | | | | | | |
| ATOM | | 2220 | OE1 | GLU | B | 166 | 25.065 | 29.822 | 16.913 | 1.00 | 30.04 |
| B | O | | | | | | | | | | |
| ATOM | | 2221 | C | GLU | B | 166 | 29.767 | 29.952 | 14.062 | 1.00 | 11.84 |
| B | C | | | | | | | | | | |
| ATOM | | 2222 | O | GLU | B | 166 | 29.164 | 30.323 | 13.054 | 1.00 | 11.51 |
| B | O | | | | | | | | | | |
| ATOM | | 2223 | N | LEU | B | 167 | 31.084 | 29.781 | 14.090 | 1.00 | 10.92 |
| B | N | | | | | | | | | | |
| ATOM | | 2224 | CA | LEU | B | 167 | 31.895 | 30.033 | 12.903 | 1.00 | 12.37 |
| B | C | | | | | | | | | | |
| ATOM | | 2225 | CB | LEU | B | 167 | 33.391 | 29.836 | 13.195 | 1.00 | 15.29 |
| B | C | | | | | | | | | | |
| ATOM | | 2226 | CG | LEU | B | 167 | 34.115 | 31.001 | 13.872 | 1.00 | 20.06 |
| B | C | | | | | | | | | | |
| ATOM | | 2227 | CD1 | LEU | B | 167 | 35.562 | 30.612 | 14.125 | 1.00 | 22.06 |
| B | C | | | | | | | | | | |
| ATOM | | 2228 | CD2 | LEU | B | 167 | 34.033 | 32.247 | 12.996 | 1.00 | 20.29 |
| B | C | | | | | | | | | | |
| ATOM | | 2229 | C | LEU | B | 167 | 31.496 | 29.137 | 11.739 | 1.00 | 13.10 |
| B | C | | | | | | | | | | |
| ATOM | | 2230 | O | LEU | B | 167 | 31.733 | 29.481 | 10.585 | 1.00 | 12.57 |
| B | O | | | | | | | | | | |
| ATOM | | 2231 | N | ASP | B | 168 | 30.901 | 27.986 | 12.034 | 1.00 | 12.74 |
| B | N | | | | | | | | | | |
| ATOM | | 2232 | CA | ASP | B | 168 | 30.478 | 27.100 | 10.960 | 1.00 | 14.28 |
| B | C | | | | | | | | | | |
| ATOM | | 2233 | CB | ASP | B | 168 | 30.058 | 25.727 | 11.515 | 1.00 | 13.57 |
| B | C | | | | | | | | | | |
| ATOM | | 2234 | CG | ASP | B | 168 | 29.185 | 25.821 | 12.760 | 1.00 | 17.07 |
| B | C | | | | | | | | | | |
| ATOM | | 2235 | OD1 | ASP | B | 168 | 28.964 | 26.930 | 13.295 | 1.00 | 14.89 |
| B | O | | | | | | | | | | |
| ATOM | | 2236 | OD2 | ASP | B | 168 | 28.723 | 24.755 | 13.218 | 1.00 | 18.76 |
| B | O | | | | | | | | | | |
| ATOM | | 2237 | C | ASP | B | 168 | 29.335 | 27.767 | 10.186 | 1.00 | 12.65 |
| B | C | | | | | | | | | | |
| ATOM | | 2238 | O | ASP | B | 168 | 29.312 | 27.747 | 8.958 | 1.00 | 13.63 |
| B | O | | | | | | | | | | |
| ATOM | | 2239 | N | LEU | B | 169 | 28.403 | 28.379 | 10.903 | 1.00 | 11.65 |
| B | N | | | | | | | | | | |
| ATOM | | 2240 | CA | LEU | B | 169 | 27.293 | 29.068 | 10.255 | 1.00 | 14.38 |
| B | C | | | | | | | | | | |
| ATOM | | 2241 | CB | LEU | B | 169 | 26.245 | 29.481 | 11.283 | 1.00 | 15.94 |
| B | C | | | | | | | | | | |
| ATOM | | 2242 | CG | LEU | B | 169 | 25.462 | 28.363 | 11.971 | 1.00 | 18.22 |
| B | C | | | | | | | | | | |
| ATOM | | 2243 | CD1 | LEU | B | 169 | 24.391 | 28.973 | 12.880 | 1.00 | 18.16 |
| B | C | | | | | | | | | | |
| ATOM | | 2244 | CD2 | LEU | B | 169 | 24.822 | 27.472 | 10.913 | 1.00 | 20.52 |
| B | C | | | | | | | | | | |
| ATOM | | 2245 | C | LEU | B | 169 | 27.791 | 30.311 | 9.520 | 1.00 | 15.25 |
| B | C | | | | | | | | | | |
| ATOM | | 2246 | O | LEU | B | 169 | 27.249 | 30.701 | 8.483 | 1.00 | 14.55 |
| B | O | | | | | | | | | | |
| ATOM | | 2247 | N | LEU | B | 170 | 28.821 | 30.944 | 10.062 | 1.00 | 15.29 |
| B | N | | | | | | | | | | |
| ATOM | | 2248 | CA | LEU | B | 170 | 29.359 | 32.133 | 9.421 | 1.00 | 16.17 |
| B | C | | | | | | | | | | |
| ATOM | | 2249 | CB | LEU | B | 170 | 30.418 | 32.795 | 10.309 | 1.00 | 16.47 |
| B | C | | | | | | | | | | |
| ATOM | | 2250 | CG | LEU | B | 170 | 31.234 | 33.908 | 9.639 | 1.00 | 18.75 |
| B | C | | | | | | | | | | |
| ATOM | | 2251 | CD1 | LEU | B | 170 | 30.309 | 34.997 | 9.152 | 1.00 | 20.23 |
| B | C | | | | | | | | | | |
| ATOM | | 2252 | CD2 | LEU | B | 170 | 32.242 | 34.479 | 10.622 | 1.00 | 20.78 |
| B | C | | | | | | | | | | |
| ATOM | | 2253 | C | LEU | B | 170 | 29.976 | 31.742 | 8.088 | 1.00 | 16.04 |
| B | C | | | | | | | | | | |
| ATOM | | 2254 | O | LEU | B | 170 | 29.754 | 32.397 | 7.069 | 1.00 | 17.27 |
| B | O | | | | | | | | | | |
| ATOM | | 2255 | N | PHE | B | 171 | 30.748 | 30.663 | 8.104 | 1.00 | 15.59 |
| B | N | | | | | | | | | | |
| ATOM | | 2256 | CA | PHE | B | 171 | 31.418 | 30.173 | 6.905 | 1.00 | 17.71 |
| B | C | | | | | | | | | | |
| ATOM | | 2257 | CB | PHE | B | 171 | 32.313 | 28.978 | 7.271 | 1.00 | 18.23 |
| B | C | | | | | | | | | | |
| ATOM | | 2258 | CG | PHE | B | 171 | 33.023 | 28.355 | 6.093 | 1.00 | 23.19 |
| B | C | | | | | | | | | | |
| ATOM | | 2259 | CD1 | PHE | B | 171 | 32.324 | 27.606 | 5.147 | 1.00 | 25.08 |
| B | C | | | | | | | | | | |
| ATOM | | 2260 | CD2 | PHE | B | 171 | 34.395 | 28.521 | 5.930 | 1.00 | 25.84 |
| B | C | | | | | | | | | | |
| ATOM | | 2261 | CE1 | PHE | B | 171 | 32.982 | 27.031 | 4.053 | 1.00 | 26.30 |
| B | C | | | | | | | | | | |
| ATOM | | 2262 | CE2 | PHE | B | 171 | 35.064 | 27.949 | 4.840 | 1.00 | 26.12 |
| B | C | | | | | | | | | | |
| ATOM | | 2263 | CZ | PHE | B | 171 | 34.353 | 27.204 | 3.902 | 1.00 | 25.36 |
| B | C | | | | | | | | | | |
| ATOM | | 2264 | C | PHE | B | 171 | 30.413 | 29.773 | 5.824 | 1.00 | 15.84 |
| B | C | | | | | | | | | | |
| ATOM | | 2265 | O | PHE | B | 171 | 30.545 | 30.158 | 4.657 | 1.00 | 15.02 |
| B | O | | | | | | | | | | |
| ATOM | | 2266 | N | MET | B | 172 | 29.411 | 28.991 | 6.207 | 1.00 | 15.66 |
| B | N | | | | | | | | | | |
| ATOM | | 2267 | CA | MET | B | 172 | 28.407 | 28.548 | 5.241 | 1.00 | 14.21 |
| B | C | | | | | | | | | | |
| ATOM | | 2268 | CB | MET | B | 172 | 27.505 | 27.484 | 5.859 | 1.00 | 14.82 |
| B | C | | | | | | | | | | |
| ATOM | | 2269 | CG | MET | B | 172 | 28.167 | 26.139 | 6.076 | 1.00 | 17.32 |
| B | C | | | | | | | | | | |
| ATOM | | 2270 | SD | MET | B | 172 | 28.576 | 25.335 | 4.523 | 1.00 | 19.02 |
| B | S | | | | | | | | | | |
| ATOM | | 2271 | CE | MET | B | 172 | 30.140 | 24.649 | 4.909 | 1.00 | 18.35 |
| B | C | | | | | | | | | | |
| ATOM | | 2272 | C | MET | B | 172 | 27.552 | 29.701 | 4.723 | 1.00 | 15.52 |
| B | C | | | | | | | | | | |
| ATOM | | 2273 | O | MET | B | 172 | 27.216 | 29.750 | 3.543 | 1.00 | 16.04 |
| B | O | | | | | | | | | | |
| ATOM | | 2274 | N | SER | B | 173 | 27.191 | 30.623 | 5.610 | 1.00 | 17.55 |
| B | N | | | | | | | | | | |
| ATOM | | 2275 | CA | SER | B | 173 | 26.368 | 31.766 | 5.225 | 1.00 | 19.47 |
| B | C | | | | | | | | | | |
| ATOM | | 2276 | CB | SER | B | 173 | 25.962 | 32.554 | 6.462 | 1.00 | 18.60 |
| B | C | | | | | | | | | | |
| ATOM | | 2277 | OG | SER | B | 173 | 25.085 | 31.797 | 7.272 | 1.00 | 20.37 |
| B | O | | | | | | | | | | |
| ATOM | | 2278 | C | SER | B | 173 | 27.123 | 32.671 | 4.262 | 1.00 | 21.94 |
| B | C | | | | | | | | | | |
| ATOM | | 2279 | O | SER | B | 173 | 26.551 | 33.201 | 3.303 | 1.00 | 22.14 |
| B | O | | | | | | | | | | |
| ATOM | | 2280 | N | LEU | B | 174 | 28.412 | 32.853 | 4.523 | 1.00 | 22.66 |
| B | N | | | | | | | | | | |
| ATOM | | 2281 | CA | LEU | B | 174 | 29.252 | 33.688 | 3.676 | 1.00 | 23.53 |
| B | C | | | | | | | | | | |
| ATOM | | 2282 | CB | ALEU | B | 174 | 30.652 | 33.804 | 4.287 | 0.50 | 23.45 |
| B | C | | | | | | | | | | |
| ATOM | | 2283 | CB | BLEU | B | 174 | 30.671 | 33.756 | 4.215 | 0.50 | 23.44 |
| B | C | | | | | | | | | | |
| ATOM | | 2284 | CG | ALEU | B | 174 | 31.647 | 34.741 | 3.608 | 0.50 | 23.66 |
| B | C | | | | | | | | | | |
| ATOM | | 2285 | CG | BLEU | B | 174 | 31.036 | 34.904 | 5.148 | 0.50 | 23.44 |
| B | C | | | | | | | | | | |
| ATOM | | 2286 | CD1 | ALEU | B | 174 | 31.128 | 36.165 | 3.669 | 0.50 | 23.07 |
| B | C | | | | | | | | | | |
| ATOM | | 2287 | CD1 | BLEU | B | 174 | 32.414 | 34.656 | 5.737 | 0.50 | 22.70 |
| B | C | | | | | | | | | | |
| ATOM | | 2288 | CD2 | ALEU | B | 174 | 32.995 | 34.633 | 4.308 | 0.50 | 22.85 |
| B | C | | | | | | | | | | |
| ATOM | | 2289 | CD2 | BLEU | B | 174 | 31.012 | 36.205 | 4.367 | 0.50 | 23.54 |
| B | C | | | | | | | | | | |
| ATOM | | 2290 | C | LEU | B | 174 | 29.340 | 33.097 | 2.274 | 1.00 | 25.13 |
| B | C | | | | | | | | | | |
| ATOM | | 2291 | O | LEU | B | 174 | 29.225 | 33.814 | 1.282 | 1.00 | 25.73 |
| B | O | | | | | | | | | | |
| ATOM | | 2292 | N | ARG | B | 175 | 29.547 | 31.785 | 2.204 | 1.00 | 25.47 |
| B | N | | | | | | | | | | |
| ATOM | | 2293 | CA | ARG | B | 175 | 29.641 | 31.074 | 0.933 | 1.00 | 26.85 |
| B | C | | | | | | | | | | |
| ATOM | | 2294 | CB | ARG | B | 175 | 29.872 | 29.584 | 1.196 | 1.00 | 28.61 |
| B | C | | | | | | | | | | |
| ATOM | | 2295 | CG | ARG | B | 175 | 30.384 | 28.818 | 0.006 | 1.00 | 31.65 |
| B | C | | | | | | | | | | |
| ATOM | | 2296 | CD | ARG | B | 175 | 30.369 | 27.331 | 0.279 | 1.00 | 35.80 |
| B | C | | | | | | | | | | |
| ATOM | | 2297 | NE | ARG | B | 175 | 29.017 | 26.786 | 0.193 | 1.00 | 37.83 |
| B | N | | | | | | | | | | |
| ATOM | | 2298 | CZ | ARG | B | 175 | 28.317 | 26.731 | −0.935 | 1.00 | 41.57 |
| B | C | | | | | | | | | | |
| ATOM | | 2299 | NH1 | ARG | B | 175 | 28.849 | 27.183 | −2.060 | 1.00 | 42.90 |
| B | N | | | | | | | | | | |
| ATOM | | 2300 | NH2 | ARG | B | 175 | 27.084 | 26.238 | −0.944 | 1.00 | 42.15 |
| B | N | | | | | | | | | | |
| ATOM | | 2301 | C | ARG | B | 175 | 28.365 | 31.264 | 0.101 | 1.00 | 26.48 |
| B | C | | | | | | | | | | |
| ATOM | | 2302 | O | ARG | B | 175 | 28.416 | 31.705 | −1.049 | 1.00 | 27.29 |
| B | O | | | | | | | | | | |
| ATOM | | 2303 | N | ASN | B | 176 | 27.216 | 30.954 | 0.690 | 1.00 | 24.26 |
| B | N | | | | | | | | | | |
| ATOM | | 2304 | CA | ASN | B | 176 | 25.943 | 31.095 | −0.010 | 1.00 | 25.29 |
| B | C | | | | | | | | | | |
| ATOM | | 2305 | CB | ASN | B | 176 | 24.831 | 30.435 | 0.798 | 1.00 | 22.74 |
| B | C | | | | | | | | | | |
| ATOM | | 2306 | CG | ASN | B | 176 | 24.682 | 28.972 | 0.469 | 1.00 | 24.13 |
| B | C | | | | | | | | | | |
| ATOM | | 2307 | OD1 | ASN | B | 176 | 25.668 | 28.285 | 0.187 | 1.00 | 22.83 |
| B | O | | | | | | | | | | |
| ATOM | | 2308 | ND2 | ASN | B | 176 | 23.447 | 28.479 | 0.503 | 1.00 | 20.65 |
| B | N | | | | | | | | | | |
| ATOM | | 2309 | C | ASN | B | 176 | 25.539 | 32.522 | −0.348 | 1.00 | 26.76 |
| B | C | | | | | | | | | | |
| ATOM | | 2310 | O | ASN | B | 176 | 24.759 | 32.753 | −1.276 | 1.00 | 26.72 |
| B | O | | | | | | | | | | |
| ATOM | | 2311 | N | ALA | B | 177 | 26.068 | 33.481 | 0.397 | 1.00 | 25.95 |
| B | N | | | | | | | | | | |
| ATOM | | 2312 | CA | ALA | B | 177 | 25.729 | 34.870 | 0.162 | 1.00 | 26.17 |
| B | C | | | | | | | | | | |
| ATOM | | 2313 | CB | ALA | B | 177 | 25.737 | 35.633 | 1.487 | 1.00 | 24.10 |
| B | C | | | | | | | | | | |
| ATOM | | 2314 | C | ALA | B | 177 | 26.663 | 35.550 | −0.826 | 1.00 | 25.49 |
| B | C | | | | | | | | | | |
| ATOM | | 2315 | O | ALA | B | 177 | 26.275 | 36.511 | −1.478 | 1.00 | 25.51 |
| B | O | | | | | | | | | | |
| ATOM | | 2316 | N | CYS | B | 178 | 27.879 | 35.033 | −0.964 | 1.00 | 27.87 |
| B | N | | | | | | | | | | |
| ATOM | | 2317 | CA | CYS | B | 178 | 28.862 | 35.671 | −1.820 | 1.00 | 27.66 |
| B | C | | | | | | | | | | |
| ATOM | | 2318 | C | CYS | B | 178 | 29.271 | 34.977 | −3.128 | 1.00 | 29.33 |
| B | C | | | | | | | | | | |
| ATOM | | 2319 | O | CYS | B | 178 | 29.732 | 35.666 | −4.035 | 1.00 | 31.34 |
| B | O | | | | | | | | | | |
| ATOM | | 2320 | CB | CYS | B | 178 | 30.123 | 35.902 | −0.991 | 1.00 | 27.05 |
| B | C | | | | | | | | | | |
| ATOM | | 2321 | SG | CYS | B | 178 | 29.912 | 37.031 | 0.419 | 1.00 | 25.07 |
| B | S | | | | | | | | | | |
| ATOM | | 2322 | N | ILE | B | 179 | 29.109 | 33.664 | −3.245 | 1.00 | 30.95 |
| B | N | | | | | | | | | | |
| ATOM | | 2323 | CA | ILE | B | 179 | 29.490 | 33.008 | −4.491 | 1.00 | 31.56 |
| B | C | | | | | | | | | | |
| ATOM | | 2324 | CB | ILE | B | 179 | 29.681 | 31.496 | −4.309 | 1.00 | 32.47 |
| B | C | | | | | | | | | | |
| ATOM | | 2325 | CG2 | ILE | B | 179 | 30.764 | 31.239 | −3.275 | 1.00 | 34.45 |
| B | C | | | | | | | | | | |
| ATOM | | 2326 | CG1 | ILE | B | 179 | 28.356 | 30.851 | −3.899 | 1.00 | 33.65 |
| B | C | | | | | | | | | | |
| ATOM | | 2327 | CD | ILE | B | 179 | 28.408 | 29.343 | −3.849 | 1.00 | 35.23 |
| B | C | | | | | | | | | | |
| ATOM | | 2328 | C | ILE | B | 179 | 28.441 | 33.220 | −5.576 | 1.00 | 31.05 |
| B | C | | | | | | | | | | |
| ATOM | | 2329 | OT1 | ILE | B | 179 | 27.279 | 33.539 | −5.240 | 1.00 | 29.84 |
| B | O | | | | | | | | | | |
| ATOM | | 2330 | OT2 | ILE | B | 179 | 28.806 | 33.043 | −6.756 | 1.00 | 32.57 |
| B | O | | | | | | | | | | |
| HETATM | | 2331 | O | HOH | W | 201 | 27.297 | 23.446 | 24.432 | 1.00 | 10.44 |
| W | O | | | | | | | | | | |
| HETATM | | 2332 | O | HOH | W | 202 | 29.001 | 24.489 | 16.199 | 1.00 | 15.87 |
| W | O | | | | | | | | | | |
| HETATM | | 2333 | O | HOH | W | 203 | 11.736 | 25.818 | 21.510 | 1.00 | 19.00 |
| W | O | | | | | | | | | | |
| HETATM | | 2334 | O | HOH | W | 204 | 24.143 | 15.462 | 11.058 | 1.00 | 16.00 |
| W | O | | | | | | | | | | |
| HETATM | | 2335 | O | HOH | W | 205 | 13.986 | 10.421 | 2.897 | 1.00 | 47.98 |
| W | O | | | | | | | | | | |
| HETATM | | 2336 | O | HOH | W | 206 | 9.264 | 23.783 | 21.316 | 1.00 | 19.55 |
| W | O | | | | | | | | | | |
| HETATM | | 2337 | O | HOH | W | 207 | 26.417 | 26.497 | 21.231 | 1.00 | 16.89 |
| W | O | | | | | | | | | | |
| HETATM | | 2338 | O | HOH | W | 208 | 25.199 | 18.812 | 10.440 | 1.00 | 16.53 |
| W | O | | | | | | | | | | |
| HETATM | | 2339 | O | HOH | W | 209 | 27.671 | 14.819 | 7.534 | 1.00 | 15.58 |
| W | O | | | | | | | | | | |
| HETATM | | 2340 | O | HOH | W | 210 | 17.220 | 31.319 | 12.529 | 1.00 | 18.46 |
| W | O | | | | | | | | | | |
| HETATM | | 2341 | O | HOH | W | 211 | 7.763 | 15.191 | 13.315 | 1.00 | 16.34 |
| W | O | | | | | | | | | | |
| HETATM | | 2342 | O | HOH | W | 212 | 25.429 | 52.025 | 16.393 | 1.00 | 21.97 |
| W | O | | | | | | | | | | |
| HETATM | | 2343 | O | HOH | W | 213 | 4.366 | 14.026 | −0.146 | 1.00 | 16.43 |
| W | O | | | | | | | | | | |
| HETATM | | 2344 | O | HOH | W | 214 | 30.378 | 15.252 | 8.290 | 1.00 | 17.71 |
| W | O | | | | | | | | | | |
| HETATM | | 2345 | O | HOH | W | 215 | 29.716 | 36.103 | 14.680 | 1.00 | 26.32 |
| W | O | | | | | | | | | | |
| HETATM | | 2346 | O | HOH | W | 216 | 29.851 | 44.950 | 27.330 | 1.00 | 16.23 |
| W | O | | | | | | | | | | |
| HETATM | | 2347 | O | HOH | W | 217 | 25.965 | 42.188 | −1.336 | 1.00 | 26.70 |
| W | O | | | | | | | | | | |
| HETATM | | 2348 | O | HOH | W | 218 | 32.742 | 40.348 | 23.501 | 1.00 | 19.93 |
| W | O | | | | | | | | | | |
| HETATM | | 2349 | O | HOH | W | 219 | 21.669 | 14.477 | 11.866 | 1.00 | 21.69 |
| W | O | | | | | | | | | | |
| HETATM | | 2350 | O | HOH | W | 220 | 34.189 | 40.928 | 21.191 | 1.00 | 21.11 |
| W | O | | | | | | | | | | |
| HETATM | | 2351 | O | HOH | W | 221 | 13.503 | 33.656 | −0.622 | 1.00 | 21.91 |
| W | O | | | | | | | | | | |
| HETATM | | 2352 | O | HOH | W | 222 | 24.529 | 39.089 | −2.108 | 1.00 | 22.69 |
| W | O | | | | | | | | | | |
| HETATM | | 2353 | O | HOH | W | 223 | 20.087 | 30.335 | −0.404 | 1.00 | 26.82 |
| W | O | | | | | | | | | | |
| HETATM | | 2354 | O | HOH | W | 224 | 21.910 | 37.842 | 18.502 | 1.00 | 21.17 |
| W | O | | | | | | | | | | |
| HETATM | | 2355 | O | HOH | W | 225 | 19.543 | 26.452 | −10.389 | 1.00 | 24.04 |
| W | O | | | | | | | | | | |
| HETATM | | 2356 | O | HOH | W | 226 | 15.561 | 31.685 | 14.681 | 1.00 | 45.81 |
| W | O | | | | | | | | | | |
| HETATM | | 2357 | O | HOH | W | 227 | 32.308 | 36.045 | 30.019 | 1.00 | 28.38 |
| W | O | | | | | | | | | | |
| HETATM | | 2358 | O | HOH | W | 228 | 17.729 | 31.816 | 1.688 | 1.00 | 18.99 |
| W | O | | | | | | | | | | |
| HETATM | | 2359 | O | HOH | W | 229 | 25.158 | 23.427 | 25.705 | 1.00 | 23.41 |
| W | O | | | | | | | | | | |
| HETATM | | 2360 | O | HOH | W | 230 | 27.991 | 29.102 | 19.752 | 1.00 | 20.66 |
| W | O | | | | | | | | | | |
| HETATM | | 2361 | O | HOH | W | 231 | 3.725 | 18.194 | 12.154 | 1.00 | 33.73 |
| W | O | | | | | | | | | | |
| HETATM | | 2362 | O | HOH | W | 232 | 37.621 | 42.067 | 21.510 | 1.00 | 27.85 |
| W | O | | | | | | | | | | |
| HETATM | | 2363 | O | HOH | W | 233 | 19.597 | 28.257 | 20.861 | 1.00 | 28.65 |
| W | O | | | | | | | | | | |
| HETATM | | 2364 | O | HOH | W | 234 | 20.056 | 40.743 | 13.648 | 1.00 | 32.86 |
| W | O | | | | | | | | | | |
| HETATM | | 2365 | O | HOH | W | 235 | 11.670 | 39.187 | 7.650 | 1.00 | 30.82 |
| W | O | | | | | | | | | | |
| HETATM | | 2366 | O | HOH | W | 236 | 18.418 | 33.049 | 20.243 | 1.00 | 26.85 |
| W | O | | | | | | | | | | |
| HETATM | | 2367 | O | HOH | W | 237 | 23.466 | 27.862 | 16.542 | 1.00 | 25.38 |
| W | O | | | | | | | | | | |
| HETATM | | 2368 | O | HOH | W | 238 | 36.544 | 43.733 | 3.953 | 1.00 | 24.65 |
| W | O | | | | | | | | | | |
| HETATM | | 2369 | O | HOH | W | 239 | 20.855 | 44.986 | 20.266 | 1.00 | 20.67 |
| W | O | | | | | | | | | | |
| HETATM | | 2370 | O | HOH | W | 240 | 6.104 | 33.041 | 6.261 | 1.00 | 34.43 |
| W | O | | | | | | | | | | |
| HETATM | | 2371 | O | HOH | W | 241 | 22.194 | 38.315 | 15.523 | 1.00 | 30.42 |
| W | O | | | | | | | | | | |
| HETATM | | 2372 | O | HOH | W | 242 | 26.322 | 47.696 | 12.361 | 1.00 | 28.82 |
| W | O | | | | | | | | | | |
| HETATM | | 2373 | O | HOH | W | 243 | 29.634 | 17.921 | 1.255 | 1.00 | 23.78 |
| W | O | | | | | | | | | | |
| HETATM | | 2374 | O | HOH | W | 244 | 12.805 | 39.529 | 5.415 | 1.00 | 48.81 |
| W | O | | | | | | | | | | |
| HETATM | | 2375 | O | HOH | W | 245 | 9.624 | 26.277 | 4.198 | 1.00 | 24.30 |
| W | O | | | | | | | | | | |
| HETATM | | 2376 | O | HOH | W | 246 | 7.395 | 13.510 | 7.017 | 1.00 | 35.04 |
| W | O | | | | | | | | | | |
| HETATM | | 2377 | O | HOH | W | 247 | 30.410 | 33.686 | 14.273 | 1.00 | 21.60 |
| W | O | | | | | | | | | | |
| HETATM | | 2378 | O | HOH | W | 248 | 36.748 | 43.355 | 23.420 | 1.00 | 28.82 |
| W | O | | | | | | | | | | |
| HETATM | | 2379 | O | HOH | W | 249 | 19.550 | 13.720 | 10.528 | 1.00 | 21.91 |
| W | O | | | | | | | | | | |
| HETATM | | 2380 | O | HOH | W | 250 | 17.891 | 27.372 | −12.427 | 1.00 | 32.70 |
| W | O | | | | | | | | | | |
| HETATM | | 2381 | O | HOH | W | 251 | 31.614 | 45.127 | 10.612 | 1.00 | 28.57 |
| W | O | | | | | | | | | | |
| HETATM | | 2382 | O | HOH | W | 252 | 36.318 | 24.599 | 24.398 | 1.00 | 27.35 |
| W | O | | | | | | | | | | |
| HETATM | | 2383 | O | HOH | W | 253 | 27.851 | 21.966 | 22.159 | 1.00 | 35.57 |
| W | O | | | | | | | | | | |
| HETATM | | 2384 | O | HOH | W | 254 | 12.195 | 26.188 | 3.549 | 1.00 | 21.42 |
| W | O | | | | | | | | | | |
| HETATM | | 2385 | O | HOH | W | 255 | 14.952 | 35.943 | 7.315 | 1.00 | 31.47 |
| W | O | | | | | | | | | | |
| HETATM | | 2386 | O | HOH | W | 256 | 25.476 | 31.892 | 15.021 | 1.00 | 42.15 |
| W | O | | | | | | | | | | |
| HETATM | | 2387 | O | HOH | W | 257 | 33.419 | 16.973 | 5.504 | 1.00 | 24.87 |
| W | O | | | | | | | | | | |
| HETATM | | 2388 | O | HOH | W | 258 | 2.676 | 21.388 | 9.316 | 1.00 | 35.75 |
| W | O | | | | | | | | | | |
| HETATM | | 2389 | O | HOH | W | 259 | 25.575 | 34.428 | −3.222 | 1.00 | 39.98 |
| W | O | | | | | | | | | | |
| HETATM | | 2390 | O | HOH | W | 260 | 14.941 | 30.863 | −2.385 | 1.00 | 26.04 |
| W | O | | | | | | | | | | |
| HETATM | | 2391 | O | HOH | W | 261 | 41.672 | 27.415 | 23.709 | 1.00 | 35.93 |
| W | O | | | | | | | | | | |
| HETATM | | 2392 | O | HOH | W | 262 | 18.136 | 30.604 | 21.993 | 1.00 | 31.10 |
| W | O | | | | | | | | | | |
| HETATM | | 2393 | O | HOH | W | 263 | 32.907 | 42.731 | 25.674 | 1.00 | 26.77 |
| W | O | | | | | | | | | | |
| HETATM | | 2394 | O | HOH | W | 264 | 21.328 | 33.022 | −5.079 | 1.00 | 32.93 |
| W | O | | | | | | | | | | |
| HETATM | | 2395 | O | HOH | W | 265 | 28.052 | 12.336 | 6.540 | 1.00 | 22.14 |
| W | O | | | | | | | | | | |
| HETATM | | 2396 | O | HOH | W | 266 | 2.099 | 19.008 | 5.021 | 1.00 | 32.55 |
| W | O | | | | | | | | | | |
| HETATM | | 2397 | O | HOH | W | 267 | 29.794 | 31.341 | 25.289 | 1.00 | 32.81 |
| W | O | | | | | | | | | | |
| HETATM | | 2398 | O | HOH | W | 268 | 20.757 | 45.357 | 22.862 | 1.00 | 36.33 |
| W | O | | | | | | | | | | |
| HETATM | | 2399 | O | HOH | W | 269 | 18.249 | 16.829 | −10.961 | 1.00 | 31.63 |
| W | O | | | | | | | | | | |
| HETATM | | 2400 | O | HOH | W | 270 | 5.371 | 13.454 | −3.003 | 1.00 | 38.53 |
| W | O | | | | | | | | | | |
| HETATM | | 2401 | O | HOH | W | 271 | 29.440 | 31.283 | 29.531 | 1.00 | 42.43 |
| W | O | | | | | | | | | | |
| HETATM | | 2402 | O | HOH | W | 272 | 28.073 | 49.822 | 16.597 | 1.00 | 34.43 |
| W | O | | | | | | | | | | |
| HETATM | | 2403 | O | HOH | W | 273 | 9.569 | 37.651 | 14.685 | 1.00 | 41.67 |
| W | O | | | | | | | | | | |
| HETATM | | 2404 | O | HOH | W | 274 | 3.582 | 23.223 | −3.241 | 1.00 | 41.20 |
| W | O | | | | | | | | | | |
| HETATM | | 2405 | O | HOH | W | 275 | 39.303 | 37.931 | 25.088 | 1.00 | 35.26 |
| W | O | | | | | | | | | | |
| HETATM | | 2406 | O | HOH | W | 276 | 27.169 | 49.230 | 14.079 | 1.00 | 27.21 |
| W | O | | | | | | | | | | |
| HETATM | | 2407 | O | HOH | W | 277 | 17.385 | 35.595 | −7.830 | 1.00 | 39.15 |
| W | O | | | | | | | | | | |
| HETATM | | 2408 | O | HOH | W | 278 | 36.093 | 19.433 | 19.171 | 1.00 | 57.03 |
| W | O | | | | | | | | | | |
| HETATM | | 2409 | O | HOH | W | 279 | 31.185 | 14.743 | 10.858 | 1.00 | 33.84 |
| W | O | | | | | | | | | | |
| HETATM | | 2410 | O | HOH | W | 280 | 16.886 | 31.999 | −1.178 | 1.00 | 30.74 |
| W | O | | | | | | | | | | |
| HETATM | | 2411 | O | HOH | W | 281 | 23.320 | 12.684 | 6.853 | 1.00 | 38.74 |
| W | O | | | | | | | | | | |
| HETATM | | 2412 | O | HOH | W | 282 | 35.109 | 41.222 | 24.391 | 1.00 | 35.21 |
| W | O | | | | | | | | | | |
| HETATM | | 2413 | O | HOH | W | 283 | 43.171 | 19.919 | 0.844 | 1.00 | 31.45 |
| W | O | | | | | | | | | | |
| HETATM | | 2414 | O | HOH | W | 284 | 21.356 | 14.695 | 23.557 | 1.00 | 28.47 |
| W | O | | | | | | | | | | |
| HETATM | | 2415 | O | HOH | W | 285 | 24.661 | 10.570 | 6.602 | 1.00 | 45.63 |
| W | O | | | | | | | | | | |
| HETATM | | 2416 | O | HOH | W | 286 | 4.247 | 12.050 | 1.832 | 1.00 | 39.46 |
| W | O | | | | | | | | | | |
| HETATM | | 2417 | O | HOH | W | 287 | 40.280 | 39.948 | 24.240 | 1.00 | 33.27 |
| W | O | | | | | | | | | | |
| HETATM | | 2418 | O | HOH | W | 288 | 20.284 | 56.897 | 0.611 | 1.00 | 44.29 |
| W | O | | | | | | | | | | |
| HETATM | | 2419 | O | HOH | W | 289 | 23.310 | 31.108 | 15.564 | 1.00 | 38.31 |
| W | O | | | | | | | | | | |
| HETATM | | 2420 | O | HOH | W | 290 | 48.053 | 26.077 | 15.288 | 1.00 | 47.19 |
| W | O | | | | | | | | | | |
| HETATM | | 2421 | O | HOH | W | 291 | 12.020 | 16.304 | 20.731 | 1.00 | 28.99 |
| W | O | | | | | | | | | | |
| HETATM | | 2422 | O | HOH | W | 292 | 41.231 | 23.934 | 2.570 | 1.00 | 33.94 |
| W | O | | | | | | | | | | |
| HETATM | | 2423 | O | HOH | W | 293 | 18.669 | 11.706 | −13.630 | 1.00 | 36.09 |
| W | O | | | | | | | | | | |
| HETATM | | 2424 | O | HOH | W | 294 | 8.109 | 34.730 | 7.563 | 1.00 | 39.26 |
| W | O | | | | | | | | | | |
| HETATM | | 2425 | O | HOH | W | 295 | 15.600 | 35.689 | −4.228 | 1.00 | 41.15 |
| W | O | | | | | | | | | | |
| HETATM | | 2426 | O | HOH | W | 296 | −4.142 | 24.573 | 20.605 | 1.00 | 42.87 |
| W | O | | | | | | | | | | |
| HETATM | | 2427 | O | HOH | W | 297 | 15.622 | 34.076 | 15.219 | 1.00 | 42.95 |
| W | O | | | | | | | | | | |
| HETATM | | 2428 | O | HOH | W | 298 | 38.169 | 40.792 | 23.402 | 1.00 | 45.01 |
| W | O | | | | | | | | | | |
| HETATM | | 2429 | O | HOH | W | 299 | 27.537 | 26.052 | −3.736 | 1.00 | 52.95 |
| W | O | | | | | | | | | | |
| HETATM | | 2430 | O | HOH | W | 300 | 35.494 | 16.860 | 10.304 | 1.00 | 43.09 |
| W | O | | | | | | | | | | |
| HETATM | | 2431 | O | HOH | W | 301 | 41.551 | 21.663 | 1.380 | 1.00 | 36.94 |
| W | O | | | | | | | | | | |
| HETATM | | 2432 | O | HOH | W | 302 | 45.225 | 29.031 | 6.035 | 1.00 | 40.03 |
| W | O | | | | | | | | | | |
| HETATM | | 2433 | O | HOH | W | 303 | 31.772 | 47.636 | 4.279 | 1.00 | 35.07 |
| W | O | | | | | | | | | | |
| HETATM | | 2434 | O | HOH | W | 304 | 16.888 | 20.002 | −11.152 | 1.00 | 39.19 |
| W | O | | | | | | | | | | |
| HETATM | | 2435 | O | HOH | W | 305 | 15.036 | 31.029 | 17.354 | 1.00 | 42.31 |
| W | O | | | | | | | | | | |
| HETATM | | 2436 | O | HOH | W | 306 | 8.035 | 38.484 | 2.278 | 1.00 | 36.39 |
| W | O | | | | | | | | | | |
| HETATM | | 2437 | O | HOH | W | 307 | 7.021 | 9.870 | 24.009 | 1.00 | 46.58 |
| W | O | | | | | | | | | | |
| HETATM | | 2438 | O | HOH | W | 308 | −0.328 | 27.092 | −6.529 | 1.00 | 39.32 |
| W | O | | | | | | | | | | |
| HETATM | | 2439 | O | HOH | W | 309 | 32.290 | 44.882 | −6.224 | 1.00 | 41.91 |
| W | O | | | | | | | | | | |
| HETATM | | 2440 | O | HOH | W | 310 | −2.886 | 27.829 | 1.400 | 1.00 | 46.46 |
| W | O | | | | | | | | | | |
| HETATM | | 2441 | O | HOH | W | 311 | 45.080 | 21.111 | 1.259 | 1.00 | 43.31 |
| W | O | | | | | | | | | | |
| HETATM | | 2442 | O | HOH | W | 312 | 34.187 | 18.021 | 11.951 | 1.00 | 33.37 |
| W | O | | | | | | | | | | |
| HETATM | | 2443 | O | HOH | W | 313 | 42.013 | 42.835 | 15.912 | 1.00 | 35.29 |
| W | O | | | | | | | | | | |
| HETATM | | 2444 | O | HOH | W | 314 | 21.506 | 9.309 | 4.752 | 1.00 | 35.56 |
| W | O | | | | | | | | | | |
| HETATM | | 2445 | O | HOH | W | 315 | 33.745 | 38.255 | 30.145 | 1.00 | 42.37 |
| W | O | | | | | | | | | | |
| HETATM | | 2446 | O | HOH | W | 316 | 26.849 | 28.079 | −7.783 | 1.00 | 42.24 |
| W | O | | | | | | | | | | |
| HETATM | | 2447 | O | HOH | W | 317 | 22.190 | 18.955 | −5.896 | 1.00 | 44.87 |
| W | O | | | | | | | | | | |
| HETATM | | 2448 | O | HOH | W | 318 | 5.056 | 11.251 | −3.217 | 1.00 | 43.78 |
| W | O | | | | | | | | | | |
| HETATM | | 2449 | O | HOH | W | 319 | 30.465 | 47.010 | −8.187 | 1.00 | 60.12 |
| W | O | | | | | | | | | | |
| HETATM | | 2450 | O | HOH | W | 320 | 25.131 | 34.069 | −7.705 | 1.00 | 40.30 |
| W | O | | | | | | | | | | |
| HETATM | | 2451 | O | HOH | W | 321 | −1.120 | 23.587 | 2.285 | 1.00 | 57.54 |
| W | O | | | | | | | | | | |
| HETATM | | 2452 | O | HOH | W | 322 | 27.796 | 32.147 | 25.994 | 1.00 | 52.28 |
| W | O | | | | | | | | | | |
| HETATM | | 2453 | O | HOH | W | 323 | 2.796 | 19.619 | −4.238 | 1.00 | 43.84 |
| W | O | | | | | | | | | | |
| HETATM | | 2454 | O | HOH | W | 324 | 4.253 | 33.074 | 8.597 | 1.00 | 38.14 |
| W | O | | | | | | | | | | |
| HETATM | | 2455 | O | HOH | W | 325 | 28.162 | 22.354 | 26.470 | 1.00 | 46.35 |
| W | O | | | | | | | | | | |
| HETATM | | 2456 | O | HOH | W | 326 | 17.658 | 41.562 | −4.831 | 1.00 | 45.26 |
| W | O | | | | | | | | | | |
| HETATM | | 2457 | O | HOH | W | 327 | 12.525 | 24.278 | 23.101 | 1.00 | 49.33 |
| W | O | | | | | | | | | | |
| HETATM | | 2458 | O | HOH | W | 328 | 1.417 | 20.936 | 21.185 | 1.00 | 51.98 |
| W | O | | | | | | | | | | |
| HETATM | | 2459 | O | HOH | W | 329 | 5.110 | 34.325 | 4.685 | 1.00 | 47.55 |
| W | O | | | | | | | | | | |
| HETATM | | 2460 | O | HOH | W | 330 | 23.854 | 24.173 | −8.875 | 1.00 | 53.09 |
| W | O | | | | | | | | | | |
| HETATM | | 2461 | O | HOH | W | 331 | 26.863 | 30.894 | 27.666 | 1.00 | 47.65 |
| W | O | | | | | | | | | | |
| HETATM | | 2462 | O | HOH | W | 332 | 32.534 | 14.831 | 7.143 | 1.00 | 39.84 |
| W | O | | | | | | | | | | |
| HETATM | | 2463 | O | HOH | W | 333 | 15.358 | 12.945 | −2.666 | 1.00 | 37.81 |
| W | O | | | | | | | | | | |
| HETATM | | 2464 | O | HOH | W | 334 | 44.680 | 33.725 | 22.138 | 1.00 | 49.77 |
| W | O | | | | | | | | | | |
| HETATM | | 2465 | O | HOH | W | 335 | 12.020 | 38.120 | 13.522 | 1.00 | 58.79 |
| W | O | | | | | | | | | | |
| HETATM | | 2466 | O | HOH | W | 336 | 42.127 | 30.661 | −3.910 | 1.00 | 44.39 |
| W | O | | | | | | | | | | |
| HETATM | | 2467 | O | HOH | W | 337 | 1.727 | 20.705 | 6.878 | 1.00 | 45.75 |
| W | O | | | | | | | | | | |
| HETATM | | 2468 | O | HOH | W | 338 | −0.384 | 35.128 | −7.818 | 1.00 | 49.01 |
| W | O | | | | | | | | | | |
| HETATM | | 2469 | O | HOH | W | 339 | 21.057 | 43.781 | 28.030 | 1.00 | 40.90 |
| W | O | | | | | | | | | | |
| HETATM | | 2470 | O | HOH | W | 340 | 11.861 | 9.923 | 3.834 | 1.00 | 41.37 |
| W | O | | | | | | | | | | |
| HETATM | | 2471 | O | HOH | W | 341 | 37.937 | 37.279 | 28.479 | 1.00 | 59.04 |
| W | O | | | | | | | | | | |
| HETATM | | 2472 | O | HOH | W | 342 | 6.363 | 12.922 | 12.257 | 1.00 | 43.48 |
| W | O | | | | | | | | | | |
| HETATM | | 2473 | O | HOH | W | 343 | 2.799 | 32.430 | 24.168 | 1.00 | 60.02 |
| W | O | | | | | | | | | | |
| HETATM | | 2474 | O | HOH | W | 344 | 29.796 | 50.785 | 1.614 | 1.00 | 53.08 |
| W | O | | | | | | | | | | |
| HETATM | | 2475 | O | HOH | W | 345 | −0.874 | 32.698 | −7.319 | 1.00 | 39.33 |
| W | O | | | | | | | | | | |
| HETATM | | 2476 | O | HOH | W | 346 | 36.654 | 46.102 | −3.751 | 1.00 | 49.64 |
| W | O | | | | | | | | | | |
| HETATM | | 2477 | O | HOH | W | 347 | 22.253 | 20.580 | 21.775 | 1.00 | 38.38 |
| W | O | | | | | | | | | | |
| HETATM | | 2478 | O | HOH | W | 348 | 23.536 | 39.353 | 28.221 | 1.00 | 50.03 |
| W | O | | | | | | | | | | |
| HETATM | | 2479 | O | HOH | W | 349 | 21.979 | 23.483 | 27.337 | 1.00 | 56.78 |
| W | O | | | | | | | | | | |
| HETATM | | 2480 | O | HOH | W | 350 | 15.688 | 9.390 | 6.304 | 1.00 | 39.68 |
| W | O | | | | | | | | | | |
| HETATM | | 2481 | O | HOH | W | 351 | 17.446 | 31.911 | 24.017 | 1.00 | 49.08 |
| W | O | | | | | | | | | | |
| HETATM | | 2482 | O | HOH | W | 352 | 20.424 | 32.203 | −1.926 | 1.00 | 52.71 |
| W | O | | | | | | | | | | |
| HETATM | | 2483 | O | HOH | W | 353 | 7.374 | 38.066 | 19.040 | 1.00 | 52.43 |
| W | O | | | | | | | | | | |
| HETATM | | 2484 | O | HOH | W | 354 | 40.616 | 34.974 | 29.409 | 1.00 | 43.05 |
| W | O | | | | | | | | | | |
| HETATM | | 2485 | O | HOH | W | 355 | 13.964 | 28.600 | 20.763 | 1.00 | 45.36 |
| W | O | | | | | | | | | | |
| HETATM | | 2486 | O | HOH | W | 356 | 21.968 | 11.935 | 4.239 | 1.00 | 34.43 |
| W | O | | | | | | | | | | |
| HETATM | | 2487 | O | HOH | W | 357 | 40.741 | 40.054 | −5.266 | 1.00 | 50.01 |
| W | O | | | | | | | | | | |
| HETATM | | 2488 | O | HOH | W | 358 | 1.011 | 22.226 | 10.873 | 1.00 | 47.98 |
| W | O | | | | | | | | | | |
| HETATM | | 2489 | O | HOH | W | 359 | 30.521 | 24.949 | −3.484 | 1.00 | 51.24 |
| W | O | | | | | | | | | | |
| HETATM | | 2490 | O | HOH | W | 360 | 30.394 | 22.663 | 13.867 | 1.00 | 35.80 |
| W | O | | | | | | | | | | |
| HETATM | | 2491 | O | HOH | W | 361 | 1.298 | 28.757 | 23.599 | 1.00 | 45.73 |
| W | O | | | | | | | | | | |
| HETATM | | 2492 | O | HOH | W | 362 | 10.037 | 16.429 | −9.605 | 1.00 | 43.70 |
| W | O | | | | | | | | | | |
| HETATM | | 2493 | O | HOH | W | 363 | 2.342 | 16.995 | 19.296 | 1.00 | 53.29 |
| W | O | | | | | | | | | | |
| HETATM | | 2494 | O | HOH | W | 364 | 18.754 | 21.800 | −12.588 | 1.00 | 56.18 |
| W | O | | | | | | | | | | |
| HETATM | | 2495 | O | HOH | W | 365 | 18.388 | 23.021 | 28.255 | 1.00 | 48.80 |
| W | O | | | | | | | | | | |
| HETATM | | 2496 | O | HOH | W | 366 | 12.812 | 40.928 | 2.593 | 1.00 | 50.81 |
| W | O | | | | | | | | | | |
| HETATM | | 2497 | O | HOH | W | 367 | 20.573 | 19.834 | −9.838 | 1.00 | 49.46 |
| W | O | | | | | | | | | | |
| HETATM | | 2498 | O | HOH | W | 368 | 3.924 | 11.192 | 4.654 | 1.00 | 47.96 |
| W | O | | | | | | | | | | |
| HETATM | | 2499 | O | HOH | W | 369 | 23.969 | 20.116 | −9.041 | 1.00 | 44.24 |
| W | O | | | | | | | | | | |
| HETATM | | 2500 | O | HOH | W | 370 | 40.480 | 32.964 | 1.998 | 1.00 | 52.22 |
| W | O | | | | | | | | | | |
| HETATM | | 2501 | O | HOH | W | 371 | 20.730 | 46.882 | 18.743 | 1.00 | 38.87 |
| W | O | | | | | | | | | | |
| HETATM | | 2502 | O | HOH | W | 372 | 29.891 | 46.902 | 11.620 | 1.00 | 41.85 |
| W | O | | | | | | | | | | |
| HETATM | | 2503 | O | HOH | W | 373 | 27.107 | 45.779 | −9.724 | 1.00 | 49.81 |
| W | O | | | | | | | | | | |
| HETATM | | 2504 | O | HOH | W | 374 | 45.521 | 35.688 | 5.405 | 1.00 | 50.64 |
| W | O | | | | | | | | | | |
| HETATM | | 2505 | O | HOH | W | 375 | 8.631 | 12.370 | 10.753 | 1.00 | 41.87 |
| W | O | | | | | | | | | | |
| HETATM | | 2506 | O | HOH | W | 376 | −1.414 | 24.184 | 8.066 | 1.00 | 45.18 |
| W | O | | | | | | | | | | |
| HETATM | | 2507 | O | HOH | W | 377 | 25.847 | 26.771 | −9.623 | 1.00 | 50.58 |
| W | O | | | | | | | | | | |
| HETATM | | 2508 | O | HOH | W | 378 | 17.044 | 35.686 | 20.995 | 1.00 | 46.08 |
| W | O | | | | | | | | | | |
| HETATM | | 2509 | O | HOH | W | 379 | 31.859 | 30.020 | 27.009 | 1.00 | 44.41 |
| W | O | | | | | | | | | | |
| HETATM | | 2510 | O | HOH | W | 380 | 15.331 | 35.172 | 17.686 | 1.00 | 42.80 |
| W | O | | | | | | | | | | |
| HETATM | | 2511 | O | HOH | W | 381 | 30.163 | 27.844 | −12.299 | 1.00 | 48.94 |
| W | O | | | | | | | | | | |
| HETATM | | 2512 | O | HOH | W | 382 | 26.459 | 20.674 | 25.974 | 1.00 | 50.90 |
| W | O | | | | | | | | | | |
| HETATM | | 2513 | O | HOH | W | 383 | 11.215 | 40.705 | −7.869 | 1.00 | 44.77 |
| W | O | | | | | | | | | | |
| HETATM | | 2514 | O | HOH | W | 384 | 39.653 | 28.993 | 25.532 | 1.00 | 42.77 |
| W | O | | | | | | | | | | |
| HETATM | | 2515 | O | HOH | W | 385 | 8.320 | 22.025 | 23.135 | 1.00 | 40.98 |
| W | O | | | | | | | | | | |
| HETATM | | 2516 | O | HOH | W | 386 | 19.109 | 57.899 | 2.281 | 1.00 | 52.58 |
| W | O | | | | | | | | | | |
| HETATM | | 2517 | O | HOH | W | 387 | 23.211 | 31.034 | 3.809 | 0.68 | 12.76 |
| W | O | | | | | | | | | | |
| HETATM | | 2518 | O | HOH | W | 388 | 23.669 | 33.864 | 2.886 | 0.68 | 17.24 |
| W | O | | | | | | | | | | |
| HETATM | | 2519 | O | HOH | W | 389 | 22.013 | 32.823 | 3.876 | 0.68 | 32.76 |
| W | O | | | | | | | | | | |

END
¹Amino acid residues correspond to residues in human IL-22, SEQ ID NO: 2.

TABLE 5
Solvent exposed residues of hIL-22.¹

| | Solvent Exposed | | Solvent Exposed |
|---|---|---|---|
| Residue | Area (Å²)² | Residue | Area (Å²) |
| SER38 | 80.00 | HIS39 | 131.00 |
| ARG41 | 112.00 | ASP43 | 91.00 |
| LYS44 | 82.00 | SER45 | 49.00 |
| ASN46 | 46.00 | GLN48 | 103.00 |
| GLN49 | 91.00 | PRO50 | 99.00 |
| TYR51 | 124.00 | ILE52 | 24.00 |
| THR53 | 27.00 | ASN54 | 81.00 |
| ARG55 | 51.00 | PHE57 | 69.00 |
| MET58 | 51.00 | LYS61 | 123.00 |
| GLU62 | 70.00 | SER64 | 42.00 |
| LEU65 | 135.00 | ALA66 | 53.00 |
| ASP67 | 27.00 | ASN68 | 141.00 |
| ASN69 | 35.00 | THR70 | 126.00 |
| ASP71 | 142.00 | VAL72 | 45.00 |
| ARG73 | 141.00 | LEU74 | 11.00 |
| ILE75 | 18.00 | GLY76 | 13.00 |
| GLU77 | 159.00 | LYS78 | 116.00 |
| PHE80 | 39.00 | HIS81 | 141.00 |
| GLY82 | 60.00 | SER84 | 49.00 |
| MET85 | 131.00 | SER86 | 103.00 |
| GLU87 | 22.00 | ARG88 | 53.00 |
| TYR90 | 31.00 | LYS93 | 20.00 |
| GLN94 | 49.00 | ASN97 | 40.00 |
| PHE98 | 10.00 | GLU101 | 113.00 |
| GLU102 | 83.00 | PHE105 | 86.00 |
| PRO106 | 72.00 | SER108 | 46.00 |
| ASP109 | 113.00 | ARG110 | 96.00 |
| PHE111 | 23.00 | GLN112 | 136.00 |
| PRO113 | 78.00 | TYR114 | 54.00 |
| GLN116 | 79.00 | GLU117 | 60.00 |
| VAL119 | 16.00 | PRO120 | 58.00 |
| PHE121 | 13.00 | ALA123 | 33.00 |
| ARG124 | 157.00 | LEU125 | 3.00 |
| SER126 | 20.00 | ASN127 | 114.00 |
| ARG128 | 126.00 | SER130 | 54.00 |
| THR131 | 117.00 | HIS133 | 105.00 |
| ILE134 | 30.00 | GLU135 | 195.00 |
| GLY136 | 39.00 | ASP137 | 102.00 |
| ASP138 | 39.00 | LEU139 | 73.00 |
| HIS140 | 48.00 | GLN142 | 115.00 |
| ARG143 | 155.00 | ASN144 | 27.00 |
| GLN146 | 80.00 | LYS147 | 109.00 |
| LYS149 | 53.00 | ASP150 | 53.00 |
| THR151 | 21.00 | LYS153 | 127.00 |
| LYS154 | 147.00 | LEU155 | 37.00 |
| GLY156 | 29.00 | GLU157 | 104.00 |
| SER158 | 32.00 | GLU160 | 35.00 |
| ILE161 | 11.00 | LYS162 | 6.00 |
| ALA163 | 11.00 | GLY165 | 11.00 |
| GLU166 | 15.00 | ASP168 | 40.00 |
| LEU169 | 38.00 | MET172 | 86.00 |
| SER173 | 15.00 | ARG175 | 70.00 |
| ASN176 | 112.00 | ALA177 | 21.00 |
| ILE179 | 89.00 | | |

Total area of chain A: 7584.
¹Amino acid residues correspond to residues in human IL-22, SEQ ID NO: 2.
²Solvent exposed areas c

We claim:

1. A method for identifying a mutant mammalian IL-22 with modified ability to dimerize and/or bind an IL-22 receptor, said method comprising the steps of:
a) constructing a three-dimensional structure of hIL-22 defined by the atomic coordinates shown in Table 4;
b) employing the three-dimensional structure and modeling methods to identify an amino acid involved in stabilizing an IL-22 dimer, and/or to identify an amino acid involved in receptor binding;
c) producing a mammalian IL-22 having a mutation at an amino acid identified in (b); and
d) assaying said mutant IL-22 to determine the ability of said mutant to dimerize as compared to an IL-22 control, wherein a difference in dimerization between said mutant and said control is indicative of a modified ability to dimerize, and/or assaying said mutant IL-22 to determine the ability of said mutant to bind to the IL-22 receptor as compared to an IL-22 control, wherein a difference in binding between said mutant and said IL-22 control is indicative of a modified ability to bind the IL-22 receptor.

2. The method of claim 1, wherein said mutation site is at a dimerization interface and/or an IL-22-receptor-binding site.

3. The method of claim 2, wherein the dimerization interface is comprised of amino acids at positions corresponding to position 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 166, 167, 168, 169, 170, 171, 172, 173, 174, 175, 176, 177, 178, or 179 of SEQ ID NO: 2, and/or wherein the IL-22-receptor binding site is Region 1 or Region 2.

4. The method of claim 2, wherein the mutant IL-22 comprises a mutation at one or more positions corresponding to position 44, 48, 49, 57, 61, 64, 73, 75, 83, 166, 168, 175, 176, or 179 of SEQ ID NO: 2, and/or wherein the mutant IL-22 comprises a mutation at one or more positions corresponding to position 61, 70, 71, 162, 166, or 169 of SEQ ID NO: 2, and/or wherein the hIL-22 mutant comprises at least a mutation at one or more positions corresponding to position 98, 99, 100, 101, 102, 103, 104, 154, 155, 156,or 157 of SEQ ID NO: 2.

5. The method of claim 1, wherein the mutant IL-22 is human.

6. A mutant IL-22 comprising at least one amino acid substitution in Region 1 or Region 2.

7. The mutant IL-22 of claim 6, wherein the mutant IL-22 comprises a mutation at one or more positions corresponding to position 61, 70, 71, 162, 166, or 169 of SEQ ID NO: 2.

8. The mutant IL-22 of claim 6, wherein the mutant IL-22 comprises a mutation at one or more position corresponding to position 98, 99, 100, 101, 102, 103, 104, 154, 155, 156, or 157 of SEQ ID NO: 2.

9. The mutant IL-22 of claim 6 comprising Region 1, wherein the mutant IL-22 comprises a mutation at one or more positions corresponding to position 61, 70, 71, 162, 166, or 169 of SEQ ID NO: 2.

10. The mutant IL-22 of claim 6 comprising Region 2, wherein the mutant IL-22 comprises a mutation at one or more positions corresponding to position 98, 99, 100, 101, 102, 103, 104, 154, 155, 156, or 157 of SEQ ID NO: 2.

11. A mutant IL-22 comprising at least one mutation at an IL-22 dimerization interface.

12. The mutant IL-22 of claim 11, wherein the dimerization interface is comprised of amino acids at positions corresponding to position 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 166, 167, 168, 169, 170, 171, 172, 173, 174, 175, 176, 177, 178, or 179 of SEQ ID NO: 2.

13. The mutant IL-22 of claim 12, wherein the mutant IL-22 comprises a mutation at one or more position corresponding to position 44, 48, 49, 57, 61, 64, 73, 75, 83, 166, 168, 175, 176 or 179 of SEQ ID NO: 2.

14. The mutant IL-22 of claim 11, wherein the mutant IL-22 comprises a mutation at one or more positions in the dimerization interface, wherein that position is involved in stabilizing a dimmer of IL-22.

15. The mutant IL-22 of claim 14, wherein the mutation is selected from one or more of the group consisting of:
a) an amino acid at a position corresponding to position 175 or 166 of SEQ ID NO: 2;
b) an amino acid at a position corresponding to position 57 or 176 of SEQ ID NO: 2;
c) an amino acid at a position corresponding to position 73 or 83 of SEQ ID NO: 2;
d) an amino acid at a position corresponding to position 44 or 64 of SEQ ID NO: 2;
e) an amino acid at a position corresponding to position 175 or 168 of SEQ ID NO: 2;
f) an amino acid at a position corresponding to position 176 or 75 of SEQ ID NO: 2;
g) an amino acid at a position corresponding to position 48 or 61 of SEQ ID NO: 2;
h) an amino acid at a position corresponding to position 44 or 166 of SEQ ID NO: 2;
i) an amino acid at a position corresponding to position 61 or 179 of SEQ ID NO: 2; and
j) an amino acid at a position corresponding to position 49 or 61 of SEQ ID NO: 2.

16. The mutant hlL-22 of claim 15, wherein the amino acid at a position corresponding to position 175 of SEQ ID NO: 2 is any amino acid except arginine and lysine.

17. The mutant IL-22 of claim 15, wherein the amino acid at a position corresponding to position 166 of SEQ ID NO: 2 is any amino acid except glutamate, aspartate, glutamine, asparagine, serine, threonine and cysteine.

18. The mutant IL-22 of claim 15, wherein the amino acid at the position corresponding to position 176 of SEQ ID NO: 2 is any amino acid except arginine, lysine, asparagine and glutamine.

19. The mutant IL-22 of claim 15, wherein the amino acid at the position corresponding to position 73 of SEQ ID NO: 2 is any amino acid except arginine and lysine.

20. The mutant IL-22 of claim 15, wherein the amino acid at the position corresponding to position 44 of SEQ ID NO: 2 is any amino acid except arginine and lysine.

21. The mutant IL-22 of claim 15, wherein the amino acid at a position corresponding to position 64 of SEQ ID NO: 2 is any amino acid except glutamate, aspartate, glutamine, asparagine, serine, threonine and cysteine.

22. The mutant IL-22 of claim 15, wherein the amino acid at a position corresponding to position 168 of SEQ ID NO: 2 is any amino acid except glutamate, aspartate, glutamine, asparagine, serine, threonine and cysteine.

23. The mutant IL-22 of claim 15, wherein the amino acid at the position corresponding to position 61 of SEQ ID NO: 2 is any amino acid except arginine and lysine.

24. The mutant IL-22 of claim 15, wherein the amino acid at the position corresponding to position 49 of SEQ ID NO: 2 is any amino acid except glutamine, asparagine, glutamate and aspartate.
